SEQUENCE LISTING (206 sequences)

SEQ ID NO: 1 - DNA, 2199 bp, Artificial Sequence
  Avi-His-Human PSMA ECD
catcatcaccatcatcatcatcatcaccatggtctgaatgacatcttcgaggctcagaaaatcgaatggcacgaacataaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaactcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcc

SEQ ID NO: 2 - protein, 733 aa, Artificial Sequence
  Avi-His-Human PSMA ECD
HHHHHHHHHHGLNDIFEAQKIEWHEHKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 3 - DNA, 2250 bp, Artificial Sequence
  Human full length PSMA
atgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaactcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcc

SEQ ID NO: 4 - protein, 750 aa, Artificial Sequence
  Human full length PSMA
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 5 - DNA, 2250 bp, Artificial Sequence
  Cyno Full length PSMA
atgtggaatctcctgcacgaaaccgactcggctgtggccaccgcgcgccgcccgcgctggctgtgcgctggggcactggtgctggcgggtggcttctttctcctcggcttcctcttcggatggtttataaaatcctccagtgaagctactaacattactccaaagcataatatgaaagcatttttggatgaactgaaagctgagaacatcaagaagttcttacataattttacacagataccacatttagcaggaacagaacaaaactttcaacttgcaaagcaaattcaatcccagtggaaagaatttggcctggattctgttgagctaactcattatgatgtcctgttgtcctacccaaataagactcatcccaactacatctcaataattaatgaagatggaaatgagattttcaacacatcattatttgaaccacctcctgcaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccacaggagtcattctctactcagaccctgctgactactttgctcctggggtaaagtcttatccagatggttggaatcttcctggaggtggtgtccagcgtggaaatatcctaaatctgaatggtgcaggagaccctctcacaccaggttacccagcaaatgaatatgcttataggcgtggaatggcagaggctgttggtcttccaagtattcccgttcatccaattgggtactatgatgcacagaagctcctagaaaaaatgggtggctcagcatcaccagatagcagctggagaggaagtctcaaagtgccctacaatgttggacctggctttactggaaacttttctacacaaaaagtcaagatgcacatccactctaccagtgaagtgacaagaatttacaatgtgataggtactctcagaggagcagtggaaccagacagatacgtcattctgggaggtcaccgggactcatgggtgtttggtggtattgaccctcagagtggagcagctgttgttcatgaaattgtgaggagctttggaacgctgaaaaaggaagggtggagacctagaagaacaattttgtttgcaagctgggatgcagaagaatttggtcttcttggttctactgaatgggcagaggagaactcaagactccttcaagagcgtggcgtggcttatattaatgctgattcgtctatagaaggaaactacactctgagagttgattgtacaccactgatgtacagcttggtatacaacctaacaaaagagctggaaagccctgatgaaggctttgaaggcaaatctctttatgaaagttggactaaaaaaagtccttcccccgagttcagtggcatgcccaggataagcaaattgggatctggaaatgattttgaggtgttcttccaacgacttggaattgcctcaggcagagcacggtatactaaaaattgggaaacaaacaaattcagcagctatccactgtatcacagtgtctatgagacatatgagttggtggaaaagttttatgatccaatgtttaaatatcacctcactgtggcccaggttcgaggagggatggtgtttgaactagccaattccgtagtgctcccttttgattgtcgagattatgctgtagttttaagaaagtatgctgacaaaatctacaatatttctatgaaacatccacaggaaatgaagacatacagtgtatcatttgattcacttttttctgcagtaaagaattttacagaaattgcttccaagttcagtgagagactccgggactttgacaaaagcaacccaatattattaagaatgatgaatgatcaactcatgtttctggaaagagcatttattgatccattagggttaccagacagacctttttataggcatgtcatctatgctccaagcagccacaacaagtatgcaggggagtcattcccaggaatttatgatgctctgtttgatatcgaaagcaaagtggacccttcccaggcctggggagaagtgaagagacagatttctattgcaaccttcacagtgcaagcagctgcagagactttgagtgaagtggcc

SEQ ID NO: 6 - protein, 750 aa, Artificial Sequence
  Cyno Full length PSMA
MWNLLHETDSAVATARRPRWLCAGALVLAGGFFLLGFLFGWFIKSSSEATNITPKHNMKAFLDELKAENIKKFLHNFTQIPHLAGTEQNFQLAKQIQSQWKEFGLDSVELTHYDVLLSYPNKTHPNYISIINEDGNEIFNTSLFEPPPAGYENVSDIVPPFSAFSPQGMPEGDLVYVNYARTEDFFKLERDMKINCSGKIVIARYGKVFRGNKVKNAQLAGATGVILYSDPADYFAPGVKSYPDGWNLPGGGVQRGNILNLNGAGDPLTPGYPANEYAYRRGMAEAVGLPSIPVHPIGYYDAQKLLEKMGGSASPDSSWRGSLKVPYNVGPGFTGNFSTQKVKMHIHSTSEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVYNLTKELESPDEGFEGKSLYESWTKKSPSPEFSGMPRISKLGSGNDFEVFFQRLGIASGRARYTKNWETNKFSSYPLYHSVYETYELVEKFYDPMFKYHLTVAQVRGGMVFELANSVVLPFDCRDYAVVLRKYADKIYNISMKHPQEMKTYSVSFDSLFSAVKNFTEIASKFSERLRDFDKSNPILLRMMNDQLMFLERAFIDPLGLPDRPFYRHVIYAPSSHNKYAGESFPGIYDALFDIESKVDPSQAWGEVKRQISIATFTVQAAAETLSEVA

SEQ ID NO: 7 - DNA, 2226 bp, Artificial Sequence
  TSC266 Anti PSMA scFv x Anti CD3 scFv
gatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtcagaggcacaacaattcttccctgaatacaggaactcagatggcaggtcattctccgaattctcaggtccagctggtggagtctgggggcggagtggtgcagcctgggcggtcactgaggctgtcctgcaaggcttctggctacacctttactagatctacgatgcactgggtaaggcaggcccctggacaaggtctggaatggattggatacattaatcctagcagtgcttatactaattacaatcagaaattcaaggacaggttcacaatcagcgcagacaaatccaagagcacagccttcctgcagatggacagcctgaggcccgaggacaccggcgtctatttctgtgcacggccccaagtccactatgattacaacgggtttccttactggggccaagggactcccgtcactgtctctagcggtggcggagggtctgggggtggcggatccggaggtggtggctctgcacaagacatccagatgacccagtctccaagcagcctgtctgcaagcgtgggggacagggtcaccatgacctgcagtgccagctcaagtgtaagttacatgaactggtaccagcagaagccgggcaaggcccccaaaagatggatttatgactcatccaaactggcttctggagtccctgctcgcttcagtggcagtgggtctgggaccgactataccctcacaatcagcagcctgcagcccgaagatttcgccacttattactgccagcagtggagtcgtaacccacccacgttcggaggggggaccaagctacaaattacatcctccagc

SEQ ID NO: 8 - protein, 742 aa, Artificial Sequence
  TSC266 Anti PSMA scFv x Anti CD3 scFv
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGQRHNNSSLNTGTQMAGHSPNSQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSS

SEQ ID NO: 9 - DNA, 1524 bp, Artificial Sequence
  TSC291 Anti PSMA scFv-linker Anti CD3 scFv- His
caggtgcagctggtcgagtctggcggcggactggtcaagcctggcgagtccctgagactgtcttgcgctgcctccggcttcaccttctccgactactacatgtactgggtccgccaggctcctggcaagggactggaatgggtggccatcatctccgacggcggctactacacctactactccgacatcatcaagggccggttcaccatctccagggacaacgccaagaactccctgtacctgcagatgaactccctgaaggccgaggacaccgccgtgtactactgcgccaggggcttcccactgctgagacacggcgccatggattactggggccagggcaccctggtcacagtgtcctctggcggaggcggaagtggaggcggaggaagcggaggcggcggatccgacatccagatgacccagtccccatcctccctgtctgcctccgtgggcgacagagtgaccatcacatgcaaggcctcccagaacgtggacaccaacgtggcatggtatcagcagaagccaggccaggcccctaagtccctgatctactctgcctcctaccggtactccgacgtgccctccaggttctctggctccgcctctggcaccgacttcaccctgaccatctcttccgtgcagtccgaggacttcgctacctactactgccagcagtacgactcctacccttacaccttcggcggaggcaccaagctggaaatcaagtccggcggagggggctctgaagtgcagctggtggaaagcggagggggactggtgcagcccgggggaagtctgaagctgtcctgtgccgccagcggctttaccttcaacaagtacgccatgaattgggtccgacaggccccagggaaaggcctggaatgggtggcacggatccggtccaagtacaacaactacgccacctactacgctgactccgtgaaggacagattcaccatcagccgggacgactctaagaacaccgcctatctgcagatgaacaacctgaaaaccgaggatacagctgtgtactattgtgtgcggcacggcaacttcggcaactcctacatctcctactgggcctattggggacagggaacactggtcaccgtgtctagcggaggtggcggaagtgggggaggcggatctggcggtggcggatcccagaccgtggtcacccaggaaccttccctgaccgtctccccaggcggcaccgtgaccctgacctgtggctcctctaccggcgctgtgacctccggcaactaccctaactgggtgcagcagaaacccggacaggctcctagaggcctgatcggcggcaccaagtttctggcccctggcacccctgccagattctccggctccctgctgggaggcaaggccgctctgaccctgtctggcgtgcagcctgaggacgaggccgagtactactgtgtgctgtggtactccaacagatgggtgttcggaggcggcacaaagctgaccgtgctgcatcatcatcatcatcaccatcatcatcac

SEQ ID NO: 10 - protein, 508 aa, Artificial Sequence
  TSC291 Anti PSMA scFv-linker Anti CD3 scFv- His
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHHHHHH

SEQ ID NO: 11 - DNA, 2271 bp, Artificial Sequence
  TRI130 Anti TA scFv x Anti CD3 scFv
gacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaaggtggaggcggttcaggcggaggtggatccggcggtggcggctccggtggcggcggatctgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtca

SEQ ID NO: 12 - protein, 757 aa, Artificial Sequence
  TRI130 Anti TA scFv x Anti CD3 scFv
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 13 - DNA, 2277 bp, Artificial Sequence
  TRI149 Anti TA scFv x Anti CD3 scFv
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcttcaggtggaggcggttcaggcggaggtggatccggcggtggcggctccggtggcggcggatctgacatcgtgatgacccagtctccagactccctggctgtgtctctgggcgagagggccaccatcaactgcaagtccagccaaagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtca

SEQ ID NO: 14 - protein, 759 aa, Artificial Sequence
  TRI149 Anti TA scFv x Anti CD3 scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEKLRYFDWLSDAFDIWGQGTMVTVSSGGGGSGGGGSGGGGSGGGGSDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPTTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 15 - DNA, 2232 bp, Artificial Sequence
  TRI 01043
gacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaaggtggaggcggttcaggcggaggtggatccggcggtggcggctccggtggcggcggatctgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcctcgagtgagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcggcgggggatccccgtcacaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacaatcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggtggtggaggtagtggtggaggcggatctggcggcggcggttcaggaggtggtggatccgatgtccagcttacccaaagtccgagcacgttgagtgcaagtgtaggagaccgcgtaacgattacttgctctgcttcaagttccgtatcctacatgaattggtatcagcaaaagcctggaaaagcccctaagcgctggatatacgattcaagtaagttggcttctggcgtcccagcacggttttctggttcaggttccggtacagaatatacgctgacaatcagctctctccaaccggatgatttcgcaacctattactgtcaacaatggtcaagaaatccgccgacattcgggcagggaacaaaagtcgaggtaaaaaggtca

SEQ ID NO: 16 - protein, 744 aa, Artificial Sequence
  TRI 01043
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSQVQLVQSGAEVKKPGASVKVSCKASGYTFTRSTMHWVRQAPGQGLEWIGYINPSSAYTNYNQKFQGRVTLTADKSTSTAYMELSSLRSEDTAVYYCASPQVHYDYNGFPYWGQGTLVTVSSGGGGSGGGGSGGGGSGGGGSDVQLTQSPSTLSASVGDRVTITCSASSSVSYMNWYQQKPGKAPKRWIYDSSKLASGVPARFSGSGSGTEYTLTISSLQPDDFATYYCQQWSRNPPTFGQGTKVEVKRS

SEQ ID NO: 17 - DNA, 2232 bp, Artificial Sequence
  TRI 01046 Anti TA scFv x Anti CD3 scFv
gacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaaggtggaggcggttcaggcggaggtggatccggcggtggcggctccggtggcggcggatctgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcctcgagtgagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcggcgggggatccccgtcacaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggtggtggaggtagtggtggaggcggatctggcggcggcggttcaggaggtggtggatccgatgtccagcttacccaaagtccgagcacgttgagtgcaagtgtaggagaccgcgtaacgattacttgctctgcttcaagttccgtatcctacatgaattggtatcagcaaaagcctggaaaagcccctaagcgctggatatacgattcaagtaagttggcttctggcgtcccagcacggttttctggttcaggttccggtacagaatatacgctgacaatcagctctctccaaccggatgatttcgcaacctattactgtcaacaatggtcaagaaatccgccgacattcgggcagggaacaaaagtcgaggtaaaaaggtca

SEQ ID NO: 18 - protein, 744 aa, Artificial Sequence
  TRI 01046 Anti TA scFv x Anti CD3 scFv
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSQVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSSGGGGSGGGGSGGGGSGGGGSDVQLTQSPSTLSASVGDRVTITCSASSSVSYMNWYQQKPGKAPKRWIYDSSKLASGVPARFSGSGSGTEYTLTISSLQPDDFATYYCQQWSRNPPTFGQGTKVEVKRS

SEQ ID NO: 19 - DNA, 1425 bp, Artificial Sequence
  PSMA 01012 Anti-PSMA scFv-Fc
gatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacgaggtggcggagggtctgggggtggcggatccggaggtggtggctctcaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - protein, 475 aa, Artificial Sequence
  PSMA 01012 Anti-PSMA scFv-Fc
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKRGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1437 bp, Artificial Sequence
  PSMA 01019  Anti-PSMA scFv-Fc
gacgtacagattactcaaagcccttcaaccctgtccgcaagtgtcggggacagagttactataacgtgcagggcaagtaagagtataagtaaatatctggcctggtatcaacagaaacccgggaaagcccctaaactcctcatacattcaggatctagccttgagtcaggtgttccgtcaagattctctggttccggaagcgggaccgagttcaccctgacaatcagttcactccagcctgacgacttcgctacttactactgccagcagcacatagagtacccctggacattcggacaaggaacgaaagtcgaaatcaagggtggtggaggtagtggtggaggcggatctggcggcggcggttcaggaggtggtggatccgaggtgcaacttctcgaatccggcggtgggctggtacaaccgggaggatcccttcgcctctcttgtgctgcctccgggtataccttcacggactactatattcactgggtgcgccaggcgccaggaaagggattggaatggatagggagctttaacccctacaacgactacacttcttacgccgactctgtaaaggggagagctactctgtccgtcgataaatcaaagaatacggcataccttcaaatgaactccctgagggctgaggataccgcagtgtactattgtgcaaggagtgatggttactacgacgcgatggactactggggccaaggcaccctggtcacagtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 22 - protein, 479 aa, Artificial Sequence
  PSMA 01019  Anti-PSMA scFv-Fc
DVQITQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKGGGGSGGGGSGGGGSGGGGSEVQLLESGGGLVQPGGSLRLSCAASGYTFTDYYIHWVRQAPGKGLEWIGSFNPYNDYTSYADSVKGRATLSVDKSKNTAYLQMNSLRAEDTAVYYCARSDGYYDAMDYWGQGTLVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1437 bp, Artificial Sequence
  PSMA 01020 Anti-PSMA scFv-Fc
gacatccagatgactcaaagcccttcaaccctgtccgcaagtgtcggggacagagttactataacgtgcagggcaagtaagagtataagtaaatatctggcctggtatcaacagaaacccgggaaagcccctaaactcctcatacattcaggatctagccttgagtcaggtgttccgtcaagattctctggttccggaagcgggaccgagttcaccctgacaatcagttcactccagcctgacgacttcgctacttactactgccagcagcacatagagtacccctggacattcggacaaggaacgaaagtcgaaatcaagggtggtggaggtagtggtggaggcggatctggcggcggcggttcaggaggtggtggatccgaggtgcaacttctcgaatccggcggtgggctggtacaaccgggaggatcccttcgcctctcttgtgctgcctccgggtataccttcacggactactatattcactgggtgcgccaggcgccaggaaagggattggaatggatagggagctttaacccctacaacgactacacttcttacgccgactctgtaaaggggagagctactctgtccgtcgataaatcaaagaatacggcataccttcaaatgaactccctgagggctgaggataccgcagtgtactattgtgcaaggagtgatggttactacgacgcgatggactactggggccaaggcaccctggtcacagtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - protein, 479 aa, Artificial Sequence
  PSMA 01020 Anti-PSMA scFv-Fc
DIQMTQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKGGGGSGGGGSGGGGSGGGGSEVQLLESGGGLVQPGGSLRLSCAASGYTFTDYYIHWVRQAPGKGLEWIGSFNPYNDYTSYADSVKGRATLSVDKSKNTAYLQMNSLRAEDTAVYYCARSDGYYDAMDYWGQGTLVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 1437 aa, Artificial Sequence
  PSMA  01021 Anti-PSMA scFv-Fc
GACGTACAGATTACTCAAAGCCCTTCAACCCTGTCCGCAAGTGTCGGGGACAGAGTTACTATAACGTGCAGGGCAAGTAAGAGTATAAGTAAATATCTGGCCTGGTATCAACAGAAACCCGGGAAAGCCCCTAAACTCCTCATACATTCAGGATCTAGCCTTGAGTCAGGTGTTCCGTCAAGATTCTCTGGTTCCGGAAGCGGGACCGAGTTCACCCTGACAATCAGTTCACTCCAGCCTGACGACTTCGCTACTTACTACTGCCAGCAGCACATAGAGTACCCCTGGACATTCGGACAAGGAACGAAAGTCGAAATCAAGGGTGGTGGAGGTAGTGGTGGAGGCGGATCTGGCGGCGGCGGTTCAGGAGGTGGTGGATCCCAGGTCCAGCTGGTACAGTCTGGGGCTGAGGTGAAGAAGCCTGGGGCTTCAGTGAAGGTCTCCTGCAAGGCTTCTGGATACACATTCACTGACTACTACATGCACTGGGTGCGACAGGCCCCTGGACAAGGGCTTGAGTGGATGGGATATTTTAATCCTTATAATGATTATACTAGATACGCACAGAAGTTCCAGGGCAGAGTCACCATGACCAGGGACACGTCTATCAGCACAGCCTACATGGAGCTGAGCAGCCTGAGATCTGACGACACGGCCGTGTATTACTGTGCAAGATCGGATGGTTACTACGATGCTATGGACTACTGGGGTCAAGGAACCACAGTCACCGTCTCCTCGAGTGAGCCCAAATCTTCTGACAAAACTCACACATGCCCACCGTGCCCAGCACCTGAAGCCGCGGGTGCACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAATACAAGTGCGCGGTCTCCAACAAAGCCCTCCCAGCCCCCATCGAGAAAACCATCTCCAAAGCCAAAGGGCAGCCCCGAGAACCACAGGTGTACACCCTGCCCCCATCCCGGGATGAGCTGACCAAGAACCAGGTCAGCCTGACCTGCCTGGTCAAAGGCTTCTATCCAAGCGACATCGCCGTGGAGTGGGAGAGCAATGGGCAGCCGGAGAACAACTACAAGACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCGGGTAAA

SEQ ID NO: 26 - protein, 479 aa, Artificial Sequence
  PSMA  01021 Anti-PSMA scFv-Fc
DVQITQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKGGGGSGGGGSGGGGSGGGGSQVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 1437 aa, Artificial Sequence
  PSMA 01022 Anti PSMA scFv-Fc
GACGTACAGATTACTCAAAGCCCTTCAACCCTGTCCGCAAGTGTCGGGGACAGAGTTACTATAACGTGCAGGGCAAGTAAGAGTATAAGTAAATATCTGGCCTGGTATCAACAGAAACCCGGGAAAGCCCCTAAACTCCTCATACATTCAGGATCTAGCCTTGAGTCAGGTGTTCCGTCAAGATTCTCTGGTTCCGGAAGCGGGACCGAGTTCACCCTGACAATCAGTTCACTCCAGCCTGACGACTTCGCTACTTACTACTGCCAGCAGCACATAGAGTACCCCTGGACATTCGGACAAGGAACGAAAGTCGAAATCAAGGGTGGTGGAGGTAGTGGTGGAGGCGGATCTGGCGGCGGCGGTTCAGGAGGTGGTGGATCCCAGGTCCAGCTGGTACAGTCTGGGGCTGAGGTGAAGAAGCCTGGGGCTTCAGTGAAGGTCTCCTGCAAGGCTTCTGGATACACATTCACTGACTACTACATGCACTGGGTGCGACAGGCCCCTGGACAAGGGCTTGAGTGGATGGGATATTTTAATCCTTATAATGATTATACTAGATACGCACAGAAGTTCCAGGGCAGAGTCACCATGACCAGGGACACGTCTACCAGCACAGTGTACATGGAGCTGAGCAGCCTGAGATCTGAGGACACGGCCGTGTATTACTGTGCAAGATCGGATGGTTACTACGATGCTATGGACTACTGGGGTCAAGGAACCACAGTCACCGTCTCCTCGAGTGAGCCCAAATCTTCTGACAAAACTCACACATGCCCACCGTGCCCAGCACCTGAAGCCGCGGGTGCACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAATACAAGTGCGCGGTCTCCAACAAAGCCCTCCCAGCCCCCATCGAGAAAACCATCTCCAAAGCCAAAGGGCAGCCCCGAGAACCACAGGTGTACACCCTGCCCCCATCCCGGGATGAGCTGACCAAGAACCAGGTCAGCCTGACCTGCCTGGTCAAAGGCTTCTATCCAAGCGACATCGCCGTGGAGTGGGAGAGCAATGGGCAGCCGGAGAACAACTACAAGACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCGGGTAAA

SEQ ID NO: 28 - protein, 479 aa, Artificial Sequence
  PSMA 01022 Anti PSMA scFv-Fc
DVQITQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKGGGGSGGGGSGGGGSGGGGSQVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1437 bp, Artificial Sequence
  PSMA 01023 Anti PSMA scFv-Fc
gacatccagatgactcaaagcccttcaaccctgtccgcaagtgtcggggacagagttactataacgtgcagggcaagtaagagtataagtaaatatctggcctggtatcaacagaaacccgggaaagcccctaaactcctcatacattcaggatctagccttgagtcaggtgttccgtcaagattctctggttccggaagcgggaccgagttcaccctgacaatcagttcactccagcctgacgacttcgctacttactactgccagcagcacatagagtacccctggacattcggacaaggaacgaaagtcgaaatcaagggtggtggaggtagtggtggaggcggatctggcggcggcggttcaggaggtggtggatcccaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctaccagcacagtgtacatggagctgagcagcctgagatctgaggacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - protein, 479 aa, Artificial Sequence
  PSMA 01023 Anti PSMA scFv-Fc
DIQMTQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKGGGGSGGGGSGGGGSGGGGSQVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 1437 bp, Artificial Sequence
  PSMA 01024 Anti-PSMA scFv-Fc
gacgtacagattactcaaagcccttcaaccctgtccgcaagtgtcggggacagagttactataacgtgcagggcaagtaagagtataagtaaatatctggcctggtatcaacagaaacccgggaaagcccctaaactcctcatacattcaggatctagccttgagtcaggtgttccgtcaagattctctggttccggaagcgggaccgagttcaccctgacaatcagttcactccagcctgacgacttcgctacttactactgccagcagcacatagagtacccctggacattcggacaaggaacgaaagtcgaaatcaagggtggtggaggtagtggtggaggcggatctggcggcggcggttcaggaggtggtggatcccaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggaatttttaatccttataatgattatactagttacgcacagaagttccagggcagagtcaccatgaccagggacacgtctaccagcacagtgtacatggagctgagcagcctgagatctgaggacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 32 - protein, 479 aa, Artificial Sequence
  PSMA 01024 Anti-PSMA scFv-Fc
DVQITQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKGGGGSGGGGSGGGGSGGGGSQVQLVQSGAEVKKPGASVKVSCKASGYTFTDYYMHWVRQAPGQGLEWMGIFNPYNDYTSYAQKFQGRVTMTRDTSTSTVYMELSSLRSEDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 1437 bp, Artificial Sequence
  PSMA 01025  Anti-PSMA scFv-Fc
gacatccagatgactcaaagcccttcaaccctgtccgcaagtgtcggggacagagttactataacgtgcagggcaagtaagagtataagtaaatatctggcctggtatcaacagaaacccgggaaagcccctaaactcctcatacattcaggatctagccttgagtcaggtgttccgtcaagattctctggttccggaagcgggaccgagttcaccctgacaatcagttcactccagcctgacgacttcgctacttactactgccagcagcacatagagtacccctggacattcggacaaggaacgaaagtcgaaatcaagggtggtggaggtagtggtggaggcggatctggcggcggcggttcaggaggtggtggatcccaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggaatttttaatccttataatgattatactagttacgcacagaagttccagggcagagtcaccatgaccagggacacgtctaccagcacagtgtacatggagctgagcagcctgagatctgaggacacggccgtgtattactgtgcaagatcggatggttactacgatgctatggactactggggtcaaggaaccacagtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 34 - protein, 479 aa, Artificial Sequence
  PSMA 01025  Anti-PSMA scFv-Fc
DIQMTQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKGGGGSGGGGSGGGGSGGGGSQVQLVQSGAEVKKPGASVKVSCKASGYTFTDYYMHWVRQAPGQGLEWMGIFNPYNDYTSYAQKFQGRVTMTRDTSTSTVYMELSSLRSEDTAVYYCARSDGYYDAMDYWGQGTTVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1434 bp, Artificial Sequence
  PSMA 01036  Anti-PSMA scFv-Fc
gacatccagatgacccagtctccttccaccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaaggtggtggaggtagtggtggaggcggatctggcggcggcggttcaggaggtggtggatcccaggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgagtgagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - protein, 478 aa, Artificial Sequence
  PSMA 01036  Anti-PSMA scFv-Fc
DIQMTQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKGGGGSGGGGSGGGGSGGGGSQVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 1440 bp, Artificial Sequence
  PSMA 01037 Anti-PSMA scFv-Fc
caggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgggtggtggaggtagtggtggaggcggatctggcggcggcggttcaggaggtggtggatccgacatccagatgacccagtctccttccaccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaatcctcgagtgagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 38 - protein, 480 aa, Artificial Sequence
  PSMA 01037 Anti-PSMA scFv-Fc
QVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSGGGGSGGGGSGGGGSGGGGSDIQMTQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 1455 bp, Artificial Sequence
  PSMA 01040 Fc-linker anti PSMA scFv
tcctcggagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcggcgggggatccccgtcagacatccagatgacccagtctccttccaccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaaggtggtggaggtagtggtggaggcggatctggcggcggcggttcaggaggtggtggatcccaggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgcgc

SEQ ID NO: 40 - protein, 485 aa, Artificial Sequence
  PSMA 01040 Fc-linker anti PSMA scFv
SSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKGGGGSGGGGSGGGGSGGGGSQVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSR

SEQ ID NO: 41 - DNA, 1455 bp, Artificial Sequence
  PSMA 01041 Fc-linker anti PSMA scFv
tcctcggagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcggcgggggatccccgtcacaggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgggtggtggaggtagtggtggaggcggatctggcggcggcggttcaggaggtggtggatccgacatccagatgacccagtctccttccaccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaacgc

SEQ ID NO: 42 - protein, 485 aa, Artificial Sequence
  PSMA 01041 Fc-linker anti PSMA scFv
SSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSQVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSGGGGSGGGGSGGGGSGGGGSDIQMTQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKR

SEQ ID NO: 43 - DNA, 2193 bp, Artificial Sequence
  PSMA 01072 Anti PSMA scFv x Anti CD3 scFv
caggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgggtggtggaggtagtggtggaggcggatctggcggcggcggttcaggaggtggtggatccgacatccagatgacccagtctccttcctccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaagagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcggcgggggatccccgtcacaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggtggtggaggtagtggtggaggcggatctggcggcggcggttcaggaggtggtggatccgatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaaaggtca

SEQ ID NO: 44 - protein, 731 aa, Artificial Sequence
  PSMA 01072 Anti PSMA scFv x Anti CD3 scFv
QVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSGGGGSGGGGSGGGGSGGGGSDIQMTQSPSSLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSPSQVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSSGGGGSGGGGSGGGGSGGGGSDIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIKRS

SEQ ID NO: 45 - DNA, 1470 bp, Artificial Sequence
  Anti TA scFv x Fc Knob (Chain 1)
gacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaaggtggaggcggttcaggcggaggtggatccggcggtggcggctccggtggcggcggatctgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcctcgagtgagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 46 - protein, 490 aa, Artificial Sequence
  Anti TA scFv x Fc Knob (Chain 1)
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 2232 bp, Artificial Sequence
  Anti TA scFv x Fc Hole x Anti CD3 scFv (Chain 2)
gacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaaggtggaggcggttcaggcggaggtggatccggcggtggcggctccggtggcggcggatctgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaattgtcaccgtctcctcgagtgagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtcttgcgctgtcaaaggcttctatccaagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccgtttcacgcagaagagcctctccctgtctccgggcggcgggggatccccgtcacaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggtggtggaggtagtggtggaggcggatctggcggcggcggttcaggaggtggtggatccgatgtccagcttacccaaagtccgagcacgttgagtgcaagtgtaggagaccgcgtaacgattacttgctctgcttcaagttccgtatcctacatgaattggtatcagcaaaagcctggaaaagcccctaagcgctggatatacgattcaagtaagttggcttctggcgtcccagcacggttttctggttcaggttccggtacagaatatacgctgacaatcagctctctccaaccggatgatttcgcaacctattactgtcaacaatggtcaagaaatccgccgacattcgggcagggaacaaaagtcgaggtaaaaaggtca

SEQ ID NO: 48 - protein, 744 aa, Artificial Sequence
  Anti TA scFv x Fc Hole x Anti CD3 scFv (Chain 2)
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTIVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGSPSQVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSSGGGGSGGGGSGGGGSGGGGSDVQLTQSPSTLSASVGDRVTITCSASSSVSYMNWYQQKPGKAPKRWIYDSSKLASGVPARFSGSGSGTEYTLTISSLQPDDFATYYCQQWSRNPPTFGQGTKVEVKRS

SEQ ID NO: 49 - DNA, 2232 bp, Artificial Sequence
  Anti TA scFv x Fc Hole x Anti CD3 scFv (Chain 2)
gacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaaggtggaggcggttcaggcggaggtggatccggcggtggcggctccggtggcggcggatctgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcctcgagtgagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtcttgcgctgtcaaaggcttctatccaagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccgtttcacgcagaagagcctctccctgtctccgggcggcgggggatccccgtcacaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacaatcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggtggtggaggtagtggtggaggcggatctggcggcggcggttcaggaggtggtggatccgatgtccagcttacccaaagtccgagcacgttgagtgcaagtgtaggagaccgcgtaacgattacttgctctgcttcaagttccgtatcctacatgaattggtatcagcaaaagcctggaaaagcccctaagcgctggatatacgattcaagtaagttggcttctggcgtcccagcacggttttctggttcaggttccggtacagaatatacgctgacaatcagctctctccaaccggatgatttcgcaacctattactgtcaacaatggtcaagaaatccgccgacattcgggcagggaacaaaagtcgaggtaaaaaggtca

SEQ ID NO: 50 - protein, 744 aa, Artificial Sequence
  Anti TA scFv x Fc Hole x Anti CD3 scFv (Chain 2)
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGSPSQVQLVQSGAEVKKPGASVKVSCKASGYTFTRSTMHWVRQAPGQGLEWIGYINPSSAYTNYNQKFQGRVTLTADKSTSTAYMELSSLRSEDTAVYYCASPQVHYDYNGFPYWGQGTLVTVSSGGGGSGGGGSGGGGSGGGGSDVQLTQSPSTLSASVGDRVTITCSASSSVSYMNWYQQKPGKAPKRWIYDSSKLASGVPARFSGSGSGTEYTLTISSLQPDDFATYYCQQWSRNPPTFGQGTKVEVKRS

SEQ ID NO: 51 - DNA, 2232 bp, Artificial Sequence
  Anti TA scFv x Fc Hole x Anti CD3 scFv (Chain 2)
gacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaaggtggaggcggttcaggcggaggtggatccggcggtggcggctccggtggcggcggatctgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaattgtcaccgtctcctcgagtgagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtcttgcgctgtcaaaggcttctatccaagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccgtttcacgcagaagagcctctccctgtctccgggcggcgggggatccccgtcacaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggtggtggaggtagtggtggaggcggatctggcggcggcggttcaggaggtggtggatccgatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaaaggtca

SEQ ID NO: 52 - protein, 744 aa, Artificial Sequence
  Anti TA scFv x Fc Hole x Anti CD3 scFv (Chain 2)
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTIVTVSSSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGSPSQVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSSGGGGSGGGGSGGGGSGGGGSDIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIKRS

SEQ ID NO: 53 - DNA, 1422 bp, Artificial Sequence
  Anti-PSMA scFv x Fc Knob (Chain 1)
gacatccagatgactcaaagcccttcaaccctgtccgcaagtgtcggggacagagttactataacgtgcagggcaagtaagagtataagtaaatatctggcctggtatcaacagaaacccgggaaagcccctaaactcctcatacattcaggatctagccttgagtcaggtgttccgtcaagattctctggttccggaagcgggaccgagttcaccctgacaatcagttcactccagcctgacgacttcgctacttactactgccagcagcacatagagtacccctggacattcggacaaggaacgaaagtcgaaatcaagggtggtggaggtagtggtggaggcggatctggcggcggcggttcaggaggtggtggatcccaggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctaccagcacagtgtacatggagctgagcagcctgagatctgaggacacggccgtgtattactgtgcaagatcggatggttactacgatgctatggactactggggtcaaggaaccacagtcaccgtcgagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 54 - protein, 474 aa, Artificial Sequence
  Anti-PSMA scFv x Fc Knob (Chain 1)
DIQMTQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKGGGGSGGGGSGGGGSGGGGSQVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - DNA, 1431 bp, Artificial Sequence
  Anti-CD3 scFv x Fc Hole (Chain 2)
caagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggtggtggaggtagtggtggaggcggatctggcggcggcggttcaggaggtggtggatccgatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaagagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtcttgcgctgtcaaaggcttctatccaagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccgtttcacgcagaagagcctctccctgtctccgggt

SEQ ID NO: 56 - protein, 477 aa, Artificial Sequence
  Anti-CD3 scFv x Fc Hole (Chain 2)
QVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSSGGGGSGGGGSGGGGSGGGGSDIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 57 - DNA, 1428 bp, Artificial Sequence
  Anti PSMA scFv x Fc Knob (Chain 1)
caggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgggtggtggaggtagtggtggaggcggatctggcggcggcggttcaggaggtggtggatccgacatccagatgacccagtctccttcctccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaagagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 58 - protein, 476 aa, Artificial Sequence
  Anti PSMA scFv x Fc Knob (Chain 1)
QVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSGGGGSGGGGSGGGGSGGGGSDIQMTQSPSSLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - DNA, 3624 bp, Artificial Sequence
  Anti PSMA scFv x Fc Hole x linker x Anti CD3 scFv (Chain 2)
caggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgggtggtggaggtagtggtggaggcggatctggcggcggcggttcaggaggtggtggatccgacatccagatgacccagtctccttcctccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaagagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtcttgcgctgtcaaaggcttctatccaagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccgtttcacgcagaagagcctctccctgtctccgggcggcgggggatccccgtcacaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggtggtggaggtagtggtggaggcggatctggcggcggcggttcaggaggtggtggatccgatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaaaggtcacaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggtggtggaggtagtggtggaggcggatctggcggcggcggttcaggaggtggtggatccgatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaagagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtcttgcgctgtcaaaggcttctatccaagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccgtttcacgcagaagagcctctccctgtctccgggt

SEQ ID NO: 60 - protein, 731 aa, Artificial Sequence
  Anti PSMA scFv x Fc Hole x linker x Anti CD3 scFv (Chain 2)
QVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSGGGGSGGGGSGGGGSGGGGSDIQMTQSPSSLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGSPSQVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSSGGGGSGGGGSGGGGSGGGGSDIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIKRS

SEQ ID NO: 61 - DNA, 2187 bp, Artificial Sequence
  Anti PSMA x Fc Hole x Anti CD3 (Chain 2)
caggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgggtggtggaggtagtggtggaggcggatctggcggcggcggttcaggaggtggtggatccgacatccagatgacccagtctccttcctccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaagagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtcttgcgctgtcaaaggcttctatccaagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccgtttcacgcagaagagcctctccctgtctccgggcggcgggggatccccgtcagatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaaggcggaggcggaagcggaggtgggggctccggaggcgggggaagcggcggaggtggctctcaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagc

SEQ ID NO: 62 - protein, 729 aa, Artificial Sequence
  Anti PSMA x Fc Hole x Anti CD3 (Chain 2)
QVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSGGGGSGGGGSGGGGSGGGGSDIQMTQSPSSLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGSPSDIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIKGGGGSGGGGSGGGGSGGGGSQVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSS

SEQ ID NO: 63 - DNA, 696 bp, Artificial Sequence
  TSC01007  PAAdel Fc
tcctcggagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 64 - protein, 232 aa, Artificial Sequence
  TSC01007  PAAdel Fc
SSEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - DNA, 687 bp, Artificial Sequence
  PAAdel Fc + Knob
gagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccg

SEQ ID NO: 66 - protein, 229 aa, Artificial Sequence
  PAAdel FC + Knob
EPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 67 - DNA, 690 bp, Artificial Sequence
  PAAdel Fc + Hole
gagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtcttgcgctgtcaaaggcttctatccaagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccactacacgcagaagagcctctccctgtctccgggt

SEQ ID NO: 68 - protein, 230 aa, Artificial Sequence
  PAAdel Fc + Hole
EPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 69 - DNA, 24 bp, Artificial Sequence
  Anti PSMA scFv HCDR1
ggatacaccttcaccgactactat

SEQ ID NO: 70 - protein, 8 aa, Artificial Sequence
  Anti PSMA scFv HCDR1
GYTFTDYY

SEQ ID NO: 71 - DNA, 24 bp, Artificial Sequence
  Anti PSMA scFv HCDR2
ttcaacccttataatgattacaca

SEQ ID NO: 72 - protein, 8 aa, Artificial Sequence
  Anti PSMA scFv HCDR2
FNPYNDYT

SEQ ID NO: 73 - DNA, 36 bp, Artificial Sequence
  Anti PSMA scFv HCDR3
gcgagatctgacggctactacgacgctatggactac

SEQ ID NO: 74 - protein, 12 aa, Artificial Sequence
  Anti PSMA scFv HCDR3
ARSDGYYDAMDY

SEQ ID NO: 75 - DNA, 18 bp, Artificial Sequence
  Anti PSMA scFv LCDR1
aagagtattagtaagtac

SEQ ID NO: 76 - protein, 6 aa, Artificial Sequence
  Anti PSMA scFv LCDR2
KSISKY

SEQ ID NO: 77 - DNA, 9 bp, Artificial Sequence
  Anti PSMA scFv LCDR2
tctggctcc

SEQ ID NO: 78 - protein, 3 aa, Artificial Sequence
  Anti PSMA scFv LCDR2
SGS

SEQ ID NO: 79 - DNA, 27 bp, Artificial Sequence
  Anti PSMA scFv LCDR3
caacagcatattgaatatccttggacg

SEQ ID NO: 80 - protein, 9 aa, Artificial Sequence
  Anti PSMA scFv LCDR3
QQHIEYPWT

SEQ ID NO: 81 - DNA, 357 bp, Artificial Sequence
  Anti PSMA scFv Heavy Chain
caggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcg

SEQ ID NO: 82 - protein, 119 aa, Artificial Sequence
  Anti PSMA scFv Heavy Chain
QVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSS

SEQ ID NO: 83 - DNA, 321 bp, Artificial Sequence
  Anti PSMA scFv Light Chain
gacatccagatgacccagtctccttcctccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaa

SEQ ID NO: 84 - protein, 107 aa, Artificial Sequence
  Anti PSMA scFv Light Chain
DIQMTQSPSSLSASVGDRVTITCRASKSISKYLAWYQQKPGKAPKLLIHSGSSLESGVPSRFSGSGSGTEFTLTISSLQPDDFATYYCQQHIEYPWTFGQGTKVEIK

SEQ ID NO: 85 - DNA, 738 bp, Artificial Sequence
  Anti PSMA scFv
caggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgggaggcggtggatcaggcggtggaggcagcggaggaggtggctccggtggcggagggagcgacatccagatgacccagtctccttcctccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaa

SEQ ID NO: 86 - protein, 246 aa, Artificial Sequence
  Anti PSMA scFv
QVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSGGGGSGGGGSGGGGSGGGGSDIQMTQSPSSLSASVGDRVTITCRASKSISKYLAWYQQKPGKAPKLLIHSGSSLESGVPSRFSGSGSGTEFTLTISSLQPDDFATYYCQQHIEYPWTFGQGTKVEIK

SEQ ID NO: 87 - DNA, 24 bp, Artificial Sequence
  Anti-CD3 scFv  HCDR1
gggtacaccttcacccggtctaca

SEQ ID NO: 88 - protein, 8 aa, Artificial Sequence
  Anti-CD3 scFv  HCDR1
GYTFTRST

SEQ ID NO: 89 - DNA, 24 bp, Artificial Sequence
  Anti-CD3 scFv  HCDR2
atcaacccttcctctgcatacacc

SEQ ID NO: 90 - protein, 8 aa, Artificial Sequence
  Anti-CD3 scFv  HCDR2
INPSSAYT

SEQ ID NO: 91 - DNA, 42 bp, Artificial Sequence
  Anti-CD3 scFv  HCDR3
gcatctccccaggtccattatgactacaacgggtttccgtac

SEQ ID NO: 92 - protein, 14 aa, Artificial Sequence
  Anti-CD3 scFv  HCDR3
ASPQVHYDYNGFPY

SEQ ID NO: 93 - DNA, 15 bp, Artificial Sequence
  Anti-CD3 scFv  LCDR1
agttccgtatcctac

SEQ ID NO: 94 - protein, 5 aa, Artificial Sequence
  Anti-CD3 scFv  LCDR1
SSVSY

SEQ ID NO: 95 - DNA, 9 bp, Artificial Sequence
  Anti-CD3 scFv  LCDR2
gattcaagt

SEQ ID NO: 96 - protein, 3 aa, Artificial Sequence
  Anti-CD3 scFv  LCDR2
DSS

SEQ ID NO: 97 - DNA, 27 bp, Artificial Sequence
  Anti-CD3 scFv  LCDR3
caacaatggtcaagaaatccgccgaca

SEQ ID NO: 98 - protein, 9 aa, Artificial Sequence
  Anti-CD3 scFv  LCDR3
QQWSRNPPT

SEQ ID NO: 99 - DNA, 363 bp, Artificial Sequence
  Anti-CD3 scFv Heavy Chain
caagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagc

SEQ ID NO: 100 - protein, 121 aa, Artificial Sequence
  Anti-CD3 scFv Heavy Chain
QVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSS

SEQ ID NO: 101 - DNA, 318 bp, Artificial Sequence
  VL
gatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaa

SEQ ID NO: 102 - protein, 106 aa, Artificial Sequence
  VL
DIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIK

SEQ ID NO: 103 - DNA, 741 bp, Artificial Sequence
  scFv
caagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggcggaggcggaagcggaggtgggggctccggaggcgggggaagcggcggaggtggctctgatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaa

SEQ ID NO: 104 - protein, 247 aa, Artificial Sequence
  scFv
QVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSSGGGGSGGGGSGGGGSGGGGSDIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIK

SEQ ID NO: 105 - DNA, 2193 bp, Artificial Sequence
  Anti PSMA x Fc Knob x Anti CD3 scFv (Chain 1)
caggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgggaggcggtggatcaggcggtggaggcagcggaggaggtggctccggtggcggagggagcgacatccagatgacccagtctccttcctccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaagagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcggcgggggatccccgtcacaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggcggaggcggaagcggaggtgggggctccggaggcgggggaagcggcggaggtggctctgatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaaaggtca

SEQ ID NO: 106 - protein, 731 aa, Artificial Sequence
  Anti PSMA x Fc Knob x Anti CD3 scFv (Chain 1)
QVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSGGGGSGGGGSGGGGSGGGGSDIQMTQSPSSLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSPSQVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSSGGGGSGGGGSGGGGSGGGGSDIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIKRS

SEQ ID NO: 107 - DNA, 1428 bp, Artificial Sequence
  Anti PSMA x Fc Hole (Chain 2)
caggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgggtggtggaggtagtggtggaggcggatctggcggcggcggttcaggaggtggtggatccgacatccagatgacccagtctccttcctccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaagagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtcttgcgctgtcaaaggcttctatccaagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccactacacgcagaagagcctctccctgtctccgggt

SEQ ID NO: 108 - protein, 476 aa, Artificial Sequence
  Anti PSMA x Fc Hole (Chain 2)
QVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSGGGGSGGGGSGGGGSGGGGSDIQMTQSPSSLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 109 - DNA, 741 bp, Artificial Sequence
  Anti-CD3 scFv
gatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaaggcggaggcggaagcggaggtgggggctccggaggcgggggaagcggcggaggtggctctcaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagc

SEQ ID NO: 110 - protein, 247 aa, Artificial Sequence
  Anti-CD3 scFv
DIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIKGGGGSGGGGSGGGGSGGGGSQVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSS

SEQ ID NO: 111 - DNA, 2187 bp, Artificial Sequence
  Anti PSMA x Fc Knob x Anti CD3 scFv (Chain 1)
caggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgggaggcggtggatcaggcggtggaggcagcggaggaggtggctccggtggcggagggagcgacatccagatgacccagtctccttcctccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaagagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcggcgggggatccccgtcagatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaaggcggaggcggaagcggaggtgggggctccggaggcgggggaagcggcggaggtggctctcaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagc

SEQ ID NO: 112 - protein, 729 aa, Artificial Sequence
  Anti PSMA x Fc Knob x Anti CD3 scFv (Chain 1)
QVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSGGGGSGGGGSGGGGSGGGGSDIQMTQSPSSLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSPSDIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIKGGGGSGGGGSGGGGSGGGGSQVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSS

SEQ ID NO: 113 - DNA, 357 bp, Artificial Sequence
  PSMA01012  VH domain
caggtccagctggtacagtctggggctgaggtgaagaagcctggggcttcagtgaaggtctcctgcaaggcttctggatacacattcactgactactacatgcactgggtgcgacaggcccctggacaagggcttgagtggatgggatattttaatccttataatgattatactagatacgcacagaagttccagggcagagtcaccatgaccagggacacgtctatcagcacagcctacatggagctgagcagcctgagatctgacgacacggccgtgtattactgtgcaagatcggatggttactacgatgctatggactactggggtcaaggaaccacagtcaccgtctcctcg

SEQ ID NO: 114 - protein, 119 aa, Artificial Sequence
  PSMA01012  VH domain
QVQLVQSGAEVKKPGASVKVSCKASGYTFTDYYMHWVRQAPGQGLEWMGYFNPYNDYTRYAQKFQGRVTMTRDTSISTAYMELSSLRSDDTAVYYCARSDGYYDAMDYWGQGTTVTVSS

SEQ ID NO: 115 - DNA, 324 bp, Artificial Sequence
  PSMA01012  VL domain
gatatccagatgacccagtctccatccgccatgtctgcatctgtaggagacagagtcaccatcacttgccgggcgagtaagagcattagcaaatatttagcctggtttcagcagaaaccagggaaagttcctaagctccgcatccattctggatctactttgcaatcaggggtcccatctcggttcagtggcagtggatctgggacagaatttactctcaccatcagcagcctgcagcctgaagattttgcaacttattactgtcaacagcatattgaatacccgtggacgttcggccaagggaccaaggtggaaatcaaacga

SEQ ID NO: 116 - protein, 108 aa, Artificial Sequence
  PSMA01012  VL domain
DIQMTQSPSAMSASVGDRVTITCRASKSISKYLAWFQQKPGKVPKLRIHSGSTLQSGVPSRFSGSGSGTEFTLTISSLQPEDFATYYCQQHIEYPWTFGQGTKVEIKR

SEQ ID NO: 117 - DNA, 357 bp, Artificial Sequence
  107-1A4 VH domain
gagatccagctgcaacagtctggacctgagctggtgaagcctggggcttcagtgaagatgtcctgcaaggcttctggatacacattcactgactactacatgcactgggtgaagcagaacaatggagagagccttgagtggattggatattttaatccttataatgattatactagatacaaccagaatttcaatggcaaggccacattgactgtagacaagtcctccagcacagcctacatgcagctcaacagcctgacatctgaggactctgcattctattactgtgcaagatcggatggttactacgatgctatggactactggggtcaaggaacctcagtcaccgtctcctcg

SEQ ID NO: 118 - protein, 119 aa, Artificial Sequence
  107-1A4 VH domain
EIQLQQSGPELVKPGASVKMSCKASGYTFTDYYMHWVKQNNGESLEWIGYFNPYNDYTRYNQNFNGKATLTVDKSSSTAYMQLNSLTSEDSAFYYCARSDGYYDAMDYWGQGTSVTVSS

SEQ ID NO: 119 - DNA, 327 bp, Artificial Sequence
  107-1A4 VL domain
gatgtccagataacccagtctccatcttatcttgctgcatctcctggagaaaccattactattaattgcagggcaagtaagagcattagcaaatatttagcctggtatcaagagaaacctgggaaagctaataagctacttatccattctggatccactttgcaatctggaataccatcaaggttcagtggcagtggatctggtacagatttcactctcaccatcagtagcctggagcctgaagattttgcaatgtattactgtcaacagcatattgaatacccgtggacgttcggtggtggcaccaaactggaaattaaacgggcc

SEQ ID NO: 120 - protein, 109 aa, Artificial Sequence
  107-1A4 VL domain
DVQITQSPSYLAASPGETITINCRASKSISKYLAWYQEKPGKANKLLIHSGSTLQSGIPSRFSGSGSGTDFTLTISSLEPEDFAMYYCQQHIEYPWTFGGGTKLEIKRA

SEQ ID NO: 121 - skipped

SEQ ID NO: 122 - protein, 115 aa, Artificial Sequence
  CRIS-7 VH domain
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGT

SEQ ID NO: 123 - skipped

SEQ ID NO: 124 - protein, 107 aa, Artificial Sequence
  CRIS-7 VL domain
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQITR

SEQ ID NO: 125 - skipped

SEQ ID NO: 126 - protein, 121 aa, Artificial Sequence
  DRA222 VH domain (TSC266 CD3 VH domain)
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS

SEQ ID NO: 127 - skipped

SEQ ID NO: 128 - protein, 106 aa, Artificial Sequence
  DRA222VL domain (TSC266 CD3 VL domain)
DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQIT

SEQ ID NO: 129 - skipped

SEQ ID NO: 130 - protein, 121 aa, Artificial Sequence
  TSC456 VH domain
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSS

SEQ ID NO: 131 - skipped

SEQ ID NO: 132 - protein, 106 aa, Artificial Sequence
  TSC456 VL domain
DIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIK

SEQ ID NO: 133 - skipped

SEQ ID NO: 134 - protein, 121 aa, Artificial Sequence; identical to SEQ ID NO: 100
  CRIS7H14 VH domain

SEQ ID NO: 135 - skipped

SEQ ID NO: 136 - protein, 103 aa, Artificial Sequence
  CRIS7H14 VL domain
DVQLTQSPSTLSASVGDRVTITCSASSSVSYMNWYQQKPGKAPKRWIYDSSKLASGVPARFSGSGSGTLTISSLQPDDFATYYCQQWSRMPPTFGQGTKVEVK

SEQ ID NO: 137 - skipped

SEQ ID NO: 138 - protein, 122 aa, Artificial Sequence
  CRIS7H15 VH domain
QVQLVQSGAEVKKPGASVKVSCKASGYTFTRSTMHWVRQAPGQGLEWIGYINPSSAYTNYNQKRFQGRVTLTADKSTSTAYMELSSLRSEDTAVYYCASPQVHYDYNGFPYWGQGTLVTVSS

SEQ ID NO: 139 - skipped

SEQ ID NO: 140 - protein, 106 aa, Artificial Sequence
  CRIS7H15 VL domain
DVQLTQSPSTLSASVGDRVTITCSASSSVSYMNWYQQKPGKAPKRWIYDSSKLASGVPARFSGSGSGTEYTLTISSLQPDDFATYYCQQWSRNPPTFGQGTKVEVK

SEQ ID NO: 141 - skipped

SEQ ID NO: 142 - protein, 121 aa, Artificial Sequence; identical to SEQ ID NO: 100
  CRIS7H16 VH domain

SEQ ID NO: 143 - skipped

SEQ ID NO: 144 - protein, 106 aa, Artificial Sequence; identical to SEQ ID NO: 102
  CRIS7H16 VL domain

SEQ ID NO: 145 - protein, 15 aa, Artificial Sequence
  sss(s)-hIgG1 hinge
EPKSSDKTHTSPPSS

SEQ ID NO: 146 - protein, 15 aa, Artificial Sequence
  csc(s)-hIgG1 hinge
EPKSCDKTHTSPPCS

SEQ ID NO: 147 - protein, 15 aa, Artificial Sequence
  ssc(s)-hIgG1 hinge
EPKSSDKTHTSPPCS

SEQ ID NO: 148 - protein, 15 aa, Artificial Sequence
  scc(s)-hIgG1 hinge
EPKSSDKTHTCPPCS

SEQ ID NO: 149 - protein, 15 aa, Artificial Sequence
  css(s)-hIgG1 hinge
EPKSCDKTHTSPPSS

SEQ ID NO: 150 - protein, 15 aa, Artificial Sequence
  scs(s)-hIgG1 hinge
EPKSSDKTHTCPPSS

SEQ ID NO: 151 - protein, 15 aa, Artificial Sequence
  ccc(s)-hIgG1 hinge
EPKSCDKTHTSPPCS

SEQ ID NO: 152 - protein, 15 aa, Artificial Sequence
  ccc(p)-hIgG1 hinge
EPKSCDKTHTSPPCP

SEQ ID NO: 153 - protein, 15 aa, Artificial Sequence
  sss(p)-hIgG1 hinge
EPKSSDKTHTSPPSP

SEQ ID NO: 154 - protein, 15 aa, Artificial Sequence
  csc(p)-hIgG1 hinge
EPKSCDKTHTSPPCP

SEQ ID NO: 155 - protein, 15 aa, Artificial Sequence
  ssc(p)-hIgG1 hinge
EPKSSDKTHTSPPCP

SEQ ID NO: 156 - protein, 15 aa, Artificial Sequence
  scc(p)-hIgG1 hinge
EPKSSDKTHTCPPCP

SEQ ID NO: 157 - protein, 15 aa, Artificial Sequence
  css(p)-hIgG1 hinge
EPKSCDKTHTSPPSP

SEQ ID NO: 158 - protein, 15 aa, Artificial Sequence
  scs(p)-hIgG1 hinge
EPKSSDKTHTCPPSP

SEQ ID NO: 159 - protein, 6 aa, Artificial Sequence
  Scppcp
SCPPCP

SEQ ID NO: 160 - protein, 20 aa, Artificial Sequence
  STD1
NYGGGGSGGGGSGGGGSGNS

SEQ ID NO: 161 - protein, 38 aa, Artificial Sequence
  STD2
NYGGGGSGGGGSGGGGSGNYGGGGSGGGGSGGGGSGNS

SEQ ID NO: 162 - protein, 2 aa, Artificial Sequence
  H1
NS

SEQ ID NO: 163 - protein, 8 aa, Artificial Sequence
  H2
GGGGSGNS

SEQ ID NO: 164 - protein, 10 aa, Artificial Sequence
  H3
NYGGGGSGNS

SEQ ID NO: 165 - protein, 13 aa, Artificial Sequence
  H4
GGGGSGGGGSGNS

SEQ ID NO: 166 - protein, 15 aa, Artificial Sequence
  H5
NYGGGGSGGGGSGNS

SEQ ID NO: 167 - protein, 18 aa, Artificial Sequence
  H6
GGGGSGGGGSGGGGSGNS

SEQ ID NO: 168 - protein, 8 aa, Artificial Sequence
  H7
GCPPCPNS

SEQ ID NO: 169 - protein, 15 aa, Artificial Sequence
  (G4S)3
GGGGSGGGGSGGGGS

SEQ ID NO: 170 - protein, 16 aa, Artificial Sequence
  H105
SGGGGSGGGGSGGGGS

SEQ ID NO: 171 - protein, 20 aa, Artificial Sequence
  (G4S)4
GGGGSGGGGSGGGGSGGGGS

SEQ ID NO: 172 - protein, 19 aa, Artificial Sequence
  H94
SGGGGSGGGGSGGGGSPNS

SEQ ID NO: 173 - protein, 19 aa, Artificial Sequence
  H111
SGGGGSGGGGSGGGGSPGS

SEQ ID NO: 174 - protein, 17 aa, Artificial Sequence
  H114
GGGGSGGGGSGGGGSPS

SEQ ID NO: 175 - protein, 5 aa, Artificial Sequence
  G4S
GGGGS

SEQ ID NO: 176 - skipped

SEQ ID NO: 177 - DNA, 2187 bp, Artificial Sequence
  Anti PSMA x Fc Knob x Anti CD3 scFv (Chain 1)
caggtgcagctggtgcagtctggggctgaggtgaagaagcctggggcctcagtgaaggtttcctgcaaggcatctggatacaccttcaccgactactatatgcactgggtgcgacaggcccctggacaagggcttgagtggatgggatatttcaacccttataatgattacacacgctacgcacagaagttccagggcagagtcaccatgaccagggacacgtccacgagcacagtctacatggagctgagcagcctgagatctgaggacacggccgtgtattactgtgcgagatctgacggctactacgacgctatggactactgggggcaagggaccacggtcaccgtctcctcgggaggcggtggatcaggcggtggaggcagcggaggaggtggctccggtggcggagggagcgacatccagatgacccagtctccttcctccctgtctgcatctgtaggagacagagtcaccatcacttgccgggccagtaagagtattagtaagtacttggcctggtatcagcagaaaccagggaaagcccctaagctcctgatccattctggctccagtttggaaagtggggtcccatcaaggttcagcggcagtggatctgggacagaattcactctcaccatcagcagcctgcagcctgatgattttgcaacttattactgccaacagcatattgaatatccttggacgttcggccaagggaccaaggtggaaatcaaagagcccaaatcttctgacaaaactcacacatgcccaccgtgcccagcacctccagccgctgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgtggtgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcggcgggggatccccgtcacaagtacaactcgttcaaagtggcgcagaagtaaagaagccaggcgccagtgttaaggtgagctgcaaggcaagcgggtacaccttcacccggtctacaatgcactgggtaagacaagcaccagggcaaggactcgaatggattggttacatcaacccttcctctgcatacaccaactacgctcaaaagttccagggccgcgttactttgacagcggataaatctacatccacggcctatatggaactgtcaagcctcaggagcgaggacacagcggtatattactgtgcatctccccaggtccattatgactacaacgggtttccgtactggggacaaggaactctggttacagtcagtagcggcggaggcggaagcggaggtgggggctccggaggcgggggaagcggcggaggtggctctgatatccagatgacccaaagtccgagctcgttgagtgcaagtgtaggagaccgcgtaacgattacttgcagagcttcaagttccgtatcctacatgaattggtatcagcaaaagcctggaaaagcccctaagcgctggatatacgattcaagtaagttggcttctggcgtcccatcacggttttctggttcaggttccggtacagattttacgctgacaatcagctctctccaaccggaagatttcgcaacctattactgtcaacaatggtcaagaaatccgccgacattcgggcagggaacaaaagtcgagataaaa

SEQ ID NO: 178 - protein, 729 aa, Artificial Sequence
  Anti PSMA x Fc Knob x Anti CD3 scFv (Chain 1)
QVQLVQSGAEVKKPGASVKVSCKASGYTFTDYYMHWVRQAPGQGLEWMGYFNPYNDYTRYAQKFQGRVTMTRDTSTSTVYMELSSLRSEDTAVYYCARSDGYYDAMDYWGQGTTVTVSSGGGGSGGGGSGGGGSGGGGSDIQMTQSPSSLSASVGDRVTITCRASKSISKYLAWYQQKPGKAPKLLIHSGSSLESGVPSRFSGSGSGTEFTLTISSLQPDDFATYYCQQHIEYPWTFGQGTKVEIKEPKSSDKTHTCPPCPAPPAAAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSPSQVQLVQSGAEVKKPGASVKVSCKASGYTFTRSTMHWVRQAPGQGLEWIGYINPSSAYTNYAQKFQGRVTLTADKSTSTAYMELSSLRSEDTAVYYCASPQVHYDYNGFPYWGQGTLVTVSSGGGGSGGGGSGGGGSGGGGSDIQMTQSPSSLSASVGDRVTITCRASSSVSYMNWYQQKPGKAPKRWIYDSSKLASGVPSRFSGSGSGTDFTLTISSLQPEDFATYYCQQWSRNPPTFGQGTKVEIK

SEQ ID NO: 179 - DNA, 2820 bp, Artificial Sequence
  mIgG Fc- human PSMA ECD
tcgagtgagcccagagggcccacaatcaagccctgtcctccatgcaaatgcccggctccaaatgctgcaggtggtccatccgtcttcatcttccctccaaagatcaaggatgtactcatgatctccctgagccccatagtcacatgtgtggtggtggatgtgagcgaggacgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcgctgcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggactaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggctcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaactcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcc

SEQ ID NO: 180 - protein, 940 aa, Artificial Sequence
  mIgG Fc- human PSMA ECD Amino Acid Sequence
SSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLAAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 181 - DNA, 2820 bp, Artificial Sequence
  mIgG Fc-cyno PSMA ECD
tcgagtgagcccagagggcccacaatcaagccctgtcctccatgcaaatgcccggctccaaatgctgcaggtggtccatccgtcttcatcttccctccaaagatcaaggatgtactcatgatctccctgagccccatagtcacatgtgtggtggtggatgtgagcgaggacgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcgctgcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggactaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggctcctccagtgaagctactaacattactccaaagcataatatgaaagcatttttggatgaactgaaagctgagaacatcaagaagttcttacataattttacacagataccacatttagcaggaacagaacaaaactttcaacttgcaaagcaaattcaatcccagtggaaagaatttggcctggattctgttgagctaactcattatgatgtcctgttgtcctacccaaataagactcatcccaactacatctcaataattaatgaagatggaaatgagattttcaacacatcattatttgaaccacctcctgcaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccacaggagtcattctctactcagaccctgctgactactttgctcctggggtaaagtcttatccagatggttggaatcttcctggaggtggtgtccagcgtggaaatatcctaaatctgaatggtgcaggagaccctctcacaccaggttacccagcaaatgaatatgcttataggcgtggaatggcagaggctgttggtcttccaagtattcccgttcatccaattgggtactatgatgcacagaagctcctagaaaaaatgggtggctcagcatcaccagatagcagctggagaggaagtctcaaagtgccctacaatgttggacctggctttactggaaacttttctacacaaaaagtcaagatgcacatccactctaccagtgaagtgacaagaatttacaatgtgataggtactctcagaggagcagtggaaccagacagatacgtcattctgggaggtcaccgggactcatgggtgtttggtggtattgaccctcagagtggagcagctgttgttcatgaaattgtgaggagctttggaacgctgaaaaaggaagggtggagacctagaagaacaattttgtttgcaagctgggatgcagaagaatttggtcttcttggttctactgaatgggcagaggagaactcaagactccttcaagagcgtggcgtggcttatattaatgctgattcgtctatagaaggaaactacactctgagagttgattgtacaccactgatgtacagcttggtatacaacctaacaaaagagctggaaagccctgatgaaggctttgaaggcaaatctctttatgaaagttggactaaaaaaagtccttcccccgagttcagtggcatgcccaggataagcaaattgggatctggaaatgattttgaggtgttcttccaacgacttggaattgcctcaggcagagcacggtatactaaaaattgggaaacaaacaaattcagcagctatccactgtatcacagtgtctatgagacatatgagttggtggaaaagttttatgatccaatgtttaaatatcacctcactgtggcccaggttcgaggagggatggtgtttgaactagccaattccgtagtgctcccttttgattgtcgagattatgctgtagttttaagaaagtatgctgacaaaatctacaatatttctatgaaacatccacaggaaatgaagacatacagtgtatcatttgattcacttttttctgcagtaaagaattttacagaaattgcttccaagttcagtgagagactccgggactttgacaaaagcaacccaatattattaagaatgatgaatgatcaactcatgtttctggaaagagcatttattgatccattagggttaccagacagacctttttataggcatgtcatctatgctccaagcagccacaacaagtatgcaggggagtcattcccaggaatttatgatgctctgtttgatatcgaaagcaaagtggacccttcccaggcctggggagaagtgaagagacagatttctattgcaaccttcacagtgcaagcagctgcagagactttgagtgaagtggcc

SEQ ID NO: 182 - protein, 940 aa, Artificial Sequence
  mIgG Fc-cyno PSMA ECD Amino Acid Sequence
SSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLAAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGSSSEATNITPKHNMKAFLDELKAENIKKFLHNFTQIPHLAGTEQNFQLAKQIQSQWKEFGLDSVELTHYDVLLSYPNKTHPNYISIINEDGNEIFNTSLFEPPPAGYENVSDIVPPFSAFSPQGMPEGDLVYVNYARTEDFFKLERDMKINCSGKIVIARYGKVFRGNKVKNAQLAGATGVILYSDPADYFAPGVKSYPDGWNLPGGGVQRGNILNLNGAGDPLTPGYPANEYAYRRGMAEAVGLPSIPVHPIGYYDAQKLLEKMGGSASPDSSWRGSLKVPYNVGPGFTGNFSTQKVKMHIHSTSEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVYNLTKELESPDEGFEGKSLYESWTKKSPSPEFSGMPRISKLGSGNDFEVFFQRLGIASGRARYTKNWETNKFSSYPLYHSVYETYELVEKFYDPMFKYHLTVAQVRGGMVFELANSVVLPFDCRDYAVVLRKYADKIYNISMKHPQEMKTYSVSFDSLFSAVKNFTEIASKFSERLRDFDKSNPILLRMMNDQLMFLERAFIDPLGLPDRPFYRHVIYAPSSHNKYAGESFPGIYDALFDIESKVDPSQAWGEVKRQISIATFTVQAAAETLSEVA

SEQ ID NO: 183 - protein, 119 aa, Artificial Sequence
  Tsc189VH Amino Acid Sequence
QVQLVQSGAEVKKPGASVKVSCKASGYTFTDYYMHWVRQAPGQGLEWMGYFNPYNDYTRYTRDTSSSTAYTRDTSSSTAYMELSSLRSDDTAVYYCARSDGYYDAMDYWGQGTTVTVSS

SEQ ID NO: 184 - protein, 119 aa, Artificial Sequence
  PSMA01019VH Amino Acid Sequence
EVQLLESGGGLVQPGGSLRLSCAASGYTFTDYYIHWVRQAPGKGLEWIGSFNPYNDYTSYADSVKGRATLSVDKSKNTAYLQMNSLRAEDTAVYYCARSDGYYDAMDYWGQGTLVTVSS

SEQ ID NO: 185 - protein, 119 aa, Artificial Sequence; identical to SEQ ID NO: 184
  PSMA01020VH Amino Acid Sequence

SEQ ID NO: 186 - protein, 119 aa, Artificial Sequence; identical to SEQ ID NO: 114
  PSMA01021VH Amino Acid Sequence

SEQ ID NO: 187 - protein, 119 aa, Artificial Sequence; identical to SEQ ID NO: 82
  PSMA01022VH Amino Acid Sequence

SEQ ID NO: 188 - protein, 119 aa, Artificial Sequence; identical to SEQ ID NO: 82
  PSMA01023VH Amino Acid Sequence

SEQ ID NO: 189 - protein, 119 aa, Artificial Sequence
  PSMA01024VH Amino Acid Sequence
QVQLVQSGAEVKKPGASVKVSCKASGYTFTDYYMHWVRQAPGQGLEWMGIFNPYNDYTSYAQKFQGRVTMTRDTSTSTVYMELSSLRSEDTAVYYCARSDGYYDAMDYWGQGTTVTVSS

SEQ ID NO: 190 - protein, 119 aa, Artificial Sequence; identical to SEQ ID NO: 189
  PSMA01025VH Amino Acid Sequence

SEQ ID NO: 191 - protein, 119 aa, Artificial Sequence; identical to SEQ ID NO: 82
  PSMA01071VH Amino Acid Sequence

SEQ ID NO: 192 - protein, 118 aa, Artificial Sequence
  IGVH1-46+J6 Amino Acid Sequence
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARYYYYYGMDVWGQGTTVTVSS

SEQ ID NO: 193 - protein, 107 aa, Artificial Sequence
  Tsc189VL Amino Acid Sequence
DVQITQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIK

SEQ ID NO: 194 - protein, 107 aa, Artificial Sequence
  PSMA01019VL Amino Acid Sequence
DIQMTQSPSTLSASVGDRVTITCRASKSISKYLAWYQQKPGKAPKLLIHSGSSLESGVPSRFSGSGSGTEFTLTISSLQPDDFATYYCQQHIEYPWTFGQGTKVEIK

SEQ ID NO: 195 - protein, 107 aa, Artificial Sequence; identical to SEQ ID NO: 193
  PSMA01020VL Amino Acid Sequence

SEQ ID NO: 196 - protein, 107 aa, Artificial Sequence; identical to SEQ ID NO: 193
  PSMA01021VL Amino Acid Sequence

SEQ ID NO: 197 - protein, 107 aa, Artificial Sequence; identical to SEQ ID NO: 194
  PSMA01022VL Amino Acid Sequence

SEQ ID NO: 198 - protein, 107 aa, Artificial Sequence; identical to SEQ ID NO: 193
  PSMA01023VL Amino Acid Sequence

SEQ ID NO: 199 - protein, 107 aa, Artificial Sequence; identical to SEQ ID NO: 194
  PSMA01024VL Amino Acid Sequence

SEQ ID NO: 200 - protein, 107 aa, Artificial Sequence; identical to SEQ ID NO: 84
  PSMA01025VL Amino Acid Sequence

SEQ ID NO: 201 - protein, 107 aa, Artificial Sequence
  PSMA01071VL Amino Acid Sequence
DIQMTQSPSTLSASVGDRVTITCRASQSISSWLAWYQQKPGKAPKLLIYDASSLESGVPSRFSGSGSGTEFTLTISSLQPDDFATYYCQQYNSYSWTFGQGTKVEIK

SEQ ID NO: 202 - protein, 107 aa, Artificial Sequence; identical to SEQ ID NO: 201
  IGKV1-5+J1 Amino Acid Sequence

SEQ ID NO: 203 - protein, 121 aa, Artificial Sequence
  CRIS7VH Amino Acid Sequence
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSS

SEQ ID NO: 204 - protein, 113 aa, Artificial Sequence
  VH1-46+J4 Amino Acid Sequence
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARYFDYWGQGTLVTVSS

SEQ ID NO: 205 - protein, 106 aa, Artificial Sequence
  CRIS7VL Amino Acid Sequence
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQIT

SEQ ID NO: 206 - protein, 107 aa, Artificial Sequence
  VK1-39+J1 Amino Acid Sequence
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIK

(19) United States
(12) Patent Application Publication
(10) Pub. No.: US 2024/0301086 A1
(43) Pub. Date: Sep. 12, 2024

(54) TUMOR-ASSOCIATED ANTIGENS AND CD3-BINDING PROTEINS, RELATED COMPOSITIONS, AND METHODS

(71) Applicant: APTEVO RESEARCH AND DEVELOPMENT LLC, Seattle, WA (US)

(72) Inventors: Peter PAVLIK, Seattle, WA (US); Jane GROSS, Seattle, WA (US); David Leonard BIENVENUE, Seattle, WA (US); Gabriela HERNANDEZ-HOYOS, Seattle, WA (US); Lynda MISHER, Seattle, WA (US)

(21) Appl. No.: 18/255,342

(22) PCT Filed: Dec. 1, 2021
(86) PCT No.: PCT/US2021/061486

§ 371 Date: May 31, 2023

Related U.S. Application Data
(60) Provisional application No. 63/120,154, filed on Dec. 1, 2020.
(60) Provisional application No. 63/129,372, filed on Dec. 22, 2020.
(60) Provisional application No. 63/166,394, filed on Mar. 26, 2021.

(51) Int. Cl. C07K 16/30; A61K 39/00; A61P 35/00; C07K 16/28

(52) U.S. Cl. CPC C07K 16/3069; A61P 35/00; C07K 16/2809; A61K 39/00; A61K 2039/505; C07K 2317/24; C07K 2317/31; C07K 2317/35; C07K 2317/52; C07K 2317/565; C07K 2317/622; C07K 2317/71; C07K 2317/73; C07K 2317/92

(57) ABSTRACT

The present disclosure relates to antibodies that specifically bind to a tumor-associated antigen (TAA) such as PSMA and/or CD3, including bispecific antibodies that bind to a TAA (e.g., PSMA) and CD3, and compositions comprising the same. These antibodies are useful for enhancing immune responses and for the treatment of disorders, including solid tumor cancers, for example, by increasing tumor localization.

### CROSS REFERENCE TO RELATED APPLICATIONS

This application claims the benefit of U.S. Provisional Application No. 63/120,154, filed Dec. 1, 2020, U.S. Provisional Application No. 63/129,372, filed Dec. 22, 2020, and U.S. Provisional Application No. 63/166,394, filed Mar. 26, 2021, each of which is herein incorporated by reference in its entirety.

### REFERENCE TO A SEQUENCE LISTING SUBMITTED ELECTRONICALLY VIA EFS-WEB

The content of the electronically submitted sequence listing (Name: 4897_005PC03_Seglisting_ST25.txt; Size: 405,168 bytes; and Date of Creation: Dec. 1, 2021) is herein incorporated by reference in its entirety.

### FIELD OF THE DISCLOSURE

The present disclosure relates to antibodies that specifically bind to a tumor-associated antigen (TAA) (e.g., PSMA, HER2, and BCMA) and/or CD3, including bispecific antibodies that bind to a TAA (e.g., PSMA, HER2, and BCMA) and CD3, and compositions comprising the same. These antibodies are useful for enhancing immune responses and for the treatment of disorders, including solid tumor cancers.

### BACKGROUND

Targeting the T cell receptor (TCR) complex on human T-cells with anti-CD3 monoclonal antibodies has been used or suggested for treatment of autoimmune disease and related disorders such as in the treatment of organ allograft rejection. Mouse monoclonal antibodies specific for human CD3, such as OKT3 (Kung et al. (1979) Science 206: 347-9), were the first generation of such treatments. Although OKT3 has strong immunosuppressive potency, its clinical use was hampered by serious side effects linked to its immunogenic and mitogenic potentials (Chatenoud (2003) Nature Reviews 3:123-132). It induced an antiglobulin response, promoting its own rapid clearance and neutralization (Chatenoud et al. (1982) Eur. J. Immunol. 137:830-8). In addition, OKT3 induced T-cell proliferation and cytokine production in vitro and led to a large scale release of cytokine in vivo (Hirsch et al. (1989) J. Immunol 142: 737-43, 1989). The cytokine release (also referred to as “cytokine storm”) in turn led to a “flu-like” syndrome, characterized by fever, chills, headaches, nausea, vomiting, diarrhea, respiratory distress, septic meningitis and hypotension (Chatenoud, 2003). Such serious side effects limited the more widespread use of OKT3 in transplantation as well as the extension of its use to other clinical fields such as autoimmunity. Id.

To reduce the side effects of the anti-CD3 monoclonal antibodies, a new generation of genetically engineered anti-CD3 monoclonal antibodies had been developed not only by grafting complementarity-determining regions (CDRs) of murine anti-CD3 monoclonal antibodies into human IgG sequences, but also by introducing non-FcR-binding mutations into the Fc to reduce occurrence of cytokine storm (Cole et al. (1999) Transplantation 68: 563; Cole et al. (1997) J. Immunol. 159: 3613). See also PCT Publication No. WO2010/042904, which is herein incorporated by reference in its entirety. Despite advances in the development of anti-CD3 antibodies and bispecific antibodies, cytokine release syndrome remains a key concern in the development of therapeutics that engage CD3.

In addition to monospecific therapeutics that target CD3, multispecific polypeptides that bind selectively to T-cells and tumor cells could offer a mechanism to redirect T-cell cytotoxicity towards the tumor cells and treatment of cancer. One problem, however, to designing a bispecific or multispecific T-cell-recruiting antibody has been to maintain specificity while simultaneously overriding the regulation of T-cell activation by multiple regulatory pathways. Additionally, because CD3 is present in blood lymphocytes there is a need to create an anti-CD3 monospecific or multispecific molecule that will not bind only to CD3 in lymphocytes, but will reach the solid tumor and bind CD3 proximal to the solid tumor.

Thus, bispecific antibodies that bind to a tumor associated antigen (TAA) and CD3 have had difficulties achieving efficacy treating solid tumors in the clinic. It is hypothesized that the difficulty may be caused by the CD3-binding domain of the bispecific antibody having high binding affinity for CD3. As a result of this affinity, most of the bispecific antibody binds to CD3 on circulating T cells in blood when administered to patients. This could result in insufficient amounts of the bispecific antibody reaching a solid tumor.

Bispecific constructs that target CD3 in combination with the TAA Prostate-specific Membrane Antigen (PSMA) have been developed. PSMA is also known as glutamate carboxypeptidase II and N-acetylated alpha-linked acidic dipeptidase 1. It is a dimeric type II transmembrane glycoprotein belonging to the M28 peptidase family encoded by the gene FOLH1 (folate hydrolase 1). The protein acts as a glutamate carboxypeptidase on different alternative substrates, including the nutrient folate and the neuropeptide N-acetyl-l-aspartyl-l-glutamate and is expressed in a number of tissues such as the prostate, and to a lesser extent, the small intestine, central and peripheral nervous system and kidney. The gene encoding PSMA is alternatively spliced to produce at least three variants. A mutation in this gene may be associated with impaired intestinal absorption of dietary folates, resulting in low blood folate levels and consequent hyperhomocysteinemia. Expression of this protein in the brain may be involved in a number of pathological conditions associated with glutamate excitotoxicity.

PSMA is a well-established, highly restricted prostate-cancer-related cell membrane antigen. In prostate cancer cells, PSMA is expressed 1000-fold higher than on normal prostate epithelium (Su et al., Cancer Res. 1995 44:1441-1443). Expression of PSMA increases with prostate cancer progression and is highest in metastatic disease, hormone refractory cases, and higher-grade lesions (Israeli et al., Cancer Res. 1994, 54:1807-1811; Wright et al., Urologic Oncology: Seminars and Original Investigations 1995 1:18-28; Wright et al., Urology 1996 48:326-332; Sweat et al., Urology 1998 52:637-A6A). Additionally, PSMA is abundantly expressed on the neovasculature of a variety of other solid tumors, including bladder, pancreas, melanoma, lung and kidney cancers, but not on normal neovasculature (Chang et al., Urology 2001 57:801-805; Divgi et al., Clin. Cancer Res. 1998 4:2729-3279).

PSMA has been shown to be an important target for immunological approaches such as vaccines or directed therapy with monoclonal antibodies. Unlike other prostate-restricted molecules that are secretory proteins (PSA, prostatic acid phosphatase), PSMA is an integral cell-surface membrane protein that is not secreted, which makes it an ideal target for antibody therapy. PROSTASCINT® (capromab pendetide) is an ¹¹¹In-labelled anti-PSMA murine monoclonal antibody approved by the FDA for imaging and staging of newly diagnosed and recurrent prostate cancer patients (Hinkle et al., Cancer 1998, 83:739-747). However, capromab binds to an intracellular epitope of PSMA, requiring internalization or exposure of the internal domain of PSMA, therefore preferentially binding apoptotic or necrosing cells (Troyer et al., Urologic Oncology: Seminars and Original Investigations 1995 1:29-37; Troyer et al., Prostate 1997 30:232-242). As a result, capromab may not be of therapeutic benefit (Liu et al., Cancer Res. 1997 57:3629-3634).

Other monoclonal antibodies that target the external domain of PSMA have been developed (e.g., J591, J415, J533, and E99) (Liu et al., Cancer Res. 1997 57:3629-3634). However, evidence suggests that PSMA may act as a receptor mediating the internalization of a putative ligand. PSMA undergoes internalization constitutively, and PSMA-specific antibodies can induce and/or increase the rate of internalization, which then causes the antibodies to accumulate in the endosomes (Liu et al., Cancer Res. 1998 58:4055-4060). While PSMA-specific internalizing antibodies may aid in the development of therapeutics to target the delivery of toxins, drugs, or radioisotopes to the interior of prostate cancer cells (Tagawa et al., Cancer 2010 116(S4):1075), PSMA-specific antibodies utilizing native or engineered effector mechanisms (e.g., antibody-dependent cell-mediated cytotoxicity (ADCC), complement-dependent cytotoxicity (CDC), antibody-dependent cell-mediated phagocytosis (ADCP), or re-directed T-cell cytotoxicity (RTCC)) have the potential to be problematic because the PSMA-specific antibody may be internalized before it is recognized by effector cells.

Thus, a need remains for TAA×CD3 bispecific antibodies (e.g., PSMA×CD3 bispecific antibodies) to be able to effectively treat solid tumor cancers, including prostate cancer, and to do so without eliciting harmful systemic cytokine release in a patient.

### SUMMARY

Provided herein are antibodies that bind to CD3 and a tumor associated antigen (TAA) such as PSMA. Such antibodies can be “detuned” to have reduced or low binding affinity for CD3 while maintaining strong binding affinity for the TAA. Such detuned antibodies are designed to retain sufficient binding affinity to CD3 to induce CD8 T cell activation and proliferation. The detuned antibodies provided herein have the benefit of potent killing of solid tumor cells and low cytokine release as compared to other anti-CD3 based therapeutics.

Reduced CD3-binding affinity in a bispecific antibody can be achieved as explained herein, e.g., by manipulating the sequence of the CD3-binding domain, by placing a CD3-binding domain on the C-terminus of the bispecific antibody (for instance, by linkage to the C-terminus of an immunoglobulin constant region), and/or by making the bispecific antibody monovalent for CD3. For instance, provided herein are antibodies that are designed to be bivalent for a TAA and monovalent for a CD3. Additionally, the TAA×CD3 antibodies provided herein can comprise a modified Fc region which prevents or reduces CDC and/or ADCC activity.

Reducing the binding affinity of the CD3-binding domain can reduce the amount of antibody bound by circulating T cells in the blood and allow the TAA×CD3 bispecific antibodies to reach the solid tumor, where T cell cytotoxicity can occur at the tumor. Such TAA×CD3 antibodies can exhibit improved killing of solid tumor cells that express the TAA as compared to a control TAA×CD3 antibody that does not have reduced binding affinity to CD3.

The TAA×CD3 antibodies provided herein elicit reduced levels of inflammatory cytokines (e.g., IFN-γ, IL-2, TNF-α, and/or IL-6) as compared to TAA×CD3 antibodies with high affinity to CD3. The TAA×CD3 antibodies provided herein elicit reduced levels of inflammatory cytokines (e.g., Granzyme B, IL-10 and/or GM-CSF) as compared to TAA×CD3 antibodies with high affinity to CD3. The TAA×CD3 bispecific antibodies disclosed herein cause no detectable levels of cytokine release or reduced levels of cytokine release in a patient. Reducing the binding affinity of the CD3-binding domain to CD3 of the TAA×CD3 antibodies can reduce the likelihood that the patient treated with a pharmaceutical composition comprising the TAA×CD3 will suffer from cytokine release syndrome.

The TAA (e.g., PSMA) binding domain can have greater binding strength, binding potency, and/or avidity to PSMA than the CD3 binding domain has to CD3. The CD3 binding domain can have reduced binding strength, binding potency, and/or avidity to CD3 as compared to TSC266 and/or PSMA01110 in a Jurkat cell assay.

In certain aspects, a bispecific antibody provided herein comprises (a) a first polypeptide from N-terminus to C-terminus comprising (i) a first single chain variable fragment (scFv) that binds to a tumor-associated antigen (TAA), (ii) an immunoglobulin constant region, and (iii) an scFv that binds to CD3; and (b) a second polypeptide from N-terminus to C-terminus comprising (i) a second scFv that binds to the TAA, and (ii) an immunoglobulin constant region,
wherein the bispecific antibody does not contain a second CD3-binding domain.

In certain aspects, the TAA is PSMA, HER2, or BCMA. In certain aspects, the TAA is PSMA.

In certain aspects, the first polypeptide and the second polypeptide are joined by at least one disulfide bond.

In certain aspects, the first scFv that binds to the TAA is in the VH-VL orientation. In certain aspects, the first scFv that binds to the TAA is in the VL-VH orientation.

In certain aspects, the second scFv that binds to the TAA is in the VH-VL orientation. In certain aspects, the second scFv that binds to the TAA is in the VL-VH orientation.

In certain aspects, the first scFv that binds to the TAA and the second scFv that binds to the TAA are the same.

In certain aspects, the scFv that binds to CD3 is in the VH-VL orientation. In certain aspects, the scFv that binds to CD3 is in the VL-VH orientation.

In certain aspects, the immunoglobulin constant region in the first polypeptide comprises a knob mutation and/or the immunoglobulin constant region in the second polypeptide comprises a hole mutation. In certain aspects, the immunoglobulin constant region in the first polypeptide comprises a hole mutation and/or the immunoglobulin constant region in the second polypeptide comprises a knob mutation.

In certain aspects, the immunoglobulin constant region comprising a knob mutation comprises the amino acid sequence of SEQ ID NO:66 and/or the immunoglobulin constant region comprising a hole mutation comprises the amino acid sequence of SEQ ID NO:68.

In certain aspects, the immunoglobulin constant region comprises one, two, three, four, five or more amino acid substitutions and/or deletions compared to a wild-type immunoglobulin constant region to prevent binding of FcγR1 and/or FcγRIIIb. In certain aspects, the immunoglobulin constant region comprises one, two, three, four, five, or more amino acid substitutions and/or deletions compared to a wild-type immunoglobulin constant region to prevent or reduce CDC activity.

In certain aspects, the immunoglobulin constant region comprises a IgG1 CH2 domain comprising the substitutions E233P, L234A, L235A, G237A, and K322A and a deletion of G236 according to the EU numbering system.

In certain aspects, the immunoglobulin constant region comprises an immunoglobulin CH2 and CH3 domains of IgG1.

In certain aspects, the bispecific antibody does not contain a CH1 domain.

In certain aspects, the bispecific antibody comprises a first scFv that binds to the TAA, the scFv that binds to CD3, and/or the second scFv that binds to the TAA comprises a glycine-serine linker.

In certain aspects, the first scFv that binds to the TAA, the scFv that binds to CD3, and/or the second scFv that binds to the TAA comprises a glycine-serine linker comprising the amino acid sequence (Gly₄Ser)ₙ, wherein n=1-5. In certain aspects, n=4.

In certain aspects, first polypeptide and/or the second polypeptide further comprises at least one linker between an scFv and an immunoglobulin constant domain. In certain aspects, the linker that comprises a hinge region. In certain aspects, the hinge is an IgG1 hinge region. In certain aspects, the hinge comprises the amino acid sequence of SEQ ID NO:156.

In certain aspects, the first scFv that binds to PSMA and/or the second scFv that binds to PSMA is capable of binding to cynomolgus PSMA. In certain aspects, the first scFv that binds to PSMA and/or the second scFv that binds to cynomolgus PSMA has an EC50 of no more than 5-times greater than the EC50 for binding to human PSMA.

In certain aspects, the bispecific antibody is capable of binding to the TAA and CD3 simultaneously.

In certain aspects, the scFv that binds to CD3 binds to CD3ε.

In certain aspects, the first scFv that binds to PSMA comprises a variable heavy (VH) complementarity-determining region (CDR)1, VH CDR2, and VH CDR3 comprising the amino acid sequences of SEQ ID NOs: 70, 72, and 74, respectively, and comprises a variable light (VL) CDR1, VL CDR2, and VL CDR3 comprising the amino acid sequences of SEQ ID NOs: 76, 78, and 80, respectively.

In certain aspects, the first scFv that binds to PSMA comprises a VH domain comprising the amino acid sequence of SEQ ID NO: 82 and comprises a VL domain comprising the amino acid sequence of SEQ ID NO: 84.

In certain aspects, the first scFv that binds to PSMA comprises the amino acid sequence of SEQ ID NO: 86.

In certain aspects, the scFv that binds to CD3 comprises a VH CDR1, VH CDR2, and VH CDR3 comprising the amino acid sequences of SEQ ID NOs: 88, 90, and 92, respectively, and comprises a VL CDR1, VL CDR2, and VL CDR3 comprising the amino acid sequences of SEQ ID NOs: 94, 96, and 98, respectively.

In certain aspects, the scFv that binds to CD3 comprises a VH domain comprising the amino acid sequence of SEQ ID NO: 100 and comprises a VL domain comprising the amino acid sequence of SEQ ID NO: 102.

In certain aspects, the scFv that binds to CD3 comprises the amino acid sequence of SEQ ID NO: 104 or 110.

In certain aspects, the second scFv that binds to PSMA comprises a VH CDR1, VH CDR2, and VH CDR3 comprising the amino acid sequences of SEQ ID NOs: 70, 72, and 74, respectively, and comprises a VL CDR1, VL CDR2, and VL CDR3 comprising the amino acid sequences of SEQ ID NOs: 76, 78, and 80, respectively.

In certain aspects, the second scFv that binds to PSMA comprises a VH domain comprising the amino acid sequence of SEQ ID NO: 82 and comprises a VL domain comprising the amino acid sequence of SEQ ID NO: 84.

In certain aspects, the second scFv that binds to PSMA comprises the amino acid sequence of SEQ ID NO: 86.

In certain aspects, the first polypeptide comprises the amino acid sequence of SEQ ID NO: 106, 178, or 112.

In certain aspects, the second polypeptide comprises the amino acid sequence of SEQ ID NO:108.

In certain aspects, the bispecific antibody is capable of promoting expansion of CD8+ T cells and/or CD4+ T cells. In certain aspects, the bispecific antibody is capable of activating CD8+ T cells and/or CD4+ T cells. In certain aspects, the bispecific antibody is capable of increasing central memory T cells (TCM) and/or effector memory T cells (TEM). In certain aspects, the bispecific antibody is capable of decreasing naïve and/or terminally differentiated T cells (Teff).

In certain aspects, the bispecific antibody is capable of decreasing secretion of IFN-γ, IL-2, IL-6, and/or TNF-α. In certain aspects, the bispecific antibody is capable of decreasing secretion of Granzyme B, IL-10, and/or GM-CSF. In certain aspects, the bispecific antibody is capable of increasing signaling of NFκB, NFAT, and/or ERK signaling pathways.

In certain aspects, an antibody or antigen-binding fragment thereof provided herein comprises a PSMA-binding domain, wherein the PSMA-binding domain comprises a VH and a VL, wherein the VH comprises the amino acid sequence of SEQ ID NO:82. In certain aspects, an antibody or antigen-binding fragment thereof provided herein comprises a PSMA-binding domain, wherein the PSMA-binding domain comprises a VH and a VL, wherein the VL comprises the amino acid sequence of SEQ ID NO:84. In certain aspects, the VH comprises the amino acid sequence of SEQ ID NO:82, and the VL comprises the amino acid sequence of SEQ ID NO:84.

In certain aspects, an antibody or antigen-binding fragment thereof provided herein comprises a CD3 antigen-binding domain, wherein the CD3 antigen-binding domain comprises a VH and a VL, wherein the VH comprises the amino acid sequence of SEQ ID NO: 100. In certain aspects, an antibody or antigen-binding fragment thereof provided herein comprises a CD3 antigen-binding domain, wherein the CD3 antigen-binding domain comprises a VH and a VL, wherein the VL comprises the amino acid sequence of SEQ ID NO:102. In certain aspects, the VH comprises an amino acid sequence of SEQ ID NO: 100, and the VL comprises an amino acid sequence of SEQ ID NO: 102.

In certain aspects, an antibody or antigen-binding fragment thereof provided herein is an IgG antibody, optionally wherein the IgG antibody is an IgG1 antibody.

In certain aspects, an antibody or antigen-binding fragment thereof provided herein, further comprises a heavy chain constant region and a light chain constant region, optionally wherein the heavy chain constant region is a human IgG1 heavy chain constant region, and/or optionally wherein the light chain constant region is a human IgGκ light chain constant region.

In certain aspects, an antibody or antigen-binding fragment thereof provided herein comprises an a Fab, Fab′, F(ab′)2, scFv, disulfide linked Fv, or scFv-Fc.

In certain aspects, an antibody or antigen-binding fragment thereof provided herein comprises a scFv.

In certain aspects, an antibody or antigen-binding fragment thereof provided herein comprises the amino acid sequence of SEQ ID NO:86.

In certain aspects, an antibody or antigen-binding fragment thereof provided herein comprises the amino acid sequence of SEQ ID NO: 104.

In certain aspects, an antibody or antigen-binding fragment thereof provided herein is bispecific.

In certain aspects, the bispecific antibody or fragment comprises an antigen-binding domain that specifically binds PSMA and an antigen-binding domain that specifically binds CD3.

In certain aspects, (i) the antigen-binding domain that specifically binds PSMA comprises the amino acid sequences of SEQ ID NOs:82 and 84, and/or (ii) the antigen-binding domain that specifically binds CD3 comprises the amino acid sequence of SEQ ID NOs:100 and 102.

In certain aspects, the antigen-binding domain that specifically binds PSMA comprises a VH and a VL in the VH-VL orientation. In certain aspects, the antigen-binding domain that specifically binds PSMA comprises a VH and a VL in the VL-VH orientation.

In certain aspects, the antigen-binding domain that specifically binds CD3 comprises a VH and a VL in the VH-VL orientation. In certain aspects, the antigen-binding domain that specifically binds CD3 comprises a VH and a VL in the VL-VH orientation.

In certain aspects, the antigen-binding domain that specifically binds PSMA comprises a scFv that comprises the amino acid sequence of SEQ ID NO:86.

In certain aspects, the antigen-binding domain that specifically binds to CD3 comprises a scFv that comprises the amino acid sequence of SEQ ID NO:104.

In certain aspects, an antibody or antigen-binding fragment thereof provided herein is monovalent for CD3. In certain aspects, an antibody or antigen-binding fragment thereof provided herein is bivalent for CD3.

In certain aspects, an antibody or antigen-binding fragment thereof provided herein is bivalent for PSMA. In certain aspects, an antibody or antigen-binding fragment thereof provided herein is monovalent for PSMA.

In certain aspects, the antibody or fragment comprises a polypeptide comprising, in order from amino-terminus to carboxyl-terminus, (i) a first single chain variable fragment (scFv), (ii) a linker, optionally wherein the linker is a hinge region, (iii) an immunoglobulin constant region, and (iv) a second scFv, wherein (a) the first scFv comprises a human CD3 antigen-binding domain, and the second scFv comprises a human PSMA antigen-binding domain or (b) the first scFv comprises a human PSMA antigen-binding domain and the second scFv comprises a human CD3 antigen-binding domain.

In certain aspects, the antibody or fragment comprises a knob mutation and a hole mutation.

In certain aspects, a bispecific antibody provided herein comprises (a) a first polypeptide from N-terminus to C-terminus comprising (i) a first single chain variable fragment (scFv) that binds to PSMA comprising the amino acid sequence of SEQ ID NO:86, (ii) a linker comprising the amino acid sequence of SEQ ID NO:156, (iii) an immunoglobulin constant region comprising the amino acid sequence of SEQ ID NO:66, and (iv) an scFv that binds to CD3 comprising the amino acid sequence of SEQ ID NO: 104; and (b) a second polypeptide from N-terminus to C-terminus comprising (i) a second scFv that binds to PSMA comprising the amino acid sequence of SEQ ID NO:86, (ii) a linker comprising the amino acid sequence of SEQ ID NO: 156, and (iii) an immunoglobulin constant region comprising the amino acid sequence of SEQ ID NO:68, wherein the bispecific antibody does not contain a second CD3-binding domain.

In certain aspects, a bispecific antibody provided herein comprises a first polypeptide comprising the amino acid sequence of SEQ ID NO: 106, 178, or 112 and a second polypeptide comprising the amino acid sequence of SEQ ID NO: 108, wherein the bispecific antibody only contains one CD3-binding domain.

In certain aspects, a bispecific antibody provided herein consists of a first polypeptide comprising the amino acid sequence of SEQ ID NO:106, 178, or 112 and a second polypeptide comprising the amino acid sequence of SEQ ID NO: 108.

In certain aspects, a polynucleotide provided herein encodes a bispecific antibody provided herein.

In certain aspects, a vector or expression vector provided herein comprises a polynucleotide encoding a bispecific antibody provided herein.

In certain aspects, a host cell provided herein comprises a polynucleotide encoding a bispecific antibody or vector encoding a bispecific antibody provided herein.

In certain aspects, a host cell provided herein comprises a combination of polynucleotides that encode a bispecific antibody provided herein. In certain aspects, the polynucleotides are encoded on a single vector. In certain aspects, the polynucleotides are encoded on multiple vectors.

In certain aspects, the host cell is selected from the group consisting of a CHO, HEK293, or COS cell.

In certain aspects, a method of producing a bispecific antibody that specifically binds to human PSMA and human CD3 as provided herein comprises culturing the host cell so that the antibody is produced, and optionally further comprises recovering the antibody.

In certain aspects, a method for detecting PSMA and CD3 in a sample comprises contacting the sample with a bispecific antibody provided herein, optionally wherein the sample comprises cells.

In certain aspects, a pharmaceutical composition provided herein comprises a bispecific antibody provided herein, and a pharmaceutically acceptable excipient.

In certain aspects, a method for increasing T cell proliferation provided herein comprises contacting a T cell with a bispecific antibody provided herein or a pharmaceutical composition provided herein. In certain aspects, the T cell is a CD4+ T cell. In certain aspects, the T cell is a CD8+ T cell.

In certain aspects, the cell is in a subject, and the contacting comprises administering the antibody or the pharmaceutical composition to the subject.

In certain aspects, a method for enhancing an immune response in a subject comprises administering to the subject an effective amount of a bispecific antibody provided herein or a pharmaceutical composition provided herein.

In certain aspects, a method for inducing redirected T-cell cytotoxicity (RTCC) against a cell expressing prostate-specific membrane antigen (PSMA) comprises contacting the PSMA-expressing cell with a bispecific antibody provided herein or a composition provided herein, wherein the contacting is under conditions whereby RTCC against the PSMA-expressing cell is induced.

In certain aspects, a method for treating a disorder characterized by overexpression of prostate-specific membrane antigen (PSMA) in a subject comprises administering to the subject a therapeutically effective amount of a bispecific antibody provided herein or a composition provided herein.

In certain aspects, a bispecific antibody provided herein or a composition provided herein induces redirected T-cell cytotoxicity (RTCC) in the subject.

In certain aspects, the bispecific antibody promotes expansion or proliferation of CD8+ and/or CD4+ T cells. In certain aspects, the bispecific antibody activates CD8+ and/or CD4+ T cells. In certain aspects, the bispecific antibody increases central memory T cells (TCM) and/or effector memory T cells (TEM). In certain aspects, the bispecific antibody decreases naïve and/or terminally differentiated T cells (Teff).

In certain aspects, the bispecific antibody decreases secretion of IFN-γ, IL-2, IL-6, and/or TNF-α. In certain aspects, the bispecific antibody is capable of decreasing secretion of Granzyme B, IL-10, and/or GM-CSF. In certain aspects, the bispecific antibody increases signaling of NFκB, NFAT, and/or ERK signaling pathways.

In certain aspects, the disorder is a cancer. In certain aspects, the cancer is selected from the group consisting of prostate cancer, PSMA(+) cancer, metastatic prostate cancer, clear cell renal carcinoma, bladder cancer, lung cancer, colorectal cancer, and gastric cancer. In certain aspects, the cancer is prostate cancer. In certain aspects, the prostate cancer is castrate-resistant prostate cancer. In certain aspects, the disorder is a prostate disorder. In certain aspects, the prostate disorder is selected from the group consisting of prostate cancer and benign prostatic hyperplasia.

### BRIEF DESCRIPTION OF THE FIGURES

FIGS. 1A-1G show cartoons depicting structures of various potential bispecific antibody constructs that bind to CD3 and a tumor associated antigen (TAA) such as PSMA. FIGS. 1A-E show examples of different formats of PSMA×CD3 bispecific formats with some that were evaluated using knob (K) and hole (H) mutations in the Fc region to improve heterodimer formation. FIG. 1A shows PSMA VH-VL Fc×CD3 VH-VL Fc. FIG. 1B shows PSMA VH-VL-Fc×PSMA VH-VL-Fc-CD3 VH-VL. FIG. 1C shows PSMA VH-VL-Fc×PSMA VH-VL-Fc-CD3 VL-VH. FIG. 1D shows PSMA VH-VL-Fc-CD3 VL-VH×PSMA VH-VL-Fc-CD3 VL-VH. FIG. 1E shows PSMA VH-VL-Fc-CD3 VH-VL×PSMA VH-VL-Fc-CD3 VH-VL. FIG. 1F shows additional PSMA×CD3 bispecific constructs. FIG. 1G shows TAA×CD3 antibody constructs with anti-TAA domains in the VH-VL orientation (top panel) and with anti-TAA domains in the VL-VH orientation (bottom panel). scFvs could be in VH-VL or VL-VH orientation. In addition to the binding domains being a scFv, the binding domain could be an extracellular domain or cytokine. Knob-In-Hole mutations could be placed on either of the Fc chains. Additional mutations could be incorporated to eliminate or enhance effector function, depending on the desired activity. (See Example 1.)

FIG. 2 shows sequences of 107-1A4 and humanized anti-PSMA-binding domains. VH sequences are shown in the top panel, and VL sequences are in the bottom panel. Differences between individual sequences and human germline sequences are shown. CDRs (IMGT definition) are indicated by brackets. (See Example 4.)

FIG. 3 shows sequences of CRIS-7 and humanized CD3ε-specific binding domains. VH sequences are shown in the top panel, and VL sequences are in the bottom panel. Differences between individual sequences and human germline sequences are shown. CDRs (IMGT definition) are indicated by brackets. (See Example 5.)

FIG. 4 shows a graph depicting levels of human PSMA expression by different cell lines. Receptor quantification was assessed by flow cytometry using a commercial anti-PSMA antibody, and results are reported in antibody bound per cell units (ABC). (See Example 7.)

FIG. 5 shows graphs depicting binding curves of humanized PSMA-binding domain variant constructs PSMA01012, PSMA01019, PSMA01020, PSMA01021, PSMA01023 to PSMA01025 on human and cynomolgus CHOK1SV/PSMA transfectants. Serial dilutions of heterodimer antibody constructs were incubated with transfected target cells and subsequently labelled with SULFO TAG-labeled goat anti-human IgG secondary antibody. Binding was quantified by MSD (Meso Scale Discovery instrument). The y-axis displays the signal in electrochemiluminescence (ECL) units. (See Example 8.)

FIG. 6 shows graphs depicting binding curves of the PSMA-binding domain PSMA01023 in different formats, scFv-Fc or Fc-scFv, and VH-VL vs VL-VH orientations, on C4-2B and 22RV1 tumor cells. Serial dilutions of the antibody constructs were incubated with PSMA (+) tumor cells and subsequently labelled with a fluorescently-conjugated goat anti-human secondary antibody. Binding was quantified by flow cytometry. The y-axis displays the median fluorescence intensity units (MFI median). (See Example 10.)

FIGS. 7A and 7B show binding of anti-tumor antigen (TA)×anti-CD3ε H14, H15 or H16 monovalent or bivalent antibody constructs on Jurkat cells. Serial dilutions of antibody constructs were incubated with Jurkat cells and subsequently labelled with a fluorescently-conjugated goat-α-human Fc secondary antibody. Binding was quantified by flow cytometry. The y-axis displays the median fluorescence intensity units (MFI median). (See Example 11.)

FIGS. 8A and 8B show the results of assays measuring tumor-antigen induced CD4 and CD8 T-cell activation by anti-TA×anti-CD3ε H14, H15 and H16 constructs at 24 hours. Purified human T cells were co-cultured with TA (+) tumor cells in the presence of serial dilutions of the antibody constructs. T-cell activation was quantified by the upregulation of CD25 and CD69 on gated CD4 or CD8 T cells using flow cytometry. Activation is expressed as the percent of CD4 or CD8 T cells expressing CD69 and CD25. (See Example 12.)

FIG. 9 shows the results of assays measuring tumor-antigen induced CD4 and CD8 T-cell proliferation by anti-TA×anti-CD3ε H14 and H16 constructs at 96 hours. Cell Trace Violet labelled human T cells were co-cultured with TA (+) tumor cells in the presence of serial dilutions of antibody constructs. T-cell proliferation was quantified by the dilution of the Cell Trace Violet dye on gated CD4 or CD8 T cells, using flow cytometry. Proliferation is expressed as the percent of CD4 or CD8 T cells that underwent at least once cell division. (See Example 12.)

FIG. 10 shows the results of assays measuring anti-TA×anti-CD3ε constructs H14 and H16 induced redirected cytotoxicity of TA-expressing target cells at 96 hours. Purified human T cells were co-cultured with TA (+) tumor cells in the presence of serial dilutions of the antibody constructs. The fraction of live TA (+) tumor cells was quantified by flow cytometry afterwards on the non-T cell population. Cytotoxicity of TA (+) tumor cells is represented as the loss of viable cells in the cultures (percent of live cells). (See Example 12.)

FIGS. 11A and 11B show graphs depicting binding curves of anti-PSMA×anti-CD3ε constructs in various formats (PSMA01026, PSMA01070, PSMA01071, PSMA01072 and PSMA01086) on (A) C4-2B and (B) Jurkat cells. Serial dilutions of the heterodimer antibody constructs were incubated with the PSMA (+) or CD3 (+) cell lines, C4-2B or Jurkat, respectively, and subsequently labelled with a fluorescently-conjugated goat anti-human Fc secondary antibody. Binding was quantified by flow cytometry. The y-axis displays the median fluorescence intensity units (MFI median). (See Example 13.)

FIGS. 12A and 12B show the results of assays measuring tumor-antigen induced CD4 and CD8 T-cell activation at 24 hrs with anti-PSMA×anti-CD3ε constructs PSMA01026, PSMA01070, PSMA01071, PSMA01072 and PSMA01086. Peripheral blood mononuclear cells (PBMC) were co-cultured with C4-2B (A) or without target cells (B) in the presence of serial dilutions of heterodimer antibody constructs. T-cell activation was quantified by the upregulation of CD25 and CD69 on gated CD4 or CD8 T cells using flow cytometry. Activation is expressed as the percent of CD4 or CD8 T cells expressing CD69 and CD25. (See Example 14.)

FIG. 13 shows the results of assays measuring cytokine secretion induced by of anti-PSMA×anti-CD3ε constructs PSMA01026, PSMA01070, PSMA01071, PSMA01072 and PSMA01086 from PBMC cultures, in the presence of C4-2B target cells at 24 hours. PBMC were co-cultured with C4-2B target cells in the presence of serial dilutions of the heterodimer antibody constructs. Secretion of cytokines in the culture supernatants was assessed using multiplexed-based assays. Cytokine levels are expressed in pg/mL units. (See Example 14.)

FIG. 14 shows the results of assays measuring tumor-antigen induced CD4 and CD8 T-cell proliferation by anti-PSMA×anti-CD3ε constructs PSMA01026, PSMA01070, PSMA01071, PSMA01072 and PSMA01086 in the presence of C4-2B target cells at 96 hours. CTV-labelled human PBMC were co-cultured with C4-2B cells in the presence of serial dilutions of the heterodimer antibody constructs. T-cell proliferation was quantified by the dilution of CTV on gated CD4 or CD8 T cells, using flow cytometry. Proliferation is expressed as the percent of CD4 or CD8 T cells that underwent at least once cell division. (See Example 14.)

FIG. 15 shows the results of assays measuring T-cell redirected cytotoxicity of PSMA-expressing target cells by of anti-PSMA×anti-CD3ε constructs PSMA01026, PSMA01070, PSMA01071, PSMA01072, PSMA01086 and control CD3 construct TRI149 at 72 and 96 hours. PBMC were co-cultured with C4-2B-luciferase (C4-2B-luc) cells in the presence of serial dilutions of the antibody constructs. The fraction of live C4-2B cells was quantified by bioluminescence after addition of luciferin substrate. Cytotoxicity of C4-2B-luc cells is expressed as the loss percent of live (luciferase expressing) cells in the cultures and is represented in RLU (relative light units). (See Example 14.)

FIG. 16 shows the tumor growth as measured by bioluminescence levels over time in a subcutaneous xenograft mouse model of prostate cancer, in animals treated with of anti-PSMA×anti-CD3ε constructs. Two million C4-2B cells mixed with one million human T cells in 50% HC matrigel were injected SC into the right flank of male NOD/scid mice (n=10/group). Treatments were administered by IV injection on days 0, 4 and 8. Mean log 10 Tumor Bioluminescence for each group is plotted±SEM. (See Example 15.)

FIG. 17 shows the tumor incidence as measured by bioluminescence levels over time in a subcutaneous xenograft mouse model of prostate cancer, in animals treated with of anti-PSMA×anti-CD3ε constructs, as described in FIG. 13. Tumor incidence is determined as the percent of animals with detectable bioluminescence per group. (See Example 15.)

FIGS. 18A-18E show graphs depicting binding curves of anti-PSMA×anti-CD3ε constructs in various formats (TSC266, PSMA01107, PSMA01108, and PSMA01110) on (FIGS. 18A and 18C) C4-2B and (FIGS. 18B and 18D) Jurkat cells, and (FIG. 18E) CHO cells overexpressing cynomolgus PSMA (CHO-CynoPSMA). Serial dilutions of bispecific antibody constructs were incubated with the PSMA (+) or CD3 (+) cell lines, C4-2B, Jurkat, or CHO-CynoPSMA, respectively, and subsequently labelled with a fluorescently-conjugated goat-α-human Fc secondary antibody. Binding was quantified by flow cytometry. The y-axis displays the median fluorescence intensity units (MFI median). (See Example 20.)

FIGS. 19A and 19B show the results of assays measuring tumor-antigen induced CD4 and CD8 T-cell activation at 24 hrs with anti-PSMA×anti-CD3ε constructs TSC266, PSMA01107, PSMA01108 and TSC291a, in the presence of (A) C4-2B target cells, or (B) without target cells. FIGS. 19C and 19D show T-cell activation induced by anti-PSMA×anti-CD3ε constructs PSMA01107, PSMA01108, and PSMA0110 in the presence of C4-2B target cells (C) or without target cells (D). FIG. 19E shows summary data at 200 pM of constructs PSMA01107, PSMA01108, and PSMA01110 in the presence of C4-2B target cells. Peripheral blood mononuclear cells (PBMC) were co-cultured with C4-2B or no target cells in the presence of serial dilutions of the bispecific antibody constructs. T-cell activation was quantified by the upregulation of CD25 and CD69 on gated CD4 or CD8 T cells using flow cytometry. Activation is expressed as the percent of CD4 or CD8 T cells expressing CD69 and CD25. (See Example 20.)

FIGS. 20A-E show the results of assays measuring cytokine secretion induced by of anti-PSMA×anti-CD3ε constructs TSC266, PSMA01107, PSMA01108, PSMA01110, and TSC291a from PBMC cultures, in the presence or absence of PSMA-expressing target cells at 24 hours. FIG. 20A shows cytokines response of anti-PSMA×anti-CD3ε constructs TSC266, PSMA01107, PSMA01108, and TSC291a from PBMCs co-cultured with C4-2B target cells. FIGS. 20B and 20C show summary data of constructs PSMA01107 and TSC291a at 200 pM from PBMCs in the presence of C4-2B target cells (FIG. 20B) or in the absence C4-2B target cells (FIG. 20C). FIG. 20D shows summary data of the constructs PSMA01107, PSMA01108, and PSMA01110 at 200 pM from PBMCs in the presence of C4-2B target cells. FIG. 20E shows cytokine responses with a serial dilution curve with the construct PSMA01107 in the presence or absence of C4-2B target cells. PBMC were co-cultured with C4-2B target cells in the presence of serial dilutions of the antibody constructs. Secretion of cytokines in the culture supernatants was assessed using multiplexed-based assays. Cytokine levels are expressed in pg/mL units. (See Example 20.)

FIGS. 21A and 21B show the results of assays measuring tumor-antigen induced CD4 and CD8 T-cell proliferation by of anti-PSMA×anti-CD3ε constructs TSC266, PSMA01107, PSMA01108, TSC291a and control CD3 construct TRI149 in the presence of C4-2B target cells at 96 hours. T-cell activation was quantified by the upregulation of CD25 and CD69 on gated CD4 or CD8 T cells using flow cytometry. (See Example 20.)

FIGS. 22A and 22B show the results of assays measuring tumor-antigen induced CD4 and CD8 T-cell proliferation of anti-PSMA×anti-CD3ε constructs TSC266, PSMA01107, PSMA01108 and PSMA01110 in the presence of C4-2B target cells at 96 hours (FIG. 22A) and summary data at 200 pM (FIG. 22B). (See Example 20.)

FIG. 23 shows the results of assays measuring T-cell redirected cytotoxicity of PSMA-expressing target cells by of anti-PSMA×anti-CD3ε constructs TSC266, PSMA01107 and PSMA01108 at 72 and 96 hours. PBMC were co-cultured with C4-2B-luciferase (C4-2B-luc) cells in the presence of serial dilutions of the antibody constructs. The fraction of live C4-2B cells was quantified by bioluminescence after addition of luciferin substrate. Cytotoxicity of C4-2B-luc cells is expressed as the loss percent of live (luciferase expressing) cells in the cultures and is represented in RLU (relative light units). (See Example 20.)

FIG. 24 shows non-specific binding analysis of the anti-PSMA×anti-CD3ε antibody constructs to various cell lines performed using the Meso Scale Discovery platform. Cell lines included into this experiment were AsPC-1, U937, K562, CHOK1SV and MDA-MB-231. C4-2B prostate cancer and Jurkat cells were used for positive binding to PSMA and CD3 respectively. (See Example 21.)

FIG. 25 shows the mean serum concentrations for anti-PSMA×anti-CD3 constructs in C57BL/6 mice. Mice (n=3 per group) were dosed intravenously with ˜10 μg (0.5 mg/kg) of PSMA01107, PSMA01108, or PSMA01110. Concentration data from one mouse dosed with PSMA01108 (mouse #4) was excluded from mean calculations due to the presence of anti-drug antibodies. (See Example 26.)

FIG. 26 shows the individual serum concentrations for anti-PSMA×anti-CD3 antibody constructs in C57BL/6 mice. Mice (n=3 per group) were dosed intravenously with ˜10 μg (0.5 mg/kg) of PSMA01107, PSMA01108 or PSMA01110. (See Example 26.)

FIG. 27 shows a graph depicting levels of human PSMA surface expression on tumor cell lines. Receptor quantification was assessed by flow cytometry using a commercial anti-PSMA antibody and results are reported in antibody bound per cell units (ABC). (See Example 28.)

FIGS. 28A-28D show graphs depicting binding curves of (A) TSC266, (B) TSC266 (re-scaled), (C) PSMA01107 and (D) PSMA01107 (re-scaled) on various PSMA-expressing cell lines. Anti-PSMA×anti-CD3ε constructs PSMA01108 and PSMA01110 exhibited identical binding profiles as PSMA01107 (data not shown). Serial dilutions of antibody constructs were incubated with the PSMA (+) cell lines, LNCaP, C4-2B MDA-PCa-2b, 22RV1, or DU145, respectively, and subsequently labelled with a fluorescently-conjugated goat-α-human Fc secondary antibody. Binding was quantified by flow cytometry. The y-axis displays the median fluorescence intensity units (MFI median). (See Example 29.)

FIG. 29 shows the results of tumor-antigen induced CD4 T-cell activation at 24 hrs with anti-PSMA×anti-CD3ε constructs TSC266, PSMA01107, PSMA01108, and PSMA01110, in the presence of various PSMA expressing target cell lines. Peripheral blood mononuclear cells (PBMC) were co-cultured with PSMA expressing cells in the presence of serial dilutions of the antibody constructs. T-cell activation was quantified by the upregulation of CD25 and CD69 on gated CD4+ T cells using flow cytometry. (See Example 29.)

FIG. 30 shows the results of tumor-antigen induced CD8+ T-cell activation at 24 hrs with anti-PSMA×anti-CD3ε constructs TSC266, PSMA01107, PSMA01108, and PSMA01110, in the presence of various PSMA expressing target cell lines. Peripheral blood mononuclear cells (PBMC) were co-cultured with PSMA expressing cells in the presence of serial dilutions of the antibody constructs. T-cell activation was quantified by the upregulation of CD25 and CD69 on gated CD8+ T cells using flow cytometry. (See Example 29.)

FIGS. 31A and 31B show the results of assays measuring T-cell redirected cytotoxicity of high PSMA-expressing target cells (FIG. 31A: C4-2B) or low PSMA-expressing target cells (FIG. 31B: MDA-PCa-2b) of anti-PSMA×anti-CD3ε constructs TSC266, PSMA01107, PSMA01108, and PSMA01110 at 72 and 96 hours. PBMC were co-cultured with C4-2B-luciferase (C4-2B-luc) cells in the presence of serial dilutions of the antibody constructs. The fraction of live C4-2B or MDA-PCa-2b cells was quantified by bioluminescence after addition of luciferin substrate. Cytotoxicity of C4-2B-luc cells is represented in RLU (relative light units). (See Example 29.)

FIGS. 32A and 32B show graphs depicting levels of human PSMA surface expression on tumor cell lines by receptor quantification or direct binding by the anti-PSMA×anti-CD3ε construct PSMA01107 (FIG. 32A). Receptor quantification was assessed by flow cytometry using a commercial anti-PSMA antibody and results are reported in antibody bound per cell units, construct binding was assessed by incubating cell lines with PSMA01107 with tumor target cell lines a detecting binding by flow cytometry. FIG. 32B shows the comparison of tumor target cell binding by the anti-PSMA×anti-CD3ε construct PSMA01107 by percent specific lysis of the matched tumor cell lines. (See Example 29.)

FIGS. 33A and 33B show binding curves of serially diluted anti-PSMA×anti-CD3ε constructs (PSMA01107 and PSMA01116) on (A) PSMA-expressing C4-2B and (B) CD3-expressing Jurkat cells. Binding was quantified by flow cytometry. The y-axis displays the median fluorescence intensity units (MFI median). (See Example 30.)

FIG. 34 shows the results of tumor-antigen induced CD4⁺ and CD8⁺ T-cell activation at 24 hrs with serial dilutions of anti-PSMA×anti-CD3ε constructs TSC266, PSMA01107, PSMA01116 and TRI149, in the presence of C4-2B target cells. PBMC activation was quantified by the percent upregulation of CD25 and CD69 on gated CD4⁺ or CD8⁺ T cells using flow cytometry. (See Example 30.)

FIG. 35 shows the level of cytokine secretion induced by anti-PSMA×anti-CD3ε constructs PSMA01107, PSMA01116 and TSC291a from PBMC cultures at 24 hours. PBMC were co-cultured with C4-2B target cells in the presence of serial dilutions of the antibody constructs. Secretion of cytokines in the culture supernatants was assessed using multiplexed-based assays. Cytokine levels are expressed in pg/ml units. (See Example 30.)

FIG. 36 shows the results of assays measuring T-cell redirected cytotoxicity of high PSMA-expressing target cells by of anti-PSMA×anti-CD3ε constructs PSMA01107 and PSMA01116 at 72 and 96 hours. PBMC were co-cultured with C4-2B-luc cells in the presence of serial dilutions of the antibody constructs. The fraction of live C4-2B cells was quantified by bioluminescence after addition of luciferin substrate and represented in RLU. (See Example 31.)

FIG. 37 shows growth of C4-2B-luciferase tumor cells in NOD/SCID mice following treatment with CD3×PSMA constructs to day 28. (See Example 32.)

FIG. 38 shows growth of C4-2B-luciferase tumor incidence cells in NOD/SCID mice following treatment with CD3×PSMA constructs. (See Example 32.)

FIGS. 39A and 39B show growth of C4-2B-luciferase tumor cells in NOD/SCID mice following treatment with CD3×PSMA constructs to study endpoint at day 63 (FIG. 39A) or at day 28 (FIG. 39B). Summary table shows the log % reduction as calculated at day 24 and the % tumor free incidence as calculated at maximal response at day 14 (FIG. 32B). (See Example 32.)

FIG. 40 shows bioluminescent imaging of NOD/SCID mice to visualize C4-2B tumor growth following treatment with CD3×PSMA constructs. Bioluminescence is displayed as increasing light density and contrast shading visualized on the bodies of each animal. (See Example 32.).

FIG. 41 shows the downstream CD3 signaling through NFAT, ERK, and NFkB with different anti-PSMA×anti-CD3ε constructs at 20 nM run in the presence or absence of C4-2B PSMA-expressing target cells to demonstrate the requirement of PSMA crosslinking and the background levels of CD3 signaling in the absence of crosslinking. Reporter activity was assessed measuring the relative light units expressed in NFAT, ERK, or NFkB reporter assays. Constructs were incubated with or without target cells and the reporter cell line for 24 hours, followed by the addition of BioGlo. The y-axis displays relative light units (RLU). (See Example 34).

FIGS. 42A and 42B show NFAT reporter assays and the CD3 downstream signaling activity with various anti-PSMA×anti-CD3ε constructs. FIG. 42A shows NFAT reporter activity on serial dilutions of construct assessed after 10 hours in culture. FIG. 42B shows the EC50 values obtained from serially diluted constructs in the NFAT reporter assay and plotted to compare the differences in EC50s at various time points. (See Example 34).

FIG. 43 shows the EC50s obtained from NFAT, ERK, and NFκB reporter signaling assays from a titration of anti-PSMA×anti-CD3ε constructs after 4, 10, and 24 hours in culture in the presence of C4-2B tumor target cells. (See Example 34).

FIGS. 44A-44C show the effect of anti-PSMA×anti-CD3ε constructs on the memory phenotype of human CD8⁺ T cells. FIGS. 44A and 44B show the in vitro effect of anti-PSMA×anti-CD3ε constructs on the memory phenotype of human CD8⁺ T cells after 72 hrs in culture with serial dilutions of PSMA01107 or PSMA1110 and C4-2B tumor target cells. Memory phenotypes were quantified as the percent surface staining of CD45RO and CD62L on gated CD5+CD8+ T cells using flow cytometry. CD45RO+ CD62L+ central memory T cells (FIG. 44A) and CD45RO− CD62L-terminally differentiated T cells (FIG. 44B) are displayed. FIG. 44C shows representative data plotted to demonstrate the differences in naïve, central memory (TCM), effector memory (TEM), and terminally differentiated (Teff) CD8+ T cells following incubation with anti-PSMA×anti-CD3ε (0.2 nM) and C4-2B target cells. (See Example 35).

### DETAILED DESCRIPTION

To facilitate an understanding of the present disclosure, a number of terms and phrases are defined below.

### I. Terminology

As used herein, the term “Prostate-specific Membrane Antigen (PSMA),” also known as glutamate carboxypeptidase II and N-acetylated alpha-linked acidic dipeptidase 1, is a dimeric type II transmembrane glycoprotein belonging to the M28 peptidase family encoded by the gene FOLH1 (folate hydrolase 1). The protein is a glutamate carboxypeptidase on different alternative substrates, including the nutrient folate and the neuropeptide N-acetyl-l-aspartyl-l-glutamate and is expressed in a number of tissues such as the prostate, and to a lesser extent, the small intestine, central and peripheral nervous system and kidney. The gene encoding PSMA is alternatively spliced to produce at least three variants. A mutation in this gene may be associated with impaired intestinal absorption of dietary folates, resulting in low blood folate levels and consequent hyperhomocysteinemia. Expression of this protein in the brain may be involved in a number of pathological conditions associated with glutamate excitotoxicity. Expression of PSMA increases with prostate cancer progression and is highest in metastatic disease, hormone refractory cases, and higher-grade lesions. Additionally, PSMA is abundantly expressed on the neovasculature of a variety of other solid tumors, including bladder, pancreas, melanoma, lung and kidney cancers, but not on normal neovasculature

As used herein, the term “CD3” is known in the art as a multi-protein complex of six chains (see, e.g., Abbas and Lichtman, 2003; Janeway et al., p. 172 and 178, 1999), which are subunits of the T cell receptor complex. In mammals, the CD3 subunits of the T cell receptor complex are a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. It is believed the immunoreceptor tyrosine-based activation motif (ITAMs) are important for the signaling capacity of a TCR complex. CD3 as used in the present disclosure can be from various animal species, including human, monkey, mouse, rat, or other mammals.

As used herein, the term “tumor infiltrating lymphocytes” or “TIL” refers to lymphocytes that directly oppose and/or surround tumor cells. Tumor infiltrating lymphocytes are typically non-circulating lymphocytes and include, CD8+ T cells, CD4+ T cells and NK cells.

As used herein, the terms “antibody” and “antibodies” are terms of art and can be used interchangeably herein and refer to a molecule or a complex of molecules with at least one antigen-binding site that specifically binds an antigen.

Antibodies can include, for example, monoclonal antibodies, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), bispecific antibodies, and multi-specific antibodies. In certain aspects, antibodies described herein refer to polyclonal antibody populations.

Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂ₐ or IgG_{2b}) of immunoglobulin molecule. In certain aspects, antibodies described herein are IgG antibodies, or a class (e.g., human IgG₁, IgG₂, or IgG₄) or subclass thereof. In a specific aspect, the antibody is a humanized monoclonal antibody. In another specific aspect, the antibody is a human monoclonal antibody, e.g., that is an immunoglobulin. In certain aspects, an antibody described herein is an IgG₁, IgG₂, or IgG₄ antibody.

“Bispecific” antibodies are antibodies with two different antigen-binding sites (exclusive of the Fc region) that bind to two different antigens. Bispecific antibodies can include, for example, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, heteroconjugate antibodies, linked single chain antibodies or linked-single-chain Fvs (scFv), camelized antibodies, affybodies, linked Fab fragments, F(ab′)₂ fragments, chemically-linked Fvs, and disulfide-linked Fvs (sdFv). Bispecific antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂ₐ or IgG_{2b}) of immunoglobulin molecule. In certain aspects, bispecific antibodies described herein are IgG antibodies, or a class (e.g., human IgG₁, IgG₂, or IgG4) or subclass thereof.

Bispecific antibodies can be e.g., monovalent for each target (e.g., an IgG molecule with one arm targeting one antigen and the other arm targeting a second antigen), bivalent for each target (e.g., when the bispecific antibody is in a homodimer ADAPTIR™ format), or monovalent for one target (e.g., CD3) and bivalent for another target (e.g., a TAA such as PSMA, HER2, or BCMA) (e.g., when the bispecific antibody is in a heterodimer ADAPTIR-FLEX™ format).

In certain aspects, bispecific antibodies described herein comprise two polypeptides, optionally identical polypeptides, each polypeptide comprising in order from amino-terminus to carboxyl-terminus, a first scFv antigen-binding domain, a linker (optionally wherein the linker is a hinge region), an immunoglobulin constant region, and a second scFv antigen-binding domain. This particular type of antibody is exemplified by homodimer ADAPTIR™ technology, which is bivalent for each target.

In certain aspects, bispecific antibodies described herein comprise a heterodimer, i.e., a dimer comprised of two non-identical polypeptides. For instance, in one aspect, the bispecific antibodies described herein comprise a first polypeptide comprising, from N-terminus to C-terminus, a first single chain variable fragment (scFv) that binds a first biological target, a linker (e.g., an immunoglobulin hinge), an immunoglobulin constant region, and a second single chain variable fragment (scFv) that binds a second biological target, and a second polypeptide comprising, from N-terminus to C-terminus, a first single chain variable fragment (scFv) that binds the first biological target, a linker (e.g., an immunoglobulin hinge), and an immunoglobulin constant region. In another aspect, the heterodimer bispecific antibodies described herein comprise a first polypeptide comprising, from N-terminus to C-terminus, a first single chain variable fragment (scFv) that binds a first biological target, a linker (e.g., an immunoglobulin hinge), an immunoglobulin constant region, and a second single chain variable fragment (scFv) that binds a second biological target, and a second polypeptide comprising, from N-terminus to C-terminus, a linker (e.g., an immunoglobulin hinge), an immunoglobulin constant region, and a second single chain variable chain (scFv) hat binds a second biological target. These particular types of antibodies that are bivalent for one target and monovalent for another target are exemplified by the ADAPTIR-FLEX™ platform technology.

As used herein, the terms “antigen-binding domain,” “antigen-binding region,” “antigen-binding site,” and similar terms refer to the portion of antibody molecules which comprises the amino acid residues that confer on the antibody molecule its specificity for the antigen (e.g., the complementarity determining regions (CDR)). The antigen-binding region can be derived from any animal species, such as rodents (e.g., mouse, rat, or hamster) and humans. An antigen-binding domain that binds to TAA can be referred to herein e.g., as a “TAA-binding domain.” An antigen-binding domain that binds to PSMA can be referred to herein e.g., as a “PSMA-binding domain.” An antigen-binding domain that binds to CD3 can be referred to herein e.g., as an “CD3-binding domain.” In some aspects, a CD3-binding domain binds to CD3ε.

As used herein, the terms “TAA/CD3 antibody,” “CD3/TAA antibody,” “anti-TAA/CD3 antibody,” “anti-CD3/TAA antibody,” “TAA×CD3 antibody” and “CD3×TAA antibody” refer to a bispecific antibody that contains an antigen-binding domain that binds to a TAA (e.g., PSMA, HER2, or BCMA) and an antigen-binding domain that binds to CD3 (e.g., human CD3).

As used herein, the terms “PSMA/CD3 antibody,” “CD3/PSMA antibody,” “anti-PSMA/CD3 antibody,” “anti-CD3/PSMA antibody,” “PSMA×CD3 antibody” and “CD3×PSMA antibody” refer to a bispecific antibody that contains an antigen-binding domain that binds to PSMA (e.g., human PSMA) and an antigen-binding domain that binds to CD3 (e.g., human CD3).

A “monoclonal” antibody refers to a homogeneous antibody population involved in the highly specific recognition and binding of a single antigenic determinant, or epitope. This is in contrast to polyclonal antibodies that typically include different antibodies directed against different antigenic determinants. The term “monoclonal” antibody encompasses both intact and full-length immunoglobulin molecules as well Fab, Fab′, F(ab′)2, Fv), single chain (scFv), fusion proteins comprising an antibody portion, and any other modified immunoglobulin molecule comprising an antigen recognition site. Furthermore, a “monoclonal” antibody refers to such antibodies made in any number of manners including but not limited to by hybridoma, phage selection, recombinant expression, and transgenic animals.

The term “chimeric” antibodies refers to antibodies wherein the amino acid sequence is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The term “humanized” antibody refers to forms of non-human (e.g., murine) antibodies that contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some aspects, the Fv framework region (FR) residues of a human immunoglobulin are replaced with the corresponding residues in an antibody from a non-human species that has the desired specificity, affinity, and capability. The humanized antibody thereof can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996).

The term “human” antibody means an antibody having an amino acid sequence derived from a human immunoglobulin gene locus, where such antibody is made using any technique known in the art.

The variable region typically refers to a portion of an antibody, generally, a portion of a light or heavy chain, typically about the amino-terminal 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain aspects, the variable region is a human variable region. In certain aspects, the variable region comprises rodent or murine CDRs and human framework regions (FRs). In particular aspects, the variable region is a primate (e.g., non-human primate) variable region. In certain aspects, the variable region comprises rodent or murine CDRs and primate (e.g., non-human primate) framework regions (FRs).

The terms “VH” and “VH domain” are used interchangeably to refer to the heavy chain variable region of an antibody.

The terms “VL” and “VL domain” are used interchangeably to refer to the light chain variable region of an antibody.

The term “Kabat numbering” and like terms are recognized in the art and refer to a system of numbering amino acid residues in the heavy and light chain variable regions of an antibody, or an antigen-binding portion thereof. In certain aspects, the CDRs of an antibody can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific aspect, the CDRs of the antibodies described herein have been determined according to the Kabat numbering scheme.

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). In a specific aspect, the CDRs of the antibodies described herein have been determined according to the Chothia numbering scheme.

The AbM hypervariable regions represent a compromise between the Kabat CDRs and Chothia structural loops, and are used by Oxford Molecular's AbM antibody modeling software. In a specific aspect, the CDRs of the antibodies described herein have been determined according to the AbM numbering scheme.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L197 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The IMGT numbering convention is described in Brochet, X, et al, Nucl. Acids Res. 36: W503-508 (2008). In a specific aspect, the CDRs of the antibodies described herein have been determined according to the IMGT numbering convention. As used herein, unless otherwise provided, a position of an amino acid residue in a variable region of an immunoglobulin molecule is numbered according to the IMGT numbering convention.

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. An immunoglobulin “constant region” or “constant domain” can contain a CH1 domain, a hinge, a CH2 domain, and a CH3 domain or a subset of these domains, e.g., a CH2 domain and a CH3 domain. In certain aspects provided herein, an immunoglobulin constant region does not contain a CH1 domain. In certain aspects provided herein, an immunoglobulin constant region does not contain a hinge. In certain aspects provided herein, an immunoglobulin constant region contains a CH2 domain and a CH3 domain.

“Fc region” or “Fc domain” refers to a polypeptide sequence corresponding to or derived from the portion of a source antibody that is responsible for binding to antibody receptors on cells and the C1q component of complement. Fc stands for “fragment crystalline,” and refers to the fragment of an antibody that will readily form a protein crystal. Distinct protein fragments, which were originally described by proteolytic digestion, can define the overall general structure of an immunoglobulin protein. An “Fc region” or “Fc domain” contains a CH2 domain, a CH3 domain, and optionally all or a portion of a hinge. An “Fc region” or “Fc domain” can refer to a single polypeptide or to two disulfide-linked polypeptides. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc includes variants of naturally occurring sequences.

A “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of a naturally occurring antibody. In certain aspects, a wild type immunoglobulin hinge region sequence is human, and can comprise a human IgG hinge region. An “altered wild-type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), or (b) a portion of a wild type immunoglobulin hinge region that has a length of about 5 amino acids (e.g., about 5, 6, 7, 8, 9, 10, 11, 12, 13, 14, 15, 16, 17, 18, 19, or 20 amino acids) up to about 120 amino acids (for instance, having a length of about 10 to about 40 amino acids or about 15 to about 30 amino acids or about 15 to about 20 amino acids or about 20 to about 25 amino acids), has up to about 30% amino acid changes (e.g., up to about 25%, 20%, 15%, 10%, 5%, 4%, 3%, 2%, or 1% amino acid substitutions or deletions or a combination thereof), and has an IgG core hinge region as disclosed in US 2013/0129723 and US 2013/0095097. As provided herein, a “hinge region” or a “hinge” can be located between an antigen-binding domain (e.g., a TAA (e.g., PSMA)- or a CD3-binding domain) and an immunoglobulin constant region.

As used herein, a “linker” refers to a moiety, e.g., a polypeptide, that is capable of joining two compounds, e.g., two polypeptides. Non-limiting examples of linkers include flexible linkers comprising glycine-serine (e.g., (Gly₄Ser)) repeats, and linkers derived from (a) an interdomain region of a transmembrane protein (e.g., a type I transmembrane protein); (b) a stalk region of a type II C-lectin; or (c) an immunoglobulin hinge. As provided herein, a linker can refer, e.g., to (1) a polypeptide region between VH and VL regions in a single-chain Fv (scFv) or (2) a polypeptide region between an immunoglobulin constant region and an antigen-binding domain. In certain aspects, a linker is comprised of 5 to about 35 amino acids, for instance, about 15 to about 25 amino acids. In some aspects, a linker is comprised of at least 5 amino acids, at least 7 amino acids or at least 9 amino acids.

As used herein, the term “heavy chain” when used in reference to an antibody can refer to any distinct type, e.g., alpha (a), delta (6), epsilon (a), gamma (γ), and mu (p), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ) based on the amino acid sequence of the constant regions. Light chain amino acid sequences are well known in the art. In specific aspects, the light chain is a human light chain.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, each of which is herein incorporated by reference in its entirety. As used herein, unless otherwise provided, a position of an amino acid residue in a constant region of an immunoglobulin molecule is numbered according to EU nomenclature (Ward et al., 1995 Therap. Immunol. 2:77-94).

As used herein, the term “dimer” refers to a biological entity that consists of two subunits associated with each other via one or more forms of intramolecular forces, including covalent bonds (e.g., disulfide bonds) and other interactions (e.g., electrostatic interactions, salt bridges, hydrogen bonding, and hydrophobic interactions), and is stable under appropriate conditions (e.g., under physiological conditions, in an aqueous solution suitable for expressing, purifying, and/or storing recombinant proteins, or under conditions for non-denaturing and/or non-reducing electrophoresis). A “heterodimer” or “heterodimeric protein,” as used herein, refers to a dimer formed from two different polypeptides. A “homodimer” or “homodimeric protein,” as used herein, refers to a dimer formed from two identical polypeptides. Thus, a heterodimer ADAPTIR-FLEX™ construct refers to a construct comprising two non-identical polypeptides, whereas a homodimer ADAPTIR™ construct refers to a construct comprising two different polypeptides.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas KA is calculated from the quotient of kₒₙ/k_{off}. kₒₙ refers to the association rate constant of, e.g., an antibody to an antigen, and k_{off} refers to the dissociation of, e.g., an antibody from an antigen. The kₒₙ and k_{off} can be determined by techniques known to one of ordinary skill in the art, such as BIAcore® or KinExA.

As used herein, “binding strength” or “binding potency” refers to the strength of a non-covalent interaction between a protein molecule in solution and the other member of the binding pair expressed on the surface of cell, or affixed to a solid surface such as a bead, SPR chip, ELISA plate, etc. These terms can be used to describe a monovalent interaction, in which one binding domain on the protein in solution binds to one ligand on the surface (a 1:1 interaction). This can be a bivalent or multivalent interaction, in which two or more binding domains on a protein molecule in solution simultaneously bind to two or more copies of the same or different ligands on the surface. Other valencies of interaction are possible, such as trivalent, tetravalent, etc. This can include binding to the same location on multiple copies of the same ligand, or different locations, or epitopes on one ligand molecule.

The numerical value associated with “binding strength” or “binding potency” is generally calculated from cell binding curves by plotting the data and performing nonlinear regression analysis to determine EC50 values (the concentration of protein required to achieve 50% of the maximum binding signal). “High binding strength” or “high binding potency” refers to protein:surface interactions with an EC50 value determined to be less than 10⁻⁷ M, less than 10⁻⁸ M, less than 10⁻⁹ M, or less than 10⁻¹⁰ M. “Low binding strength” or “low binding potency” protein:surface interactions refer to those binding domains with an EC50 than 10⁻⁷ M, greater than 10⁻⁶ M, or greater than 10⁻⁵ M.

As used herein, “binding avidity” generally refers to a non-covalent interaction between a binding pair in which the points of contact between the binding domain and ligand may be greater than 1. Whereas binding affinity represents the strength of a single, non-covalent interaction between a binding pair, avidity reflects the total binding strength of interactions where there may be more than one point of interaction between the pair. For example, this could be a 2:1, or 2:2 interaction between the protein and the surface binding partner, respectively. Other ratios of interaction are possible and are included within this definition. When the ratio of interaction is 1:1, then the values of affinity and avidity are considered equal. When the ratio of the interaction exceeds 1:1, this is consider an avid interaction, and the strength of the interaction may be greater than the affinity of a 1:1 interaction.

As used herein, the terms “immunospecifically binds,” “immunospecifically recognizes,” “specifically binds,” and “specifically recognizes” are analogous terms in the context of antibodies. These terms indicate that the antibody binds to an epitope via its antigen-binding domain and that the binding entails some complementarity between the antigen-binding domain and the epitope. Accordingly, an antibody that “specifically binds” to a TAA (e.g., human PSMA, HER2, or BCMA) and/or CD3 may also, but the extent of binding to an un-related protein is less than about 10% of the binding of the antibody to the TAA (e.g., PSMA, HER2, or BCMA) and/or CD3 as measured, e.g., by a radioimmunoassay (RIA).

Binding domains can be classified as “high affinity” binding domains and “low affinity” binding domains. “High affinity” binding domains refer to those binding domains with a K_{D} value less than 10⁻⁷ M, less than 10⁻⁸ M, less than 10⁻⁹ M, less than 10⁻¹⁰ M. “Low affinity” binding domains refer to those binding domains with a KD greater than 10⁻⁷ M, greater than 10⁻⁶ M, or greater than 10⁻⁵ M. “High affinity” and “low affinity” binding domains bind their targets, while not significantly binding other components present in a test sample.

As used herein, an antibody is “capable of binding” if it will specifically bind its target (e.g., a TAA (e.g., human PSMA) and/or or human CD3) when in close proximity to the target and under conditions one of skill in the art would consider to be necessary for binding. A “TAA-binding domain” should be understood to mean a binding domain that specifically binds to a TAA. A “PSMA-binding domain” should be understood to mean a binding domain that specifically binds to PSMA. A “CD3 antigen-binding domain” should be understood to mean a binding domain that specifically binds to CD3.

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain aspects, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody:antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can be engineered to incorporate various binding domains, including, for instance, one or more binding domains derived from a single chain variable fragment, a cytokine, or an extracellular domain.

It is understood that, because polypeptides provided herein are related to antibodies, in certain aspects, the polypeptides can occur as single chains or as associated chains. Two polypeptides or proteins can bond to each other to form a “homodimer” or “heterodimer.” A homodimer can be formed when two identical polypeptides bond together. A heterodimer can be formed when two non-identical polypeptides bond together. An example of a homodimer polypeptide is one comprising, from N-terminus to C-terminus, a first binding domain, a linker (such as an immunoglobulin hinge), an immunoglobulin constant region, and a second binding domain. In one aspect provided herein, the binding domains of a homodimer are single chain variable fragments.

A heterodimer can be formed, for instance, when a first polypeptide comprising, from N-terminus to C-terminus, a first binding domain, a linker (such as an immunoglobulin hinge), an immunoglobulin constant region, and a second binding domain bonds with a second polypeptide comprising, from N-terminus to C-terminus, a first binding domain, a linker (such as an immunoglobulin hinge), and an immunoglobulin constant region. Two polypeptides can bond to form a heterodimer by incorporating knob-in-hole mutations in the Fc region of the polypeptide chains. In one aspect provided herein, the binding domains of a heterodimer are single chain variable fragments. A heterodimer construct can be a monospecific, bispecific, or multispecific construct depending on the number of binding domains and target. A bispecific heterodimer construct can be designed to be bivalent for one biological target (i.e., two scFvs bind the target) or monovalent (i.e., a single scFv binds the target).

As used herein, the terms “nucleic acid,” “nucleic acid molecule,” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. As used herein, the terms “nucleic acid,” “nucleic acid molecule,” or “polynucleotide” are intended to include DNA molecules (e.g., cDNA or genomic DNA), RNA molecules (e.g., mRNA), analogs of the DNA or RNA generated using nucleotide analogs, and derivatives, fragments and homologs thereof.

The term “expression vector,” as used herein, refers to a nucleic acid molecule, linear or circular, comprising one or more expression units. In addition to one or more expression units, an expression vector can also include additional nucleic acid segments such as, for example, one or more origins of replication or one or more selectable markers. Expression vectors are generally derived from plasmid or viral DNA, or can contain elements of both.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

As used herein, a “conservative amino acid substitution” is one in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain aspects, one or more amino acid residues within a CDR(s) or within a framework region(s) of an antibody can be replaced with an amino acid residue with a similar side chain.

As used herein, a polypeptide or amino acid sequence “derived from” a designated polypeptide refers to the origin of the polypeptide. In certain aspects, the polypeptide or amino acid sequence which is derived from a particular sequence (sometimes referred to as the “starting” or “parent” or “parental” sequence) has an amino acid sequence that is essentially identical to the starting sequence or a portion thereof, wherein the portion consists of at least 10-20 amino acids, at least 20-30 amino acids, or at least 30-50 amino acids, or at least 50-150 amino acids, or which is otherwise identifiable to one of ordinary skill in the art as having its origin in the starting sequence. For example, a binding domain can be derived from an antibody, e.g., a Fab, F(ab′)₂, Fab′, scFv, single domain antibody (sdAb), etc.

Polypeptides derived from another polypeptide can have one or more mutations relative to the starting polypeptide, e.g., one or more amino acid residues which have been substituted with another amino acid residue or which has one or more amino acid residue insertions or deletions. The polypeptide can comprise an amino acid sequence which is not naturally occurring. Such variations necessarily have less than 100% sequence identity or similarity with the starting polypeptide. In one aspect, the variant will have an amino acid sequence from about 60% to less than 100% amino acid sequence identity or similarity with the amino acid sequence of the starting polypeptide. In another aspect, the variant will have an amino acid sequence from about 75% to less than 100%, from about 80% to less than 100%, from about 85% to less than 100%, from about 90% to less than 100%, from about 95% to less than 100% amino acid sequence identity or similarity with the amino acid sequence of the starting polypeptide.

As used herein, the term “host cell” can be any type of cell, e.g., a primary cell, a cell in culture, or a cell from a cell line. In specific aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants). In some aspects, a material is at least 90% pure, at least 95% pure, at least 98% pure, or at least 99% pure.

The term “pharmaceutical formulation” or “pharmaceutical composition” refers to a preparation which is in such form as to permit the biological activity of the active ingredient to be effective, and which contains no additional components which are unacceptably toxic to a subject to which the formulation would be administered. The formulation can be sterile.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

The terms “administer”, “administering”, “administration”, and the like, as used herein, refer to methods that may be used to enable delivery of a drug, e.g., a TAA/CD3 antibody (such as a PSMA/CD3 antibody) to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human. As used herein, the term “patient in need” or “subject in need” refers to a patient at risk of, or suffering from, a disease, disorder or condition that is amenable to treatment or amelioration, e.g., with a TAA/CD3 antibody (such as a PSMA/CD3 antibody) provided herein. A patient in need can, for instance, be a patient diagnosed with a cancer. For instance, the patient can be diagnosed with PSMA(+) tumors and/or prostate cancer, including, for instance, metastatic castration-resistant prostate cancer.

The term “therapeutically effective amount” refers to an amount of a drug, e.g., an anti-TAA/CD3 antibody (e.g., anti-PSMA/CD3 antibody) effective to treat a disease or disorder in a subject. In the case of cancer, the therapeutically effective amount of the drug can reduce the number of cancer cells; reduce the tumor size or burden; inhibit (i.e., slow to some extent and in a certain aspect, stop) cancer cell infiltration into peripheral organs; inhibit (i.e., slow to some extent and in a certain aspect, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP), or any combination thereof.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In certain aspects, a subject is successfully “treated” for cancer according to the methods of the present disclosure if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof.

The terms “cancer” and “cancerous” refer to or describe the physiological condition in mammals in which a population of cells are characterized by unregulated cell growth. Examples of cancer include, but are not limited to, prostate cancer, colorectal cancer, and gastric cancer. The cancer may be a primary tumor or may be advanced or metastatic cancer.

A cancer can be a solid tumor cancer. The term “solid tumor” refers to an abnormal mass of tissue that usually does not contain cysts or liquid areas. Examples of solid tumors are sarcomas, carcinomas, and lymphomas. Leukemias (cancers of the blood) generally do not form solid tumors.

It should be understood that the terms “a” and “an” as used herein refer to “one or more” of the enumerated components unless otherwise indicated.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially” are open-ended, allowing for the presence of more than that which is recited so long as basic or novel characteristics of that which is recited is not changed by the presence of more than that which is recited, but excludes prior art aspects.

As used herein, the terms “about” and “approximately,” when used to modify a numeric value or numeric range, indicate that deviations of up to 5% above or 5% below the value or range remain within the intended meaning of the recited value or range. It is understood that wherever aspects are described herein with the language “about” o “approximately,” a numeric value or range, otherwise analogous aspects referring to the specific numeric value or range (without “about”) are also provided.

Any domains, components, compositions, and/or methods provided herein can be combined with one or more of any of the other domains, components, compositions, and/or methods provided herein.

### II. TAA and CD3 Antibodies

Provided herein are CD3 antibodies, CD3×TAA (e.g., PSMA, HER2, or BCMA) antibodies, and PSMA antibodies.

The CD3 antibodies and the CD3×TAA (e.g., PSMA, HER2, or BCMA) bispecific antibodies can comprise an antigen-binding domain that binds to human CD3. The antigen-binding domain that binds to human CD3 can bind to human CD3ε. The antigen-binding domain that binds to human CD3 can be a humanized or a human antigen-binding domain that binds to CD3.

In one aspect provided herein, the CD3 antibodies and CD3×TAA (e.g., PSMA, HER2, or BCMA) exhibit reduced or low binding affinity to CD3. Also provided are CD3×TAA antibodies that exhibit reduced or low binding affinity to CD3 and which also promote CD8 T cell activation and proliferation. The TAA (e.g., PSMA) binding domain can have greater binding strength, binding potency, and/or avidity to PSMA than the CD3 binding domain has to CD3.

The antigen-binding domain that binds to human CD3 (e.g., a humanized antigen-binding domain that binds to CD3) can have reduced affinity for CD3 as compared to the parental antibody (e.g., as compared to the CRIS 7 murine monoclonal antibody (VH SEQ ID NO: 122; VL SEQ ID NO: 124) or the SP34 murine monoclonal antibody). In one aspect provided herein, the humanized or human antigen-binding domain has reduced binding affinity to human CD3 as compared to the CD3 binding domain of DRA222 (VH SEQ ID NO: 126; VL SEQ ID NO: 128) and/or the CD3 binding domain of TSC456 (VH SEQ ID NO: 130; VL SEQ ID NO: 132. In one aspect provided herein, the CD3 antibodies and CD3×TAA antibodies exhibit reduced binding affinity for Jurkat cells compared to comparator CD3 antibodies and CD3×TAA antibodies.

In one aspect provided herein, the antibody is a TAA (e.g., PSMA)×CD3 targeting antibody wherein the CD3 binding domain binds to human CD3 with reduced affinity as compared to the binding affinity of the TAA binding domain to the TAA. In one aspect provided herein, the binding affinity of the CD3 binding domain to CD3 is 1.5 fold, 2 fold, 2.5 fold, 3 fold, 3.5 fold, 4 fold, 4.5 fold, 5 fold or more less than the binding affinity of the TAA binding domain to TAA. The differential in binding affinities (i.e., greater binding affinity of the TAA binding domain to TAA as compared to the binding affinity of the CD3 binding domain to CD3) improves binding of the TAA×CD3 antibodies to tumor cells expressing the TAA and/or reduces binding of the TAA×CD3 antibodies to circulating T cells.

The antigen-binding domain that binds to human CD3 (e.g., a humanized or human antigen-binding domain that binds to CD3) can be on the C-terminus of the construct. In one aspect provided herein, the antigen binding domain that binds to human CD3 is on the C-terminus of a polypeptide chain and the binding domain proximal to an Fc domain. In one aspect provided herein, the CD3 binding domain is on the C-terminus of a homodimer comprising two identical polypeptides, each polypeptide comprising, in order from amino-terminus to carboxyl-terminus, a first scFv antigen-binding domain capable of binding TAA, a linker (optionally wherein the linker is an immunoglobulin hinge), an immunoglobulin constant region, and a second scFv antigen-binding domain capable of binding CD3. The location of the CD3 binding domain on the C-terminus of a TAA×CD3 scFv-Fc-scFv homodimer exhibits reduced binding affinity to CD3 as compared to a similar TAA×CD3 scFc-Fc-scFv homodimer with the CD3 binding domain on the N-terminus. Without wishing to be bound by a theory, it is hypothesized that the proximity of the immunoglobulin constant region to the CD3 binding domain can interfere with the ability of the CD3 binding domain to tightly bind to CD3. The homodimer antibody structure described herein can be used with CD3 binding domains with modified sequences to further reduce CD3 binding affinity.

In one aspect provided herein, the CD3 binding domain is on the C-terminus of a heterodimer comprising two non-identical polypeptides, a first polypeptide comprising, from N-terminus to C-terminus, a first single chain variable fragment (scFv) that binds a TAA, a linker (e.g., an immunoglobulin hinge), an immunoglobulin constant region, and a second single chain variable fragment (scFv) that binds CD3, and a second polypeptide comprising, from N-terminus to C-terminus, a first single chain variable fragment (scFv) that binds the TAA, a linker (e.g., an immunoglobulin hinge), and an immunoglobulin constant region. This format is exemplified in the ADAPTIR-FLEX™ technology. In this aspect, the binding domain that binds TAA is bivalent, whereas the binding domain that binds the CD3 is monovalent. The heterodimer antibody structure described herein comprising a monovalent CD3 binding domain can be designed to incorporate any CD3 binding domain to reduce CD3 binding affinity as compared to a BiTE or D.A.R.T. TAA×CD3 comprising the same binding domains. The heterodimer antibody structure described herein can incorporate a CD3 binding domain with a modified sequence designed to further reduce CD3 binding affinity (e.g., a CD3 binding domain with a VH comprising the amino acid sequence of SEQ ID NO: 134 and a VL comprising the amino acid sequence of SEQ ID NO:136 or a CD3 binding domain with a VH comprising the amino acid sequence of SEQ ID NO:138 and a VL comprising the amino acid sequence of SEQ ID NO: 140 or a CD3 binding domain with a VH comprising the amino acid sequence of SEQ ID NO:142 and a VL comprising the amino acid sequence of SEQ ID NO:144).

In one aspect provided herein, the CD3 binding domain on the C-terminus is a humanized antibody binding domain derived from the murine monoclonal antibody CRIS-7. In one aspect provided herein, the CD3 binding domain on the C-terminus is a humanized antibody binding domain derived from the murine monoclonal antibody SP34 (e.g., I2C).

The PSMA antibodies and the CD3×PSMA bispecific antibodies can comprise an antigen-binding domain that binds to human PSMA. The antigen-binding domain that binds to human PSMA can be a humanized or human antigen-binding domain that binds to PSMA.

The CD3×TAA (e.g., PSMA, HER2, or BCMA) bispecific antibodies can comprise a humanized TAA (e.g., PSMA, HER2, or BCMA)-binding domain and/or a humanized CD3-binding domain. The CD3×TAA (e.g., PSMA, HER2, or BCMA) bispecific antibodies can comprise a human TAA (e.g., PSMA, HER2, or BCMA)-binding domain and/or a human CD3-binding domain. The CD3×TAA (e.g., PSMA, HER2, or BCMA) bispecific antibodies can be monovalent for one target (e.g., CD3) and bivalent for the other target (e.g., the TAA such as PSMA, HER2, or BCMA). An example of TAA×CD3 bispecific antibody is an antibody comprising a first polypeptide from N-terminus to C-terminus comprising (i) a first single chain variable fragment (scFv) that binds to a TAA, (ii) an immunoglobulin constant region, and (iii) an scFv that binds to CD3; and a second polypeptide comprising (i) a second scFv that binds to a TAA, and (ii) an immunoglobulin constant region, wherein the bispecific antibody does not contain a second CD3-binding domain. The CD3×TAA (e.g., PSMA, HER2, or BCMA) bispecific antibodies can be monovalent for one target (e.g., CD3) and bivalent for the other target (e.g., the TAA such as PSMA, HER2, or BCMA). An example of TAA×CD3 bispecific antibody is an antibody comprising a first polypeptide from N-terminus to C-terminus comprising (i) a first single chain variable fragment (scFv) that binds to a TAA, (ii) a hinge region, (iii) an immunoglobulin constant region, and (iv) an scFv that binds to CD3; and a second polypeptide comprising (i) a second scFv that binds to a TAA, (ii) a hinge region, and (iii) an immunoglobulin constant region, wherein the bispecific antibody does not contain a second CD3-binding domain. In one aspect provided herein, the CD3×TAA bispecific antibodies comprise a “null” Fc, i.e., no or significantly reduced CDC and ADCC activity. In one aspect provided herein, the CD3×TAA antibodies bind to Jurkat cells with reduced binding affinity as compared to CD3×TAA antibodies with identical CD3 and TAA antibodies but in the D.A.R.T. or B.i.T.E. formats.

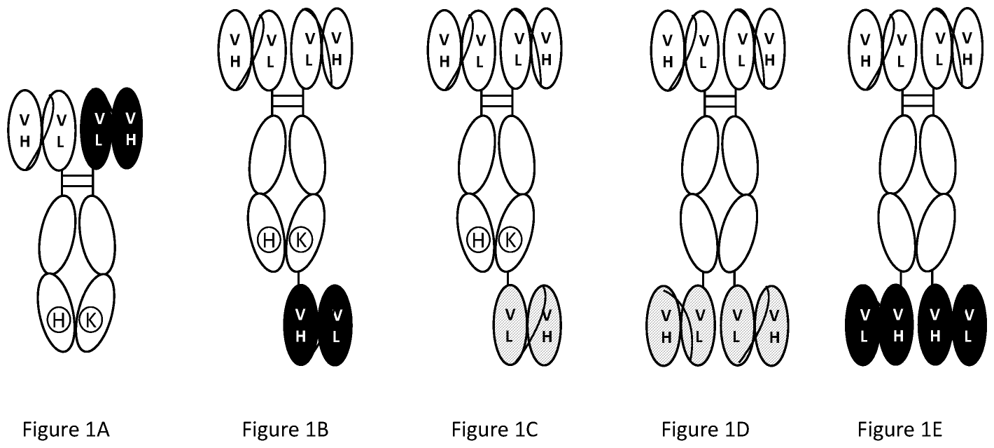

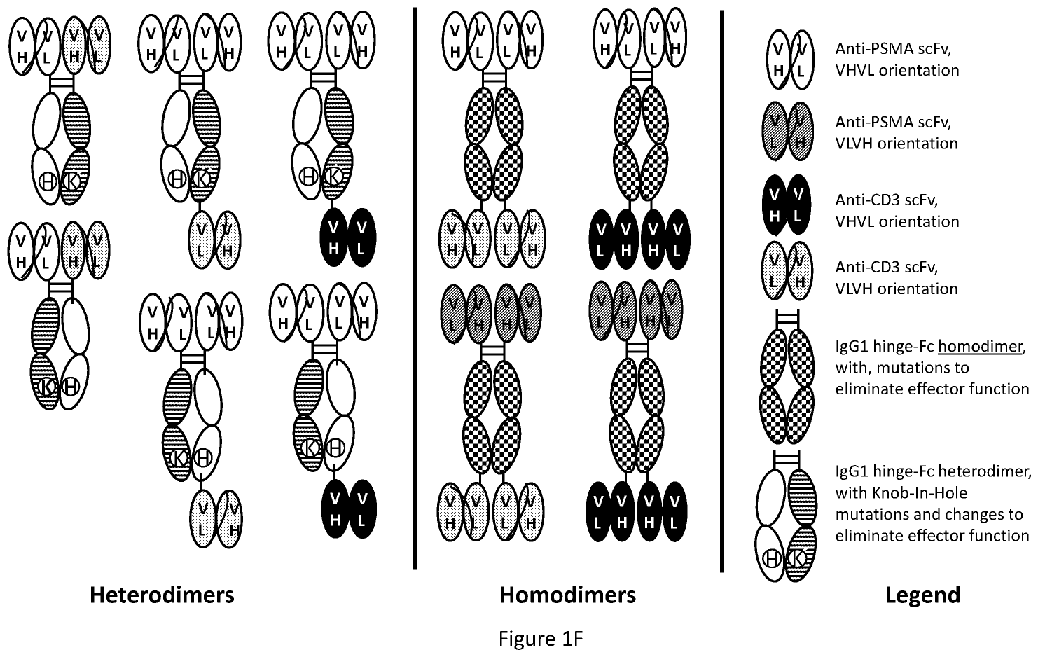

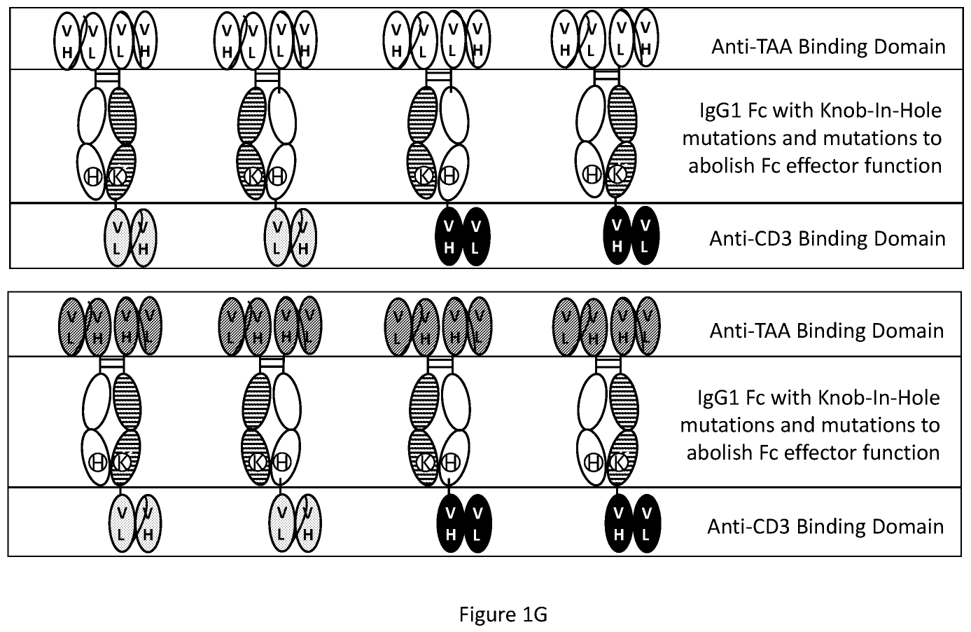

The CD3×TAA bispecific antibodies can comprise a humanized TAA-binding domain and/or a humanized CD3-binding domain. The CD3×TAA bispecific antibodies can be monovalent for one target (e.g., CD3) and bivalent for the other target (e.g., PSMA). Several exemplary (non-limiting) PSMA×CD3 bispecific antibody formats are shown in FIGS. 1A-1F. In addition to the binding domains being a scFv, the binding domains could be an extracellular domain or cytokine.

### A. PSMA-Binding Domains

Provided herein are antigen-binding domains that bind to human PSMA (i.e., PSMA-binding domains) that can be used to assemble PSMA×CD3 bispecific antibodies. A PSMA-binding domain can bind to PSMA from other species, e.g., cynomolgus monkey and/or mouse PSMA, in addition to binding to human PSMA. In certain aspects, the PSMA-binding domains bind to human PSMA and to cynomolgus monkey PSMA. In certain aspects, the first scFv that binds to PSMA and/or the second scFv that binds to cynomolgus PSMA has an EC50 of no more than 5-times greater than the EC50 for binding to human PSMA.

A PSMA-binding domain can comprise six complementarity determining regions (CDRs), i.e., a variable heavy chain (VH) CDR1, a VH CDR2, a VH CDR3, a variable light chain (VL) CDR1, a VL CDR2, and a VL CDR3. A PSMA-binding domain can comprise a variable heavy chain (VH) and a variable light chain (VL). The VH and the VL can be separate polypeptides or can parts of the same polypeptide (e.g., in an scFv).

In certain aspects, a PSMA-binding domain described herein comprises a combination of six CDRs listed in Tables A and B (e.g., SEQ ID NOs:70, 72, 74, 76, 78, and 80).

TABLE A
PSMA VH CDR Amino Acid Sequence¹

| VH CDR1 | VH CDR2 | VH CDR3 |
|---|---|---|
| (SEQ ID NO:) | (SEQ ID NO:) | (SEQ ID NO:) |
| GYTFTDYY | FNPYNDYT | ARSDGYYDAMDY |
| (SEQ ID NO: 70) | (SEQ ID NO: 72) | (SEQ ID NO: 74) |

¹The CDRs are determined according to IMGT.

TABLE B
PSMA VL CDR Amino Acid Sequence²

| VL CDR1 | VL CDR2 | VL CDR3 |
|---|---|---|
| (SEQ ID NO:) | (SEQ ID NO:) | (SEQ ID NO:) |
| KSISKY | SGS | QQHIEYPWT |
| (SEQ ID NO: 76) | (SEQ ID NO: 78) | (SEQ ID NO: 80) |

²The CDRs are determined according to IMGT.

A PSMA×CD3 bispecific antibody that is monovalent for PSMA can comprise a single PSMA-binding domain with a combination of six CDRs listed in Tables A and B above (e.g., SEQ ID NOs:70, 72, 74, 76, 78, and 80). A PSMA×CD3 bispecific antibody that is bivalent for PSMA can comprise two PSMA-binding domains, each comprising a combination of six CDRs listed in Tables A and B above (e.g., SEQ ID NOs: SEQ ID NOs:70, 72, 74, 76, 78, and 80).

As described herein, a PSMA-binding can comprise the VH of an antibody listed in Table C.

TABLE C
PSMA Variable Heavy Chain (VH) Amino Acid Sequence

| SEQ ID NO; | VH Amino Acid Sequence |
|---|---|
| 82 | QVQLVQSGAEVKKPGASVKVSCKASGYTFTDYYMHW |
| | VRQAPGQGLEWMGYFNPYNDYTRYAQKFQGRVTMTR |
| | DTSTSTVYMELSSLRSEDTAVYYCARSDGYYDAMDY |
| | WGQGTTVTVSS |

As described herein, a PSMA-binding domain can comprise a VH having at least about 70%, at least about 75%, at least about 80%, at least about 85%, at least about 90%, at least about 95%, at least about 96%, at least about 97%, at least about 98%, at least about 99%, or 100% sequence identity to a sequence in Table C, optionally wherein the VH comprises VH CDR1, VH CDR2, and VH CDR3 sequences of SEQ ID NOs:70, 72, and 74, respectively.

As described herein, a PSMA-binding domain can comprise a VH comprising the CDRs of a VH sequence in Table C, e.g., the IMGT-defined CDRs, the Kabat-defined CDRs, the Chothia-defined CDRs, or the AbM-defined CDRs.

As described herein, a PSMA-binding domain can comprise the VL of an antibody listed in Table D.

TABLE D
PSMA Variable Light Chain (VL) Amino Acid Sequence

| SEQ ID NO | VL Amino Acid Sequence |
|---|---|
| 84 | DIQMTQSPSSLSASVGDRVTITCRASKSISKYLAWYQQ |
| | KPGKAPKLLIHSGSSLESGVPSRFSGSGSGTEFTLTIS |
| | SLQPDDFATYYCQQHIEYPWTFGQGTKVEIK |

As described herein, a PSMA-binding domain can comprise a VL having at least about 70%, at least about 75%, at least about 80%, at least about 85%, at least about 90%, at least about 95%, at least about 96%, at least about 97%, at least about 98%, at least about 99%, or 100% sequence identity to a sequence in Table D, optionally wherein the VL comprises VL CDR1, VL CDR2, and VL CDR3 sequences of SEQ ID NOs:76, 78, and 80.

As described herein, a PSMA-binding domain can comprise a VL comprising the CDRs of a VL sequence in Table D, e.g., the IMGT-defined CDRs, the Kabat-defined CDRs, the Chothia-defined CDRs, or the AbM-defined CDRs.

As described herein, a PSMA-binding domain can comprise a VH listed in Table C and a VL listed in Table D. A PSMA×CD3 bispecific antibody that is monovalent for PSMA can comprise a single PSMA-binding domain comprising a VH listed in Table C and a VL listed in Table D. A PSMA×CD3 bispecific antibody that is bivalent for PSMA can comprise two PSMA-binding domains, each comprising a VH listed in Table C and a VL listed in Table D. The VH listed in Table C and the VL listed in table D can be different polypeptides or can be on the same polypeptide. When the VH and VL are on the same polypeptide, they can be in either orientation (i.e., VH-VL or VL-VH), and they can be connected by a linker (e.g., a glycine-serine linker). In certain aspects, the VH and VL are connected a glycine-serine linker that is at least 15 amino acids in length (e.g., 15-50 amino acids 15-40 amino acids, 15-30 amino acids, 15-25 amino acids or 15-20 amino acids). In certain aspects, the VH and VL are connected a glycine-serine linker that is at least 20 amino acids in length (e.g., 20-50 amino acids 20-40 amino acids, 20-30 amino acids, or 20-25 amino acids).

As described herein, a PSMA-binding domain can comprise a VH comprising the CDRs of a VH sequence in Table C, e.g., the IMGT-defined CDRs, the Kabat-defined CDRs, the Chothia-defined CDRs, or the AbM-defined CDRs and a VL comprising the CDRs of a VL sequence in Table D, e.g., the IMGT-defined CDRs, the Kabat-defined CDRs, the Chothia-defined CDRs, or the AbM-defined CDRs.

In certain aspects, a PSMA-binding domain comprises (i) a VH comprising the amino acid sequence of SEQ ID NO:82 (or a sequence that is at least about 70%, at least about 75%, at least about 80%, at least about 85%, at least about 90%, at least about 95%, at least about 96%, at least about 97%, at least about 98%, at least about 99% identical to SEQ ID NO:82, optionally wherein the VH comprises VH CDR1, VH CDR2, and VH CDR3 sequences of SEQ ID NOs:70, 72, and 74, respectively) and (ii) a VL comprising the amino acid sequence of SEQ ID NO:84 (or a sequence that is at least about 70%, at least about 75%, at least about 80%, at least about 85%, at least about 90%, at least about 95%, at least about 96%, at least about 97%, at least about 98%, at least about 99% identical to SEQ ID NO:84, optionally wherein the VL comprises VL CDR1, VL CDR2, and VL CDR3 sequences of SEQ ID NOs:76, 78, and 80, respectively).

In certain aspects, a PSMA-binding domain (e.g., an scFv) described herein binds to human PSMA and comprises one of the amino acid sequences set forth in Table E.

TABLE E
PSMA-Binding Sequences

| | scFv SEQ | VH SEQ | VL SEQ |
|---|---|---|---|
| PSMA-Binding Construct | ID NO | ID NO | ID NO |
| PSMA01107, 01108, 01116 | 86 | 82 | 84 |

As described herein, a PSMA×CD3 bispecific antibody that is monovalent for PSMA can comprise a single PSMA-binding domain comprising a sequence listed in Table E. A PSMA×CD3 bispecific antibody that is bivalent for PSMA can comprise two PSMA-binding domains, each comprising a sequence listed in Table E.

As described herein, a PSMA-binding domain can comprises an amino acid sequence at least about 70%, at least about 75%, at least about 80%, at least about 85%, at least about 90%, at least about 95%, at least about 96%, at least about 97%, at least about 98%, at least about 99% identical to a sequence in Table E, optionally wherein the sequence comprises VH CDR1, VH CDR2, and VH CDR3 sequences of SEQ ID NOs:70, 72, and 74, respectively, and VL CDR1, VL CDR2, and VL CDR3 sequences of SEQ ID NOs:76, 78, and 80, respectively.

In certain aspects, a PSMA-binding domain provided herein competitively inhibits binding of an antibody comprising a VH sequence in Table C (e.g., a VH comprising SEQ ID NO:82) and a VL sequence in Table D (e.g., a VL comprising SEQ ID NO:84) to human PSMA.

In certain aspects, a PSMA-binding domain provided herein specifically binds to the same epitope of human PSMA as an antibody comprising a VH sequence in Table C (e.g., a VH comprising SEQ ID NO:82) and a VL sequence in Table D (e.g., a VL comprising SEQ ID NO:84) to human PSMA.

### B. CD3-Binding Domains

Provided herein are antigen-binding domains that bind to human CD3 (i.e., CD3-binding domains) that can be used to assemble TAA (e.g., PSMA, HER2, or BCMA)×CD3 bispecific antibodies. A CD3-binding domain can bind to CD3 from other species, e.g. cynomolgus monkey and/or mouse CD3, in addition to binding to human CD3. In certain aspects, the CD3-binding domains bind to human CD3 and to cynomolgus monkey CD3. In certain aspects, the CD3-binding domains bind to human, cynomolgus monkey, and/or mouse CD3ε. The CD3 binding domain can have reduced binding strength, binding potency, and/or avidity to CD3 as compared to TSC266 and/or PSMA01110 in a Jurkat cell assay.

A CD3-binding domain can comprise six complementarity determining regions (CDRs), i.e., a variable heavy chain (VH) CDR1, a VH CDR2, a VH CDR3, a variable light chain (VL) CDR1, a VL CDR2, and a VL CDR3. A CD3-binding domain can comprise a variable heavy chain (VH) and a variable light chain (VL). The VH and the VL can be separate polypeptides or can parts of the same polypeptide (e.g., in an scFv).

In certain aspects, a CD3-binding domain described herein comprises the six CDRs listed in Tables F and G.

TABLE F
CD3 VH CDR Amino Acid Sequences³

| VH CDR1 | VH CDR2 | VH CDR3 |
|---|---|---|
| (SEQ ID NO:) | (SEQ ID NO:) | (SEQ ID NO:) |
| GYTFTRST | INPSSAYT | ASPQVHYDYNGFPY |
| (SEQ ID NO: 88) | (SEQ ID NO: 90) | (SEQ ID NO: 92) |

³The CDRs are determined according to IMGT.

TABLE G
CD3 VL CDR Amino Acid Sequences⁴

| VL CDR1 | VL CDR2 | VL CDR3 |
|---|---|---|
| (SEQ ID NO:) | (SEQ ID NO:) | (SEQ ID NO:) |
| SSVSY | DSS | QQWSRNPPT |
| (SEQ ID NO: 94) | (SEQ ID NO: 96) | (SEQ ID NO: 98) |

⁴The CDRs are determined according to IMGT.

A TAA (e.g., PSMA, HER2, or BCMA)×CD3 bispecific antibody that is monovalent for CD3 can comprise a single CD3-binding domain with the six CDRs listed in Tables F and G above. A TAA (e.g., PSMA, HER2, or BCMA)×CD3 bispecific antibody that is bivalent for CD3 can comprise two CD3-binding domains, each comprising the six CDRs listed in Tables F and G above.

As described herein, a CD3-binding can comprise the VH of an antibody listed in Table H.

TABLE H
CD3 Variable Heavy Chain (VH) Amino Acid Sequences

| SEQ ID NO | VH Amino Acid Sequence |
|---|---|
| 100 | QVQLVQSGAEVKKPGASVKVSCKASGYTFTRSTMHWVR |
| | QAPGQGLEWIGYINPSSAYTNYAQKFQGRVTLTADKST |
| | STAYMELSSLRSEDTAVYYCASPQVHYDYNGFPYWGQG |
| | TLVTVSS |

As described herein, a CD3-binding domain can comprise a VH having at least about 70%, at least about 75%, at least about 80%, at least about 85%, at least about 90%, at least about 95%, at least about 96%, at least about 97%, at least about 98%, at least about 99%, or 100% sequence identity to a sequence in Table H, optionally wherein the VH comprises VH CDR1, VH CDR2, and VH CDR3 sequences of SEQ ID NOs:88, 90, and 92, respectively.

As described herein, a CD3-binding domain can comprise a VH comprising the CDRs of a VH sequence in Table H, e.g., the IMGT-defined CDRs, the Kabat-defined CDRs, the Chothia-defined CDRs.

As described herein, a CD3-binding domain can comprise the VL of an antibody listed in Table I.

TABLE I
Variable Light Chain (VL) Amino Acid Sequences

| SEQ ID NO. | VL Amino Acid Sequence |
|---|---|
| 102 | DIQMTQSPSSLSASVGDRVT |
| | ITCRASSSVSYMNWYQQKPGKAPKRWIYDSSKLASGV |
| | PSRFSGSGSGTDFTLTISSLQPE |
| | DFATYYCQQWSRNPPTFGQGTKVEIK |

As described herein, a CD3-binding domain can comprise a VL having at least about 70%, at least about 75%, at least about 80%, at least about 85%, at least about 90%, at least about 95%, at least about 96%, at least about 97%, at least about 98%, at least about 99%, or 100% sequence identity to a sequence in Table I, optionally wherein the VL comprises VL CDR1, VL CDR2, and VL CDR3 sequences of SEQ ID NOs:94, 96, and 98, respectively.

As described herein, a CD3-binding domain can comprise a VL comprising the CDRs of a VL sequence in Table I, e.g., the IMGT-defined CDRs, the Kabat-defined CDRs, the Chothia-defined CDRs, or the AbM-defined CDRs.

As described herein, a CD3-binding domain can comprise a VH listed in Table H and a VL listed in Table I. A TAA (e.g., PSMA, HER2, or BCMA)×CD3 bispecific antibody that is monovalent for CD3 can comprise a single CD3-binding domain comprising a VH listed in Table H and a VL listed in Table I. A TAA (e.g., PSMA, HER2, or BCMA)×CD3 bispecific antibody that is bivalent for CD3 can comprise two CD3-binding domains, each comprising a VH listed in Table H and a VL listed in Table I. The VH listed in Table H and the VL listed in Table I can be different polypeptides or can be on the same polypeptide. When the VH and VL are on the same polypeptide, they can be in either orientation (i.e., VH-VL or VL-VH), and they can be connected by a linker (e.g., a glycine-serine linker). In certain aspects, the VH and VL are connected a glycine-serine linker that is at least 15 amino acids in length (e.g., 15-50 amino acids 15-40 amino acids, 15-30 amino acids, 15-25 amino acids or 15-20 amino acids). In certain aspects, the VH and VL are connected a glycine-serine linker that is at least 20 amino acids in length (e.g., 20-50 amino acids 20-40 amino acids, 20-30 amino acids, or 20-25 amino acids).

As described herein, a CD3-binding domain can comprise a VH comprising the CDRs of a VH sequence in Table H, e.g., the IMGT-defined CDRs, the Kabat-defined CDRs, the Chothia-defined CDRs, or the AbM-defined CDRs and a VL comprising the CDRs of a VL sequence in Table I, e.g., the IMGT-defined CDRs, the Kabat-defined CDRs, the Chothia-defined CDRs, or the AbM-defined CDRs.

In certain aspects, a CD3-binding domain comprises a (i) VH comprising the amino acid sequence of SEQ ID NO: 100 (or a sequence that is at least about 70%, at least about 75%, at least about 80%, at least about 85%, at least about 90%, at least about 95%, at least about 96%, at least about 97%, at least about 98%, at least about 99% identical to SEQ ID NO: 100, optionally wherein the VH comprises VH CDR1, VH CDR2, and VH CDR3 sequences of SEQ ID NOs:88, 90, and 92, respectively) and (ii) a VL comprising the amino acid sequence of SEQ ID NO: 102 (or a sequence that is at least about 70%, at least about 75%, at least about 80%, at least about 85%, at least about 90%, at least about 95%, at least about 96%, at least about 97%, at least about 98%, at least about 99% identical to SEQ ID NO:102, optionally wherein the VL comprises VL CDR1, VL CDR2, and VL CDR3 sequences of SEQ ID NOs:94, 96, and 98, respectively).

In certain aspects, a CD3-binding domain (e.g., an scFv) described herein binds to human CD3 and comprises one of the amino acid sequences set forth in Table J.

TABLE J
CD3-Binding Sequences

| | scFv SEQ | VH SEQ | VL SEQ |
|---|---|---|---|
| CD3-Binding Construct | ID NO | ID NO | ID NO |
| PSMA01107 | 104 | 100 | 102 |
| PSMA01108 | 110 | 100 | 102 |
| PSMA01116 | 104 | 100 | 102 |

As described herein, a TAA (e.g., PSMA, HER2, or BCMA)×CD3 bispecific antibody that is monovalent for CD3 can comprise a single CD3-binding domain comprising a sequence listed in Table J. A TAA (e.g., PSMA, HER2, or BCMA)×CD3 bispecific antibody that is bivalent for CD3 can comprise two CD3-binding domains, each comprising a sequence listed in Table J.

As described herein, a CD3-binding domain can comprises an amino acid sequence at least about 70%, at least about 75%, at least about 80%, at least about 85%, at least about 90%, at least about 95%, at least about 96%, at least about 97%, at least about 98%, at least about 99% identical to a sequence in Table J, optionally wherein the sequence comprises VH CDR1, VH CDR2, and VH CDR3 sequences of SEQ ID NOs:88, 90, and 92, respectively, and VL CDR1, VL CDR2, and VL CDR3 sequences of SEQ ID NOs:94, 96, and 98, respectively.

In certain aspects, a CD3-binding domain provided herein competitively inhibits binding of an antibody comprising a VH sequence in Table H (e.g., a VH comprising SEQ ID NO:100) and a VL sequence in Table I (e.g., a VL comprising SEQ ID NO:102) to human CD3.

In certain aspects, a CD3-binding domain provided herein specifically binds to the same epitope of human CD3 as an antibody comprising a VH sequence in Table H (e.g., a VH comprising SEQ ID NO: 100) and a VL sequence in Table I (e.g., a VL comprising SEQ ID NO: 102) to human CD3.

C. TAA and/or CD3-Binding Domains

In a TAA (e.g., PSMA, HER2, or BCMA) or CD3-binding domain, the VH CDRs or VH and the VL CDRs or VL can be separate polypeptides or can be on the same polypeptide. When the VH CDRs or VH and the VL CDRs or VL are on the same polypeptide, they can be in either orientation (i.e., VH-VL or VL-VH).

When the VH CDRs or VH and the VL CDRs or VL are on the same polypeptide, they can be connected by a linker (e.g., a glycine-serine linker). The VH can be positioned N-terminally to a linker sequence, and the VL can be positioned C-terminally to the linker sequence. Alternatively, the VL can be positioned N-terminally to a linker sequence, and the VH can be positioned C-terminally to the linker sequence.

The use of peptide linkers for joining VH and VL regions is well-known in the art, and a large number of publications exist within this particular field. In some aspects, a peptide linker is a 15mer consisting of three repeats of a Gly-Gly-Gly-Gly-Ser amino acid sequence ((Gly₄Ser)₃) (SEQ ID NO: 169). Other linkers have been used, and phage display technology, as well as selective infective phage technology, has been used to diversify and select appropriate linker sequences (Tang et al., J. Biol. Chem. 271, 15682-15686, 1996; Hennecke et al., Protein Eng. 11, 405-410, 1998). In certain aspects, the VH and VL regions are joined by a peptide linker having an amino acid sequence comprising the formula (Gly₄Ser)ₙ, wherein n=1-5. In certain aspects, n=3-10. In certain aspects, n=3-5. In certain aspects, n=4-10. In certain aspects, n=4-5. In certain aspects, n=4. Other suitable linkers can be obtained by optimizing a simple linker (e.g., (Gly₄Ser)ₙ), wherein n=1-5 through random mutagenesis.

The TAA (e.g., PSMA, HER2, or BCMA) and/or CD3-binding domain can be a humanized binding domain. The TAA (e.g., PSMA, HER2, or BCMA) and/or CD3-binding domain can be a rat binding domain. The TAA (e.g., PSMA, HER2, or BCMA) and/or CD3-binding domain can be a murine binding domain. In certain aspects, a TAA (e.g., PSMA, HER2, or BCMA)×CD3 bispecific antibody comprises a humanized TAA (e.g., PSMA, HER2, or BCMA)-binding domain and a rat CD3-binding domain. In certain aspects, a TAA (e.g., PSMA, HER2, or BCMA)×CD3 bispecific antibody comprises a humanized TAA (e.g., PSMA, HER2, or BCMA)-binding domain and a murine CD3-binding domain. In certain aspects, a TAA (e.g., PSMA, HER2, or BCMA)×CD3 bispecific antibody comprises a rat TAA (e.g., PSMA, HER2, or BCMA)-binding domain and a humanized CD3-binding domain. In certain aspects, a TAA (e.g., PSMA, HER2, or BCMA)×CD3 bispecific antibody comprises a murine TAA (e.g., PSMA, HER2, or BCMA)-binding domain and a humanized CD3-binding domain. In certain aspects, a TAA (e.g., PSMA, HER2, or BCMA)×CD3 bispecific antibody comprises a humanized TAA (e.g., PSMA, HER2, or BCMA)-binding domain and a humanized CD3-binding domain.

The TAA (e.g., PSMA, HER2, or BCMA) and/or CD3-binding domain can be an scFv. In certain aspects, all of the TAA (e.g., PSMA, HER2, or BCMA) and CD3-binding domains in a TAA (e.g., PSMA, HER2, or BCMA)×CD3 bispecific antibody are scFvs. In certain aspects, a TAA (e.g., PSMA, HER2, or BCMA) binding domain and a CD3-binding domain in a TAA (e.g., PSMA, HER2, or BCMA)×CD3 bispecific antibody are scFvs. In certain aspects, at least one TAA (e.g., PSMA, HER2, or BCMA) or CD3-binding domain in a TAA (e.g., PSMA, HER2, or BCMA)×CD3 bispecific antibody is an scFv. In certain aspects, a polypeptide comprises a TAA (e.g., PSMA, HER2, or BCMA)-binding domain (e.g., an scFv) and a CD3-binding domain (e.g., an scFv). Such a polypeptide can also contain an Fc domain. In certain aspects, a polypeptide comprises a TAA (e.g., PSMA, HER2, or BCMA)-binding domain (e.g., an scFv) and does not comprise a CD3-binding domain. Such a polypeptide can also contain an Fc domain. The TAA (e.g., PSMA, HER2, or BCMA) and/or CD3-binding domain can comprise a VH and a VL on separate polypeptide chains. In certain aspects, all of the TAA (e.g., PSMA, HER2, or BCMA) and CD3-binding domains in a TAA (e.g., PSMA, HER2, or BCMA)×CD3 bispecific antibody comprise a VH and a VL on separate polypeptide chains. In certain aspects, at least one TAA (e.g., PSMA, HER2, or BCMA) or CD3-binding domain in a TAA (e.g., PSMA, HER2, or BCMA)×CD3 bispecific antibody comprises a VH and a VL on separate polypeptide chains. In certain aspects, all of the TAA (e.g., PSMA, HER2, or BCMA) and CD3-binding domains in a TAA (e.g., PSMA, HER2, or BCMA)×CD3 bispecific antibody comprise a VH and a VL on the same polypeptide chains.

The TAA (e.g., PSMA) binding domain can have greater binding strength, binding potency, and/or avidity to PSMA than the CD3 binding domain has to CD3. The CD3 binding domain can have reduced binding strength, binding potency, and/or avidity to CD3 as compared to TSC266 in a Jurkat cell assay. The CD3 binding domain can have reduced binding strength, binding potency, and/or avidity to CD3 as compared to PSMA01110 in a Jurkat cell assay.

### D. TAA×CD3 Bispecific Antibodies

Provided herein are bispecific antibodies that bind to a TAA expressed on a solid tumor (e.g., PSMA, HER2, or BCMA) and CD3, wherein the CD3-binding domain has a low affinity for CD3. Such bispecific antibodies can have increased tumor localization (and decreased binding to CD3 on circulating T cells in the blood).

Provided herein are bispecific antibodies that bind to a TAA expressed on a solid tumor (e.g., PSMA, HER2, or BCMA) and CD3, wherein the bispecific is monovalent for CD3. Such bispecific antibodies can have increased tumor localization (and decreased binding to CD3 on circulating T cells in the blood).

Provided herein are bispecific antibodies that bind to a TAA expressed on a solid tumor (e.g., PSMA, HER2, or BCMA) and CD3, wherein the bispecific is monovalent for CD3 and wherein the CD3-binding domain has a low affinity for CD3. In some aspects, the bispecific antibodies provided herein can be monovalent for CD3 and bivalent for the TAA. Such bispecific antibodies can have increased tumor localization (and decreased binding to CD3 on circulating T cells in the blood).

Provided herein are bispecific antibodies that bind to a TAA (e.g., PSMA, HER2, or BCMA) and to human CD3 (PSMA×CD3 bispecific antibodies). Such bispecific antibodies comprise at least one humanized TAA (e.g., PSMA, HER2, or BCMA) binding domain and at least one humanized CD3-binding domain (e.g., humanized antibody derived from CRIS-7 or SP34). The TAA (e.g., PSMA, HER2, or BCMA) binding domain in the bispecific antibody can be any humanized or human TAA (e.g., PSMA, HER2, or BCMA) binding domain, including, e.g., any TAA (e.g., PSMA, HER2, or BCMA) binding domain discussed above. The CD3-binding domain in the bispecific antibody can be any humanized or human CD3-binding domain, including, e.g., any CD3-binding domain discussed above.

In certain aspects, the TAA (e.g., PSMA, HER2, or BCMA)×CD3 bispecific antibodies provided herein can bind to the TAA (e.g., PSMA, HER2, or BCMA) and CD3 simultaneously.

In certain aspects, the TAA (e.g., PSMA, HER2, or BCMA)×CD3 bispecific antibodies provided herein can increase T cell proliferation. In certain aspects, the TAA (e.g., PSMA, HER2, or BCMA)×CD3 bispecific antibodies provided herein can increase CD8 T cell proliferation. In certain aspects, the TAA (e.g., PSMA, HER2, or BCMA)×CD3 bispecific antibodies provided herein can increase CD4 T cell proliferation. In certain aspects, the TAA (e.g., PSMA, HER2, or BCMA)×CD3 bispecific antibodies provided herein can increase CD8 T cell proliferation and CD4 T cell proliferation. In certain aspects, the TAA (e.g., PSMA, HER2, or BCMA)×CD3 bispecific antibodies provided herein can increase T cell proliferation.

In certain aspects, the TAA (e.g., PSMA, HER2, or BCMA)×CD3 bispecific antibodies provided herein elicit decreased or no cytokine production when administered to a patient as compared to TAA×CD3 constructs with high CD3 affinity. In certain aspects, the TAA (e.g., PSMA, HER2, or BCMA)×CD3 bispecific antibodies provided herein elicit decreased or no can increase cytokine production when administered to a patient as compared to TAA×CD3 constructs with the same binding domains but in the BiTE format.

In some aspects, the TAA (e.g., (e.g., PSMA, HER2, or BCMA)×CD3 bispecific antibodies in the heterodimer format disclosed herein (e.g., ADAPTIR-FLEX™ format) elicit the production of none to reduced levels of one or more of IFN-γ, IL-2, TNF-α, and IL-6 compared to that in a mammal receiving a TAA×CD3 in the BiTE format. In some aspects, the TAA (e.g., (e.g., PSMA, HER2, or BCMA)×CD3 bispecific antibodies in the heterodimer format disclosed herein (e.g., ADAPTIR-FLEX™ format) elicit the production of none to reduced levels of one or more of Granzyme B, IL-10 and granulocyte-macrophage colony-stimulating factor (GM-CSF). In certain aspects, the TAA (e.g., PSMA, HER2, or BCMA)×CD3 bispecific antibodies provided herein (e.g., those ADAPTIR-FLEX™ format) cause insignificant to no IFN-γ production. In certain aspects, the TAA (e.g., PSMA, HER2, or BCMA)×CD3 bispecific antibodies provided herein (e.g., those in ADAPTIR-FLEX™ format) cause insignificant to no IL-2 production. In certain aspects, the TAA (e.g., PSMA, HER2, or BCMA)×CD3 bispecific antibodies provided herein (e.g., those in ADAPTIR-FLEX™ format) cause insignificant to no TNF-α production. In certain aspects, the TAA (e.g., PSMA, HER2, or BCMA)×CD3 bispecific antibodies provided herein (e.g., those in ADAPTIR-FLEX™ format) can insignificant to no IL-6 production. In certain aspects, the TAA (e.g., PSMA, HER2, or BCMA)×CD3 bispecific antibodies provided herein (e.g., those in ADAPTIR-FLEX™ format) can insignificant to no Granzyme B production. In certain aspects, the TAA (e.g., PSMA, HER2, or BCMA)×CD3 bispecific antibodies provided herein (e.g., those in ADAPTIR-FLEX™ format) can insignificant to no IL-10 production. In certain aspects, the TAA (e.g., PSMA, HER2, or BCMA)×CD3 bispecific antibodies provided herein (e.g., those in ADAPTIR-FLEX™ format) can insignificant to no GM-CSF production.

In one aspect provided herein, a PSMA×CD3 bispecific antibody comprising the amino acid sequences of SEQ ID NO:106 and SEQ ID NO: 108 or amino sequences at least about 90%, at least about 95%, at least about 96%, at least about 97%, at least about 98%, or at least about 99% identical to SEQ ID NO: 106 and SEQ ID NO:108 elicit the production of none to reduced levels of one or more of IFN-γ, IL-2, TNF-α, and IL-6 compared to that in a mammal receiving a TAA×CD3 in the BiTE format. In one aspect provided herein, a PSMA×CD3 bispecific antibody comprising the amino acid sequences of SEQ ID NO:112 and SEQ ID NO: 108 or amino sequences at least about 90%, at least about 95%, at least about 96%, at least about 97%, at least about 98%, or at least about 99% identical to SEQ ID NO: 112 and SEQ ID NO: 108 elicit the production of none to reduced levels of one or more of IFN-γ, IL-2, TNF-α, and IL-6 compared to that in a mammal receiving a TAA×CD3 in the BiTE format.

In one aspect provided herein, a PSMA×CD3 bispecific antibody comprising the amino acid sequences of SEQ ID NO:106 and SEQ ID NO: 108 or amino sequences at least about 90%, at least about 95%, at least about 96%, at least about 97%, at least about 98%, or at least about 99% identical to SEQ ID NO: 106 and SEQ ID NO:108 elicit the production of none to reduced levels of one or more of Granzyme B, IL-10 and granulocyte-macrophage colony-stimulating factor (GM-CSF) compared to that in a mammal receiving a TAA×CD3 in the BiTE format. In one aspect provided herein, a PSMA×CD3 bispecific antibody comprising the amino acid sequences of SEQ ID NO: 112 and SEQ ID NO: 108 or amino sequences at least about 90%, at least about 95%, at least about 96%, at least about 97%, at least about 98%, or at least about 99% identical to SEQ ID NO: 112 and SEQ ID NO:108 elicit the production of none to reduced levels of one or more of Granzyme B, IL-10 and granulocyte-macrophage colony-stimulating factor (GM-CSF) compared to that in a mammal receiving a TAA×CD3 in the BiTE format.

In certain aspects, the TAA (e.g., PSMA, HER2, or BCMA)×CD3 bispecific antibodies provided herein can increase cytotoxicity of a TAA-expressing cell. In certain aspects, the TAA (e.g., PSMA, HER2, or BCMA)×CD3 bispecific antibodies provided herein can increase re-directed T-cell cytotoxicity ADCC.

In certain aspects, the TAA (e.g., PSMA, HER2, or BCMA)×CD3 bispecific antibodies provided herein can increase tumor cell death in a TAA-expressing cell. In certain aspects, the TAA (e.g., PSMA, HER2, or BCMA)×CD3 bispecific antibodies provided herein can increase tumor cell death in vitro in a TAA-expressing cell. In certain aspects, the TAA (e.g., PSMA, HER2, or BCMA)×CD3 bispecific antibodies provided herein can increase tumor cell death in vivo in a TAA-expressing cell.

In certain aspects, a TAA (e.g., PSMA, HER2, or BCMA)×CD3 bispecific antibody comprises two TAA (e.g., PSMA, HER2, or BCMA) binding domains and one CD3-binding domain. In certain aspects, the two TAA (e.g., PSMA, HER2, or BCMA) binding domains comprise the same amino acid sequence. In certain aspects, the two TAA (e.g., PSMA, HER2, or BCMA) binding domains comprise different amino acid sequences. For instance, in one aspect provided herein a bispecific antibody comprises two PSMA binding domains and one CD3-binding domain, wherein the two PSMA binding domains are the same amino acid sequence or different amino acid sequences.

A TAA (e.g., PSMA, HER2, or BCMA)×CD3 bispecific antibody as provided herein can be prepared by chemically linking two different monoclonal antibodies or by fusing two hybridoma cell lines to produce a hybrid-hybridoma. Other multivalent formats that can be used include, for example, quadromas, Kλ-bodies, dAbs, diabodies, TandAbs, nanobodies, Small Modular ImmunoPharmaceutials (SMIPs™), DOCK-AND-LOCKs® (DNLs®), CrossMab Fabs, CrossMab VH-VLs, strand-exchange engineered domain bodies (SEEDbodies), Affibodies, Fynomers, Kunitz Domains, Albu-dabs, two engineered Fv fragments with exchanged VHs (e.g., a dual-affinity re-targeting molecules (D.A.R.T.s)), scFv×scFv (e.g., BiTE), DVD-IG, Covx-bodies, peptibodies, scFv-Igs, SVD-Igs, dAb-Igs, Knobs-in-Holes, IgG1 antibodies comprising matched mutations in the CH3 domain (e.g., DuoBody antibodies) and triomAbs. Exemplary bispecific formats are discussed in Garber et al., Nature Reviews Drug Discovery 13:799-801 (2014), which is herein incorporated by reference in its entirety. Additional exemplary bispecific formats are discussed in Liu et al. Front. Immunol. 8:38 doi: 10.2289/fimmu.2017.00038, and Brinkmann and Kontermann, MABS 9: 2, 182-212 (2017), each of which is herein incorporated by reference in its entirety. In certain aspects, a bispecific antibody can be a F(ab′)₂ fragment. A F(ab′)₂ fragment contains the two antigen-binding arms of a tetrameric antibody molecule linked by disulfide bonds in the hinge region.

TAA (e.g., PSMA, HER2, or BCMA)×CD3 bispecific antibodies disclosed herein can incorporate a multi-specific binding protein scaffold. Multi-specific binding proteins using scaffolds are disclosed, for instance, in PCT Application Publication No. WO 2007/146968, U.S. Patent Application Publication No. 2006/0051844, PCT Application Publication No. WO 2010/040105, PCT Application Publication No. WO 2010/003108, U.S. Pat. Nos. 7,166,707, and 8,409,577, each of which is herein incorporated by reference in its entirety. A TAA (e.g., PSMA, HER2, or BCMA)×CD3 bispecific antibody can comprise two binding domains (the domains can be designed to specifically bind the same or different targets), a hinge region, a linker (e.g., a carboxyl-terminus or an amino-terminus linker), and an immunoglobulin constant region. A TAA (e.g., PSMA, HER2, or BCMA)×CD3 bispecific antibody can be a homodimeric protein comprising two identical, disulfide-bonded polypeptides. A TAA (e.g., PSMA, HER2, or BCMA)×CD3 bispecific antibody can be a heterodimeric protein comprising two disulfide-bonded polypeptides.

In one aspect, the TAA (e.g., PSMA, HER2, or BCMA)×CD3 bispecific antibody comprises two polypeptides, each polypeptide comprising, in order from amino-terminus to carboxyl-terminus, a first antigen-binding domain, a linker (e.g., wherein the linker is a hinge region), an immunoglobulin constant region, and a second antigen-binding domain.

In one aspect, the bispecific antibody can comprise (a) a first polypeptide from N-terminus to C-terminus comprising (i) a first single chain variable fragment (scFv) that binds to a tumor-associated antigen (TAA), (ii) an immunoglobulin constant region, and (iii) an scFv that binds to CD3; and (b) a second polypeptide from N-terminus to C-terminus comprising (i) a second scFv that binds to a TAA, and (ii) an immunoglobulin constant region, wherein the bispecific antibody does not contain a second CD3-binding domain. The TAA can be, e.g., PSMA. Such antibodies are exemplified, e.g., by the schematics provided in FIGS. 1B, 1C, 1F (heterodimers), and 1G.

In one aspect, the bispecific antibody can comprise (a) a first polypeptide from N-terminus to C-terminus comprising (i) a first scFv that binds to PSMA(ii) an immunoglobulin constant region, and (iii) a first scFv that binds to CD3; and (b) a second polypeptide comprising (i) a second scFv that binds to PSMA, (ii) an immunoglobulin constant region, and (iii) a second scFv that binds to CD3. Such antibodies are exemplified, e.g., by schematics provided in FIGS. 1D, 1E, and 1F (homodimers).

In one aspect, the bispecific antibody can comprise (a) a first polypeptide from N-terminus to C-terminus comprising (i) a scFv that binds to PSMA and (ii) an immunoglobulin constant region; and (b) a second polypeptide from N-terminus to C-terminus comprising (i) a scFv that binds to a CD3 and (ii) an immunoglobulin constant region. Such antibodies are exemplified, e.g., by schematics provided in FIGS. 1A, 1F (two left-most heterodimers) and 1G (A-B construct).

In some aspects, a TAA (e.g., PSMA, HER2, or BCMA)×CD3 bispecific antibody comprises a polypeptide comprising in order from amino-terminus to carboxyl-terminus, a TAA (e.g., PSMA, HER2, or BCMA) binding domain (e.g., scFv), a linker (e.g., wherein the linker is a hinge region), an immunoglobulin constant region, a linker, and a CD3-binding domain (e.g., scFv). In certain aspects, the TAA (e.g., PSMA, HER2, or BCMA) binding domain (e.g., scFv) comprises in order from amino-terminus to carboxyl-terminus a VH, a linker (e.g., glycine-serine linker), and a VL. In certain aspects, the linker between the TAA (e.g., PSMA, HER2, or BCMA) binding domain and the immunoglobulin constant region is a hinge, and the hinge is an IgG₁ hinge. In certain aspects, the immunoglobulin constant region comprises a CH2 domain and a CH3 domain. In certain aspects, the CD3-binding domain (e.g., scFv) comprises in order from amino-terminus to carboxyl-terminus a VL, a linker (e.g., glycine-serine linker), and a VH.

Accordingly, in some aspects, a TAA (e.g., PSMA, HER2, or BCMA)×CD3 bispecific antibody comprises a polypeptide comprising in order from amino-terminus to carboxyl-terminus a VH of a TAA (e.g., PSMA, HER2, or BCMA) binding domain, a linker (e.g., a glycine-serine linker), a VL of a TAA (e.g., PSMA, HER2, or BCMA) binding domain, an IgG1 hinge, an immunoglobulin constant region comprising a CH2 domain and a CH3 domain, a linker (e.g., a glycine-serine linker), a VL of a CD3-binding domain, a linker (e.g., a glycine-serine linker), and a VH of a CD3-binding domain. In some aspects, a TAA (e.g., PSMA, HER2, or BCMA)×CD3 bispecific antibody comprises a homodimer or heterodimer of such polypeptides.

In some aspects, a TAA (e.g., PSMA, HER2, or BCMA)×CD3 bispecific antibody comprises a protein scaffold as generally disclosed in, for example, in US Patent Application Publication Nos. 2003/0133939, 2003/0118592, and 2005/0136049. A TAA (e.g., PSMA, HER2, or BCMA)×CD3 bispecific antibody may comprise a dimer (e.g., a homodimer) of two peptides, each comprising, in order from amino-terminus to carboxyl-terminus: a first antigen-binding domain, a linker (e.g., wherein the linker is a hinge region), and an immunoglobulin constant region. A TAA (e.g., PSMA, HER2, or BCMA)×CD3 antibody may comprise a dimer (e.g., a homodimer) of two peptides, each comprising, in order from amino-terminus to carboxyl-terminus: an immunoglobulin constant region, a linker (e.g., wherein the linker is a hinge region) and a first antigen-binding domain.

In some aspects, a TAA (e.g., PSMA, HER2, or BCMA)×CD3 bispecific antibody comprises two antigen-binding domains that are scFvs and two antigen-binding domains that comprises VHs and VLs on separate polypeptides. In such aspects, the scFvs can be fused to the N- or C-terminal of the polypeptide comprising the VH. The scFvs can also be fused to the N- or C-terminal of the polypeptide comprising the VL.

Additional exemplary bispecific antibody molecules provided herein comprise (i) an antibody that has two arms, each comprising two different antigen-binding regions, one with a specificity to a TAA (e.g., PSMA, HER2, or BCMA) and one with a specificity to CD3, (ii) an antibody that has one antigen-binding region or arm specific to a TAA (e.g., PSMA, HER2, or BCMA) and a second antigen-binding region or arm specific to CD3, (iii) a single chain antibody that has a first specificity to a TAA (e.g., PSMA, HER2, or BCMA) and a second specificity to CD3, e.g., via two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)₂ fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fab-arm; and (x) a diabody.

Examples of different classes of bispecific antibodies include but are not limited to IgG-like molecules with complementary CH3 domains to force heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

Examples of Fab fusion bispecific antibodies include but are not limited to F(ab)₂ (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). Examples of ScFv-, diabody-based and domain antibodies include but are not limited to Bispecific T Cell Engager (BiTE) (Micromet, Tandem Diabody (Tandab) (Affimed), Dual Affinity Retargeting Technology (D.A.R.T.) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), and dual targeting heavy chain only domain antibodies.

In some aspects, the bispecific antibody can comprise (a) a first polypeptide from N-terminus to C-terminus comprising (i) a first single chain variable (scFv) that binds to a TAA (e.g., PSMA, HER2, or BCMA), (ii) an immunoglobulin constant region, and (iii) an scFv that binds to CD3; and (b) a second polypeptide from N-terminus to C-terminus comprising (i) a second scFv that binds to the TAA (e.g., PSMA, HER2, or BCMA), and (ii) an immunoglobulin constant region, wherein the bispecific antibody does not contain a second CD3-binding domain. Such an antibody can comprise a linker in the first and/or second polypeptide, e.g., between one or more scFvs and immunoglobulin constant regions. Accordingly, in one aspect, the bispecific antibody comprises (a) a first polypeptide from N-terminus to C-terminus comprising (i) a first single chain variable (scFv) that binds to a TAA (e.g., PSMA, HER2, or BCMA), (ii) an optional linker, (iii) an immunoglobulin constant region, (iv) an optional linker, and (iv) an scFv that binds to CD3; and (b) a second polypeptide from N-terminus to C-terminus comprising (i) a second scFv that binds to the TAA (e.g., PSMA, HER2, or BCMA), (ii) an optional linker, and (iii) an immunoglobulin constant region, wherein the bispecific antibody does not contain a second CD3-binding domain.

As provided herein, a PSMA×CD3 bispecific antibody can comprise the PSMA VH CDR1, CDR2, and CDR3 sequences of SEQ ID NOs:70, 72, and 74, respectively, the PSMA VL CDR1, CDR2, and CDR3 sequences of SEQ ID NOs:76, 78, and 80, respectively, the CD3 VH CDR1, CDR2, and CDR3 sequences of SEQ ID NOs:88, 90, and 92, respectively, and the CD3 VL CDR1, CDR2, and CDR3 sequences of SEQ ID NOs:94, 96, and 98, respectively.

As provided herein, a PSMA×CD3 bispecific antibody can comprise any combination of PSMA VH and VL sequences and CD3 VH and VL sequences provided herein.

For example, a PSMA×CD3 bispecific antibody can comprise a PSMA-binding domain and a CD3-binding domain, wherein the PSMA-binding domain comprises a VH comprising the amino acid sequence of SEQ ID NO:82 and a VL comprising the amino acid sequence of SEQ ID NO:84, and wherein the CD3-binding domain comprises a VH comprising the amino acid sequence of SEQ ID NO: 100 and a VL comprising the amino acid sequence of SEQ ID NO: 102. In some aspects, both VH sequences and both VL sequences are on a single polypeptide chain (e.g., a single polypeptide containing one PSMA scFv and one CD3 scFv). In some aspects, one polypeptide comprises both VH sequences and another polypeptide comprises both VL sequences.

A PSMA×CD3 bispecific antibody can comprise a PSMA-binding domain and a CD3-binding domain, wherein the PSMA-binding domain comprises a VH comprising the amino acid sequence of SEQ ID NO:82 (or a sequence that is at least about 70%, at least about 75%, at least about 80%, at least about 85%, at least about 90%, at least about 95%, at least about 96%, at least about 97%, at least about 98%, at least about 99% identical to SEQ ID NO:82, optionally wherein the VH comprises VH CDR1, VH CDR2, and VH CDR3 sequences of SEQ ID NOs:70, 72, and 74, respectively) and a VL comprising the amino acid sequence of SEQ ID NO:84 (or a sequence that is at least about 70%, at least about 75%, at least about 80%, at least about 85%, at least about 90%, at least about 95%, at least about 96%, at least about 97%, at least about 98%, at least about 99% identical to SEQ ID NO:84, optionally wherein the VL comprises VL CDR1, VL CDR2, and VL CDR3 sequences of SEQ ID NOs:76, 78, and 80, respectively), and wherein the CD3-binding domain comprises a VH comprising the amino acid sequence of SEQ ID NO: 100 or a sequence that is at least about 70%, at least about 75%, at least about 80%, at least about 85%, at least about 90%, at least about 95%, at least about 96%, at least about 97%, at least about 98%, at least about 99% identical to SEQ ID NO: 100, optionally wherein the VH comprises VH CDR1, VH CDR2, and VH CDR3 sequences of SEQ ID NOs:88, 90, and 92, respectively) and a VL comprising the amino acid sequence of SEQ ID NO: 102 (or a sequence that is at least about 70%, at least about 75%, at least about 80%, at least about 85%, at least about 90%, at least about 95%, at least about 96%, at least about 97%, at least about 98%, at least about 99% identical to SEQ ID NO: 102, optionally wherein the VL comprises VL CDR1, VL CDR2, and VL CDR3 sequences of SEQ ID NOs:94, 96, and 98, respectively). In some aspects, both VH sequences and both VL sequences are on a single polypeptide chain (e.g., a single polypeptide containing one PSMA scFv and one CD3 scFv). In some aspects, one polypeptide comprises both VH sequences and another polypeptide comprises both VL sequences.

As provided herein, a PSMA×CD3 bispecific antibody can comprise any combination of PSMA scFv sequences and CD3 scFv sequences provided herein. For example, a PSMA×CD3 bispecific antibody can comprise the scFvs of SEQ ID NOs:86 and 104. A PSMA×CD3 bispecific antibody can comprise the scFvs of SEQ ID NOs:86 and 110. Such scFv pairs can be on the same polypeptide or on separate polypeptides. Where the scFv pairs are on the same polypeptide, the PSMA scFv can be N-terminal to the CD3 scFv or the PSMA scFv can be C-terminal to the CD3 scFv.

As provided herein, an antibody or polypeptide comprising any of the CDR, VH, VL, and/or scFv sequences provided herein may further comprise a hinge. A hinge can be located, for example between a TAA (e.g., PSMA, HER2, or BCMA) binding domain (e.g., an scFv) and an immunoglobulin constant region. In some aspects, a polypeptide comprises, in order from amino-terminus to carboxyl-terminus, an antigen-binding domain (e.g., an scFv), a hinge region, and an immunoglobulin constant region. In some aspects, a polypeptide comprises, in order from amino-terminus to carboxyl-terminus, a TAA binding domain (e.g., an scFv), a hinge region, an immunoglobulin constant region, and a CD3-binding domain (e.g., an scFv). In some aspects, a heterodimer comprises two polypeptides wherein the first polypeptide comprises, in order from amino terminus to carboxyl-terminus, a TAA binding domain (e.g., an scFv that binds PSMA), a hinge region, an immunoglobulin constant region, and a CD3-binding domain (e.g., an scFv) and the second polypeptide comprises, in order from amino terminus to carboxyl-terminus, a TAA binding domain (e.g., an scFv that binds PSMA), a hinge region, and an immunoglobulin constant region.

The hinge can be an immunoglobulin hinge, e.g., a human IgG hinge. In some aspects, the hinge is a human IgG₁ hinge. In some aspects, the hinge comprises amino acids 216-230 (according to EU numbering) of human IgG₁ or a sequence that is at least 90% identical thereto. For example, the hinge can comprise a substitution at amino acid C220 according to EU numbering of human IgG₁. If derived from a non-human source, a hinge can be humanized. In some aspects, the hinge comprises amino acids of SEQ ID NO:156. Non-limiting examples of hinges are provided in Tables K and L below.

In certain aspects, a hinge comprises or is a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a wild type immunoglobulin hinge region, such as a wild type human IgG1 hinge, a wild type human IgG2 hinge, or a wild type human IgG4 hinge.

Exemplary altered immunoglobulin hinges include an immunoglobulin human IgG1 hinge region having one, two or three cysteine residues found in a wild type human IgG1 hinge substituted by one, two or three different amino acid residues (e.g., serine or alanine). An altered immunoglobulin hinge can additionally have a proline substituted with another amino acid (e.g., serine or alanine). For example, the above-described altered human IgG1 hinge can additionally have a proline located carboxyl-terminal to the three cysteines of wild type human IgG1 hinge region substituted by another amino acid residue (e.g., serine, alanine). In one aspect, the prolines of the core hinge region are not substituted.

In certain aspects, hinge comprises about 5 to 150 amino acids, 5 to 10 amino acids, 10 to 20 amino acids, 20 to 30 amino acids, 30 to 40 amino acids, 40 to 50 amino acids, 50 to 60 amino acids, 5 to 60 amino acids, 5 to 40 amino acids, 8 to 20 amino acids, or 10 to 15 amino acids. The hinge can be primarily flexible, but can also provide more rigid characteristics or can contain primarily a-helical structure with minimal P-sheet structure. The lengths or the sequences of the hinges can affect the binding affinities of the binding domains to which the hinges are directly or indirectly (via another region or domain) connected as well as one or more activities of the Fc region portions to which the hinges or linkers are directly or indirectly connected.

In certain aspects, a hinge is stable in plasma and serum and is resistant to proteolytic cleavage. The first lysine in the IgG1 upper hinge region can be mutated to minimize proteolytic cleavage. For instance, the lysine can be substituted with methionine, threonine, alanine or glycine, or it can be deleted.

In some aspects, a TAA (e.g., PSMA, HER2, or BCMA)×CD3 bispecific antibody does not comprise a hinge. For instance, in some aspects, a TAA (e.g., PSMA, HER2, or BCMA)×CD3 bispecific antibody comprises a linker in the place of a hinge.

As provided herein, an antibody or polypeptide comprising any of the CDR, VH, VL, scFv, and/or hinge provided herein can further comprise an immunoglobulin constant region. An immunoglobulin constant region can be located, for example between a hinge and a PSMA-binding domain (e.g., a PSMA-binding scFv). An immunoglobulin constant region can also be located between a hinge and a CD3-binding domain (e.g., a CD3-binding scFv). In some aspects, a polypeptide comprises, in order from amino-terminus to carboxyl-terminus, a hinge region, an immunoglobulin constant region, and an antigen-binding domain (e.g., an scFv).

In some aspects, the immunoglobulin constant region comprises immunoglobulin CH2 and CH3 domains of IgG1, IgG2, IgG3, IgG4, IgA1, IgA2 or IgD, optionally wherein the IgG is human. In some cases, the immunoglobulin constant region comprises immunoglobulin CH2 and CH3 domains of IgG1 (e.g., human IgG1). In some aspects, the polypeptide does not contain a CH1 domain.

In some aspects, the immunoglobulin constant region comprises one, two, three, four, five or more amino acid substitutions and/or deletions to prevent binding to FcγRI, FcγRIIa, FcγRIIb, FcγRIIa, and FcγRIIIb.

In certain aspects, the immunoglobulin constant region comprises one, two, three or more amino acid substitutions to prevent or reduce Fc-mediated T-cell activation.

In some aspects, the immunoglobulin constant region comprises one, two, three, four or more amino acid substitutions and/or deletions to prevent or reduce CDC and/or ADCC activity. In some aspects, the immunoglobulin constant region comprises one, two, three, four, five or more amino acid substitutions and/or deletions to prevent or abate FcγR or C1q interactions.

Also provided herein is an antibody with a humanized TAA (e.g., PSMA, HER2, or BCMA) binding domain and a humanized CD3 antigen-binding domain containing the CDRs of the VH of SEQ ID NO:100 and CDRs of the VL of SEQ ID NO:102. The disclosure also includes an antibody with a humanized PSMA antigen-binding domain containing the CDRs of the VH of SEQ ID NO:82 and the CDRs of the VL of SEQ ID NO:84 and a humanized CD3 antigen-binding domain containing the CDRs of the VH of SEQ ID NO:100 and CDRs of the VL of SEQ ID NO: 102. In these aspects, the humanized TAA (e.g., PSMA, HER2, or BCMA) antigen-binding domain and the humanized CD3-binding domain can be separated by a “null” constant region that contains mutations that prevent binding to FcγRI, FcγRIIa, FcγRIIb, FcγRIIa, and FcγRIIIb. Such a “null” constant region allows the bispecific antibodies of the disclosure to activate tumor infiltrating lymphocytes while at the same time not activating or minimally activating other effector cells. The presence of the constant region extends the half-life of the bispecific antibody as compared to a similar bispecific antibody without a constant region.

In certain aspects, the immunoglobulin constant region comprises a human IgG1 CH2 domain comprising the substitutions L234A, L235A, G237A, and K322A, according to the EU numbering system.

In certain aspects, the immunoglobulin constant region comprises a human IgG1 CH2 domain comprising one or more of the following substitutions: E233P, L234A, L234V, L235A, G237A, E318A, K320A, and K322A, and/or a deletion of G236, according to the EU numbering system.

In certain aspects, the immunoglobulin constant region comprises a human IgG1 CH2 domain comprising one or more of the following substitutions: E233P, L234A, L234V, L235A, G237A, and K322A, and/or a deletion of G236, according to the EU numbering system.

In certain aspects, the immunoglobulin constant region comprises a human IgG1 CH2 domain comprising the substitutions L234A, L235A, G237A, E318A, K320A, and K322A, according to the EU numbering system.

In certain aspects, the immunoglobulin constant region comprises a human IgG1 CH2 domain comprising the substitutions L234A, L235A, G237A, and K322A, according to the EU numbering system.

In certain aspects, the immunoglobulin constant region comprises a human IgG1 CH2 domain comprising the substitutions E233P, L234V, L235A, G237A, and K322A, according to the EU numbering system.

In certain aspects, the immunoglobulin constant region comprises a human IgG1 CH2 domain comprising the substitutions E233P, L234V, L235A, G237A, and K322A, and a deletion of G236, according to the EU numbering system.

In certain aspects, the immunoglobulin constant region comprises a human IgG1 CH2 domain comprising the substitutions E233P, L234A, L235A, G237A, and K322A, according to the EU numbering system. For instance, the disclosure includes a bispecific antibody comprising, from amino terminus to carboxyl terminus, a first scFV, an immunoglobulin hinge, an IgG1 CH2 domain comprising the substitutions E233P, L234A, L235A, G237A, and K322A, according to the EU numbering system, an IgG1 CH3, and a second scFv. In one aspect, the first scFv specifically binds to a human TAA (e.g., PSMA, HER2, or BCMA) and the second scFv specifically binds to human CD3.

In certain aspects, the immunoglobulin constant region comprises a human IgG1 CH2 domain comprising the substitutions E233P, L234A, L235A, G237A, and K322A, and a deletion of G236, according to the EU numbering system. For instance, the disclosure includes a bispecific antibody comprising, from amino terminus to carboxyl terminus, a first scFv, an immunoglobulin hinge, an IgG1 CH2 comprising the substitutions E233P, L234A, L235A, G237A, and K322A, and a deletion of G236, according to the EU numbering system, an IgG1 CH3, and a second scFv. In one aspect, the first scFv specifically binds to a human TAA (e.g., PSMA, HER2, or BCMA) and the second scFv specifically binds to human CD3.

In certain aspects, the immunoglobulin constant region comprises a human IgG1 CH3 domain.

In certain aspects, the immunoglobulin constant region comprises the amino acids of SEQ ID NOs:64, 66, or 68.

Additional immunoglobulin constant regions that can be present in the TAA (e.g., PSMA, HER2, or BCMA)×CD3 antibodies provided herein are discussed in more detail below.

In some aspects, the hinge and the immunoglobulin constant region comprise the amino acid sequence of any one of SEQ ID NOs:64, 66, or 68. In some aspects, the hinge and the immunoglobulin constant region comprise the amino acid sequence of SEQ ID NO:66 or 68. In some aspects, the hinge comprises the amino acid sequence of any one of SEQ ID NOs:145-158. In some aspects, the hinge comprises the amino acid sequence of SEQ ID NO:156.

In some aspects, a TAA (e.g., PSMA, HER2, or BCMA)×CD3 bispecific antibody does not comprise an immunoglobulin constant region. In some aspects, a TAA (e.g., PSMA, HER2, or BCMA)×CD3 bispecific antibody does not comprise a hinge and does not comprise an immunoglobulin constant region.

As provided herein, an antibody or polypeptide comprising any of the CDR, VH, VL, scFv, hinge, and/or immunoglobulin constant region provided herein may further comprise a linker. A linker can be located, for example between an immunoglobulin constant region and a C-terminus binding domain. For instance, a linker can be located between an immunoglobulin constant region and a C-terminus TAA (e.g., PSMA, HER2, or BCMA) binding domain. A linker can also be located between an immunoglobulin constant region and a C-terminus CD3-binding domain In some aspects, a polypeptide comprises, in order from amino-terminus to carboxyl-terminus, an immunoglobulin constant region, a linker, and an antigen-binding domain.

In some aspects, the linker (e.g., between an immunoglobulin constant region and an antigen-binding domain) comprises 3-30 amino acids, 3-15 amino acids, or about 3-10 amino acids. In some aspects, the linker (e.g., between an immunoglobulin constant region and an antigen-binding domain) comprises 5-30 amino acids, 5-15 amino acids, or about 5-10 amino acids. In some aspects, the linker (e.g., between an immunoglobulin constant region and an antigen-binding domain) comprises the amino acid sequence (Gly₄Ser)ₙ, wherein n=1-5, optionally wherein n=1. In some aspects, the linker comprises the amino acid sequence of any one of SEQ ID NOs: 159-175. In some aspects, the linker (e.g., between an immunoglobulin constant region and an antigen-binding domain) comprises the amino acid sequence (G₄S)₄ (SEQ ID NO:171).

Non-limiting examples of linkers are provided in Tables K and L below.

TABLE K
Exemplary hinges and linkers

| | | SEQ |
|---|---|---|
| Name | Amino Acid Sequence | ID NO |
| sss(s)- | EPKSSDKTHTSPPSS | 145 |
| hIgG1 | | |
| hinge | | |
| csc(s)- | EPKSCDKTHTSPPCS | 146 |
| hIgG1 | | |
| hinge | | |
| ssc(s)- | EPKSSDKTHTSPPCS | 147 |
| hIgG1 | | |
| hinge | | |
| scc(s)- | EPKSSDKTHTCPPCS | 148 |
| hIgG1 | | |
| hinge | | |
| css(s)- | EPKSCDKTHTSPPSS | 149 |
| hIgG1 | | |
| hinge | | |
| scs(s)- | EPKSSDKTHTCPPSS | 150 |
| hIgG1 | | |
| hinge | | |
| ccc(s)- | EPKSCDKTHTSPPCS | 151 |
| hIgG1 | | |
| hinge | | |
| ccc(p)- | EPKSCDKTHTSPPCP | 152 |
| hIgG1 | | |
| hinge | | |
| sss(p)- | EPKSSDKTHTSPPSP | 153 |
| hIgG1 | | |
| hinge | | |
| csc(p)- | EPKSCDKTHTSPPCP | 154 |
| hIgG1 | | |
| hinge | | |
| ssc(p)- | EPKSSDKTHTSPPCP | 155 |
| hIgG1 | | |
| hinge | | |
| scc(p)- | EPKSSDKTHTCPPCP | 156 |
| hIgG1 | | |
| hinge | | |
| css(p)- | EPKSCDKTHTSPPSP | 157 |
| hIgG1 | | |
| hinge | | |
| scs(p)- | EPKSSDKTHTCPPSP | 158 |
| hIgG1 | | |
| hinge | | |
| Scppcp | SCPPCP | 159 |
| STD1 | NYGGGGSGGGGSGGGGSGNS | 160 |
| STD2 | NYGGGGGGGGSGGGGSGNYGGGGSGGGGSGG | 161 |
| | GGSGNS | |
| H1 | NS | 162 |
| H2 | GGGGSGNS | 163 |
| H3 | NYGGGGSGNS | 164 |
| H4 | GGGGSGGGGSGNS | 165 |
| H5 | NYGGGGSGGGGSGNS | 166 |
| H6 | GGGGSGGGGSGGGGSGNS | 167 |
| H7 | GCPPCPNS | 168 |
| (G₄S)₃ | GGGGSGGGGSGGGGS | 169 |
| H105 | SGGGGSGGGGSGGGGS | 170 |
| (G₄S)₄ | GGGGSGGGGSGGGGSGGGGS | 171 |
| H94 | SGGGGSGGGGSGGGGSPNS | 172 |
| H111 | SGGGGSGGGGSGGGGSPGS | 173 |
| H114 | GGGGSGGGGSGGGGSPS | 174 |
| G4S | GGGGS | 175 |

In some aspects, a PSMA×CD3 antibody comprises a polypeptide comprising in order from amino-terminus to carboxyl-terminus (i) a VH comprising the amino acids sequence of SEQ ID NO: 82, (ii) a linker (e.g., glycine-serine linker), (iii) a VL comprising the amino acid sequence of SEQ ID NO: 84, (iv) an IgG₁ hinge comprising a C220S substitution according to EU numbering, (v) an immunoglobulin constant region comprising a CH2 domain comprising the following substitutions: E233P, L234A, L234V, L235A, G237A, and K322A, and a deletion of G236, according to the EU numbering system) and a wild-type CH3 domain, (vi) a VL comprising the amino acid sequence of SEQ ID NO: 102, (vii) a linker (e.g., glycine-serine linker), and (viii) a VH comprising the amino acid sequence of SEQ ID NO: 100. In some aspects, a PSMA×CD3 antibody comprises a polypeptide comprising in order from amino-terminus to carboxyl-terminus (i) a VH comprising the amino acids sequence of SEQ ID NO: 82, (ii) a linker (e.g., glycine-serine linker), (iii) a VL comprising the amino acid sequence of SEQ ID NO: 84, (iv) an immunoglobulin constant region comprising a CH2 domain comprising the following substitutions: E233P, L234A, L234V, L235A, G237A, and K322A, and a deletion of G236, according to the EU numbering system) and a wild-type CH3 domain, (v) a VL comprising the amino acid sequence of SEQ ID NO: 102, (vi) a linker (e.g., glycine-serine linker), and (vii) a VH comprising the amino acid sequence of SEQ ID NO: 100. In some aspects, a PSMA×CD3 antibody comprises a heterodimer or homodimer of such a polypeptide.

In some aspects, a PSMA×CD3 antibody comprises a polypeptide comprising in order from amino-terminus to carboxyl-terminus (i) a VH comprising the amino acids sequence of SEQ ID NO:82, (ii) a linker (e.g., glycine-serine linker), (iii) a VL comprising the amino acid sequence of SEQ ID NO:84, (iv) an IgG₁ hinge comprising a C220S substitution according to EU numbering, (v) an immunoglobulin constant region comprising a CH2 domain comprising the following substitutions: E233P, L234A, L234V, L235A, G237A, and K322A, and a deletion of G236, according to the EU numbering system) and a wild-type CH3 domain, (vi) a VH comprising the amino acid sequence of SEQ ID NO: 100, (vii) a linker (e.g., glycine-serine linker), and (viii) a VL comprising the amino acid sequence of SEQ ID NO: 102. In some aspects, a PSMA×CD3 antibody comprises a heterodimer or homodimer of such a polypeptide.

In some aspects, a PSMA×CD3 bispecific antibody comprises the amino acid sequence of any one of SEQ ID NOs:78-100.

TABLE L
PSMA × CD3 Bispecific Antibody SEQ ID NOs

| PSMA × CD3 | | | | | | | | |
|---|---|---|---|---|---|---|---|---|
| bispecific | | | PSMA | PSMA | PSMA | CD3 | CD3 | CD3 |
| antibody | Chain 1 | Chain 2 | VH | VL | scFv | VH | VL | scFv |
| PSMA01107 | 106 | 108 | 82 | 84 | 86 | 100 | 102 | 104 |
| PSMA01108 | 106 | 112 | 82 | 84 | 86 | 100 | 102 | 110 |
| PSMA01116 | 178 | 108 | 82 | 84 | 86 | 100 | 102 | 104 |

In some aspects, a PSMA×CD3 bispecific antibody comprises a first polypeptide chain of amino acid sequence of SEQ ID NO: 106 and a second polypeptide chain of amino acid sequence SEQ ID NO: 108. In some aspects, a PSMA×CD3 bispecific antibody comprises a first polypeptide chain of amino acid sequence of SEQ ID NO: 106 and a second polypeptide chain of amino acid sequence SEQ ID NO: 112. In some aspects, a PSMA×CD3 bispecific antibody comprises a first polypeptide chain of amino acid sequence of SEQ ID NO: 178 and a second polypeptide chain of amino acid sequence SEQ ID NO: 108.

In some aspects, a PSMA×CD3 bispecific antibody is a heterodimer capable of binding to human PSMA and human CD3 and comprising two different polypeptides, with each polypeptide comprising an amino acid sequence that is at least 75%, 80%, 85%, 90%, 95%, 96%, 97%, 98%, or 99% or more to the amino acid sequences of SEQ ID NOs: 106 and 108, 178 and 108, or SEQ ID NOs:106 and 112. In some aspects, a PSMA×CD3 bispecific antibody is a heterodimer comprising two polypeptides, wherein each polypeptide comprises the amino acid sequences of SEQ ID NOs: 106 and 108, 178 and 108, or SEQ ID NOs:106 and 112. In some aspects, a bispecific antibody that binds to human PSMA and human CD3 is a heterodimer consisting essentially of or consisting of two polypeptides, wherein each polypeptide comprises the amino acid sequences of SEQ ID NOs: 106 and 108, 178 and 108, or SEQ ID NOs:106 and 112.

### E. PSMA and CD3 Monospecific Antibodies

Provided herein are monospecific antibodies that bind to either human PSMA or to human CD3. An anti-PSMA antibody provided herein can comprise one or more of any of the PSMA-binding domains described herein. An anti-CD3 antibody provided herein can comprise one or more of any of the CD3-binding domain described herein.

In some aspects, an anti-PSMA antibody or an anti-CD3 antibody provided herein is an IgG antibody. In some aspects, an anti-PSMA antibody or an anti-CD3 antibody provided herein is an IgG₁ antibody.

In some aspects, an anti-PSMA antibody comprises the six CDRs of SEQ ID NOs:70, 72, 74, 76, 78, and 80 or a combination of PSMA-binding VH and VL sequences provided herein and a heavy chain constant region. In some aspects, an anti-PSMA antibody comprises the six CDRs of SEQ ID NOs: 70, 72, 74, 76, 78, and 80, or a combination of PSMA-binding VH and VL sequences provided herein and a light chain constant region. In some aspects, an anti-PSMA antibody comprises the six CDRs of SEQ ID NOs: 70, 72, 74, 76, 78, and 80, or a combination of PSMA-binding VH and VL sequences provided herein and, a heavy chain constant region, and a light chain constant region.

In some aspects, an anti-CD3 antibody comprises the six CDRs of SEQ ID NOs:88, 90, 92, 94, 96, and 98, or a combination of CD3-binding VH and VL sequences provided herein and a heavy chain constant region. In some aspects, an anti-CD3 antibody comprises the six CDRs of SEQ ID NOs:88, 90, 92, 94, 96, and 98, or a combination of CD3-binding VH and VL sequences provided herein and a light chain constant region. In some aspects, an anti-CD3 antibody comprises the six CDRs of SEQ ID NOs:88, 90, 92, 94, 96, and 98, or a combination of CD3-binding VH and VL sequences provided herein and, a heavy chain constant region, and a light chain constant region.

The constant region of an anti-PSMA antibody or a CD3 antibody can be any constant region discussed herein. Constant regions that can be present in these antibodies are discussed in more detail below.

In some aspects, an anti-PSMA antibody or an anti-CD3 antibody is a Fab, Fab′, F(ab′)₂, scFv, disulfide linked Fv, or scFv-Fc. In some aspects, an anti-PSMA antibody or an anti-CD3 antibody comprises a Fab, Fab′, F(ab′)₂, scFv, disulfide linked Fv, or scFv-Fc. For instance, the disclosure includes an anti-PSMA antibody or an anti-CD3 antibody in the SMIP format (i.e., scFv-Fc) as disclosed in U.S. Pat. No. 9,005,612. A SMIP antibody may comprise, from amino-terminus to carboxyl-terminus, an scFv and a modified constant domain comprising an immunoglobulin hinge and a CH2/CH3 region. The disclosure also includes an anti-PSMA antibody or an anti-CD3 antibody in the PIMS format as disclosed in published US patent application 2009/0148447. A PIMS antibody may comprise, from amino-terminus to carboxyl-terminus, a modified constant domain comprising an immunoglobulin hinge and CH2/CH3 region, and an scFv.

An anti-PSMA antibody can be monovalent for PSMA (i.e., contain one PSMA-binding domain), bivalent for PSMA (i.e., contain two PSMA-binding domains), or can have three or more PSMA-binding domains.

An anti-CD3 antibody can be monovalent for CD3 (i.e., contain one CD3-binding domain), bivalent for CD3 (i.e., contain two CD3-binding domains), or can have three or more CD3-binding domains.

### F. Constant Regions

As discussed above antibodies provided herein, including monospecific antibodies that bind to PSMA or CD3 as well as TAA (e.g., PSMA, HER2, or BCMA)×CD3 or PSMA×CD3 bispecific antibodies, can comprise immunoglobulin constant regions. In certain aspects, the immunoglobulin constant region does not interact with Fc gamma receptors.

In a specific aspect, an antibody described herein, which immunospecifically binds to a TAA (e.g., PSMA, HER2, or BCMA) and/or CD3 comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In another specific aspect, an antibody described herein, which immunospecifically binds to TAA (e.g., PSMA, HER2, or BCMA) and/or CD3 comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In a particular aspect, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

In one aspect, the heavy chain constant region is a human IgG₁ heavy chain constant region, and the light chain constant region is a human IgGκ light chain constant region.

In some aspects, the constant region comprises one, two, three or more amino acid substitutions to prevent binding to FcγRI, FcγRIIa, FcγRIIb, FcγRIIa, and FcγRIIIb.

In certain aspects, the constant region comprises one, two, three or more amino acid substitutions to prevent or reduce Fc-mediated T-cell activation.

In some aspects, the constant region comprises one, two, three or more amino acid substitutions to prevent or reduce CDC and/or ADCC activity.

In some aspects, one, two, or more mutations (e.g., amino acid substitutions) are introduced into the Fc region of an antibody or antigen-binding fragment thereof described herein (e.g., CH2 domain (residues 231-340 of human IgG₁) and/or CH3 domain (residues 341-447 of human IgG₁) and/or the hinge region, with numbering according to the Kabat numbering system (e.g., the EU index in Kabat)) to alter one or more functional properties of the antibody or antigen-binding fragment thereof, such as serum half-life, complement fixation, Fc receptor binding, and/or antigen-dependent cellular cytotoxicity.

In certain aspects, one, two, or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain may be altered to, e.g., facilitate assembly of the light and heavy chains, or to alter (e.g., increase or decrease) the stability of the antibody or antigen-binding fragment thereof.

In some aspects, one, two, or more mutations (e.g., amino acid substitutions) are introduced into the Fc region of an antibody or antigen-binding fragment thereof described herein (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody or antigen-binding fragment thereof for an Fc receptor (e.g., an activated Fc receptor) on the surface of an effector cell. Mutations in the Fc region that decrease or increase affinity for an Fc receptor and techniques for introducing such mutations into the Fc receptor or fragment thereof are known to one of skill in the art. Examples of mutations in the Fc receptor that can be made to alter the affinity of the antibody or antigen-binding fragment thereof for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In a specific aspect, one, two, or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody or antigen-binding fragment thereof in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody or antigen-binding fragment thereof in vivo. In some aspects, one, two or more amino acid mutations (i.e., substitutions, insertions, or deletions) are introduced into an IgG constant domain, or FcRn-binding fragment thereof (preferably an Fc or hinge-Fc domain fragment) to decrease the half-life of the antibody or antigen-binding fragment thereof in vivo. In other aspects, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody or antigen-binding fragment thereof in vivo. In a specific aspect, the antibodies or antigen-binding fragments thereof may have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In a specific aspect, the constant region of the IgG1 comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In certain aspects, an antibody or antigen-binding fragment thereof comprises an IgG constant domain comprising one, two, three or more amino acid substitutions of amino acid residues at positions 251-257, 285-290, 308-314, 385-389, and 428-436, numbered according to the EU index as in Kabat.

In a further aspect, one, two, or more amino acid substitutions are introduced into an IgG constant domain Fc region to alter the effector function(s) of the antibody or antigen-binding fragment thereof. For example, one or more amino acids selected from amino acid residues 234, 235, 236, 237, 297, 318, 320 and 322, numbered according to the EU index as in Kabat, can be replaced with a different amino acid residue such that the antibody or antigen-binding fragment thereof has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260. In some aspects, the deletion or inactivation (through point mutations or other means) of a constant region domain may reduce Fc receptor binding of the circulating antibody or antigen-binding fragment thereof thereby increasing tumor localization. See, e.g., U.S. Pat. Nos. 5,585,097 and 8,591,886 for a description of mutations that delete or inactivate the constant domain and thereby increase tumor localization. In certain aspects, one or more amino acid substitutions can be introduced into the Fc region to remove potential glycosylation sites on Fc region, which may reduce Fc receptor binding (see, e.g., Shields R L et al., (2001) J Biol Chem 276: 6591-604).

In certain aspects, one or more amino acids selected from amino acid residues 329, 331, and 322 in the constant region, numbered according to the EU index as in Kabat, can be replaced with a different amino acid residue such that the antibody or antigen-binding fragment thereof has altered C1q binding and/or reduced or abolished complement dependent cytotoxicity (CDC). This approach is described in further detail in U.S. Pat. No. 6,194,551 (Idusogie et al). In some aspects, one or more amino acid residues within amino acid positions 231 to 238 in the N-terminal region of the CH2 domain are altered to thereby alter the ability of the antibody to fix complement. This approach is described further in International Publication No. WO 94/29351. In certain aspects, the Fc region is modified to increase the ability of the antibody or antigen-binding fragment thereof to mediate antibody dependent cellular cytotoxicity (ADCC) and/or to increase the affinity of the antibody or antigen-binding fragment thereof for an Fcg receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU index as in Kabat. This approach is described further in International Publication No. WO 00/42072.

In certain aspects, an antibody or antigen-binding fragment thereof described herein comprises the constant domain of an IgG1 with a mutation (e.g., substitution) at position 267, 328, or a combination thereof, numbered according to the EU index as in Kabat. In certain aspects, an antibody or antigen-binding fragment thereof described herein comprises the constant domain of an IgG1 with a mutation (e.g., substitution) selected from the group consisting of S267E, L328F, and a combination thereof. In certain aspects, an antibody or antigen-binding fragment thereof described herein comprises the constant domain of an IgG1 with a S267E/L328F mutation (e.g., substitution). In certain aspects, an antibody or antigen-binding fragment thereof described herein comprising the constant domain of an IgG1 with a S267E/L328F mutation (e.g., substitution) has an increased binding affinity for FcγRIIA, FcγRIIB, or FcγRIIA and FcγRIIB.

In certain aspects, any of the constant region mutations or modifications described herein can be introduced into one or both heavy chain constant regions of an antibody or antigen-binding fragment thereof described herein having two heavy chain constant regions.

### III. Antibody Production

Antibodies that immunospecifically bind to a TAA (e.g., PSMA, HER2, or BCMA) and/or human CD3 can be produced by any method known in the art for the synthesis of antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

Bispecific antibodies as provided herein can be prepared by expressing a polynucleotide in a host cell, wherein the polynucleotide encodes a polypeptide comprising, in order from amino-terminus to carboxyl-terminus, a first scFv, a hinge region, an immunoglobulin constant region, and a second scFv, wherein (a) the first scFv comprises a humanized TAA (e.g., PSMA, HER2, or BCMA) antigen-binding domain, and the second scFv comprises a humanized CD3 antigen-binding domain or (b) the first scFv comprises a humanized CD3 antigen-binding domain and the second scFv comprises a humanized TAA (e.g., PSMA, HER2, or BCMA) antigen-binding domain. The polypeptide can be expressed in the host cell as a homodimer or heterodimer.

Bispecific antibodies as provided herein can be prepared by chemically linking two different monoclonal antibodies or by fusing two hybridoma cell lines to produce a hybrid-hybridoma. Bispecific, bivalent antibodies, and methods of making them, are described, for instance in U.S. Pat. Nos. 5,731,168, 5,807,706, 5,821,333, and U.S. Appl. Publ. Nos. 2003/020734 and 2002/0155537; each of which is herein incorporated by reference in its entirety. Bispecific tetravalent antibodies, and methods of making them are described, for instance, in Int. Appl. Publ. Nos. WO02/096948 and WO00/44788, the disclosures of both of which are herein incorporated by reference in its entirety. See generally, Int. Appl. Publ. Nos. WO93/17715, WO92/08802, WO91/00360, and WO92/05793; Tutt et al., J. Immunol. 147:60-69 (1991); U.S. Pat. Nos. 4,474,893; 4,714,681; 4,925,648; 5,573,920; and 5,601,819; and Kostelny et al., J. Immunol. 148:1547-1553 (1992); each of which is herein incorporated by reference in its entirety.

A bispecific antibody as described herein can be generated according to the DuoBody technology platform (Genmab A/S) as described, e.g., in International Publication Nos. WO 2011/131746, WO 2011/147986, WO 2008/119353, and WO 2013/060867, and in Labrijn A F et al., (2013) PNAS 110(13): 5145-5150. The DuoBody technology can be used to combine one half of a first monospecific antibody containing two heavy and two light chains with one half of a second monospecific antibody containing two heavy and two light chains. The resultant heterodimer contains one heavy chain and one light chain from the first antibody paired with one heavy chain and one light chain from the second antibody. When both of the monospecific antibodies recognize different epitopes on different antigens, the resultant heterodimer is a bispecific antibody.

The DuoBody technology requires that each of the monospecific antibodies includes a heavy chain constant region with a single point mutation in the CH3 domain. The point mutations allow for a stronger interaction between the CH3 domains in the resultant bispecific antibody than between the CH3 domains in either of the monospecific antibodies. The single point mutation in each monospecific antibody is at residue 366, 368, 370, 399, 405, 407, or 409, numbered according to the EU numbering system, in the CH3 domain of the heavy chain constant region, as described, e.g., in International Publication No. WO 2011/131746. Moreover, the single point mutation is located at a different residue in one monospecific antibody as compared to the other monospecific antibody. For example, one monospecific antibody can comprise the mutation F405L (i.e., a mutation from phenylalanine to leucine at residue 405), while the other monospecific antibody can comprise the mutation K409R (i.e., a mutation from lysine to arginine at residue 409), numbered according to the EU numbering system. The heavy chain constant regions of the monospecific antibodies can be an IgG₁, IgG₂, IgG₃, or IgG₄ isotype (e.g., a human IgG₁ isotype), and a bispecific antibody produced by the DuoBody technology can retain Fc-mediated effector functions.

Another method for generating bispecific antibodies has been termed the “knobs-into-holes” strategy (see, e.g., Intl. Publ. WO2006/028936). The mispairing of Ig heavy chains is reduced in this technology by mutating selected amino acids forming the interface of the CH3 domains in IgG. At positions within the CH3 domain at which the two heavy chains interact directly, an amino acid with a small side chain (hole) is introduced into the sequence of one heavy chain and an amino acid with a large side chain (knob) into the counterpart interacting residue location on the other heavy chain. In some aspects, compositions of the disclosure have immunoglobulin chains in which the CH3 domains have been modified by mutating selected amino acids that interact at the interface between two polypeptides so as to preferentially form a bispecific antibody. The bispecific antibodies can be composed of immunoglobulin chains of the same subclass (e.g., IgG1 or IgG3) or different subclasses (e.g., IgG1 and IgG3, or IgG3 and IgG4).

In one aspect, a bispecific antibody that binds to TAA (e.g., PSMA, HER2, or BCMA) and CD3 comprises a T366W mutation in the “knobs chain” and T366S, L368A, Y407V mutations in the “hole chain,” and optionally an additional interchain disulfide bridge between the CH3 domains by, e.g., introducing a Y349C mutation into the “knobs chain” and a E356C mutation or a S354C mutation into the “hole chain;” R409D, K370E mutations in the “knobs chain” and D399K, E357K mutations in the “hole chain;” R409D, K370E mutations in the “knobs chain” and D399K, E357K mutations in the “hole chain;” a T366W mutation in the “knobs chain” and T366S, L368A, Y407V mutations in the “hole chain;” R409D, K370E mutations in the “knobs chain” and D399K, E357K mutations in the “hole chain;” Y349C, T366W mutations in one of the chains and E356C, T366S, L368A, Y407V mutations in the counterpart chain; Y349C, T366W mutations in one chain and S354C, T366S, L368A, Y407V mutations in the counterpart chain; Y349C, T366W mutations in one chain and S354C, T366S, L368A, Y407V mutations in the counterpart chain; and Y349C, T366W mutations in one chain and S354C, T366S, L368A, Y407V mutations in the counterpart chain (numbering according to the EU numbering system). In certain aspects, the Fe region can comprise SEQ ID NOs: 64, 66, or 68. In certain aspects, the Fc region can have PAA deleted and can have amino acid alterations to allow for Knob and Hole connections.

Bispecific antibodies that bind to TAA (e.g., PSMA, HER2, or BCMA) and CD3 can, in some aspects, contain, IgG4 and IgG1, IgG4 and IgG2, IgG4 and IgG2, IgG4 and IgG3, or IgG1 and IgG3 chain heterodimers. Such heterodimeric heavy chain antibodies, can routinely be engineered by, for example, modifying selected amino acids forming the interface of the CH3 domains in human IgG4 and the IgG1 or IgG3 so as to favor heterodimeric heavy chain formation.

Bispecific antibodies described herein can be generated by any technique known to those of skill in the art. For example, F(ab′)₂ fragments described herein can be produced by proteolytic cleavage of immunoglobulin molecules, using enzymes such as pepsin.

In a certain aspect, provided herein is a method of making an antibody which immunospecifically binds to a human TAA (e.g., PSMA, HER2, or BCMA) and/or human CD3 comprising culturing a cell or cells described herein. In a certain aspect, provided herein is a method of making an antibody that immunospecifically binds to a human TAA (e.g., PSMA, HER2, or BCMA) and/or human CD3 comprising expressing (e.g., recombinantly expressing) the antibody using a cell or host cell described herein (e.g., a cell or a host cell comprising polynucleotides encoding an antibody described herein). In a particular aspect, the cell is an isolated cell. In a particular aspect, the exogenous polynucleotides have been introduced into the cell. In a particular aspect, the method further comprises the step of purifying the antibody from the cell or host cell.

Monoclonal antibodies can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981). The term “monoclonal antibody” as used herein is not limited to antibodies produced through hybridoma technology. For example, monoclonal antibodies can be produced recombinantly from host cells exogenously expressing an antibody described herein. Monoclonal antibodies described herein can, for example, be made by the hybridoma method as described in Kohler G & Milstein C (1975) Nature 256: 495 or can, e.g., be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel F M et al., supra).

Further, the antibodies described herein can also be generated using various phage display methods known in the art. In phage display methods, proteins are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antibody that binds to a particular antigen can be selected or identified with antigen, e.g., using labeled antigen or antigen bound or captured to a solid surface or bead. Examples of phage display methods that can be used to make the antibodies described herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT Application No. PCT/GB91/001 134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108.

As described in the above references, after phage selection, the antibody coding regions from the phage can be isolated and used to generate antibodies, including human antibodies, and expressed in any desired host, including mammalian cells, insect cells, plant cells, yeast, and bacteria, e.g., as described below. Techniques to recombinantly produce antibodies such as Fab, Fab′ and F(ab′)₂ fragments can also be employed using methods known in the art such as those disclosed in PCT publication No. WO 92/22324; Mullinax R L et al., (1992) BioTechniques 12(6): 864-9; Sawai H et al., (1995) Am J Reprod Immunol 34: 26-34; and Better M et al., (1988) Science 240: 1041-1043.

In one aspect, to generate antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express antibodies, e.g., IgG, using techniques known to those of skill in the art.

A humanized antibody is capable of binding to a predetermined antigen and comprises a framework region having substantially the amino acid sequence of a human immunoglobulin and CDRs having substantially the amino acid sequence of a non-human immunoglobulin (e.g., a murine immunoglobulin). In particular aspects, a humanized antibody also comprises at least a portion of an immunoglobulin constant region (Fc), typically that of a human immunoglobulin. The antibody also can include the CH1, hinge, CH2, CH3, and CH4 regions of the heavy chain. A humanized antibody can be selected from any class of immunoglobulins, including IgM, IgG, IgD, IgA and IgE, and any isotype, including IgG₁, IgG₂, IgG₃ and IgG₄. Humanized antibodies can be produced using a variety of techniques known in the art, including but not limited to, CDR-grafting (European Patent No. EP 239400; International Publication No. WO 91/09967; and U.S. Pat. Nos. 5,225,539, 5,530,101, and 5,585,089), veneering or resurfacing (European Patent Nos. EP 592106 and EP 519596; Padlan E A (1991) Mol Immunol 28(4/5): 489-498; Studnicka G M et al., (1994) Prot Engineering 7(6): 805-814; and Roguska M A et al., (1994) PNAS 91: 969-973), chain shuffling (U.S. Pat. No. 5,565,332), and techniques disclosed in, e.g., U.S. Pat. Nos. 6,407,213, 5,766,886, International Publication No. WO 93/17105; Tan P et al., (2002) J Immunol 169: 1119-25; Caldas C et al., (2000) Protein Eng. 13(5): 353-60; Morea V et al., (2000) Methods 20(3): 267-79; Baca M et al., (1997) J Biol Chem 272(16): 10678-84; Roguska M A et al., (1996) Protein Eng 9(10): 895 904; Couto J R et al., (1995) Cancer Res. 55 (23 Supp): 5973s-5977s; Couto J R et al., (1995) Cancer Res 55(8): 1717-22; Sandhu J S (1994) Gene 150(2): 409-10 and Pedersen J T et al., (1994) J Mol Biol 235(3): 959-73. See also U.S. Application Publication No. US 2005/0042664 A1 (Feb. 24, 2005), which is herein incorporated by reference in its entirety.

### IV. Polynucleotides Encoding Antibodies

In certain aspects, the disclosure encompasses polynucleotides comprising a nucleic acid that encodes an antibody that binds to a TAA (e.g., PSMA, HER2, or BCMA) and/or CD3, or polypeptide of such an antibody, e.g., a VH, a VL, a VH with a VL (e.g., in an scFv), a heavy chain, a light chain, a heavy chain with an scFv, a light chain with an scFv, a fusion protein comprising an scFv, a linker (e.g., wherein the linker is a hinge), an immunoglobulin constant region, and an scFv, a constant region, or a constant region with an scFv.

In certain aspects, the disclosure encompasses polynucleotides comprising a nucleic acid that encodes an antibody that binds to PSMA and/or CD3, or polypeptide of such an antibody, e.g., a VH, a VL, a VH with a VL (e.g., in an scFv), a heavy chain, a light chain, a heavy chain with an scFv, a light chain with an scFv, a fusion protein comprising an scFv, a linker (e.g., wherein the linker is a hinge), an immunoglobulin constant region, and an scFv, a constant region, or a constant region with an scFv.

Accordingly, provided herein are polynucleotides or combinations of polynucleotides encoding the six CDRs of the PSMA-binding domain of SEQ ID NOs:70, 72, 74, 76, 78, and 80, respectively. The polynucleotides can comprise the nucleotide sequences set forth as SEQ ID NOs:69, 71, 73, 75, 77, and 79, respectively.

Provided herein are also polynucleotides or combinations of polynucleotides encoding the six CDRs of the CD3-binding domain of SEQ ID NOs:88, 90, 92, 94, 96, and 98, respectively. The polynucleotides can comprise the nucleotide sequences set forth as SEQ ID NOs:87, 89, 91, 93, 95, and 97.

Also provided herein are polynucleotides encoding a VH of the PSMA-binding domains provided herein, e.g., a VH comprising the amino acid sequence of SEQ ID NO:82. The polynucleotides can comprise the nucleotide sequences set forth as SEQ ID NO:81.

Also provided herein are polynucleotides encoding a VL of the PSMA-binding domain provided herein, e.g., a VL comprising the amino acid sequence of SEQ ID NO:84. The polynucleotides can comprise the nucleotide sequences set forth as SEQ ID NO:83.

Also provided herein are polynucleotides encoding a VH of the CD3-binding domains provided herein, e.g., a VH comprising the amino acid sequence of SEQ ID NO:100. The polynucleotides can comprise the nucleotide sequences set forth as SEQ ID NO:99.

Also provided herein are polynucleotides encoding a VL of the CD3-binding domain provided herein, e.g., a VL comprising the amino acid sequence of SEQ ID NO: 102. The polynucleotides can comprise the nucleotide sequences set forth as SEQ ID NO:101.

Also provided herein are polynucleotides encoding a PSMA-binding sequence (e.g., scFv) provided herein, e.g., a PSMA-binding sequence comprising the amino acid sequence of SEQ ID NO:86. The polynucleotides can comprise the nucleotide sequences set forth as SEQ ID NO:85.

Also provided herein are polynucleotides encoding a CD3-binding sequence (e.g., scFv) provided herein, e.g., a CD3-binding sequence comprising the amino acid sequence of SEQ ID NOs:104 or 110. The polynucleotides can comprise the nucleotide sequences set forth as SEQ ID NOs:103 or 109.

Also provided herein are polynucleotides encoding PSMA×CD3 bispecific antibodies provided herein, e.g., an antibody comprising the first and second polypeptide chains of amino acid sequence of SEQ ID NOs:106 and 108, 178 and 108, or 112 and 108. The polynucleotides can comprise the nucleotide sequences set forth in any one of SEQ ID NOs:105 and 107, 177 and 107, or 111 and 107.

In certain aspects, a polynucleotide encodes a polypeptide comprising, in order from amino-terminus to carboxyl-terminus, a first scFv, a linker (e.g., wherein the linker is a hinge region), an immunoglobulin constant region, and a second scFv, wherein (a) the first scFv comprises a humanized TAA (e.g., PSMA, HER2, or BCMA)-binding domain, and the second scFv comprises a humanized CD3-binding domain or (b) the first scFv comprises a humanized CD3-binding domain and the second scFv comprises a humanized TAA (e.g., PSMA, HER2, or BCMA)-binding domain.

As discussed in more detail below, vectors comprising the polynucleotides disclosed herein are also provided.

The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some aspects, the polynucleotide is a cDNA or a DNA lacking one more endogenous introns.

In some aspects, a polynucleotide is a non-naturally occurring polynucleotide. In some aspects, a polynucleotide is recombinantly produced.

In certain aspects, the polynucleotides are isolated. In certain aspects, the polynucleotides are substantially pure. In some aspects, a polynucleotide is purified from natural components.

In some aspects, a polynucleotide provided herein is codon optimized for expression in a particular host (change codons in the human mRNA to those preferred by a bacterial host such as E. coli).

### V. Cells and Vectors

Vectors and cells comprising the polynucleotides described herein are also provided herein.

In certain aspects, provided herein are cells (e.g., host cells) expressing (e.g., recombinantly) antibodies described herein which specifically bind to a TAA (e.g., PSMA, HER2, or BCMA) and/or CD3 and comprising related polynucleotides and expression vectors. Provided herein are vectors (e.g., expression vectors) comprising polynucleotides comprising nucleotide sequences encoding antibodies that specifically bind to a TAA (e.g., PSMA, HER2, or BCMA) and/or CD3 for recombinant expression in host cells, e.g., mammalian host cells. Also provided herein are host cells comprising such vectors for recombinantly expressing antibodies that specifically bind to a TAA (e.g., PSMA, HER2, or BCMA) and/or CD3 described herein. In a particular aspect, provided herein are methods for producing an antibody that specifically bind to a TAA (e.g., PSMA, HER2, or BCMA) and/or CD3 described herein, comprising expressing such antibody in a host cell.

Recombinant expression of an antibody that specifically bind to a TAA (e.g., PSMA, HER2, or BCMA) and/or CD3 described herein involves construction of an expression vector containing a polynucleotide that encodes the antibody or a polypeptide thereof (e.g., a fusion protein comprising an scFv, a linker (e.g., wherein the linker is a hinge), an immunoglobulin constant region; a heavy or light chain; a polypeptide comprising one or more variable domains; a polypeptide comprising one or more antigen-binding domains (e.g., scFvs), optionally fused to a linker (e.g., wherein the linker is a hinge), immunoglobulin constant region and/or linker, etc.). Once a polynucleotide encoding an antibody or a polypeptide thereof described herein has been obtained, the vector for the production of the antibody or polypeptide thereof can be produced by recombinant DNA technology using techniques well known in the art. Thus, methods for preparing a protein by expressing a polynucleotide a nucleotide sequence encoding an antibody or fragment thereof are described herein. Methods which are well known to those skilled in the art can be used to construct expression vectors containing coding sequences for an antibody or a polypeptide thereof and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody or a fragment thereof, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464), and variable domains of the antibody can be cloned into such a vector for expression of the entire heavy, the entire light chain, or both the entire heavy and light chains. A nucleotide sequence encoding an additional variable domain, a TAA (e.g., PSMA, HER2, or BCMA) binding domain (e.g., scFv), and/or a CD3-binding domain can also be cloned into such a vector for expression of fusion proteins comprising a heavy or light chain fused to an additional variable domain, a TAA (e.g., PSMA, HER2, or BCMA) binding domain (e.g., scFv), and/or a CD3-binding domain.

To direct a recombinant protein into the secretory pathway of a host cell, a secretory signal sequence (also known as a leader sequence) can be provided in the expression vector. The secretory signal sequence can be that of the native form of the recombinant protein, or can be derived from another secreted protein or synthesized de novo. The secretory signal sequence can be operably linked to the polypeptide-encoding DNA sequence. Secretory signal sequences are commonly positioned 5′ to the DNA sequence encoding the polypeptide of interest, although certain signal sequences can be positioned elsewhere in the DNA sequence of interest (see, e.g., Welch et al., U.S. Pat. No. 5,037,743; Holland et al., U.S. Pat. No. 5,143,830).

An expression vector can be transferred to a cell (e.g., host cell) by conventional techniques and the resulting cells can then be cultured by conventional techniques to produce an antibody or polypeptide thereof (e.g., a fusion protein comprising an scFv, a linker (e.g., wherein the linker is a hinge), an immunoglobulin constant region; a heavy or light chain; a polypeptide comprising one or more variable domains; a polypeptide comprising one or more antigen-binding domains (e.g., scFvs), optionally fused to a hinge, immunoglobulin constant region and/or linker, etc.) described herein. Thus, provided herein are host cells containing a polynucleotide encoding an antibody or a polypeptide thereof described herein operably linked to a promoter for expression of such sequences in the host cell.

In certain aspects, for the expression of multiple-polypeptide antibodies, vectors encoding all of polypeptides, individually, can be co-expressed in the host cell for expression of the entire antibody.

In certain aspects, a host cell contains a vector comprising polynucleotides encoding all of the polypeptides of an antibody described herein. In specific aspects, a host cell contains multiple different vectors encoding all of the polypeptides of an antibody described herein.

A vector or combination of vectors can comprise polynucleotides encoding two or more polypeptides that interact to form an antibody described herein: e.g., a first polynucleotide encoding a heavy chain and a second polynucleotide encoding a light chain; a first polynucleotide encoding a fusion protein comprising a heavy chain and an scFv with a second polynucleotide encoding a light chain; a first polynucleotide encoding a fusion protein comprising a light chain and an scFv with a second polynucleotide encoding a heavy chain; a first polynucleotide encoding a fusion protein comprising a heavy chain and a VH with a second polynucleotide encoding a fusion protein comprising a light chain and a VL, etc. Where the two polypeptides are encoded by polynucleotides in two separate vectors, the vectors can be transfected into the same host cell.

A variety of host-expression vector systems can be utilized to express antibodies or polypeptides thereof (e.g., a fusion protein comprising an scFv, a linker (e.g., wherein the linker is a hinge), an immunoglobulin constant region; a heavy or light chain; a polypeptide comprising one or more variable domains; a polypeptide comprising one or more antigen-binding domains (e.g., scFvs), optionally fused to a hinge, immunoglobulin constant region and/or linker, etc.) described herein. Such host-expression systems represent vehicles by which the coding sequences of interest can be produced and subsequently purified, but also represent cells which can, when transformed or transfected with the appropriate nucleotide coding sequences, express an antibody or polypeptide thereof described herein in situ. These include but are not limited to microorganisms such as bacteria (e.g., E. co/i and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NSO, PER.C6, VERO, CRL7030, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

Once an antibody or a polypeptide thereof (e.g., a fusion protein comprising an scFv, a linker (e.g., wherein the linker is a hinge), an immunoglobulin constant region; a heavy or light chain; a polypeptide comprising one or more variable domains; a polypeptide comprising one or more antigen-binding domains (e.g., scFvs), optionally fused to a hinge, immunoglobulin constant region and/or linker, etc.) described herein has been produced by recombinant expression, it can be purified by any method known in the art for purification of an antibody, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein (e.g., a FLAG tag, a his tag, or avidin) or otherwise known in the art to facilitate purification.

### VI. Compositions and Kits

Provided herein are compositions comprising an antibody described herein having the desired degree of purity in a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed.

A pharmaceutical composition may be formulated for a particular route of administration to a subject. For example, a pharmaceutical composition can be formulated for parenteral, e.g., intravenous, administration. The compositions to be used for in vivo administration can be sterile. This is readily accomplished by filtration through, e.g., sterile filtration membranes.

The pharmaceutical compositions described herein are in one aspect for use as a medicament. Pharmaceutical compositions described herein can be useful in enhancing an immune response. Pharmaceutical compositions described herein can be useful in increasing T cell (e.g., CD4 T cell and/or CD8 T cell) proliferation and/or activation in a subject.

Pharmaceutical compositions described herein can be useful in treating a condition such as cancer or a prostate disorder. Examples of cancer that can be treated as described herein include, but are not limited to, prostate cancer, castrate-resistant prostate cancer, colorectal cancer, clear cell renal carcinoma, colorectal cancer, bladder cancer, lung cancer, and gastric cancer. In certain aspects, the cancer is a solid tumor. In certain aspects, the prostate disorder is benign prostatic hyperplasia or a neovascular disorder.

### VI. Methods and Uses

The antibodies of the disclosure that bind to a TAA (e.g., PSMA, HER2, or BCMA) and/or CD3 are useful in a variety of applications including, but not limited to, therapeutic treatment methods, such as the treatment of cancer. In certain aspects, the agents are useful for inhibiting tumor growth and/or reducing tumor volume. The methods of use may be in vitro or in vivo methods. The disclosure includes the use of any of the disclosed antibodies (and pharmaceutical compositions comprising the disclosed antibodies) for use in therapy.

The present disclosure provides for methods of treating cancer in a subject comprising administering a therapeutically effective amount of an antibody that binds to TAA (e.g., PSMA, HER2, or BCMA) and/or CD3 to the subject. The disclosure includes the use of any of the disclosed antibodies for treatment of cancer, including but not limited to treatment with the disclosed heterodimer constructs capable of bivalent TAA binding and monovalent CD3 binding (e.g., constructs in ADAPTIR-FLEX™ format).

In certain aspects, the cancer is a cancer including, but are not limited to, PSMA(+) cancer, prostate cancer, metastatic prostate cancer, castrate-resistant prostate cancer, colorectal cancer, clear cell renal carcinoma, colorectal cancer, bladder cancer, lung cancer, and gastric cancer. The cancer may be a primary tumor or may be advanced or metastatic cancer. In certain aspects, the cancer is a solid tumor. For instance, the present disclosure includes use of the bispecific antibodies for treatment of PSMA (+) cancer, prostate cancer, metastatic prostate cancer, castrate-resistant prostate cancer, colorectal cancer, clear cell renal carcinoma, colorectal cancer, bladder cancer, lung cancer, and gastric cancer. The disclosure includes, for instance, treating a human subject with PSMA(+) cancer, prostate cancer, metastatic prostate cancer, castrate-resistant prostate cancer, colorectal cancer, clear cell renal carcinoma, colorectal cancer, bladder cancer, lung cancer, and gastric cancer by administering to the subject a therapeutically effective amount of a pharmaceutical composition of the disclosure (e.g., a pharmaceutical composition comprising a bispecific antibody that comprises a first and second polypeptide chain that specifically binds human PSMA and human CD3 and comprising an amino acid sequence at least 85%, 90%, 95%, or 99% identical to an amino acid sequence of SEQ ID NOs: 106 and 108, 178 and 108, or 112 and 108).

The disclosure includes methods of treating a human subject with a disorder, wherein the said disorder is characterized by the overexpression of PSMA by administering to the subject a therapeutically effective amount of an PSMA×CD3 bispecific antibody that comprises a first and second polypeptide chain comprising SEQ ID NOs: 106 and 108, 178 and 108, or 112 and 108. In one aspect, the disclosure includes administering to a human subject with a disorder a therapeutically effective amount of a pharmaceutical composition comprising an anti-PSMA×anti-CD3 bispecific antibody wherein the humanized PSMA-binding domain comprises a VH comprising an amino acid sequence at least 85%, 90%, 95%, or 99% identical to an amino acid sequence SEQ ID NO:82 and a VL comprising an amino acid sequence at least 85%, 90%, 95%, or 99% identical to an amino acid sequence of SEQ ID NO:84 and wherein the humanized CD3-binding domain comprises a VH comprising an amino acid sequence at least 85%, 90%, 95%, or 99% identical to an amino acid sequence SEQ ID NO: 100 and a VL comprising an amino acid sequence at least 85%, 90%, 95%, or 99% identical to an amino acid sequence SEQ ID NO: 102. For instance, the disclosure includes administering to a human subject with a disorder a therapeutically effective amount of a pharmaceutical composition comprising an anti-PSMA×anti-CD3 bispecific antibody wherein the humanized PSMA-binding domain comprises an amino acid sequence at least 85%, 90%, 95%, or 99% identical to an amino acid sequence SEQ ID NO:86 and wherein the humanized CD3-binding domain comprises an amino acid sequence at least 85%, 90%, 95%, or 99% identical to an amino acid sequence SEQ ID NO:104 or 110.

The present disclosure provides for treating a subject comprising administering a therapeutically effective amount of an antibody that binds to a TAA (e.g., PSMA, HER2, or BCMA) and/or CD3 to the subject without inducing high levels of cytokines (e.g., cytokine release syndrome). For instance, the present disclosure provides for treating a patient with a TAA×CD3 antibody without inducing high levels of IFN-gamma, TNF-alpha, IL-6 and/or IL-2. In one aspect provided herein, the present disclosure provides for treating a patient with a TAA×CD3 provided herein, including, for instance, heterodimer constructs in the ADAPTIR-FLEX™ format, without co-administration of drugs necessary for the treatment for cytokine release (e.g., IFN-gamma, TNF-alpha, IL-6 and/or IL-2). For instance, the present disclosure provides for treating a patient with a TAA×CD3 antibody without inducing high levels of Granzyme B, IL-10, and/or GM-CSF. In one aspect provided herein, the present disclosure provides for treating a patient with a TAA×CD3 provided herein, including, for instance, heterodimer constructs in the ADAPTIR-FLEX™ format, without co-administration of drugs necessary for the treatment for cytokine release (e.g., Granzyme B, IL-10, and/or GM-CSF).

The present disclosure provides for methods of increasing the proliferation and/or activation of T cells (e.g., CD4+ T cells and/or CD8+ T cells) in a subject comprising administering a therapeutically effective amount of an antibody that binds to a TAA (e.g., PSMA, HER2, or BCMA) and/or CD3 to the subject. The present disclosure provides for methods of increasing the proliferation and/or activation of CD4+ T cells and CD8+ T cells in a subject comprising administering a therapeutically effective amount of an antibody that binds to a TAA (e.g., PSMA, HER2, or BCMA) and CD3 to the subject.

The present disclosure provides for methods of increasing the proliferation and/or activation of T cells (e.g., CD4+ T cells and/or CD8+ T cells) in a subject comprising administering a therapeutically effective amount of an antibody that binds to PSMA and/or CD3 to the subject. The present disclosure provides for methods of increasing the proliferation and/or activation of CD4+ T cells and CD8+ T cells in a subject comprising administering a therapeutically effective amount of an antibody that binds to PSMA and CD3 to the subject. For instance, the disclosure includes methods for increasing the proliferation and/or activation of CD4+ T cells and CD8+ T cells in a subject comprising administering a therapeutically effective amount of a pharmaceutical composition comprising a bispecific antibody that comprises a first and second polypeptide chain that specifically binds human PSMA and human CD3 and comprising an amino acid sequence at least 85%, 90%, 95%, or 99% identical to an amino acid sequence selected from the group of SEQ ID NOs: 106 and 108, 178 and 108, or 112 and 108.

The present disclosure provides for methods of inducing redirected T-cell cytotoxicity (RTCC) against a cell expressing a TAA (e.g., PSMA, HER2, or BCMA) by contacting a bispecific antibody or composition comprising said bispecific antibody that binds to a TAA (e.g., PSMA, HER2, or BCMA).

The present disclosure provides for methods of inducing redirected T-cell cytotoxicity (RTCC) against a cell expressing PSMA by contacting a bispecific antibody or composition comprising said bispecific antibody, wherein the bispecific antibody comprises a first and second polypeptide chain that specifically binds human PSMA and human CD3 and comprising an amino acid sequence at least 85%, 90%, 95%, or 99% identical to an amino acid sequence selected from the group of SEQ ID NOs: 106 and 108, 178 and 108, or 112 and 108.

In certain aspects, the subject is a human.

Administration of an antibody that binds to a TAA (e.g., PSMA, HER2, or BCMA) and/or CD3 can be parenteral, including intravenous, administration.

In some aspects, provided herein are antibodies that bind to a TAA (e.g., PSMA, HER2, or BCMA) and/or CD3, or pharmaceutical compositions comprising the same, for use as a medicament. In some aspects, provided herein are antibodies that bind to a TAA (e.g., PSMA, HER2, or BCMA) and/or CD3, or pharmaceutical compositions comprising the same for use in a method for the treatment of cancer. For instance, the disclosure includes a pharmaceutical composition comprising a bispecific antibody containing a human PSMA-binding domain comprises a VH comprising an amino acid sequence at least 85%, 90%, 95%, or 99% identical to an amino acid sequence SEQ ID NO:82 and a VL comprising an amino acid sequence at least 85%, 90%, 95%, or 99% identical to an amino acid sequence of SEQ ID NO:84 and wherein the human CD3-binding domain comprises a VH comprising an amino acid sequence at least 85%, 90%, 95%, or 99% identical to an amino acid sequence SEQ ID NO: 100 and a VL comprising an amino acid sequence at least 85%, 90%, 95%, or 99% identical to an amino acid sequence SEQ ID NO: 102.

In one aspect, antibodies that bind to a TAA (e.g., PSMA, HER2, or BCMA) and/or CD3 provided herein are useful for detecting the presence of a TAA (e.g., PSMA, HER2, or BCMA) and/or CD3, e.g., in a biological sample. The term “detecting” as used herein encompasses quantitative or qualitative detection. In certain aspects, a biological sample comprises a cell or tissue. In certain aspects, the method of detecting the presence of PSMA and/or CD3 in a biological sample comprises contacting the biological sample with an antibody that binds to PSMA and/or CD3 provided herein under conditions permissive for binding of the antibody, and detecting whether a complex is formed between the antibody and PSMA and/or CD3.

In certain aspects, an antibody that binds to PSMA and/or CD3 provided herein is labeled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction.

Aspects of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, can be practiced without departing from the scope of the present disclosure.

### EXAMPLES

It is understood that the examples and aspects described herein are for illustrative purposes only and that various modifications or changes in light thereof will be suggested to persons skilled in the art and are to be included within the spirit and purview of this application.

### Example 1. Bispecific Proteins with Different Structures and Binding Valency

While the optimal distance and geometry to form an immune synapse between a PSMA-expressing cell and a CD3-expressing T cell is unknown, and epitopes of the anti-PSMA-specific and anti-CD3-specific binding domains are pre-determined and immovable, testing of different bispecific structures was performed to achieve the optimal formation of an immune synapse. In addition to making sequence changes to modulate the affinity of individual binding domains of bispecific constructs, geometry and binding valency were also investigated by producing and testing molecules with heterodimeric structures that contained one or two binding domains against PSMA and CD3 and located on either the N- or C-terminal positions on the Fc region (FIG. 1A-E). Additionally, the effect of changing the order of the scFv domains (VH-VL versus VL-VH) was examined. These structural changes, in addition to impacting the binding affinity and functional performance of ADAPTIR™, can also have a significant impact on the expression levels and stability of the bispecific protein and were examined as part of this work.

TABLE 1
Design of the anti-PSMA × anti-CD3 constructs.

| Construct | PSMA BD | CD3 BD | CD3 BD location |
|---|---|---|---|
| TSC266* | VH-VL, bivalent | VH-VL, bivalent | N-terminus |
| PSMA01026 | VL-VH, | VH-VL, monovalent | N-terminus |
| | monovalent | | |
| PSMA01070 | VH-VL, | VH-VL, monovalent | N-terminus |
| | monovalent | | |
| PSMA01071 | VH-VL, bivalent | VH-VL, monovalent | C-terminus |
| PSMA01072 | VH-VL, bivalent | VH-VL, bivalent | C-terminus |
| PSMA01086 | VH-VL, bivalent | VL-VH monovalent | C-terminus |

*TSC266 control (PSMA × CD3 bispecific protein)

### Example 2. Generation of PSMA-Expressing CHO Cells and Recombinant Extracellular Domain Proteins

The nucleotide sequences defining the human and non-human primate PSMA full length and extracellular domains (ECDs) were obtained from the Genbank database and are listed in Table 2.

TABLE 2
SEQ ID NOs of constructs for production
of cell lines and recombinant proteins

| Construct Name | Construct Description | SEQ ID NO: |
|---|---|---|
| TSC033 | Human PSMA ECD-AFH | 2 |
| TSC435 | Full-length Human PSMA | 4 |
| TSC308 | Full-length cynomolgus monkey | 6 |
| | PSMA | |

The soluble human PSMA ECD (HuPSMA-AFH) construct contained C-terminal tags for purification, detection and biotin-based labeling purposes. The DNA construct containing the nucleotide sequences for HuPSMA-AFH was synthesized and inserted into an expression vector appropriate for mammalian cell expression and secretion. The DNA construct encoding full-length human and non-human primate full-length PSMA proteins were inserted into an expression vector appropriate for cell-surface expression that included the ability to apply selective pressure to generate stable transfectants. These reagents were used to assess the cross reactivity and binding strength of anti-PSMA-binding domains to human PSMA and the species to be used in potential toxicology assessments. The DNA expression vector encoding HuPSMA-AFH was used to transiently transfect human embryonic kidney fibroblast (HEK)-293 cells grown in suspension culture. After several days in culture, the conditioned media was clarified via centrifugation and sterile filtration. Protein purification was performed utilizing a combination of Immobilized Metal Affinity Chromatography (IMAC) followed by size exclusion chromatography (SEC). A mixture of monomeric and dimeric PSMA was present in the sample after the IMAC capture step. SEC removed monomeric PSMA, as well as aggregated and clipped product and other host cell contaminants. SEC was also used to buffer-exchange the protein into phosphate-buffered saline (PBS). Final purity was determined by analytical SEC and typically exceeded 90%. Protein batches were sterile-filtered and stored at 4° C. if the intent was to use within the next week. Otherwise, the pure PSMA ECD dimer was frozen in aliquots in a −80° C. freezer.

Plasmid DNA encoding full-length Human PSMA was digested with a restriction enzyme and ethanol precipitated, then dissolved in ultrapure water, then Maxcyte Electroporation Buffer. Linearized DNA was transfected into CHO-K1SV cells (CDACF-CHO-K1SV cells (ID code 269-W3), Lonza Biologics) by electroporation. Transfected cells were transferred from the electroporation cuvette to a T75 culture flask, rested, and then gently resuspended in the flask with 15 mL of CD CHO media supplemented with 6 mM L-Glutamine. The flask was put in a 37° C., 5% C02 incubator and allowed to recover for 24 hours prior to placing in the selection conditions. On the day following transfection, the cells were centrifuged for 5 minutes at 1000 RPM and resuspended in CD CHO medium with 1×GS (Glutamine synthetase) supplement and 50 μM MSX (Methionine Sulfoximine). After the bulk populations were recovered from initial selection, cells were evaluated for surface expression with commercially available reagents, and representative vials were frozen. To obtain clones with varying levels of expression, cells were sorted by flow cytometry, plated by limiting dilution, and allowed to grow for 2 weeks. Wells were imaged with a Clone Select Imager during the incubation to identify growth positive wells. Only wells with good quality images were selected for further expansion and characterization for surface expression by flow cytometry. All clones were frozen in banks at up to 30 vials per clone.

### Example 3. General Expression and Purification of PSMA- and CD3-Binding Molecules and Antibodies

Monospecific and bispecific PSMA- and CD3-binding molecules disclosed herein were produced by transient transfection of either HEK293 or Chinese Hamster Ovary (CHO) cells. Cultures were clarified of cells, cell debris, and insoluble matter by centrifugation and/or filtration. Recombinant homodimeric proteins were captured from the clarified, conditioned media using Protein A affinity chromatography (ProA). Preparative Size exclusion chromatography (Prep SEC) was typically performed to further purify the protein to homogeneity and buffer-exchange into PBS. Protein purity was verified by analytical size exclusion chromatography (analytical SEC) on an Agilent HPLC after each of the ProA and Prep SEC purification steps.

Heteromeric proteins in which two or more peptide chains assemble to form a soluble protein complex were expressed using transiently transfected CHO cells using separate plasmids for each peptide chain. In some instances, the plasmids were transfected in equal ratios. If it was observed that one peptide chain expressed significantly better than the other(s), the plasmid ratio was altered to transfect a greater quantity of the lower-expressing plasmid. The protein was captured from cell culture supernatant using ProA with a wash step and low pH elution step. Prep SEC was used to remove aggregated protein and exchange the sample into PBS. In some instances, a second ProA chromatography step was performed. After washing the column with PBS, the protein was eluted using a decreasing pH gradient (from neutral to acidic). In some cases, cation exchange chromatography was used to further purify heterodimers to remove low MW, homodimer and unpaired peptide chain contaminants.

In most instances, final protein batches were buffer-exchanged into PBS as part of the SEC purification process, adjusted to 1 mg/mL, sterile-filtered and stored at 4° C. until needed or otherwise specified. Protein concentration was determined from the absorbance at 280 nm using the theoretical extinction coefficient calculated from the amino acid sequence.

Endotoxin levels were determined with the Endosafe PTS instrument, using the manufacturer's instructions. This assured that the in vitro activity assay results would not be confounded by the presence of endotoxin. Analytical SEC was used along with peak area integration to quantify the purity of the samples. In some instances, the resolving power of analytical SEC was insufficient to separate the desired heterodimeric product from product-related contaminants, Capillary Electrophoresis-Sodium Dodecyl Sulphate (CE-SDS) was used as a secondary method to assess product purity. Reduced and non-reduced SDS-PAGE (Sodium Dodecyl Sulfate-PolyAcrylamide Gel Electrophoresis) gels were run along with molecular weight (MW) standards to confirm the purity and estimate MW of the product.

### Example 4. Humanization of PSMA-Specific Clone 107-1A4 in scFv Format

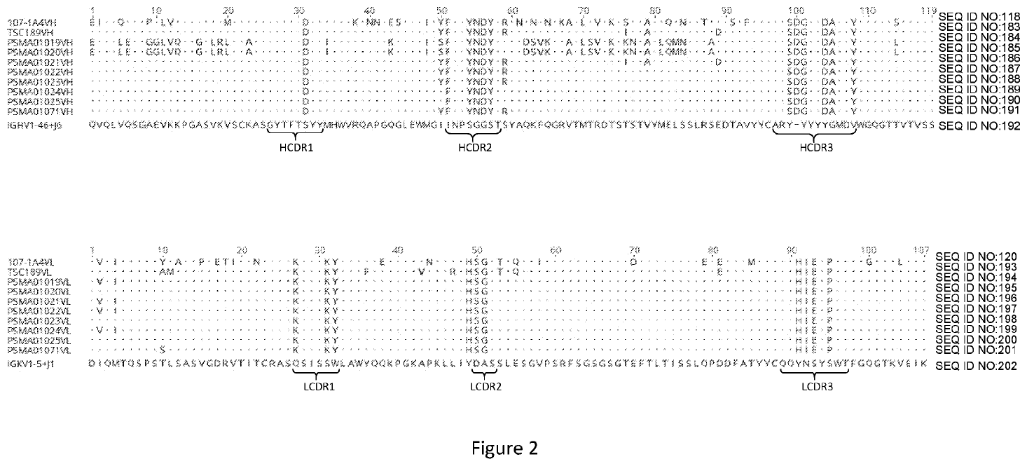

Mouse monoclonal antibody 107-1A4 (VH SEQ ID NO:118; VL SEQ ID NO: 120) was humanized resulting in PSMA-specific scFv binding domain TSC189, PSMA01012, (VH SEQ ID NO: 114; VL SEQ ID NO: 116) present in molecule TSC266 as described in US 2018/0100021. While TSC189 binding properties, like specificity to PSMA antigen are satisfactory, multiple biophysical and manufacturing properties were considered suboptimal. Re-humanization of 107-1A4 in scFv format was carried out to optimize all binding, functional and manufacturing properties. Humanization was performed in multiple stages. The BioLuminate software package release 2018-2 (Schrodinger, LLC, New York, USA) was utilized. A homology model of mouse clone 107-1A4 was created based on PDB ID 1JHL, and the most geometrically suitable and homologous human frameworks for CDR grafting were identified using the software's default and modified settings. Seven initial CDR-grafted molecules based on different target human germlines were produced and tested for binding to cells expressing full length human- or cyno-PSMA (data not shown). Subsequently, framework residues were mutated in sets and combinations of sets to convert mouse residues to human germline sequences IGHV1-46*01 and IGHJ6*01 for heavy chain and IGKV1-5*01 and IGKJ1*01 for light chain. Molecule PSMA01023 containing germlining set G11, SEQ ID NO:30, was identified to carry the best combination of binding and developability properties. Finally, to further improve biophysical properties the order of domains in the anti-PSMA scFv from VL-VH to VH-VL and introduced mutation T10S to remove an O-linked glycosylation site. The sequence of PSMA01071 molecule is 91.8% identical to IGHV1-46*01 and 89.4% identical to IGKV1-5*01. Amino acid alignment of VH and VL regions of PSMA-specific binding domains is shown in FIG. 2 (Sequences of 107-1A4 and humanized anti-PSMA-binding domains).

All antibody protein engineering was performed at the protein sequence level. Genes corresponding to designed proteins were synthesized by Integrated DNA Technologies Inc., Coralville, Iowa USA using their online gene design tool for optimized expression in mammalian system. Synthetic genes were combined with each other or with expression vectors using either NEBuilder HiFi DNA Assembly Cloning Kit (New England Biolabs, Beverly MA) or using standard molecular biology techniques and methods generally disclosed in, e.g., PCT Application Publication No. WO 2007/146968, U.S. Patent Application Publication No. 2006/0051844, PCT Application Publication No. WO 2010/040105, PCT Application Publication No. WO 2010/003108, and U.S. Pat. No. 7,166,707. DNA sequences were verified using Sanger sequencing at GENEWIZ, South Plainfield, NJ, USA.

### Example 5. Humanization, Attenuation and Optimization of CD3-Specific Clone CRIS-7 in scFv Format

The CRIS-7 mouse monoclonal antibody (VH SEQ ID NO: 122; VL SEQ ID NO: 124) was humanized resulting in CD3ε-specific scFv binding domain found, for example, in TSC266 as DRA222 (VH SEQ ID NO: 126; VL SEQ ID NO:128) and as TSC456 (VH SEQ ID NO:130; VL SEQ ID NO: 132) as described in US 2018/0273622, which is herein incorporated by reference in its entirety. The goal of the new humanization strategy was to increase percentage of human amino acid sequence content as high as possible, while keeping binding and signaling properties as close as possible to parental clone CRIS-7.

Initial attempts to attenuate binding of CD3ε-specific scFv domain while keeping functional CD3 signaling intact, a humanized anti-CD3ε scFv binding domain (TSC456) was used and followed protocols established in the art, such as parsimonious mutagenesis of CDR residues (Balint, R. F., and J. W. Larrick. 1993. Antibody engineering by parsimonious mutagenesis. Gene 137:109). A linear correlation of binding affinity to signaling functional properties of anti-CD3ε scFv binding domains was observed (data not shown).

To circumvent the observed linear correlation of binding and signaling and to identify variants with non-linear characteristics, re-humanization of CRIS-7 was attempted. The goal of re-humanization of CRIS-7 described in aspects of this disclosure was to increase percentage of human amino acid sequence as high as possible, increase thermal stability of the scFv domain, decrease binding affinity to CD3ε while keeping the CD3 signaling properties similar to the parental molecule CRIS-7. This empirical process included multiple rounds of molecular modeling using the BioLuminate software package (Schrodinger, LLC, New York, USA), followed by designing and building libraries of binding domains in scFv format and testing these in binding, signaling, and biophysical stability assays.

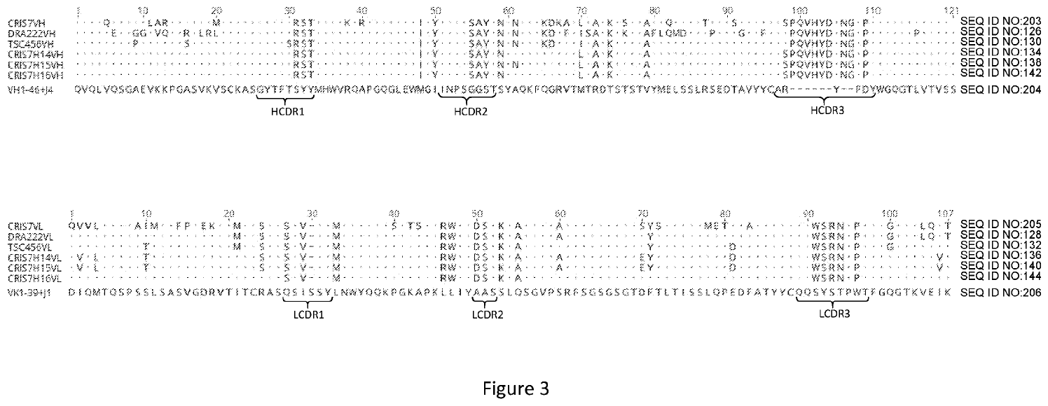

In addition to mutating framework residues, several CDR residues were mutated and tested both orders of VH and VL sequences in scFv domain. The sequence of CRIS7H16 binding domain is 86.6% identical to IGHV1-46*01 and 85.3% identical to IGKV1-39*01. Amino acid alignment of CD3ε-specific sequences from mouse CRIS-7 antibody and from molecules TSC266, TSC456, together with sequences of variants CRIS7H14 (VH SEQ ID NO: 134; VL SEQ ID NO:136), CRIS7H15 (VH SEQ ID NO: 138; VL SEQ ID NO: 140) and CRIS7H16 (VH SEQ ID NO: 142; VL SEQ ID NO: 144) and human germline sequences VH (IGHV1-46*01; IGHJ4*01) and VL (IGKV1-39*01; IGKJ1*01) is shown in FIG. 3 (Sequences of CRIS-7 and humanized CD3ε-specific binding domains).

### Example 6. Methodology for Using Surface Plasmon Resonance to Determine the Binding Affinity of Anti-PSMA-Binding Domains

SPR binding affinity studies of mono- and bispecific proteins binding to recombinant dimeric PSMA ECD were conducted at 25° C. in dPBS with 0.2% BSA buffer on a Biacore T200 system. Mouse anti-human IgG (GE, BR-1008-39) at 25 μg/ml in 10 mM sodium acetate pH 5.0 was immobilized at a density of ˜2,000-4,000 response units (RU) onto each flow cell of a CM5 research-grade sensor chip (GE) by standard amine coupling chemistry. Each anti-PSMA protein at approximately 40 nM in dPBS with 0.2% BSA buffer was captured in a flow cell with the immobilized anti-human IgG at a flow rate of L/min for up to 30 seconds, leaving one flow cell surface unmodified as the reference. Using a multi-cycle kinetics mode, a buffer blank and five different concentrations of ECD ranging from 1 nM to 243 nM were sequentially injected through each flow cell at 30 μL/min with association times varying from 300-600 seconds and dissociation times varying from 600-1200 seconds. Regeneration was achieved by injection of 3 M MgCl₂ at a flow rate of 30 μL/min for up to 40 seconds followed by dPBS with 0.2% BSA buffer stabilization for 1 min.

Sensorgrams obtained from kinetic SPR measurements were analyzed using the double subtraction method. The signal from the reference flow cell was subtracted from the analyte binding response obtained from flow cells with captured ligands. The buffer blank response was then subtracted from analyte binding responses and the final double-referenced data were analyzed with Biacore T200 Evaluation software (2.0, GE), globally fitting data to derive kinetic parameters. All sensorgrams were fitted using a simple one-to-one binding model.

Several monospecific anti-PSMA scFv-Fc proteins were evaluated for their binding affinity to dimeric PSMA ECD using SPR. All the scFvs in this set were constructed in the VL-VH orientation. Two variants, PSMA01024 and PSMA01025 showed much lower binding affinity compared to the other constructs tested (Table 3). The remaining proteins all bound to PSMA with a KD<50 nM and were similar to anti-PSMA-binding domain, PSMA01012.

TABLE 3
Biacore affinity measurements on monospecific,
bivalent anti-PSMA-binding domains.

| Name | KD (nM) | ka (1/Ms) | kd (1/s) |
|---|---|---|---|
| PSMA01012 | 40 | 2.7E+04 | 1.1E−03 |
| PSMA01019 | 15 | 1.3E+04 | 1.9E−04 |
| PSMA01020 | 16 | 1.2E+04 | 1.9E−04 |
| PSMA01021 | 18 | 9.4E+03 | 1.7E−04 |
| PSMA01023 | 40 | 4.5E+03 | 1.8E−04 |
| PSMA01024 | 299 | 8.5E+03 | 2.6E−03 |
| PSMA01025 | 271 | 4.0E+04 | 1.1E−02 |

### Example 7. Expression of Human PSMA by Cell Lines

Cell lines expressing human PSMA were used for binding and functional characterization of PSMA constructs. The following cell lines were used: 22RV1, human prostate carcinoma cell line (ATCC), C4-2B, androgen-independent human prostate cancer line (Wu et al., 1994 Int. J. Cancer 57:406-12; obtained from MD Anderson Cancer Center (Houston, TX), and CHOK1SV cells stably transfected with human PSMA (CHOK1SV/huPSMA). The levels of surface PSMA expression on these cells were determined by flow cytometry.

Cells were plated at approximately 100,000 cells per well, in 96-U bottom plates, and labeled at 4° C., with a saturating concentration of PE-conjugated antibodies: anti-PSMA antibody (LNI-17 clone, Biolegend #342504) and isotype control (MOPC-21 clone, mouse IgG isotype, Biolegend #400140). Following a one-hour incubation, cells were washed and analyzed by flow cytometry. All incubations and washes were done in staining buffer (PBS buffer with 0.2% BSA and 2 mM EDTA). Samples were collected using an BD™ LSR-II flow (BD Biosciences) and analyzed by FlowJo flow cytometry analysis software. Mean fluorescence intensity (MFI) of bound molecules on cells was determined after exclusion of doublets. Quantibrite™ beads (BD Bioscience #340495) were used to determine receptors numbers as described by the manufacturer.

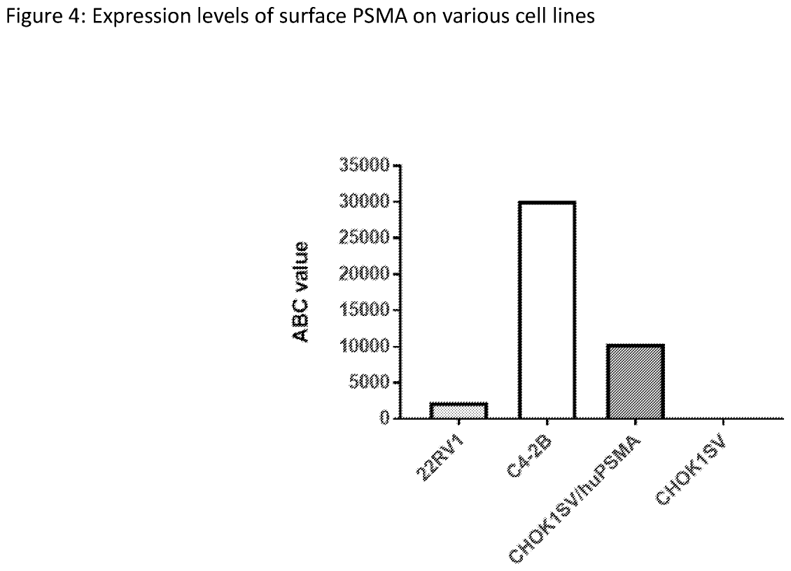

FIG. 4 shows the levels of expression of human PSMA in 22RV1, C4-2B, CHOK1SV/huPSMA, and parental CHOK1SV cells. The graph shows receptor levels in units of antibody bound per cell (ABC). On average, 22RV1 cells express less than 3,000 receptors/cell, while C4-2B cells express over 30,000 PSMA receptors/cell and CHOK1SV/huPSMA cells express approximately 10,000 receptors/cell. Therefore 22RV1 and C4-2B cells are PSMA(low) and PSMA(high) cells, respectively.

### Example 8. Binding of PSMA-Binding Molecules to Human and Cynomolgus PSMA-Expressing CHO Cell Lines

To measure the relative binding activity of humanized PSMA-binding domain variants, constructs were tested in cell binding assays on CHOK1SV cells transfected with human or cynomolgus PSMA. The generation of CHOK1SV/huPSMA cells was described earlier; CHOK1SV/cynoPSMA cells were generated using the same method. Bivalent PSMA-binding domains variants in scFv-Fc format (constructs PSMA01019, PSMA01020, PSMA01021, PSMA01023, PSMA01024 and PSMA01025) were tested in these assays.

Binding studies on CHOK1SV transfectants were performed in live cell-based ELISA using electrochemiluminescence (Meso Scale Discovery). CHOK1SV cells were washed and seeded at 50,000 cells/well in 1× Hank's Balanced Salt Solution (HBSS) on 96-well Multi-Array High Bind plates (Meso Scale Discovery) and incubated at 37° C. for one hour. Following a blocking step in PBS buffer with 20% FBS, serial dilutions of PSMA-binding constructs (from 0.002 to 100 nM) were added in PBS buffer with 10% FBS and incubated at room temperature for one hour. Plates were washed with PBS and the specific binding levels were detected by SULFO TAG-labeled goat anti-human IgG antibody (Meso Scale Discovery #R32AJ). Following an hour incubation and wash steps, 150 μL/well surfactant free 1× Read Buffer T were added and samples were analyzed on MSD Sector Imager (Meso Scale Discovery). Resulting electrochemiluminescence (ECL) values versus concentrations were plotted and nonlinear regression analysis to determine EC50 values was performed in GraphPad Prism 7® graphing and statistics software.

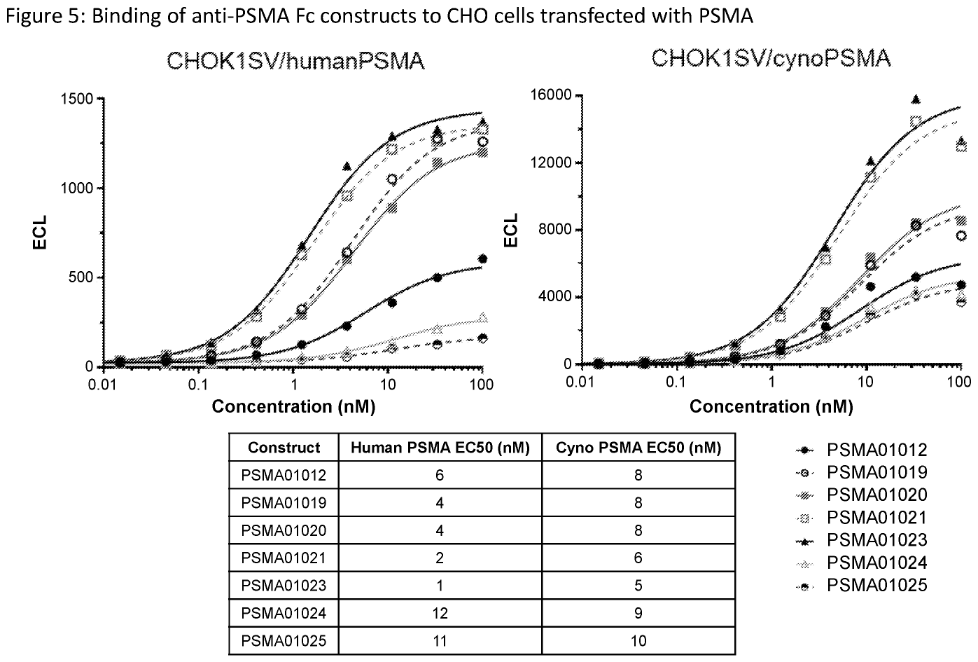

FIG. 5 shows the binding curves of the humanized PSMA-binding domain constructs PSMA01019, PSMA01020, PSMA01021, PSMA01023, PSMA01024, and PSMA01025 on human and cynomolgus CHOK1SV/PSMA transfectants, compared to the parent construct PSMA01012. Construct PSMA01023 displayed the highest binding strength on both human and cynomolgus PSMA transfectants.

### Example 9. Evaluation of Stability of Anti-PSMA Humanization Variants to Select Preferred Binding Domain

In addition to cell binding, constructs PSMA01019, PSMA01020, PSMA01021, PSMA01023, PSMA01024 and PSMA01025 were also evaluated for biophysical stability. Following purification, the samples were formulated in PBS buffer at 1 mg/mL. 100 μL aliquots were stored at 4, 40 and −20° C. Sample purity was determined at the start of the study using analytical SEC. After one week of storage, % purity was determined again. The sample at −20° C. was thawed on the benchtop prior to analysis. All samples showed minimal change in purity after a week of storage at 4 and 40° C. as shown in Table 4. Conversely, examination of the samples submitted to −20° C. freeze/thaw showed varying resistance to cryo-aggregation. PSMA01012, showed a 8.9% decrease in purity due to aggregation. PSMA01024 also showed a large decrease in purity (˜20%), whereas PSMA01023 did not exhibit any measurable change in purity. This indicated that PSMA01023 had greater resistance to freezing-induced aggregation than the parent construct, PSMA01012, from which it derived its CDR regions from. Resistance to aggregate formation during freezing is a preferred characteristic of therapeutic proteins.

In addition to the storage stability evaluation at 4, 40 and −20° C., the mid-point of the first melting transition (Tₘₗ) was measured using Differential Scanning Fluorimetry. Tₘₗ was used to reflect the temperature required to unfold the first, or least-stable, binding domain in the construct. DSF was performed using the Uncle instrument from Unchained Laboratories. Samples were analyzed at 1 mg/mL in PBS using intrinsic fluorescence (no additional dyes were used to assess protein unfolding). PSMA01012, the parent construct, had the lowest recorded Tₘₗ of 54.5° C., whereas the derivative constructs all had values greater than 61° C., indicating that they are all more thermostable. Both the storage and DSF data supported further evaluation of PSMA01023.

TABLE 4
Stability data obtained with anti-PSMA humanization variants

| | Change in | Change in | Change in | |
|---|---|---|---|---|
| | % Purity, 1 | % Purity, 1 | % Purity −20° C. | Tm1 |
| Construct | week @ 4° C. | week @ 40° C. | Freeze/Thaw | (° C.) |
| PSMA01012 | 0.4 | 0.8 | −8.9 | 54.5 |
| PSMA01019 | −0.1 | −0.5 | −1.8 | 66.7 |
| PSMA01020 | −0.1 | −0.5 | −3.8 | 67.9 |
| PSMA01021 | 0.1 | −0.7 | −0.1 | 62 |
| PSMA01023 | 0.1 | −0.6 | 0 | 65 |
| PSMA01024 | −0.1 | −1.3 | −20.5 | 63.8 |
| PSMA01025 | 0.5 | −0.5 | −0.8 | 63.9 |

### Example 10. Binding of PSMA-Binding Domains in Different Orientations to Human and Cynomolgus PSMA-Expressing CHO Cell Lines

The PSMA-binding domain PSMA01023 was evaluated in several different structural formats, including an alternative Fc region with different mutations to eliminate effector function. The PSMA01023 binding domain was configured in scFv-Fc format in VL-VH (PSMA01036) and VH-VL (PSMA01037) orientations and in Fc-scFv format in VL-VH (PSMA01040) and VH-VL (PSMA01041) orientations. These molecules were evaluated for the impact of the orientation of the scFv domains (VH-VL versus VL-VH) and position on the Fc (N- vs. C-terminus) on binding to PSMA (+) tumor cells.

C4-2B and 22RV1 cells were labelled at approximately 100,000 cells per well, in 96-well plates, with serial dilutions of PSMA-binding constructs ranging from of 0.1 to 300 nM for 30 min on ice, followed by washes and incubation with PE-labeled minimum cross species reactive secondary antibody, goat anti-human IgG Fcγ, F(ab′)2 (Jackson Laboratory) for 30 minutes on ice. Washes and incubation were done in staining buffer (PBS with 0.2% BSA and 2 mM EDTA). Cells were collected using a BDM LSRII or a BD FACSymphony™ flow cytometer (BD Biosciences) and analyzed by FlowJo flow cytometry analysis software. Median fluorescence intensity (MFI) of bound molecules on cells was determined after exclusion of doublets. Graphs were plotted using GraphPad Prism 7®.

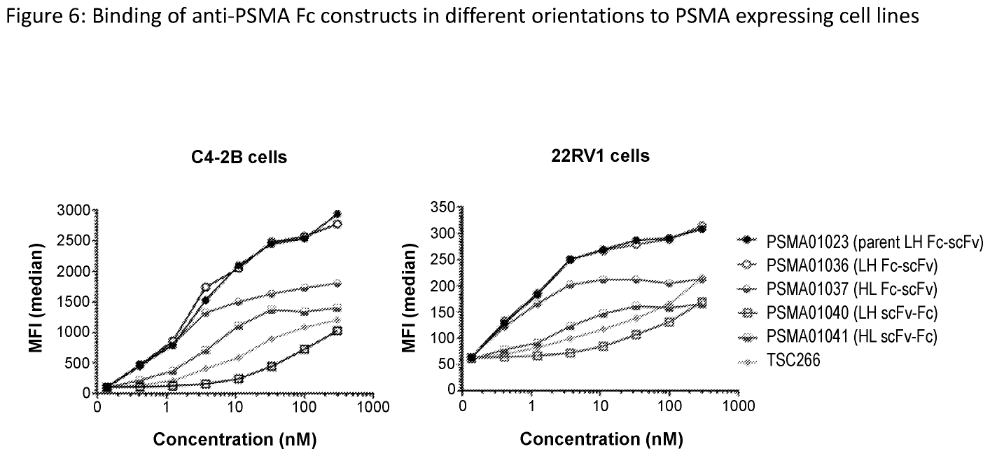

FIG. 6 shows the binding curves of the various constructs PSMA01036, PSMA010137, PSMA01040 and PSMA01041, on PSMA(high) and PSMA(low) expressing cells, C4-2B and 22RV1, respectively. PSMA01036 represents a codon-optimized version of PSMA01023 disclosed in Example 9, with an alternative Fe region. As shown in FIG. 6, both PSMA01023 and PSMA01036 have highly similar binding data. TSC266, which contains parent version of the anti-PSMA domain in PSMA01023 and related constructs was also evaluated for binding. Overall, better binding was observed when the PSMA-binding domain was the in scFv-Fc format (PSMA01036 and 01037) than in Fc-scFv format (PSMA01040 and PSMA01041). When present in scFv-Fc format, the PSMA-binding domain displayed slightly higher max binding in the VL-VH (PSMA01036) than in the VH-VL (PSMA01037) orientation. Therefore, the PSMA01036 and PSMA01037 domains were selected for incorporation into anti-PSMA×anti-CD3 bispecific constructs.

### Example 11. Binding of Anti-TA×Anti-CD3R Bivalent and Monovalent Constructs to CD3(+) Cells

In order to test their function, humanized and affinity-optimized CD3ε binding domain variants H14, H15 and H16 were fused to a tumor antigen (TA) binding domain. Constructs were designed with bivalent binding to the TA, and either bivalent or monovalent binding to CD3ε. CD3ε is expressed as part of the TCR/CD3 complex on cells of the T-cell lineage and surface expression of CD3ε requires the presence of the entire TCR/CD3 complex. Therefore, the designed anti-TA×CD3ε constructs were tested in CD3ε binding assays using the human T-lymphoblastic Jurkat cell line (clone E6-1, ATCC) which expresses a functional T-cell receptor. Constructs had a mutated Fc to eliminate Fc interactions with Fey receptors.

Jurkat cells were labelled at approximately 100,000 cells per well, in 96-well plates, with serial dilutions of bispecific constructs ranging in concentration from of 0.1 to 400 nM, for 30 min on ice. Primary label was followed by washes and incubation with PE-labeled minimum cross species reactive secondary antibody, goat anti-human IgG Fcγ, F(ab′)2 (Jackson Laboratory) for 30 minutes on ice. Washes and incubation were done in staining buffer (PBS with 0.2% BSA and 2 mM EDTA). Cells were collected using an BD™ LSRII or a BD FACSymphony™ flow cytometer (BD Biosciences) and analyzed by FlowJo flow cytometry analysis software. Median fluorescence intensity (MFI) of bound molecules on cells was determined after exclusion of doublets. Results were plotted and nonlinear regression analysis to determine EC50 values was performed using GraphPad Prism 7® graphing and statistics software.

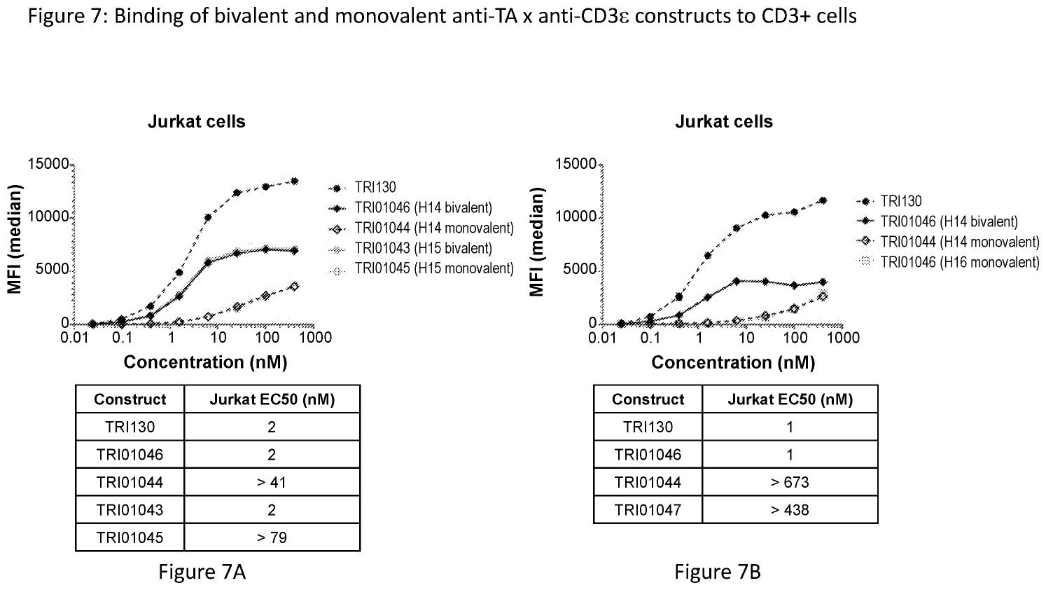

FIG. 7A shows the binding curves of the H14 and H15 binding domain constructs in bivalent and monovalent format. A control anti-TA×CD3ε bispecific construct with a high affinity CD3ε binding and bivalent for both TA and CD3 targets, TRI130, was included for comparison. When compared to TRI130, both H14 and H15 binding domains showed reduced binding affinity on Jurkat cells in the bivalent format (TRI01046 and TRI01043, respectively). As expected, binding potency on Jurkat cells was further reduced when H14 and H15 were present in monovalent format (TRI01044 and TRI01045, respectively). FIG. 7B shows the binding curves of the H14 and H16 binding domain constructs. Similarly low binding on Jurkat cells is observed with the H16 bearing construct in monovalent format (TRI01044 and TRI01047).

In conclusion, the H14, H15 and H16 humanized anti-CD3-binding domains show reduced binding affinity on CD3ε-expressing cells; as expected overall affinity is lower when binding domains are present in monovalent than in bivalent format.

### Example 12. Human T-Cell Activation, Proliferation and Target Cell Cytotoxicity in Response to Monovalent and Bivalent Anti-TA×Anti-CD3F Constructs

In order to induce tumor rejection, tumor targeting anti-CD3ε bispecific molecules elicit activation and proliferation of T cells, along with cytotoxicity of the TA-expressing target cells. The effectiveness of the affinity-optimized anti-TA×CD3ε constructs bearing H14, H15 and H16 CD3-binding domains at inducing target-dependent T-cell activation and proliferation, was compared to that of the unoptimized TRI130 construct. Constructs had a mutated Fc to eliminate Fc interactions with Fey receptors.

T-cell activation and proliferation were assessed using human T-cells isolated from PBMC. PBMC were obtained from healthy volunteers and isolated using standard density gradient centrifugation. Isolated PBMC were used either immediately after isolation of after thawing from cryopreserved cells banks. T cells were isolated using negative isolation kits (Pan T cell isolation kit, Miltenyibiotec #130-096-535) using the manufacturer's instructions.

For activation assays, T cells were plated in U-bottom 96-well plates at about 100,000 cells/well with 30,000 TA(+) cells/well, to achieve approximate T-cell to tumor cell ratios of 3:1. Serial dilutions of test molecules at concentrations ranging from 0.02 to 2,000 μM were added to the cell mixtures to a final volume of 200 μl/well in RPMI 1640 media supplemented with 10% Fetal bovine serum (FBS, SIGMA) sodium pyruvate, antibiotics and non-essential amino acids. Plates were incubated at 37° C., 5% CO₂ in humidified incubators. After 20 to 24 hours, cells were labeled at 4° C., with antibodies for flow cytometric analysis in original plates to minimize cell losses, using saline buffer with 0.10% bovine serum albumin and 2 mM EDTA. After centrifugation and removal of supernatant, the cell pellets were resuspended in 50 μl volumes containing a mixture of fluorescently-labeled antibodies to the surface antigens CD5, CD8, CD4, CD25, and CD69 (Biolegend), and the viability dye 7AAD (SIGMA), and incubated for 30 min on ice. Cells were washed twice and resuspended immediately prior to acquisition of 50% of each well in a BD™ LSRII or a BD FACSymphony™ flow cytometer (BD Biosciences). The sample files were analyzed using FlowJo software to calculate the percentages of CD4⁺ or CD8⁺ T-cells that had upregulated CD69 and CD25, by gating sequentially on forward vs side scatter, 7AAD⁻, CD5⁺, CD4⁺ or CD8⁺ T-cells (7AAD⁻, CD5⁺ CD4⁺ or 7AAD⁻ CD5⁺ CD8⁺, respectively). Results were plotted and nonlinear regression analysis to determine EC50 values was performed using GraphPad Prism 7© graphing and statistics software.

For assessment of T-cell proliferation, T cells were labeled with CellTrace™ Violet dye (CTV, Thermofisher). CTV-labeled T-cells were plated in U-bottom 96-well plates at about 100,000 cells/well, respectively, with 30,000 TA(+) tumor cells/well, to achieve approximate T-cell to tumor cell ratios of 3:1 as described for the T-cell activation assays above. Plates were incubated at 37° C., 5% CO₂ in humidified incubators. After 4 days, cells were labeled at 4° C., with antibodies for flow cytometric analysis in original plates to minimize cell losses, using flow cytometry buffer with 0.2% bovine serum albumin and 2 mM EDTA. After centrifugation and removal of supernatant, the cell pellets were resuspended in 50 μl volumes containing a mixture of fluorescently-labeled antibodies to the surface antigens CD5, CD8, CD4, and CD25 (Biolegend), and the viability dye 7AAD (SIGMA), and incubated for 30 min on ice. Cells were washed twice and resuspended immediately prior to acquisition of 50% of each well in a BD™ LSRII or a BD FACSymphony™ flow cytometer (BD Biosciences). The sample files were analyzed using FlowJo software to calculate the percentages of CD4⁺ (CD8⁻) or CD8⁺ T-cells that had undergone at least one cell division, according to their CTV profile, by gating sequentially on forward vs side scatter, 7AAD⁻, CD5⁺, CD4⁺ or CD8⁺ T-cells (7AAD⁻, CD5⁺ CD8⁻ or 7AAD⁻ CD5⁺ CD8⁺, respectively). Results were plotted and nonlinear regression analysis to determine EC50 values was performed using GraphPad Prism 7® graphing and statistics software.

To assess cytotoxic function, assays were set up as described above for the proliferation assays, except that the fraction of live target cells was identified by gating sequentially on forward vs side scatter, 7AAD⁻, and CD5⁻ cells.

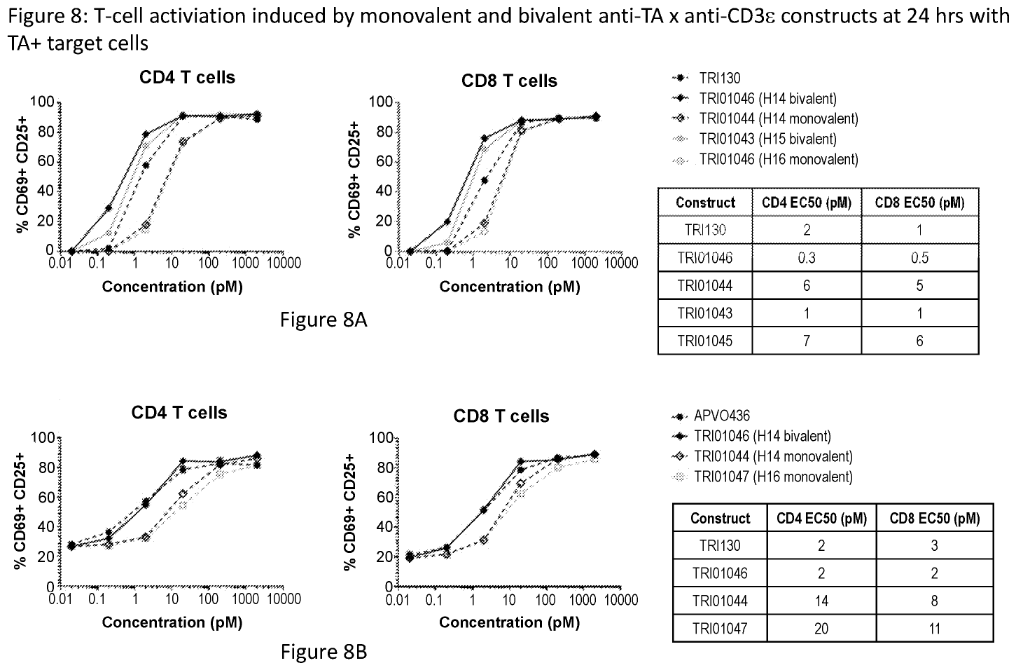

FIG. 8 shows the activation of CD4⁺ and CD8⁺ T cells as defined by the upregulation of CD69 and CD25. FIG. 8A shows that the bispecific constructs bearing the H14 and H15 CD3-binding domains in the bivalent format (TRI1046 and TRI01043) show slightly lower EC50 than the TRI130 control, and further reduced potency when present in the monovalent format (TRI01044 and TRI01045). FIG. 8B shows that the monovalent constructs bearing the H14 and H16 CD3-binding domains show similarly reduced potency compared to the TRI130 control construct. However, all constructs induce similar maximum levels of T-cell activation.

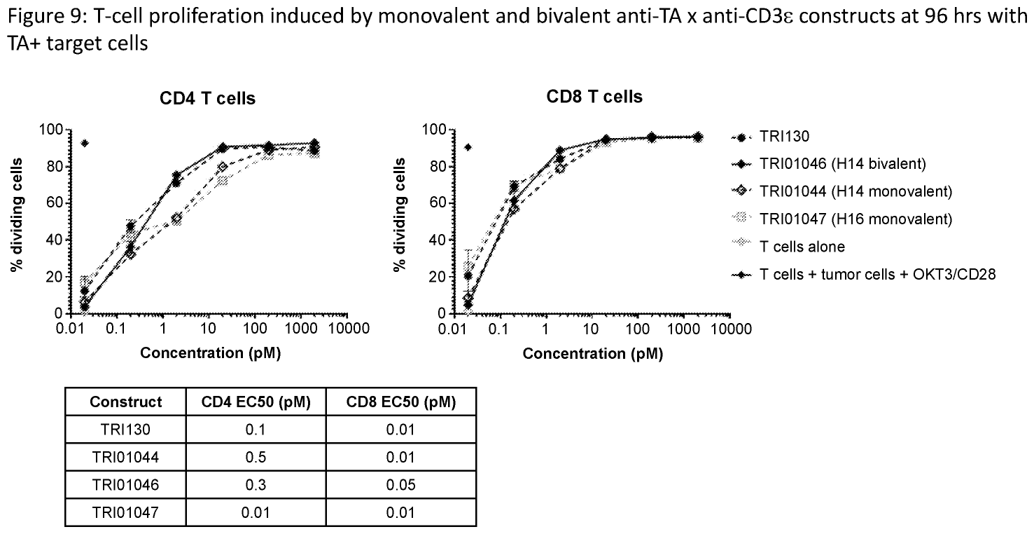

FIG. 9 shows the proliferation of CD4 and CD8 T cells defined by the dilution of CTV dye. The bispecific anti-TA×CD3ε constructs bearing the H14 or H16 CD3-binding domain in monovalent format induced slightly attenuated T-cell proliferation when compared to the TRI130 control construct. However, all constructs induce similar maximum levels of T-cell proliferation.

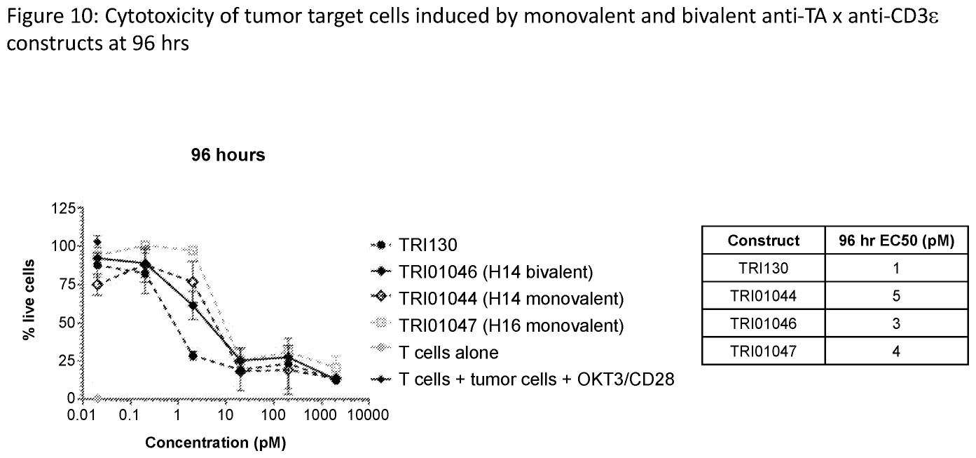

Redirected T-cell cytotoxicity was assessed on TA(+) tumor cells by flow cytometry at 96 hours. FIG. 10 shows that bispecific anti-TA×CD3ε constructs bearing the H14 or H16 CD3-binding domain in monovalent format show reduced potency compared to the TRI130, however they both show equivalent maximum inhibition of tumor growth.

In conclusion, the affinity-optimized H14, H15 and H16 CD3-binding domains show reduced binding to CD3 on a T cell line. As expected, lowest binding to CD3 is observed in the monovalent format. However, H14, H15 and H16 bearing monovalent constructs induce robust T-cell activation and proliferation as well as efficient target cell cytotoxicity, showing slightly reduced potency but similar maximum values as compared to the control construct.

### Example 13. Binding of Anti-PSMA×Anti-CD3F Constructs in Various Formats to PSMA (+) and CD3 (+) Cells

Anti-PSMA×anti-CD3ε constructs were generated in several formats and valencies (mono- and bivalent) to determine the best configuration to induce desired function. The humanized and affinity optimized CD3-binding domain H16 and the optimized PSMA-binding domains PSMA01036 and PSMA01037 were used to build these constructs. The objective was to achieve a construct with limited binding to T cells (CD3ε) alone, but strong functional interaction with T cells in the presence of PSMA-expressing cells.

The anti-PSMA×anti-CD3ε constructs PSMA01026, PSMA01070, PSMA01071, PSMA01072 and PSMA01086 were initially tested in CD3ε and PSMA binding assays on Jurkat and C4-2B cells.

Jurkat and C4-2B cells were labelled at approximately 100,000 cells per well, in 96-well plates, with serial dilutions of bispecific constructs ranging in concentration from of 0.1 to 300 nM, for 30 min on ice. Primary label was followed by washes and incubation with PE-labeled minimum cross species reactive secondary antibody, goat anti-human IgG Fcγ, F(ab′)2 (Jackson Laboratory) for 30 minutes on ice. Washes and incubation were done in staining buffer (PBS with 0.2% BSA and 2 mM EDTA). Cells were collected using an BD™ LSRII or a BD FACSymphony™ flow cytometer (BD Biosciences) and analyzed by FlowJo flow cytometry analysis software. Median fluorescence intensity (MFI) of bound molecules on cells was determined after exclusion of doublets. Results were plotted and nonlinear regression analysis to determine EC50 values was performed using GraphPad Prism 7® graphing and statistics software.

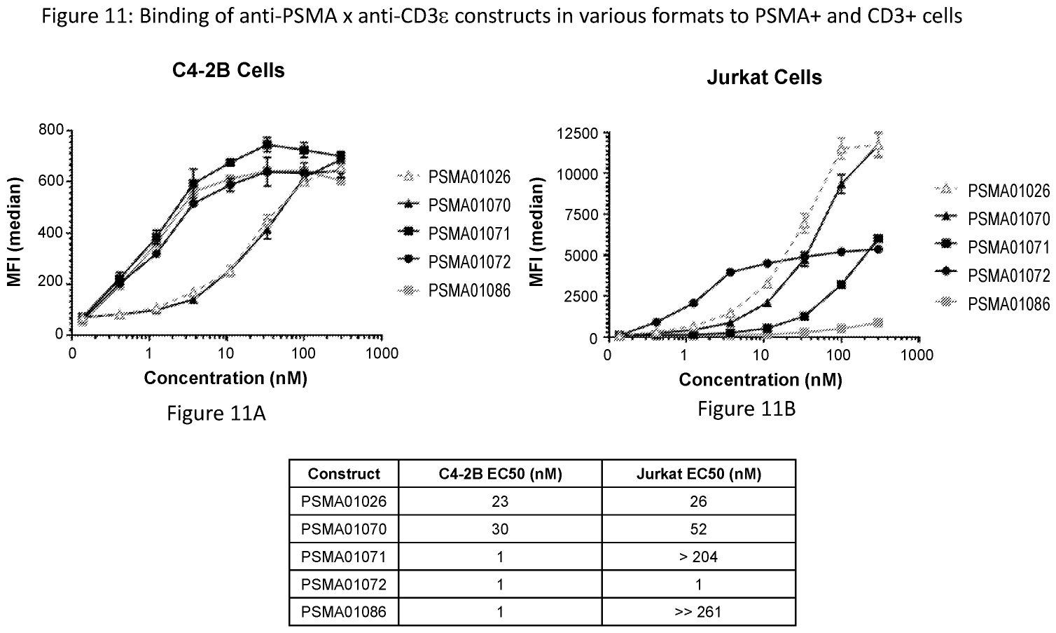

FIG. 11 shows the binding curves of the anti-PSMA×anti-CD3ε constructs on C4-2B and Jurkat cells. On C4-2B cells (FIG. 11A), the constructs with monovalent PSMA binding (PSMA01026 and PSMA01070) displayed less potent binding than constructs with bivalent PSMA binding (PSMA01071, PSMA1072 and PSMA01086). The constructs with swapped VH orientation but otherwise identical format (PSMA01026 and PSMA01070) showed comparable PSMA binding. On Jurkat cells (FIG. 11B), a range of binding potencies was observed. The bivalent CD3-binding domain (PSMA01072) showed the strongest binding, compared to all monovalent CD3 binders. The monovalent CD3 binders showed weaker binding when the CD3-binding domain was located at the C-terminus (PSMA01071 and PSMA01086), compared to the N-terminus (PSMA01026 and PSMA01070). Swapping the orientation of the CD3-binding domain from VH-VL (PSMA01071) to VL-VH (PSMA01086) further reduced binding to CD3 in the C-terminus. The maximum CD3-binding signal differed between the constructs with N-terminal CD3-binding domain constructs showing the highest maximum binding. However, potency (EC50) and not maximum binding correlated with function, as will be shown in the next example.

In conclusion, the format and VH orientation of the CD3-binding domain had a profound impact on the binding potency of the anti-PSMA×anti-CD3 constructs. The constructs with the lowest binding to CD3 were those bearing the CD3-binding domain in monovalent format in the C-terminus of the construct.

### Example 14: Human T-Cell Activation and Proliferation, Cytokine Release and Target-Cell Cytotoxicity in Response to Anti-PSMA×Anti-CD3F Constructs in Various Formats

The effectiveness of the anti-TA×CD3ε constructs in different formats to induce target-dependent T-cell activation and proliferation, was compared to that of the unoptimized TSC266 parent construct.

The following assays were assessed in cultures with unseparated human PBMC. PBMC were obtained from healthy volunteers and isolated using standard density gradient centrifugation, and used immediately after isolation or after thawing from cryopreserved cells banks. Constructs had a mutated Fc to eliminate Fc interactions with Fey receptors.

For activation assays, PBMC were plated in U-bottom 96-well plates at about 100,000 cells/well, with or without 30,000 C4-2B cells/well, to achieve approximate T-cell to tumor cell ratios of 3:1. Serial dilutions of test molecules at concentrations ranging from 0.02 to 2,000 μM were added to the cell mixtures to a final volume of 200 μl/well in RPMI 1640 media supplemented with 10% FBS (SIGMA) sodium pyruvate, antibiotics and non-essential amino acids. Control wells received anti-CD3 (OKT3 clone, Biolegend, Ultra-LEAF) and anti-CD28 (clone CD28.2, Biolegend, Ultra-LEAF). Plates were incubated at 37° C., 5% CO₂ in humidified incubators. After 20 to 24 hours, cells were labeled at 4° C., with antibodies for flow cytometric analysis in original plates to minimize cell losses, using saline buffer with 0.10% bovine serum albumin and 2 mM EDTA. After centrifugation and removal of supernatant, the cell pellets were resuspended in 50 μl volumes containing a mixture of fluorescently-labeled antibodies to the surface antigens CD5, CD8, CD4, CD25, and CD69 (Biolegend), and the viability dye 7AAD (SIGMA), and incubated for 30 min on ice. Cells were washed twice and resuspended immediately prior to acquisition of 50% of each well in a BD™ LSRII or a BD FACSymphony™ flow cytometer (BD Biosciences). The sample files were analyzed using FlowJo software to calculate the percentages of CD4⁺ or CD8⁺ T-cells that had upregulated CD69 and CD25, by gating sequentially on forward vs side scatter, 7AAD⁻, CD5⁺, CD4⁺ or CD8⁺ T-cells (7AAD⁻, CD5⁺ CD4⁺ or 7AAD⁻ CD5⁺ CD8⁺, respectively). Results were plotted and nonlinear regression analysis to determine EC50 values was performed using GraphPad Prism 7© graphing and statistics software.

To quantify cytokine release, the culture supernatants from the activation assays were harvested at 20 to 24 hours prior to labeling the cells. The levels of selected cytokines (e.g. IFNγ, IL-2, TNFα and IL-6) were determined using multiplexed analyte assays (Milliplex cytokine kits, Millipore/SIGMA) following the manufacturer's instructions. The processed samples were collected using a MAGPIX™ instrument (Thermofisher). Results were plotted using GraphPad Prism 7® graphing and statistics software.

For assessment of T-cell proliferation, T cells were labeled with CellTrace™ Violet (CTV) dye (Thermofisher). CTV-labeled T-cells were plated in U-bottom 96-well plates at about 100,000 cells/well with 30,000 C4-2B tumor cells/well, to achieve approximate T-cell to tumor cell ratios of 3:1 as described for the T-cell activation assays above. Plates were incubated at 37° C., 5% CO₂ in humidified incubators. After 4 days, cells were labeled at 4° C., with antibodies for flow cytometric analysis in original plates to minimize cell losses, using flow cytometry buffer with 0.2% bovine serum albumin and 2 mM EDTA. After centrifugation and removal of supernatant, the cell pellets were resuspended in 50 μl volumes containing a mixture of fluorescently-labeled antibodies to the surface antigens CD5, CD8, CD4, and CD25 (Biolegend), and the viability dye 7AAD (SIGMA), and incubated for 30 min on ice. Cells were washed twice and resuspended immediately prior to acquisition of 50% of each well in a BD™ LSRII or a BD FACSymphony™ flow cytometer (BD Biosciences). The sample files were analyzed using FlowJo software to calculate the percentages of CD4⁺ (CD8⁻) or CD8⁺ T-cells that had undergone at least one cell division, according to their CFSE profile, by gating sequentially on forward vs side scatter, 7AAD⁻, CD5⁺, CD4⁺ or CD8⁺ T-cells (7AAD⁻, CD5⁺ CD8⁻ or 7AAD⁻ CD5⁺ CD8⁺, respectively). Results were plotted and nonlinear regression analysis to determine EC50 values was performed using GraphPad Prism 7® graphing and statistics software.

To assess target-cell cytotoxicity, viability of C4-2B target cells was measured by their expression of luciferase. C4-2B cells were transduced to express firefly luciferase using RediFect™ Red-FLuc-Puromycin Lentiviral Particles (PerkinElmer). Approximately 60,000 PBMC/well were co-cultured with 12,000 C4-2B-luciferase cells/well in 96-well black bottom plates (Corning #4591). Serial dilutions of test molecules at concentrations ranging from 1 to 1,000 μM were added to the cell mixtures to a final volume of 200 μl/well in RPMI 1640 media supplemented with 10% FBS (SIGMA) sodium pyruvate, antibiotics and non-essential amino acids. Plates were incubated at 37° C., 5% CO₂ in humidified incubators for up to 96 hours. Cells were removed from incubator and 20 μl of luciferin reagent (D-Luciferin Firefly, PerkinElmer #122799) diluted at 1:10, was added to each well. Plates were covered and incubated for 10 min at room temperature. Luminescence signal was collected on MicroBeta plate reader (PerkinElmer). Results were plotted and nonlinear regression analysis to determine EC50 values was performed using GraphPad Prism 7® graphing and statistics software.

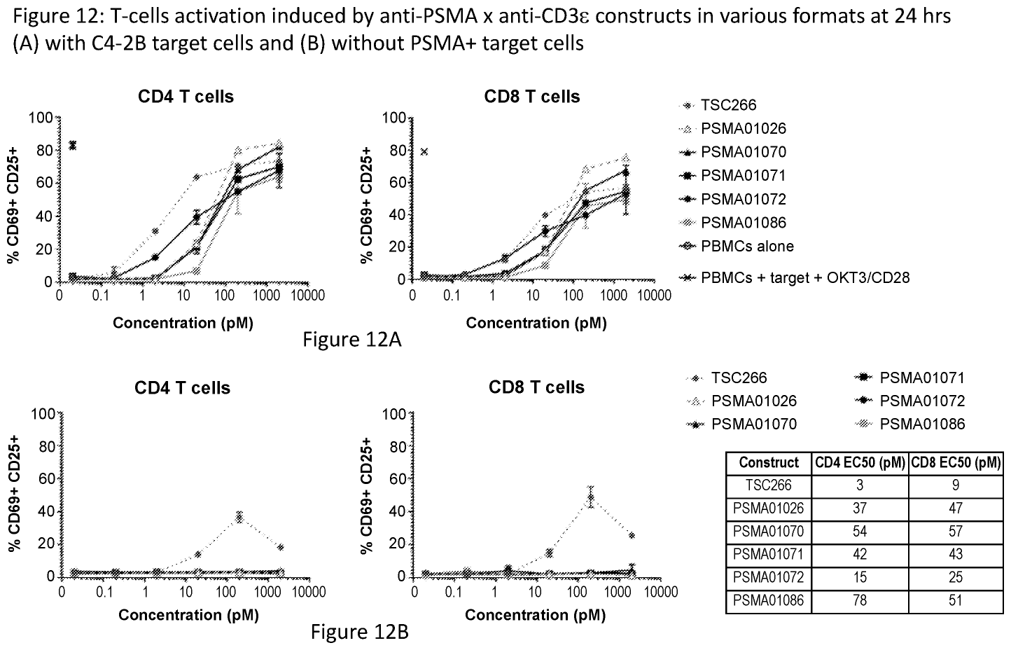

CD4⁺ and CD8⁺ T-cell activation induced by the anti-PSMA×anti-CD3 constructs was assessed at 24 hours, as defined by the upregulation of CD69 and CD25. In the presence of C4-2B target cells (FIG. 12A), all constructs induced robust T-cell activation with a diversity of potencies. The two constructs with bivalent binding to both CD3 and PSMA targets (parent construct TSC266 and PSMA01072) showed the highest potency, whereas the PSMA01086 construct was the least potent. This ranking correlates with the ranking in the cell binding results. However, all constructs induced similar maximum levels of T-cell activation, comparable to the levels reached in the OKT3/CD28 antibody control, which induces optimal T-cell activation. In the absence of target cells (FIG. 12B) there was no measurable T-cell activation. In contrast, the unoptimized parent construct TSC266 shows variable levels of T-cell activation depending on the human donor (FIG. 12B and data not shown).

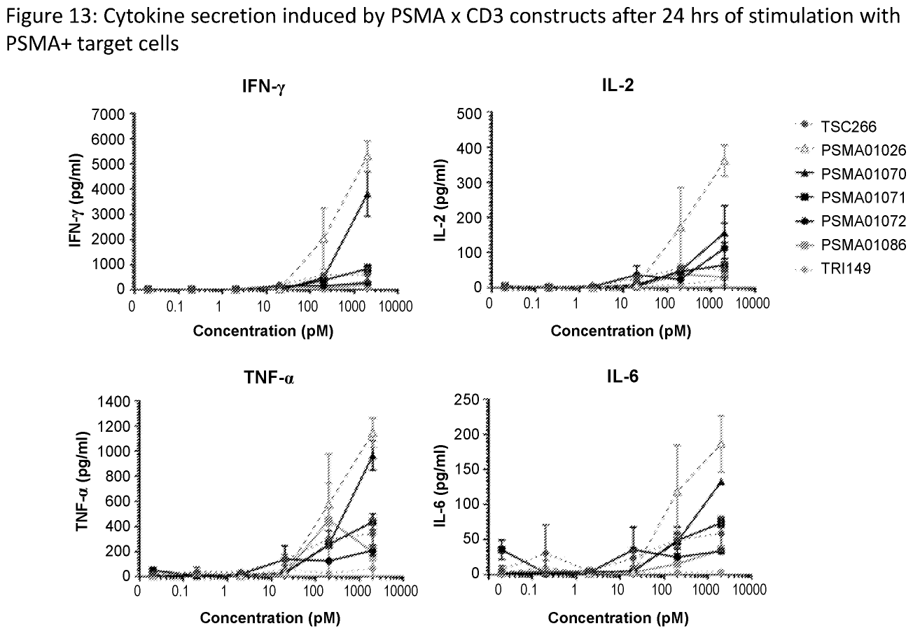

Cytokines are secreted during T-cell activation. The levels of cytokines secreted in the culture supernatant in the T-cell activation assay described above were quantified (FIG. 13). Constructs with the CD3-binding domain in the N-terminus, whether bivalent (TSC266 and PSMA01072) or monovalent (PSMA01071 and PSMA01086), induced lower levels of cytokine secretion than constructs with CD3-binding domain in the C-terminus (PSMA01026 and PSMA01070). This is despite fact that the latter are monovalent for the CD3-binding domain. This is consistent with previous observations indicating that the localization of the CD3-binding domain within the ADAPTIR™ format impacts function (Hemandez-Hoyos et al. Mol Cancer Ther. (2016) 15:2155-65).

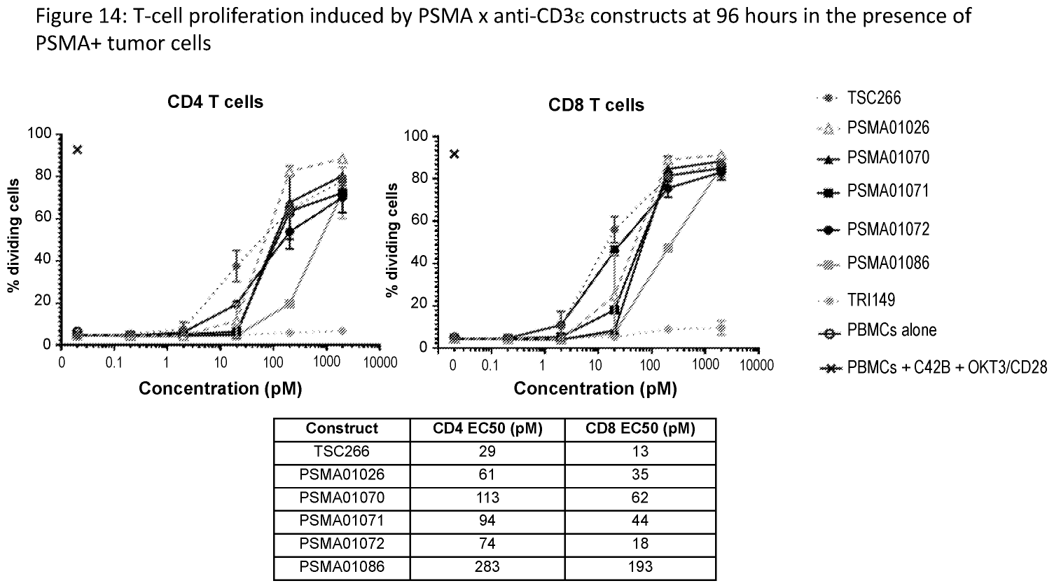

All the anti-PSMA×CD3 constructs also induced T-cell proliferation at 96 hours (FIG. 14). TSC266 showed the highest potency, whereas PSMA01086 was the least potent, correlating with the ranking in the T-cell activation assays. Once again, all the constructs induced proliferation of the majority of the CD4 and CD8 T cells.

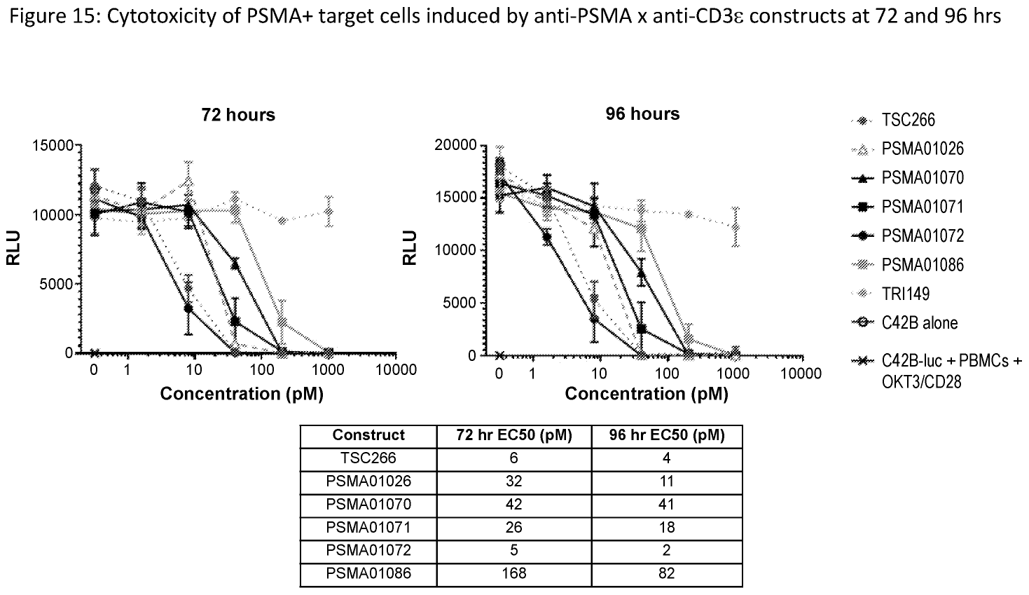

Cytotoxicity assays using C4-2B as target cells demonstrated a range of potencies (FIG. 15). The two constructs with bivalent binding to both CD3 and PSMA targets (parent construct TSC266 and PSMA01072) showed the highest potency, whereas the PSMA01086 construct was the least potent, correlating with the potency in the T-cell activation and proliferation assays. The negative control ADAPTIR™ TRI149 showed no impact on the C4-2B cell growth.

Four main conclusions can be drawn from these examples. 1) There is a correlation between CD3-binding and function, with lower CD3-binding potency resulting in lower T-cell activation, proliferation and target cytotoxicity potency. 2) Dramatic changes in CD3 affinity can be attenuated during T cell:Target cell interactions due to the multivalent nature of the interaction, which compensates for the low affinity to CD3. The reduction in function was not proportional to the reduction in binding potency, e.g.: a binding reduction of ˜200-fold comparing PSMA01072 vs PSMA 1071 resulted in a ˜3-fold reduction in T-cell activation and proliferation, and a ˜10-fold reduction in cytotoxic activity (these are approximate calculations). 3) In spite of the lower potency, all constructs (high or low CD3 affinity) induced maximum levels of T-cell activation, proliferation and cytotoxicity. This may reach a limit depending on the CD3 affinity and can be impacted by the PSMA binding affinity 4) Levels of cytokine secretion correlate with the localization of the CD3-binding domain in the N- or C-terminus.

### Example 15: Anti-Tumor Efficacy in Response to Anti-PSMA×Anti-CD3F Bispecific Protein Treatment

The function and potency of the anti-PSMA×CD3ε constructs was assessed in vivo in a prophylactic xenograft tumor model using human T cells as effector cells.

Male NOD/scid mice (NOD.CB17-Prkdcscid/J) from Jackson Laboratory, Bar Harbor, ME were acclimated for one week before initiation of the study. Animals were checked daily for general health. Treatment of study animals was in accordance with conditions specified in the Guide for the Care and Use of Laboratory Animals, and the study protocol was approved by the Institutional Animal Care and Use Committee (IACUC).

C4-2B-luc cells were transduced to express firefly luciferase using RediFect™ Red-FLuc-Puromycin Lentiviral Particles (PerkinElmer) to enable in vivo quantification. C4-2B-luc cells were thawed and expanded in culture. Human T cells were isolated from frozen leukopak PBMCs using Pan T Cell Isolation Kit (Miltenyi Biotec). NOD/scid mice were challenged on day 0 by injecting 2×10⁶ C4-2B-luc human prostate cancer cells mixed with 1×10⁶ human leukopak T cells in 100 μL of 50% high Content Matrigel (Coming) subcutaneously on their right flank. Starting 2 hours after tumor challenge, mice were treated with either vehicle (PBS), TSC266 and PSMA01072 at dosages of 3 and 0.3 μg/mouse (n=10/group) or PSMA01070 and PSMA01071 at 30, 3 and 0.3 μg/mouse (n=10/group). Treatments were given IV on days 0, 4 and 8. Tumor growth was monitored by bioluminescent imaging (BLI) using an IVIS® Spectrum imager (PerkinElmer) and caliper measurements three times/week. Tumor bioluminescence was calculated using Living Image® software version 4.5.5 (PerkinElmer). Individual tumors were selected using the Auto ROI (region of interest) with the threshold set at 5%. Bioluminescent imaging (BLI) value are expressed as photons/sec and converted by log 10. Tumor free animals were assigned a BLI value of 4 which is just below the log 10 value for the level of detection.

Statistical analyses are performed using SAS/JMP software (SAS Institute). A repeated measures ANOVA model is fitted using Fit Model Standard Least Squares to evaluate overall effects of treatment, day and treatment-by-day interactions on tumor volumes for in vivo studies. Significant differences in tumor size between treatment groups for the s.c. xenograft model was evaluated by a Tukey multiple comparison test using the LSMeans platform and further time and treatment combinations are evaluated using the LSMeans Tukey multiple comparison test for each treatment-by-day combination as needed.

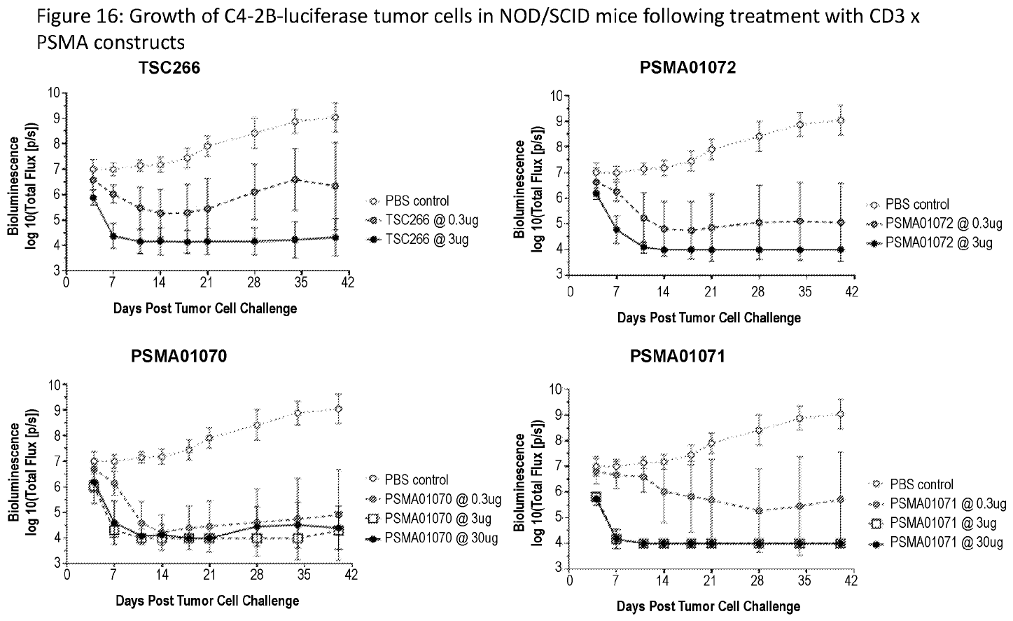

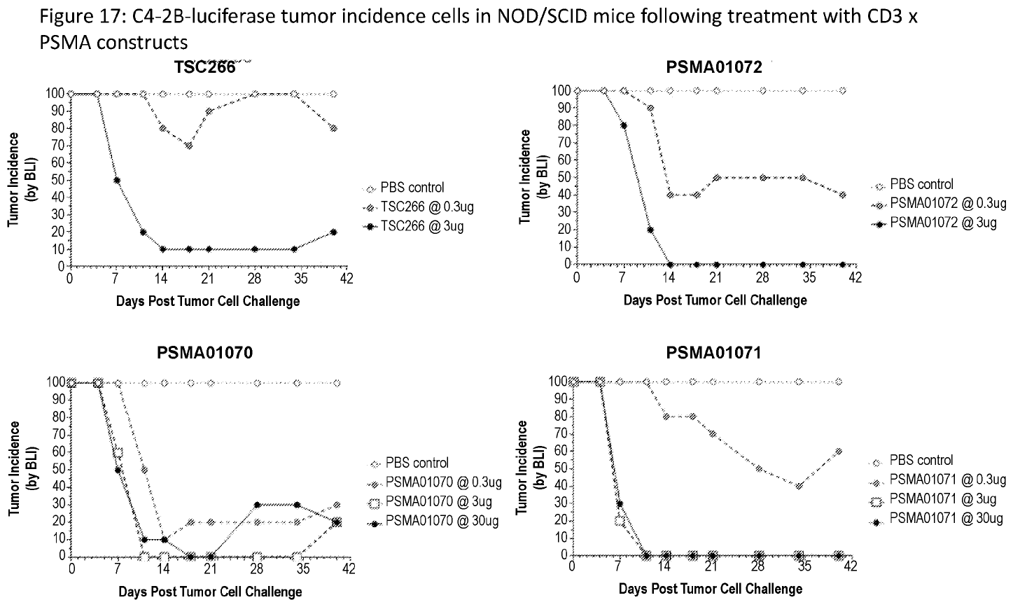

Treatment with all ant-PSMA×anti-CD3 ADAPTIR™ molecules resulted in a statistically significant reduction of C4-21B-luc tumor growth as determined by bioluminescence in NOD/scid mice (FIGS. 16 and 17; Table 5). The reduction in tumor bioluminescence was observed at the first imaging time point on day 4 after receiving only a single injection. Further reduction in tumor bioluminescence was observed over the course of the treatments. Treatments at 0.3 μg/mouse were less effective compared to the 3 and 30 μg/mouse dosages. Therefore, treatment with all PSMA×CD3 ADAPTIR™ molecules resulted in a statistically significant reduction in tumor volume and prevented the outgrowth of tumors in C4-21B-luc challenged mice (FIGS. 16 and 17; Table 5).

Differences in mean tumor bioluminescence from Day 4 through Day 40 for the study groups were determined using JMP repeated measures analysis with Tukey multiple comparison test. Values of p <0.05 were considered significant.

TABLE 5
Statistical Comparison of Mean log10 Tumor Bioluminescence
through Day 4
JMP One-way ANOVA Analysis with Tukey-Kramer HSD Method

| Treatment | p-Value |
|---|---|
| PBS Control vs. TSC266 3 μg | <0.0001 |
| PBS Control vs. TSC266 0.3 μg | <0.0001 |
| PBS Control vs. PSMA01072 3 μg | <0.0001 |
| PBS Control vs. PSMA01072 0.3 μg | <0.0001 |
| PBS Control vs. PSMA01070 30 μg | <0.0001 |
| PBS Control vs. PSMA01070 3 μg | <0.0001 |
| PBS Control vs. PSMA01070 0.3 μg | <0.0001 |
| PBS Control vs. PSMA01071 30 μg | <0.0001 |
| PBS Control vs. PSMA01071 3 μg | <0.0001 |
| PBS Control vs. PSMA01071 0.3 μg | <0.0001 |

### Example 16: Fc Mutations to Eliminate Binding to Fc Receptors and Complement

It may be advantageous in an ADAPTIR™ bispecific molecule to make mutations to the Fc region to eliminate the ability to interact and signal through interactions with the Fc receptors and compliment. Table 6 below shows mutations that could be made to the Fc regions included in an ADAPTIR™ bispecific construct (TSC1007), compared to the sequence of a wild type Fc (WT).

TABLE 6
Fc Mutations for ADAPTIR ™ bispecific construct
to reduce effector function and compliment binding.

| Fc AA Position | | | | | | |
|---|---|---|---|---|---|---|
| according to | | | | | | |
| EU/Kabat/IMGT | 233/246/3 | 234/247/4 | 235/248/5 | 236/249/6 | 237/250/7 | 322/341/92 |
| WT IgG1 | Glu | Leu | Leu | Gly | Gly | Lys |
| TSC1007 | Pro | Ala | Ala | Deletion | Ala | Ala |
| EU-1 | E233P | L234A | L235A | | G237A | K322A |
| EU-3 | Glu233Pro | Leu234Ala | Leu235Ala | Gly | Gly237Ala | Lys322Ala |
| Kabat-1 | E246E | L247A | L248A | | G250A | K341A |
| Kabat-3 | Glu246Pro | Leu247Ala | Leu248Ala | Gly | Gly250Ala | Lys341Ala |
| IMGT-1 | 3 | 4 | 5 | 6 | 7 | 92 |
| IMGT-3 | 3 | 4 | 5 | 6 | 7 | 92 |

### Example 17: SPR Analysis Impact of Fc Mutations on Binding to Fcγ Receptors

The Fc region incorporated into anti-PSMA bispecific constructs contained mutations intended to reduce or abolish binding to common human and cynomolgus Fc gamma receptors. SPR experiments were conducted at 25° C. in HBS-EP+ with 0.2% BSA buffer on a Biacore T200 system to evaluate the impact of these mutations on binding. For these experiments, three flow cells of a CM5 sensor chip were immobilized with anti-PSMA bispecifics by standard amine coupling to a response level of ˜2000 RU. A blank immobilization was performed on flow cell #1 for purposes of background signal subtraction. Both human and cynomolgus monkey Fcγ receptors (purchased from R&D Systems) were diluted in HBS-EP+ with 0.2% BSA to 4-6 μM and then flowed as analytes at 30 μL/min for 120 seconds followed by a 240 second dissociation step. No regeneration step was required. Blank-subtracted sensorgrams were visually inspected for binding to each of the Fcγ receptor proteins. As indicated in Table 7, PSMA01107 and PSMA01108 exhibited no detectable binding to human Fcγ receptors I, IIIA (both polymorphic variants), or IIIB. BLOD indicates that the binding signal, if there was any, was below the limits of detection. Attenuated binding to Fcγ receptors IIA (both polymorphic variants) and IIB/C was observed. PSMA01107 and PSMA01108 share the same Fc amino acid sequence and therefore equivalent Fcγ receptor binding behavior was expected.

Binding affinities to Type II Fcγ receptors were measured for PSMA01107 on a Biacore T200 system using the same experimental conditions above with the addition of a five-point titration of Fcγ receptors from 375-6000 nM in multi-cycle kinetics mode. TSC266 (PSMA×CD3 bispecific antibody) and another protein containing a wild-type IgG1 Fc sequence were also analyzed for comparison. Sensorgrams obtained from kinetic SPR measurements were analyzed by the double subtraction method in the Biacore T200 evaluation software. Kinetic parameters were derived from a one-to-one binding fit model and reported below in Table 8. Both TSC266 and PSMA01107 show reduced binding compared to a wild type IgG1 Fc. The Fc mutations present in PSMA01107 appear to be more effective at weakening the interaction between Type IIA R167 and RIIB/C Fey receptors, whereas the binding affinity to the RIIA H167 variant is comparable.

TABLE 7
Results summary of human Fcγ Receptor binding

| | Binding to different human Fcγ Receptors | | | | | | |
|---|---|---|---|---|---|---|---|
| | | RIIA | RIIA | | RIIIA | RIIIA | |
| Sample | RI | R167 | H167 | RIIB/C | V176 | F176 | RIIIB |
| TSC266 | <BLOD | + | + | + | <BLOD | <BLOD | <BLOD |
| PSMA01107 | <BLOD | + | + | + | <BLOD | <BLOD | <BLOD |
| PSMA01108 | <BLOD | + | + | + | <BLOD | <BLOD | <BLOD |

TABLE 8
Binding affinity to human Fcγ Receptors.

| | KD (μM) | | |
|---|---|---|---|
| Sample | RIIA R167 | RIIA H167 | RIIB/C |
| Wild type Fc | 0.9 | 1.2 | 1.2 |
| TSC266 | 1.5 | 20.0 | 2.7 |
| PSMA01107 | 5.3 | 19.9 | 6.5 |

Binding of three different PSMA×CD3 bispecific proteins to recombinant, soluble nonhuman primate Fey receptors was also evaluated. TSC266, PSMA01107 and PSMA01108 did not show any detectable binding when the soluble receptors were injected at concentration (Table 9).

TABLE 9
Results summary of Cyno Fcγ Receptor binding

Binding

| Sample | Cyno RIIA | Cyno RIIB | Cyno RIII |
|---|---|---|---|
| TSC266 | <BLOD | <BLOD | <BLOD |
| PSMA01107 | <BLOD | <BLOD | <BLOD |
| PSMA01108 | <BLOD | <BLOD | <BLOD |

### Example 18: SPR Experiments of Binding to the Neonatal Fc Receptor (FcRn)

The neonatal Fc receptor, FcRn, is responsible for extending the serum half-life of immunoglobulins and Fc-containing proteins by reducing degradation in the lysosomal compartment of cells. For FcRn to properly bind to immunoglobulins, it must be complexed with another protein, beta-2-macroglobulin. For simplicity, this complex will just be referred to as FcRn for the remainder of the document. IgGs and other serum proteins are continually internalized by cells through pinocytosis. They are transported from the endosome to the lysosome for degradation. However, serum albumin and IgG bind to FcRn under the acidic condition that is present in the vesicle and avoid the lysosome. Upon returning to the cell surface, IgG is unable to bind to FcRn under neutral pH and is released back into circulation. This recycling leads to IgG having serum half-lives >7 days but can be impacted by other mechanisms of serum clearance (target-mediated disposition, degradation, aggregation, etc.).

For antibody-like protein therapeutics that contain an Fc region, it is critical that they bind to FcRn under acidic conditions. PSMA×CD3 bispecific constructs with different Fc mutations were evaluated for their binding to FcRn to verify that the mutations did not impact the FcRn binding under acidic conditions using SPR at pH 6.0.

Recombinant FcRn/b2M protein was generated via transient transfection of HEK-293 cells with a bi-cistronic vector containing the genes for both FcRn and beta-2-macroglobulin. The complex was purified using IMAC chromatography and subsequently buffer exchanged into PBS buffer after verifying purity of the IMAC eluate by analytical SEC. Purified hFcRn/b2M at 5 μg/ml in 10 mM sodium acetate (pH 5.0) was immobilized on a CM5 chip by direct amine coupling chemistry to a level of ˜400 RU. A reference flow cell was left blank.

Different concentrations of the Fc variant protein (1-81 nM by 3-fold dilutions in HBS-EP+ with 0.2% BSA running buffer at pH 6.0) including running buffer as blank were injected in randomized order at 30 μL/min for 180 seconds followed by a 180 second dissociation period. Optimal regeneration was achieved by two injections of HBS-EP+ with 0.2% BSA at pH 7.5 at a flow rate of 30 L/min for 30 seconds followed by running buffer stabilization for 1 minute.

Sensorgrams obtained from kinetic SPR measurements were analyzed by the double subtraction method. The signal from the reference flow cell was subtracted from the analyte binding response obtained from flow cell with immobilized ligands. Buffer reference was subtracted from analyte binding responses, and the final double-referenced data were analyzed with Biacore T200 Evaluation software (2.0, GE), globally fitting data to derive kinetic parameters. All sensorgrams were fitted using two-state reaction model, as described in Weirong Wang et al, Drug Metab Dispos.: 39(9): 1469-77 (2011). A steady-state affinity model was also applied for comparison purposes and yielded similar values.

As shown in Table 10 below, the KD values for the PSMA×CD3 bispecifics are within a range consistent with that reported in the literature for monoclonal antibodies containing a wild-type IgG1 Fc.

TABLE 10
FcRn Dissociation Constant (KD) for
ADAPTIR ™ PSMA × CD3 bispecifics

| | KD (nM) | KD (nM) |
|---|---|---|
| | Two-state reaction | Steady-state |
| Construct ID | model fit | affinity model |
| PSMA01107 | 47 | 59 |
| PSMA01108 | 19 | 27 |

### Example 19: SPR Evaluation of PSMA×CD3 Bispecific Proteins Binding to Recombinant PSMA ECD

The KD was determined for a set of PSMA×CD3 bispecific proteins binding to recombinant dimeric PSMA ECD using SPR. In comparison to the constructs analyzed in Table 3, which all had the anti-PSMA scFv in the VL-VH orientation, the constructs in Table 11 have sequences with the reverse the order of the variable domains. The constructs below utilize the PSMA01023 anti-PSMA sequence as a basis. The reverse orientation of the scFv led to measurably tighter binding than PSMA01023, which was determined to have a KD of 40 nM. PSMA01107 and PSMA01108 both bound with binding affinities <10 nM.

TABLE 11
Biacore affinity measurements on optimized
anti-PSMA-binding domains.

| Name | KD (nM) | ka (1/Ms) | kd (1/s) |
|---|---|---|---|
| PSMA01107 | 3.4 | 2.9E+04 | 8.5E−05 |
| PSMA01108 | 3.1 | 2.9E+04 | 9.0E−05 |

### Example 20: In Vitro Evaluation of PSMA×CD3 Bispecific Proteins

Next, anti-PSMA×CD3ε constructs were evaluated with alterations to the Fc region that were intended to reduce Fcγ receptor binding (PSMA01107, PSMA01108, and PSMA01110) in their ability to induce target-dependent T-cell activation and proliferation.

Constructs were initially tested in CD3ε and PSMA binding assays on Jurkat and C4-2B cells. Cells were labelled at approximately 100,000 cells per well, in 96-well plates, with serial dilutions of bispecific constructs ranging in concentration from of 0.1 to 300 nM, for 30 min on ice. Primary label was followed by washes and incubation with PE-labeled minimum cross species reactive secondary antibody, goat anti-human IgG Fcγ, F(ab′)2 (Jackson Laboratory) for 30 minutes on ice. Washes and incubation were done in staining buffer (PBS with 0.2% BSA and 2 mM EDTA). Cells were collected using an BD™ LSRII or a BD FACSymphony™ flow cytometer (BD Biosciences) and analyzed by FlowJo flow cytometry analysis software. Median fluorescence intensity (MFI) of bound molecules on cells was determined after exclusion of doublets. Results were plotted and nonlinear regression analysis to determine EC50 values was performed using GraphPad Prism 7© graphing and statistics software.

For activation assays, PBMC were plated in U-bottom 96-well plates at about 100,000 cells/well, with or without 30,000 C4-2B cells/well, to achieve approximate T-cell to tumor cell ratios of 3:1. Serial dilutions of test molecules at concentrations ranging from 0.02 to 2,000 μM were added to the cell mixtures to a final volume of 200 μl/well in RPMI 1640 media supplemented with 10% FBS (SIGMA) sodium pyruvate, antibiotics and non-essential amino acids. Control wells received anti-CD3 (OKT3 clone, Biolegend, Ultra-LEAF) and anti-CD28 (clone CD28.2, Biolegend, Ultra-LEAF). Plates were incubated at 37° C., 5% CO₂ in humidified incubators. After 20 to 24 hours, cells were labeled at 4° C., with antibodies for flow cytometric analysis in original plates to minimize cell losses, using saline buffer with 0.10% bovine serum albumin and 2 mM EDTA. After centrifugation and removal of supernatant, the cell pellets were resuspended in 50 μl volumes containing a mixture of fluorescently-labeled antibodies to the surface antigens CD5, CD8, CD4, CD25, and CD69 (Biolegend), and the viability dye 7AAD (SIGMA), and incubated for 30 min on ice. Cells were washed twice and resuspended immediately prior to acquisition of 50% of each well in a BD™ LSRII or a BD FACSymphony™ flow cytometer (BD Biosciences). The sample files were analyzed using FlowJo software to calculate the percentages of CD4⁺ or CD8⁺ T-cells that had upregulated CD69 and CD25, by gating sequentially on forward vs side scatter, 7AAD⁻, CD5⁺, CD4⁺ or CD8⁺ T-cells (7AAD⁻, CD5⁺ CD4⁺ or 7AAD⁻ CD5⁺ CD8⁺, respectively). Results were plotted and nonlinear regression analysis to determine EC50 values was performed using GraphPad Prism 7® graphing and statistics software.

To quantify cytokine release, the culture supernatants from the activation assays were harvested at 20 to 24 hours prior to labeling the cells. The levels of selected cytokines (e.g. IFNγ, IL-2, TNFα and IL-6) were determined using multiplexed analyte assays (Milliplex cytokine kits, Millipore/SIGMA) following the manufacturer's instructions. The processed samples were collected using a MAGPIX™ instrument (Thermofisher). Results were plotted using GraphPad Prism 7® graphing and statistics software.

For assessment of T-cell proliferation, T cells were labeled with CellTrace™ Violet (CTV) dye (Thermofisher). CTV-labeled T-cells were plated in U-bottom 96-well plates at about 100,000 cells/well with 30,000 C4-2B tumor cells/well, to achieve approximate T-cell to tumor cell ratios of 3:1 as described for the T-cell activation assays above. Plates were incubated at 37° C., 5% CO₂ in humidified incubators. After 4 days, cells were labeled at 4° C., with antibodies for flow cytometric analysis in original plates to minimize cell losses, using flow cytometry buffer with 0.2% bovine serum albumin and 2 mM EDTA. After centrifugation and removal of supernatant, the cell pellets were resuspended in 50 μl volumes containing a mixture of fluorescently-labeled antibodies to the surface antigens CD5, CD8, CD4, and CD25 (Biolegend), and the viability dye 7AAD (SIGMA), and incubated for 30 min on ice. Cells were washed twice and resuspended immediately prior to acquisition of 50% of each well in a BD™ LSRII or a BD FACSymphony™ flow cytometer (BD Biosciences). The sample files were analyzed using FlowJo software to calculate the percentages of CD4⁺ (CD8⁻) or CD8⁺ T-cells that had undergone at least one cell division, according to their CFSE profile, by gating sequentially on forward vs side scatter, 7AAD⁻, CD5⁺, CD4⁺ or CD8⁺ T-cells (7AAD⁻, CD5⁺ CD8⁻ or 7AAD⁻ CD5⁺ CD8⁺, respectively). Results were plotted and nonlinear regression analysis to determine EC50 values was performed using GraphPad Prism 7© graphing and statistics software.

To assess target-cell cytotoxicity, viability of C4-2B target cells was measured by their expression of luciferase. C4-2B cells were transduced to express firefly luciferase using RediFect™ Red-FLuc-Puromycin Lentiviral Particles (PerkinElmer). Approximately 60,000 PBMC/well were co-cultured with 12,000 C4-2B-luciferase cells/well in 96-well black bottom plates (Corning #4591). Serial dilutions of test molecules at concentrations ranging from 1 to 1,000 μM were added to the cell mixtures to a final volume of 200 μl/well in RPMI 1640 media supplemented with 10% FBS (SIGMA) sodium pyruvate, antibiotics and non-essential amino acids. Plates were incubated at 37° C., 5% CO₂ in humidified incubators for up to 96 hours. Cells were removed from incubator and 20 μl of luciferin reagent (D-Luciferin Firefly, PerkinElmer #122799) diluted at 1:10, was added to each well. Plates were covered and incubated for 10 min at room temperature. Luminescence signal was collected on MicroBeta plate reader (PerkinElmer). Results were plotted and nonlinear regression analysis to determine EC50 values was performed using GraphPad Prism 7® graphing and statistics software.

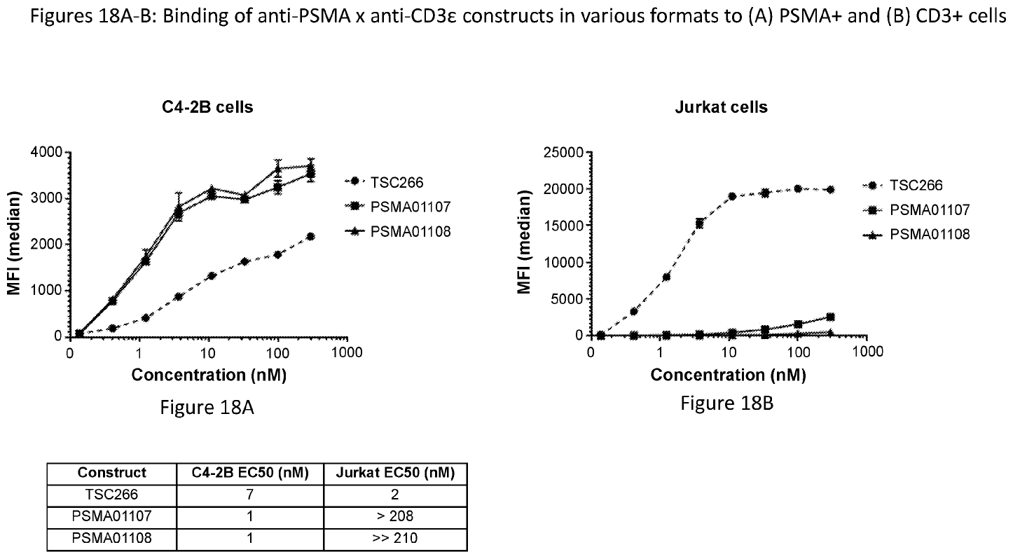

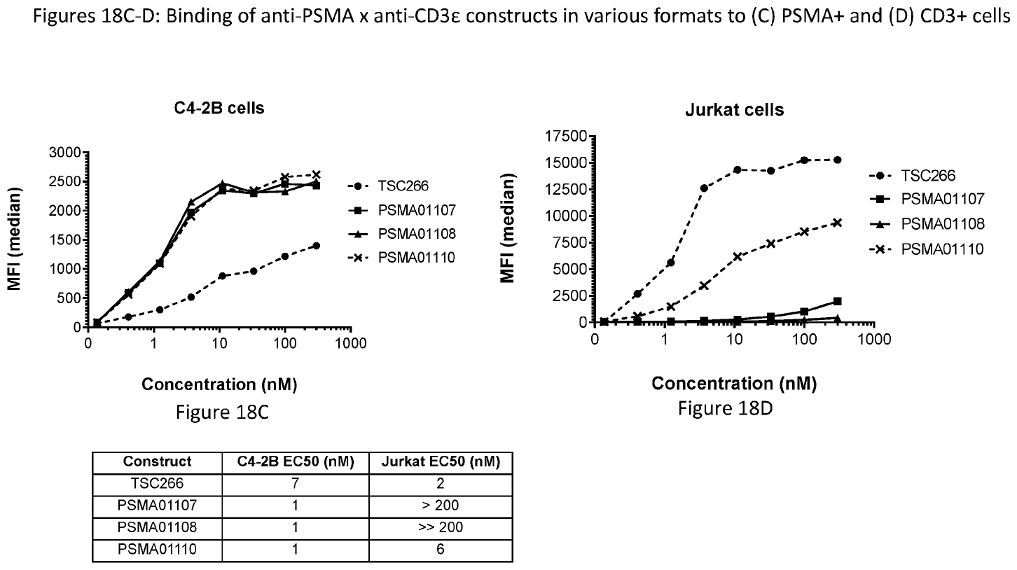

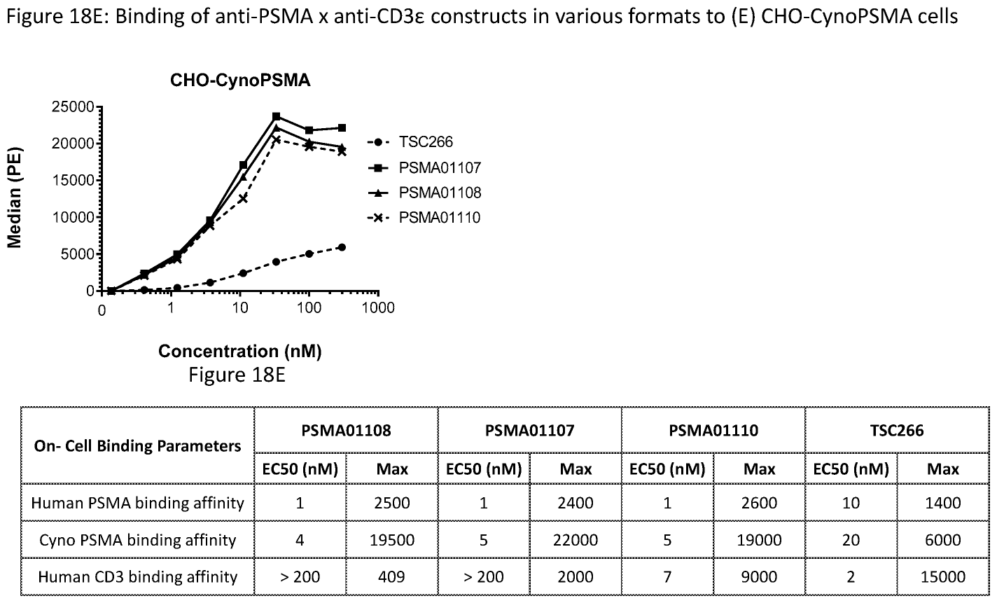

FIG. 18A-E show the binding curves of the anti-PSMA×anti-CD3ε constructs on C4-2B, Jurkat cells, and CHO-CynoPSMA cells. Despite all constructs being bivalent for PSMA binding, TSC266 displayed less potent binding than PSMA01107, PSMA01108, or PSMA01110 to the PSMA expressing target, C4-2B (FIGS. 18A and 18C) or the overexpression cynoPSMA cell line CHO-CynoPSMA (FIG. 18E). On Jurkat cells (FIGS. 18B and 18D), the high affinity CD3 binders, TSC266, and PSMA01110 had stronger binding than did either low affinity binder. PSMA01107 was slightly better than PSAM01108, but neither were able to show saturatable curves at the concentrations tested.

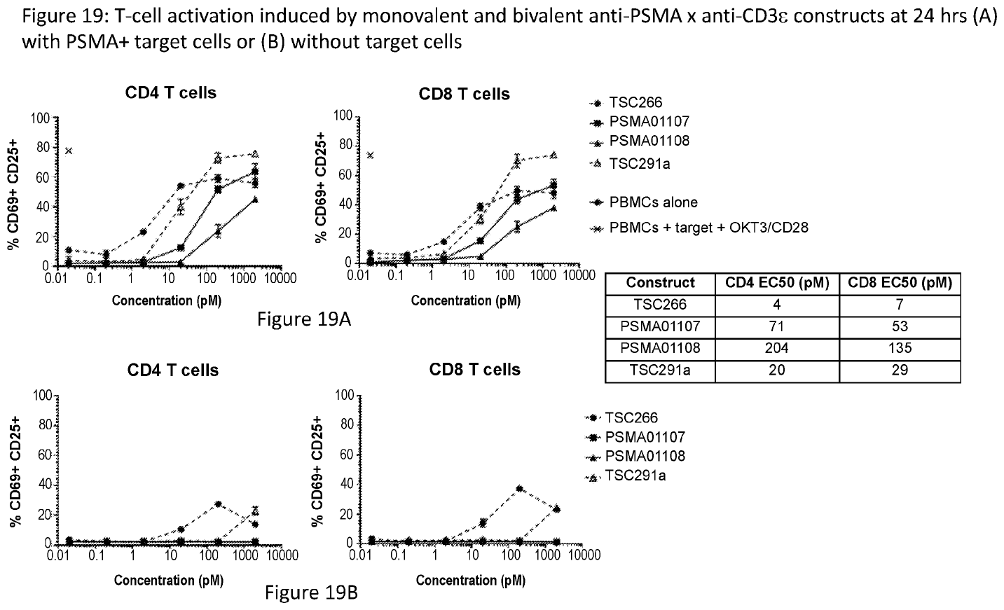

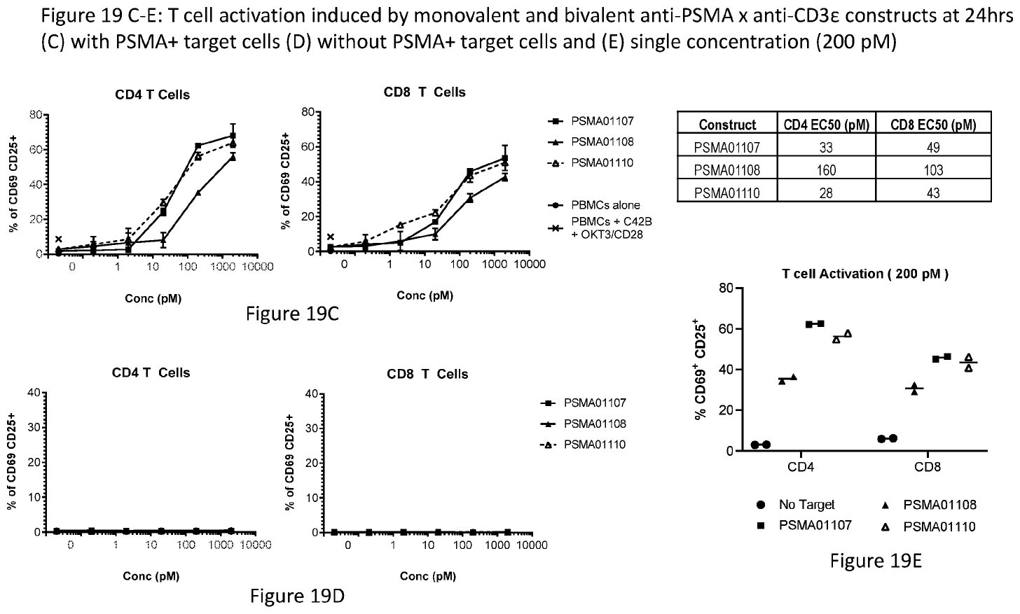

CD4⁺ and CD8⁺ T-cell activation induced by the anti-PSMA×anti-CD3 constructs was assessed at 24 hours, as defined by the upregulation of CD69 and CD25. In the presence of C4-2B target cells (FIG. 19A), all constructs induced robust T-cell activation with a diversity of potencies. The parent construct TSC266 showed the highest potency (lowest EC50), whereas the TSC291a BiTE induced the highest percentage of CD69⁺ and CD25⁺ cells comparable to the levels reached in the OKT3/CD28 antibody control, which induces optimal T-cell activation. PSMA01 107 induced similar levels of activation, but at a higher concentration than TSC266. Although lower than the other test molecules, PSMA01108 promoted the activation of both CD4 and CD8 T cells, despite having nearly undetectable cell binding. In the absence of target cells (FIG. 19B) there was no measurable T-cell activation with either PSMA01107 or PSMA01108. In contrast, the unoptimized parent construct TSC266 and the TSC291a BiTE showed moderate levels of T-cell activation (FIG. 19B). The high affinity CD3 construct, PSMA01110, showed similar activity to the low affinity CD3 construct, PSMA01107, in the presence of C4-2B (FIG. 19C). No activity was observed in the absence of C4-2B target crosslinking (FIG. 19D). A comparison of PSMA01107, PSMA01108, and PSMA01110 shows that PSMA01107 retains comparable T cell activation as the high affinity CD3 construct, PSMA01110, despite differences in CD3 binding. In contrast, PSMA01108, which displays the lowest CD3 binding does not induce T cell activation to the level of PSMA01107 or PSMA01110 (FIG. 19E).

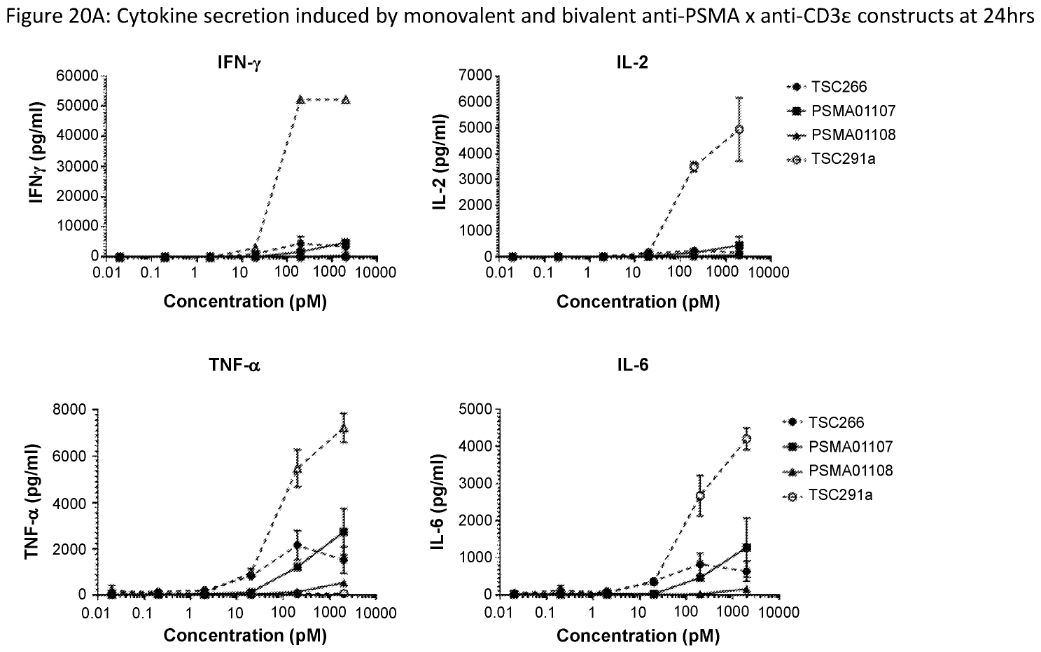

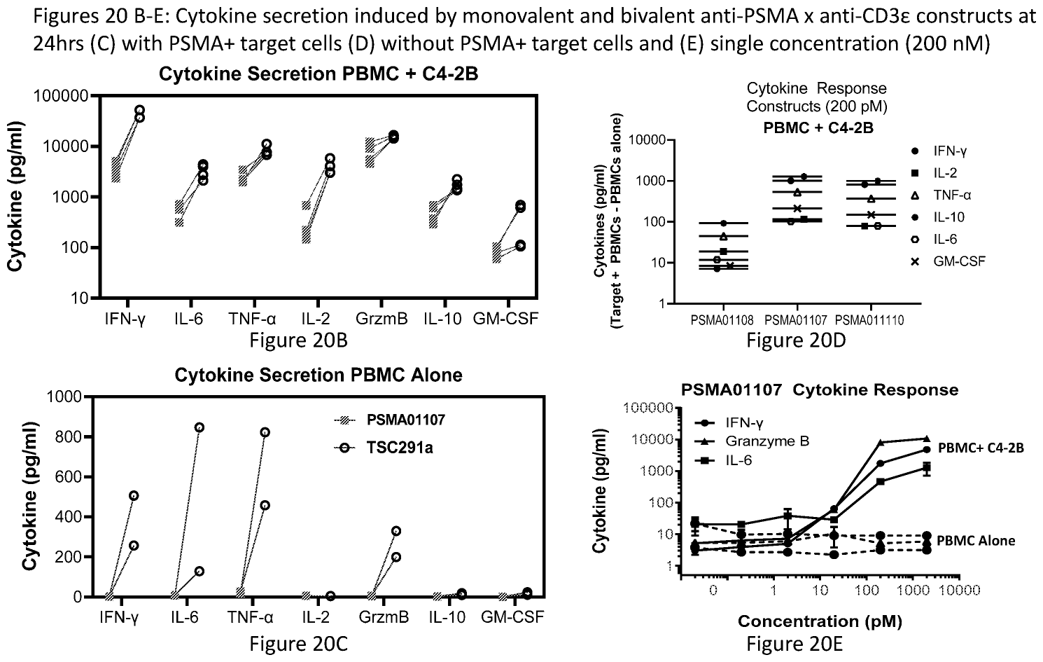

Cytokines are secreted during T-cell activation. The levels of cytokines secreted in the culture supernatant in the T-cell activation assay described above were quantified (FIG. 20). TSC291a induced T cells to secrete abundant IFN-γ, IL-2, TNF-α and IL-6 at significantly higher levels than TSC266, PSMA01107 or PSMA01108 (FIG. 20A). A comparison of PSMA01107 and TSC291a at 200 μM demonstrated a similar cytokine response profile, demonstrating that despite overall higher responses by TSC291a, PSMA01107 induces a functional response in the presence of C4-2B target cells (FIG. 20B). This cytokine activity was associated with the strength of CD3 binding as PSMA01108 and PSMA01110 showed similar responses in the presence of C4-2B target cells, whose magnitude tracks with binding to CD3 (FIG. 20D). In contrast, in the absence of C4-2B target cells, PSMA01107 does not elicit any measureable cytokine response, whereas the response by TSC291a is evident (FIG. 20C). This activity is dose dependent as PSMA01107 shows a titratable cytokine induction only in the presence of C4-2B target crosslinking (FIG. 20E).

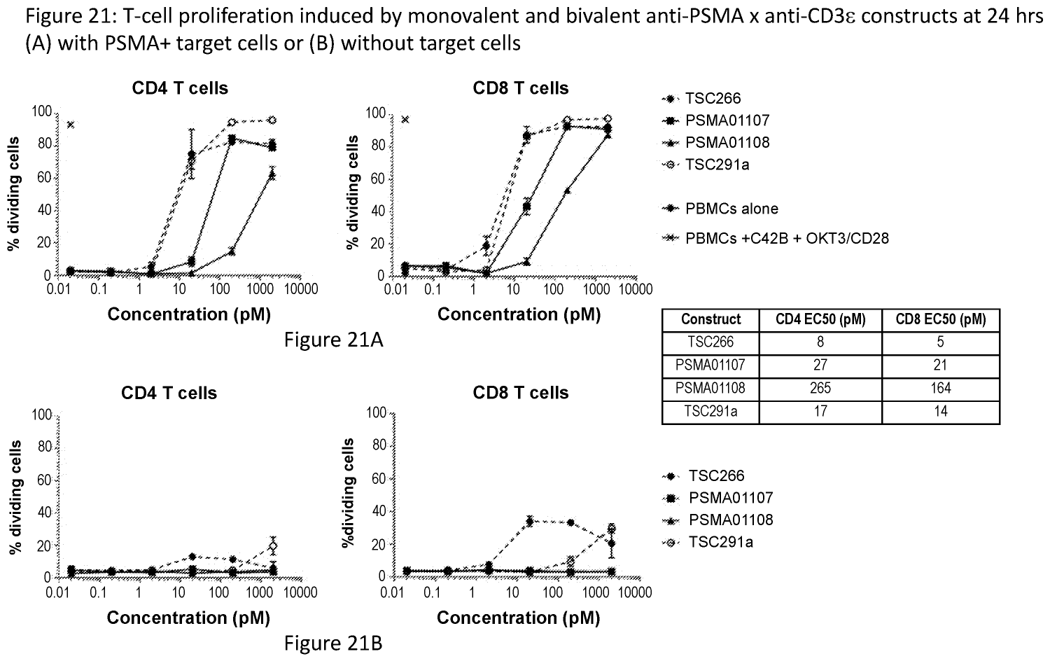

All of the anti-PSMA×anti-CD3 constructs induced T-cell proliferation at 96 hours (FIG. 21A). Both TSC266 and TSC291a showed the highest potency, whereas PSMA01108 was the least potent and PSMA01107 fell inbetween, correlating with the ranking in the T-cell activation assays. All constructs induced proliferation of the majority of the CD4 and CD8 T cells in the dose range tested. TSC266 stimulated moderate cell proliferation at doses as low as 20 μM (FIG. 21B).

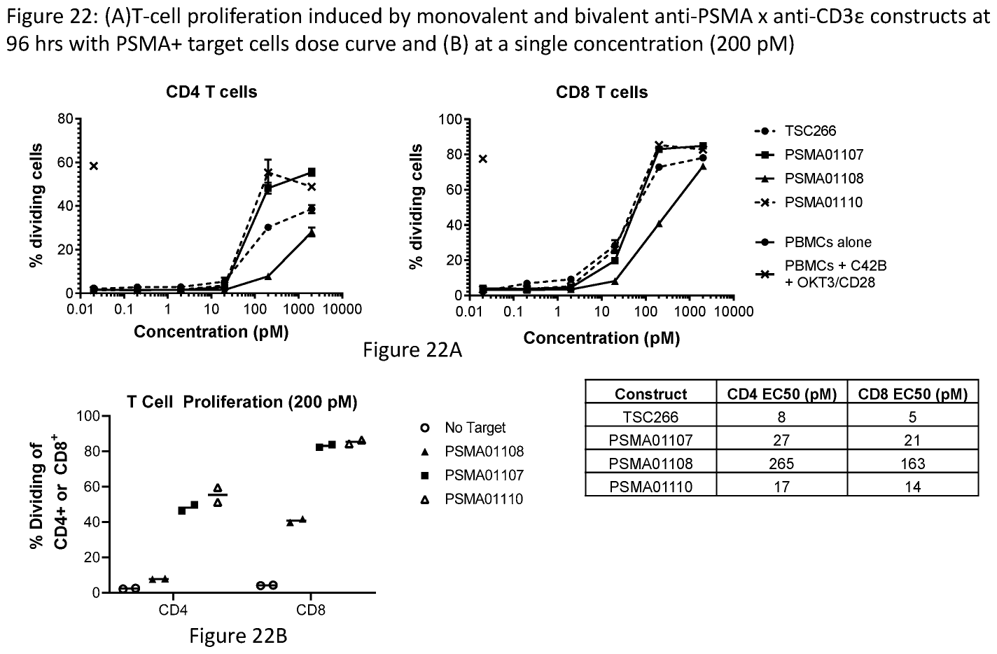

All the anti-PSMA×anti-CD3 constructs induced T-cell proliferation at 96 hours (FIG. 22A). TSC266, PSMA01107, and PSMA01110 showed similar potency on CD4 T cells. TSC266 showed slightly higher potency than PSMA01107 and PSMA01110 on CD8 T cells. PSMA01108 showed reduced potency compared to all constructs tested and is in line with its overall reduced ability to bind CD3 (FIG. 18). In contrast, despite reduced binding to CD3 by PSMA01107 compared to PSMA0110 (FIG. 18), PSMA01107 was sufficient to induce a similar potency for proliferation of both CD4 and CD8 T cells as compared to PSMA01110. A comparison of PSMA01107, PSMA01108, and PSMA01110 shows that PSMA01107 retains comparable T cell proliferation potency as the high affinity CD3 PSMA01110, despite differences in CD3 binding. In contrast, PSMA01108 displays the lowest CD3 binding, and does not induce T cell proliferation to the level of PSMA01107 or PSMA01110 (FIG. 22B).

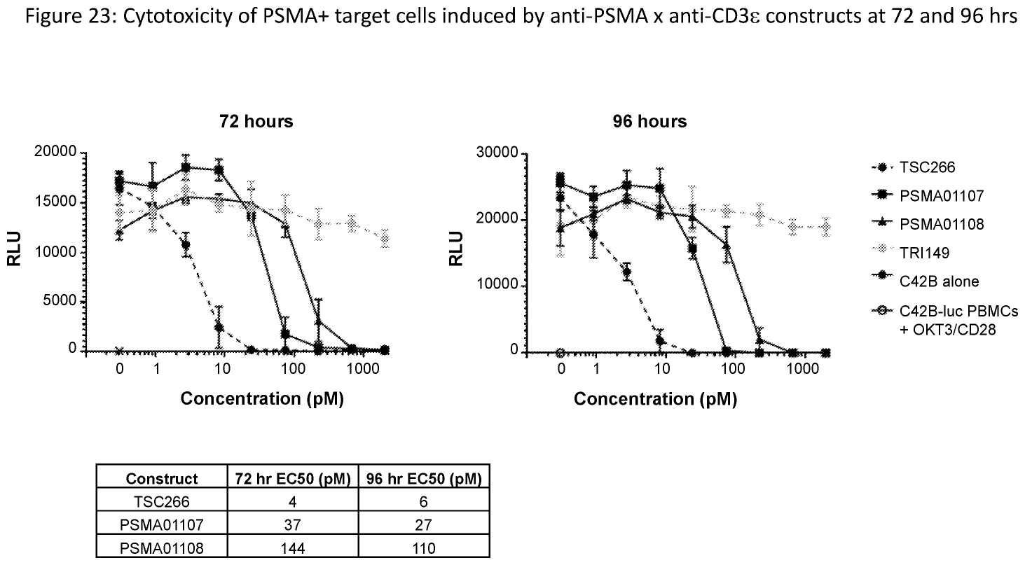

Cytotoxicity assays using C4-2B as target cells demonstrated a range of potencies (FIG. 23). Parent construct TSC266 showed the highest potency, whereas the PSMA01108 construct was the least potent, correlating with the potency in the T-cell activation and proliferation assays. Despite having low affinity binding to CD3, PSMA01108 was able to show robust antitumor activity over the dose range tested. The negative control ADAPTIR™ TRI149 showed no impact on the C4-2B cell growth.

In conclusion: 1) There is a correlation between CD3-binding and function, with lower CD3-binding potency resulting in lower T-cell activation, proliferation, cytokine secretion and target cytotoxicity potency; 2) In spite of the lower potency, all constructs (high or low CD3 affinity) induced significant levels of T-cell activation, proliferation, cytokine secretion and cytotoxicity.

Low affinity to CD3 would enable an anti-CD3×TA molecule to ignore peripheral T cells (where there is no TA expression), and preferentially accumulate at TA (+) tumor sites, where it can induce T-cell function and TA (+) cell cytotoxicity.

### Example 21: Binding of Anti-PSMA×Anti-CD3, Bispecific Protein to Various Cell Lines

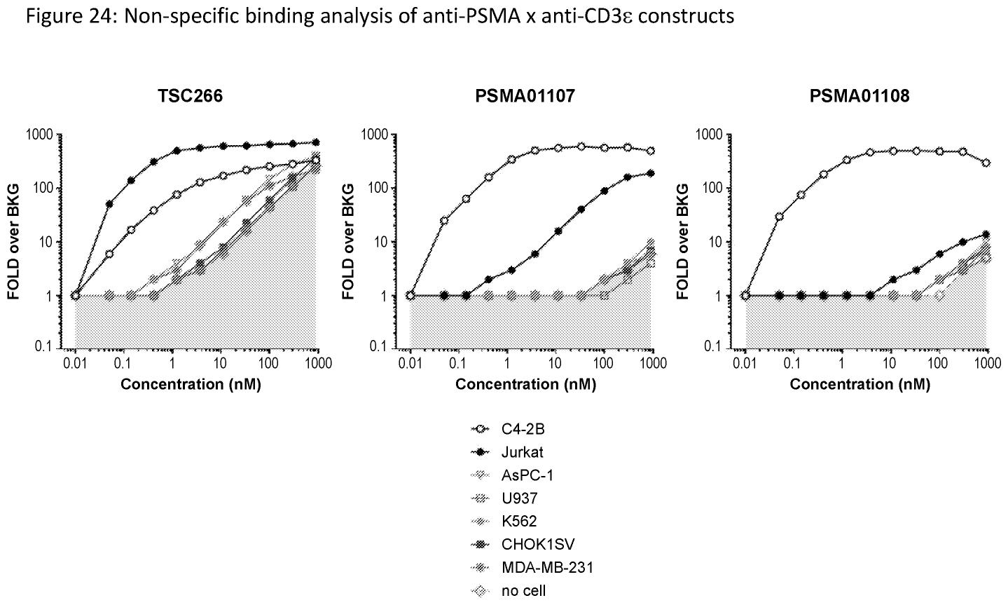

Cell binding studies were completed to demonstrate that the ADAPTIR™ scFv binding domains bound sufficiently to cells expressing PSMA or CD3 (C4-2B prostate cancer and Jurkat T cells, respectively), but not to cells without expression of PSMA or CD3 (AsPC-1, U937, K562, CHOK1SV and MDA-MB-231). Binding studies were performed using the sensitive Meso Scale Discovery assay platform. These data show that PSMA01107 and PSMA01108 have stronger PSMA binding to C4-2B, a prostate cancer cell line, than TSC266. In contrast, TSC266 has significantly stronger binding to CD3-expressing Jurkat cells than either PSMA01107 or PSMA01108. In addition, the PSMA01107 and PSM01108 proteins did not show any non-specific binding to five cell lines, not known to express PSMA or CD3, above the binding seen in the wells without target cells (FIG. 24). TSC266 had more non-specific binding to empty wells, as well as to the cell lines tested. Thus, there this no detriment to binding with these scFv or the change in the Fc.

Cells were washed and seeded at 50,000 cells/well in 1×HBSS on 96-well multi-array high bind plates (Meso Scale Discovery) and incubated at 37° C. for one hour. Following a blocking step in PBS buffer with 20% FBS, serial dilutions of binding constructs (from 0.05 to 900 nM) were added in PBS buffer with 10% FBS and incubated at room temperature for one hour. Plates were washed with PBS and the specific binding levels were detected using SULFO TAG-labeled goat anti-human IgG antibody (Meso Scale Discovery #R32AJ). Following a one-hour incubation and wash steps, 150 μL/well surfactant-free 1× Read Buffer T was added and samples were analyzed on MSD Sector Imager (Meso Scale Discovery). Resulting electrochemiluminescence (ECL) values were first divided by background of each cell line and then fold over background versus concentrations were plotted using GraphPad Prism 7® graphing software.

As FIG. 24 shows, non-specific binding of PSMA01107 and PSMA01108 had minimal binding to non-specific cell lines, whereas TSC266 bound to all cell lines tested at concentrations as low as 1 nM.

### Example 22. Incorporation of Binding Domains into Other Protein Formats, Characterization of Biophysical, Stability, Binding, and Activity

In addition to utilizing the anti-PSMA and anti-CD3-binding domains in the ADAPTIR™ scFv-Fc/Sc-Fc-scFv heterodimer format, they can also be incorporated into other protein structures that enable binding to PSMA and CD3 individually or simultaneously and can cause signaling via engaging both receptors. These other formats include but are not limited to those described by Spiess et al, Mol. Immun. 67: 95-106(2015). This also includes formats such as the RUBY™, Azymetric™ and TriTAC™ bispecific platforms. Generating alternative compositions of the anti-PSMA and anti-CD3-binding domains disclosed herein can be performed by using molecular biology techniques to amplify the genetic sequences encoding the variable heavy and/or variable light domains or the CDR regions of the anti-PSMA and anti-CD3-binding domains. These genetic fragments can then be spliced into the appropriate frameworks of the intended bispecific formats in a DNA plasmid appropriate for protein expression. Following expression, purification techniques can be employed to isolate the bispecific protein. These techniques could include affinity purification steps such as Protein A, Protein L, Protein G, anion exchange, cation exchange, or hydrophobic interaction chromatography. After protein purification, the molecules can be examined by biophysical techniques such as those described earlier, including differential scanning fluorimetry or differential scanning calorimetry. These alternative protein structures can also be assessed for solubility and resistance to aggregation by incubation in serum from different species, different salt concentrations, mechanical force, etc. The alternative protein formats can be assessed for binding to cells expressing one or both targets. Additionally, the alternative protein formats can be evaluated for biological activity by measuring the stimulation of cells expressing CD3. Stimulation, or activation of these cell populations can be measured, among other outputs, by determining the increase in concentration of interferon gamma or other cytokines, measuring the expression of other cell surface markers that are indicative of activation, such as CD25 or CD69. Following in vitro analysis, these formats can also be developed as therapeutics for the treatment of human diseases such as cancer.

### Example 23. Use of Optimized CD3-Binding Domain to Target Other Tumor-Associated Antigens

In addition to utilizing the optimized anti-CD3-binding domains described herein to treat PSMA (+) tumors, they can be used to generate additional therapeutic proteins to target other tumor associated antigens (TAAs). Cancerous cells expressing proteins such as Her2 (erbB-2) or B-Cell Maturation Antigen (BCMA) on the surface, for example, could be successfully treated with an anti-CD3 ADAPTIR™ bispecific protein to treat illness such as breast cancer and multiple myeloma, respectively.

Binding domains against other TAAs could be generated by immunizing rabbits, rodents, Llamas or other animals with DNA encoding the TAA of interest, with cells expressing the TAA on the surface, or recombinant versions of the TAA. Alternatively, binding domains could be isolated by panning libraries of binding domains, such as phage or yeast display libraries, to isolate sequences that bind specifically to the TAA of interest. After these binding domains have been identified, they could be further optimized to achieve the desired affinity, stability and biological activity when paired with the anti-CD3-binding used in constructs such as PSMA01107 or PSMA01108. The TAA-binding domains may also require humanization if they were derived from antibodies from the species that was immunized in order to reduce the risk of immunogenicity in humans.

The optimized anti-TAA sequences could be placed on the N-terminus of the Fc region in place of the anti-PSMA-binding domains used in the examples described above. Alternatively, different structural formats could be used to improve the activity or biophysical properties of the molecule. Alternative structures would include those described in the preceding example.

Bispecific proteins targeting CD3 and other TAAs could be assessed in vitro for their ability to induce T-cells to cause lysis of tumor cells or cell lines expressing the TAA on the surface. Other measures of T-cell activity could be measured, such as T-cell activation via upregulation of cell surface markers like CD69. Induction of T-cell proliferation is another way these therapeutic molecules could be assessed. In addition to these in vitro assessments, the ability of other anti-TAA×anti-CD3 bispecific proteins to cause tumor reduction could be measured using different animal models of disease, such as the mouse xenograft model described in the example above. The bispecific proteins can also be compared for their expression levels when produced by CHO cells, their stability, propensity to aggregate or degrade, or their shelf life when stored at different temperatures in order to select the construct with the best properties to advance into human clinical trials.

### Example 24: Use Modeling to Assess the Potential Biodistribution Differences Between Constructs

In addition to evaluating the distribution and pharmacokinetic properties of PSMA×CD3 bispecific proteins using studies in mice and nonhuman primates, it may be desirable to perform mathematical modeling to compare different therapeutic constructs. This could include Model Aided Drug Intervention (MADI) that has been developed by Applied Biomath. Modeling may provide data to help define starting dose, identify potential improvement in the therapeutic dosing window of one construct versus another based on binding affinity differences, as well as other useful information.

In the case of PSMA×CD3 bispecific proteins, the antigen PSMA is expected to be expressed on solid tumors. Conversely, the CD3 T-cell receptor is expressed on all circulating T-cells as well as resident T cells at the site of the tumor. Mathematical modeling may be able to provide data based on the relative expression of these two targets to help determine which bispecific candidate would likely have the most therapeutic benefit when evaluated in human clinical trials.

### Example 25: Differential Scanning Calorimetry (DSC) on Select PSMA×CD3 Bispecific Proteins

DSC was performed to determine the mid-point of the temperature-induced unfolding (Tm) of certain bispecific proteins using a MicroCal VP-Capillary DSC system (Malvern Instrument). Dulbecco's PBS (dPBS) was used as the buffer reference. 300 μL of a 1 mg/mL solution of each protein sample with buffer reference was loaded on the instrument and heated from 25° C. to 100° C. at a rate of one degree Celsius per minute. Melting curves were analyzed using Origin 7 platform software MicroCal VP-Capillary DSC Automated Analysis Software to derive the Tm values.

DSC thermograms of PSMA01107, PSMA01108, PSMA01110, and PSMA01116 consisted of a series of overlapping melting transitions. In order to determine the Tm values of individual domains, additional proteins were produced and tested that consisted of just the Fc region (hinge, CH2, CH3, with or without the Knob-in-Holes mutations), anti-PSMA-Fc or Fc-anti-CD3 (data not shown).

Both anti-PSMA and anti-CD3 domains were thermally stable and unfolded at ˜66 and ˜61 (° C.), respectively (Table 12). The same anti-PSMA domain is utilized in all three constructs and this domain has similar Tm values (66.4, 66.2 and 66.5) in each construct. Similarly, the same Fc region containing the KIH mutations to aid in heterodimer formation is used for all three bispecific constructs and yielded a transition at −71° C. that consists of both the CH2 and CH3 domains. The Knob-into-Holes mutations had a destabilizing effect on the CH3 domain such that the melting transition occurs near/on top of the CH2 transition. A single value for both domains are reported in the table below. There were slight differences observed in the Tm of the anti-CD3 domains. The Tm for the anti-CD3 domain in PSMA01108 did not yield a clear inflection in the thermogram to allow for assignment or fitting, as it appears to be significantly overlapping with the unfolding of the anti-PSMA scFv.

TABLE 12
Tm values determined by DSC

| | Anti-PSMA | Anti-CD3 | CH2, CH3 |
|---|---|---|---|
| Construct ID | Tm (° C.) | Tm (° C.) | Tm (° C.) |
| PSMA01107 | 66.4 | 62.6 | 71.5 |
| PSMA01108 | 66.2 | Not determined | 71.2 |
| PSMA01110 | 66.5 | 61.2 | 71.5 |
| PSMA01116 | 66.3 | 61.7 | 71.8 |

### Example 26: Pharmacokinetics in Mice

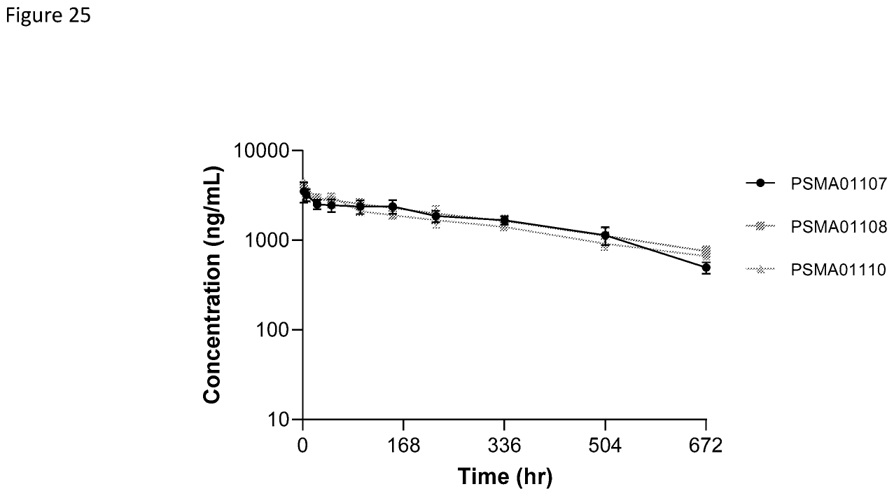

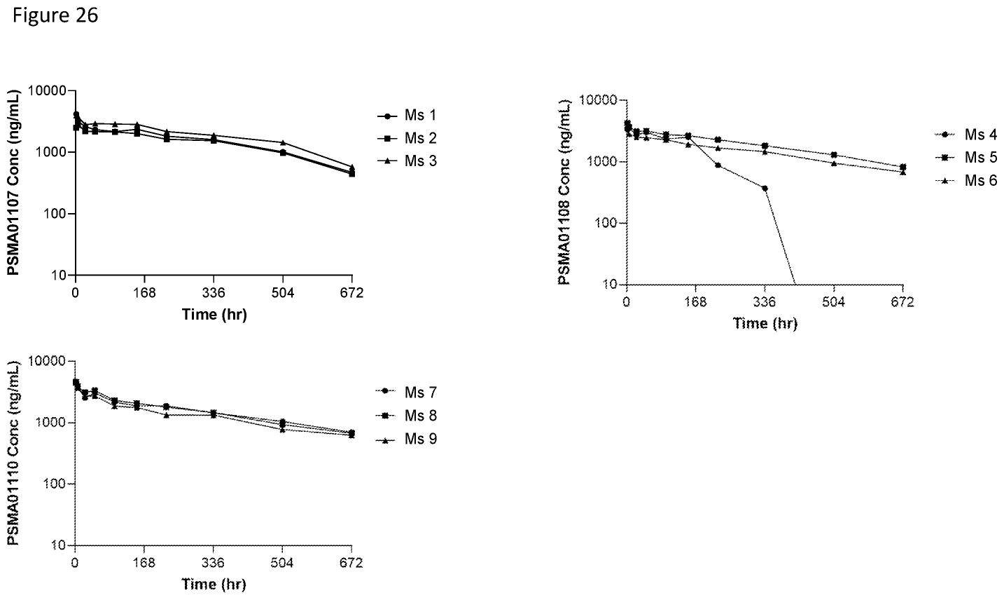

Pharmacokinetics were evaluated in C57BL/6 mice injected intravenously (IV) at time 0 with a single dose of 10 μg of PSMA01107, PSMA01108, or PSMA01110. Three mice were injected per group, and samples were collected by tail vein bleed at ten time points per animal (2, 6, 24, 48, 96, 150, 222, 336, 504, and 672 hours) via a serial sampling protocol. Concentrations of PSMA×CD3 bispecifics in samples were determined with a semi-specific ECLA method, using anti-PSMA binding domain monoclonal antibody (5B1 mAb) to capture the anti-PSMA BD, and a goat anti-human IgG polyclonal antibody (SouthemBiotech, cat #2049-08) conjugated to biotin to detect the Fc region of the bispecifics. A streptavidin-SULFOTAG reagent (SST, MSD cat #R32AD-1) was added to facilitate an electrochemiluminescent response. Mean systemic concentrations for the constructs are shown in FIG. 25 and individual timepoint data are shown in FIG. 26. Estimated PK parameters from non-compartmental analysis (NCA) using Phoenix WinNonlin™ (v8.1) license are listed in Table 13.

TABLE 13
NCA Parameters for anti-PSMA × anti-CD3 Constructs

| | | Cmax | Tmax | Tlast | HL | HL | AUClast | AUCinf | AUC % | Cl | Vss |
|---|---|---|---|---|---|---|---|---|---|---|---|
| Mouse | Construct | (μg/mL) | (hr) | (hr) | (hr) | (days) | (hr*μg/mL) | (hr*μg/mL) | extrap | (mL/hr/kg) | (mL/kg) |
| 1 | PSMA01107 | 4.16 | 2 | 672 | 190.0 | 7.9 | 1051.4 | 1180.3 | 10.9 | 0.424 | 138.3 |
| 2 | PSMA01107 | 2.78 | 6 | 672 | 186.5 | 7.8 | 966.1 | 1084.4 | 10.9 | 0.461 | 152.7 |
| 3 | PSMA01107 | 3.92 | 2 | 672 | 294.4 | 12.3 | 1298.7 | 1543.7 | 15.9 | 0.324 | 126.7 |
| Mean | | 3.62 | 3.3 | 672 | 223.6 | 9.3 | 1105.4 | 1269.5 | 12.6 | 0.403 | 139.2 |
| 4* | PSMA01108 | 3.54 | 6 | 336 | 69.2 | 2.9 | 600.6 | 637.8 | 5.8 | 0.784 | 102.8 |
| 5 | PSMA01108 | 4.26 | 2 | 672 | 293.5 | 12.2 | 1166.6 | 1493.7 | 21.9 | 0.335 | 143.5 |
| 6 | PSMA01108 | 3.95 | 6 | 672 | 345.6 | 14.4 | 1267.0 | 1686.4 | 24.9 | 0.296 | 140.8 |
| Mean | | 4.11 | 4 | 672 | 319.6 | 13.3 | 1216.8 | 1590.1 | 23.4 | 0.316 | 142.2 |
| 7 | PSMA01110 | 4.58 | 2 | 672 | 319.3 | 13.3 | 1067.8 | 1389.5 | 23.2 | 0.360 | 164.4 |
| 8 | PSMA01110 | 4.65 | 2 | 672 | 319.6 | 13.3 | 1080.2 | 1391.2 | 22.4 | 0.359 | 158.5 |
| 9 | PSMA01110 | 4.47 | 2 | 672 | 352.9 | 14.7 | 914.7 | 1233.3 | 25.8 | 0.405 | 197.2 |

| | | | | | | | | | | |
|---|---|---|---|---|---|---|---|---|---|---|
| Mean | 4.57 | 2 | 672 | 330.6 | 13.8 | 1020.9 | 1338.0 | 23.8 | 0.375 | 173.4 |

*not included in mean NCA parameter analysis due to impact by ADA

Cmax: Maximum observed concentration, occurring at Tmax; Tmax: Time of maximum observed concentration; Tlast: Time of last observed concentrations; HL: Apparent terminal elimination half-life; AUClast: Area under the curve from the time of dosing to the last detectable concentration; AUCINF: Area under the curve from the time of dosing extrapolated to infinity; AUC % extrap: the % of the AUCinf value that is extrapolated; CL: Serum clearance; Vss: An estimate of the volume of distribution at steady state.

A precompiled model for IV dosing was used during NCA, which resulted in an apparent mean terminal elimination half-life of approximately 223.6 hr (9.3 days) for PSMA1107, 319.6 hr (13.3 days) for PSMA1108 and 330.6 hr (13.8 days) for PSMA1110 (using best fit as determined by the software). Mean clearance and volume of distribution (at steady state) estimates for PSMA01107, PSMA01108 and PSMA01110 were approximately 0.403, 0.316 and 0.375 mL/kg/hr, and 139.2, 142.2 and 173.4 mL/kg, respectively. Overall, PK parameter values were similar for PSMA01107, PSMA01108 and PSMA01110.

To determine the presence anti-drug antibodies (ADA), serum samples collected from mice at pre-dose and 840 hours were analyzed using a sandwich ECLA format. Briefly, PSMA×CD3 constructs were coated on MSD 96-well plates followed by incubation with mouse serum samples to capture construct-specific ADA. A goat anti-mIgG antibody (SouthemBiotech, cat #1031-08) conjugated to biotin was used to detect ADA present in serum samples and a streptavidin-SULFOTAG reagent (SST, MSD cat #R32AD-1) was added to facilitate an electrochemiluminescent response. A mouse anti-human IgG Fc antibody (Jackson ImmunoResearch, cat #209-005-098) was used as a positive control. Response values and post-dose:pre-dose ratios for ADA results are listed in Table 14. For PSMA01108, there was a rapid decrease in exposure for one animal approximately 10 days after dosing. Post- to pre-dose ratios confirmed the presence of anti-PSMA01108 antibodies in one mouse (mouse #4), consistent with the observed decrease in serum concentrations. All other individual animals s were negative for anti-PSMA×CD3 construct antibodies. Based on this data, results from mouse #4 were excluded from mean concentration values as well as NCA parameter analysis.

TABLE 14
Individual ADA Results for anti-PSMA × anti-CD3 Constructs

| | | Pre-dose | | | Post-dose | | | |
|---|---|---|---|---|---|---|---|---|
| | | Mean | | | Mean | | | |
| | | Response | | | Response | | | Post:Pre |
| Construct | Mouse | Value | SD | % CV | Value | SD | % CV | Ratio |
| PSMA01107 | 1 | 225.7 | 6.0 | 2.7 | 221.3 | 10.7 | 4.8 | 0.98 |
| | 2 | 217.3 | 10.1 | 4.6 | 223.0 | 19.7 | 8.8 | 1.03 |
| | 3 | 188.0 | 4.6 | 2.4 | 216.7 | 29.0 | 13.4 | 1.15 |
| PSMA01108 | 4 | 162.0 | 5.3 | 3.3 | 1379.0 | 100.0 | 7.3 | 8.51 |
| | 5 | 170.3 | 3.2 | 1.9 | 167.7 | 22.0 | 13.1 | 0.98 |
| | 6 | 215.0 | 14.9 | 6.9 | 217.7 | 22.6 | 10.4 | 1.01 |
| PSMA01110 | 7 | 123.7 | 3.5 | 2.8 | 140.3 | 7.4 | 5.3 | 1.13 |
| | 8 | 139.3 | 4.7 | 3.4 | 169.0 | 9.9 | 5.9 | 1.21 |
| | 9 | 126.3 | 6.4 | 5.1 | 162.0 | 8.7 | 5.4 | 1.28 |

SD: Standard Deviation
% CV: Coefficient of Variation
Bold indicates Post:Pre-dose ratio above ration cut point (RCP = 2)

### Example 27: SPR Evaluation of Binding to Human and Cyno Murine Fc-Tagged PSMA ECD

SPR studies were performed on a subset of bispecific constructs binding to Human and cynomolgus primate PSMA ectodomain (ECD) fused to the c-terminus of a murine IgG1 Fc region. These experiments were conducted at 25° C. in dPBS (Gibco, 14040-133) with 0.2% BSA buffer on a Biacore T200 system. Bispecific constructs were immobilized at a density of ˜2,000-4,000 response units (RU) onto individual flow cells of a CM5 research-grade sensor chip (GE) by standard amine coupling chemistry, leaving one flow cell surface unmodified as the reference. Using a multi-cycle kinetics mode, a buffer blank and four different concentrations of the PSMA dimer ranging from 1 nM to 81 nM in dPBS with 0.2% BSA were sequentially injected through each flow cell at 30 μL/min for 300 seconds followed by a 600 second dissociation phase. Regeneration was achieved by injection of 10 mM glycine pH 2.0 at a flow rate of 30 μL/min for 40 seconds followed by dPBS with 0.2% BSA buffer stabilization for 1 min.

Sensorgrams obtained from kinetic SPR measurements were analyzed by the double subtraction method. The signal from the reference flow cell was subtracted from the analyte binding response obtained from flow cells with captured ligands. The buffer blank response was then subtracted from analyte binding responses and the final double-referenced data were analyzed with Biacore T200 Evaluation software (2.0, GE), globally fitting data to derive kinetic parameters. All sensorgrams were fitted using a simple one-to-one binding model.

For each construct, measured binding affinities to human and cyno PSMA ECD were near equivalent and fell within the range of 2 to 5 nM. Binding affinity to human PSMA was not impacted by the purification tag.

TABLE 15
Biacore affinity measurements of select anti-PSMA × anti-
CD3 bispecifics to murine Fc-tagged human and cyno PSMA ECD.

| | Construct | | |
|---|---|---|---|
| | Affinity to human | Affinity to | Affinty to |
| | PSMA dimer ECD | mFc-Hu | mFc-Cyno |
| | 10X His (TSC033) | PSMA ECD | PSMA ECD |
| Name | KD (nM) | KD (nM) | KD (nM) |
| PSMA01107 | 2.6 | 3.0 | 4.4 |
| PSMA01116 | 5.2 | 4.1 | 6.2 |

### Example 28: Expression of Human PSMA on Primary Tumor Cell Lines

Human PSMA tumor cell lines were used for binding and functional characterization of PSMA constructs. The following cell lines were used: 22RV1, human prostate carcinoma cell line (ATCC), C4-2B, androgen-independent human prostate cancer line (Wu et al., 1994 Int. J. Cancer 57:406-12; obtained from MD Anderson Cancer Center (Houston, TX), LNCaP, human prostate carcinoma cell line (ATCC), MDA-PCa-2b, human prostate carcinoma cell line (ATCC) and DU-145, human prostate carcinoma cell line (ATCC). The levels of surface PSMA expression on these cells were determined by flow cytometry.

Cells were plated at approximately 100,000 cells per well, in 96 well-U bottom plates, and incubated at 4° C. with a saturating concentration of PE-conjugated antibodies: anti-PSMA antibody (LNI-17 clone, Biolegend #342504) or isotype control (MOPC-21 clone, mouse IgG isotype, Biolegend #400140). Following a one-hour incubation, cells were washed and analyzed by flow cytometry. All incubations and washes were done in staining buffer (PBS buffer with 0.2% BSA and 2 mM EDTA). Samples were collected using an BD™ LSR-II flow cytometer (BD Biosciences) and analyzed by FlowJo flow cytometry analysis software. Mean fluorescence intensity (MFI) was determined after exclusion of doublets. Quantibrite™ beads (BD Bioscience #340495) were used to determine receptor numbers as described by the manufacturer.

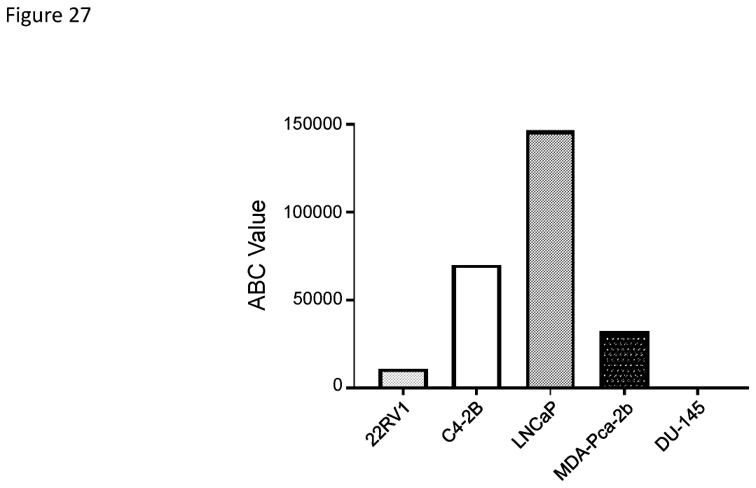

FIG. 27 shows the levels of expression of human PSMA in 22RV1, C4-2B, LNCaP, MDA-PCa-2b and DU-145 cells. The graph shows receptor levels in units of antibody bound per cell (ABC). On average, 22RV1 cells express around 10,000 receptors/cell, MDA-PCa-2b cells express over 30,000 PSMA receptors/cell, C4-2B cells express over 70,000 PSMA receptors/cell, LNCaP cells express over 140,000 PSMA receptors/cell, and DU145 cells express less than 20 receptors/cell. Therefore, LNCaP and C4-2B cells both are considered PSMA (high) cells, MDA-PCa-2b and 22RV1 cells are considered PSMA (low), and DU-145 cells are considered PSMA (negative) cells.

### Example 29: In Vitro Analysis of Anti-PSMA×Anti-CD3F Constructs on Differentially PSMA-Expressing Tumor Cell Lines

Anti-PSMA×anti-CD3ε constructs (TSC266, PSMA01107, PSMA01108 and PSMA01110) were examined for the correlation of binding affinity using cell lines expressing various levels of surface human PSMA.

The tumor cell lines were labelled at approximately 100,000 cells per well, in 96-well plates, with serial dilutions of bispecific constructs ranging in concentration from of 0.1 to 300 nM. PE-labelled secondary antibody was used for detection and cells were collected using flow cytometry as described previously.

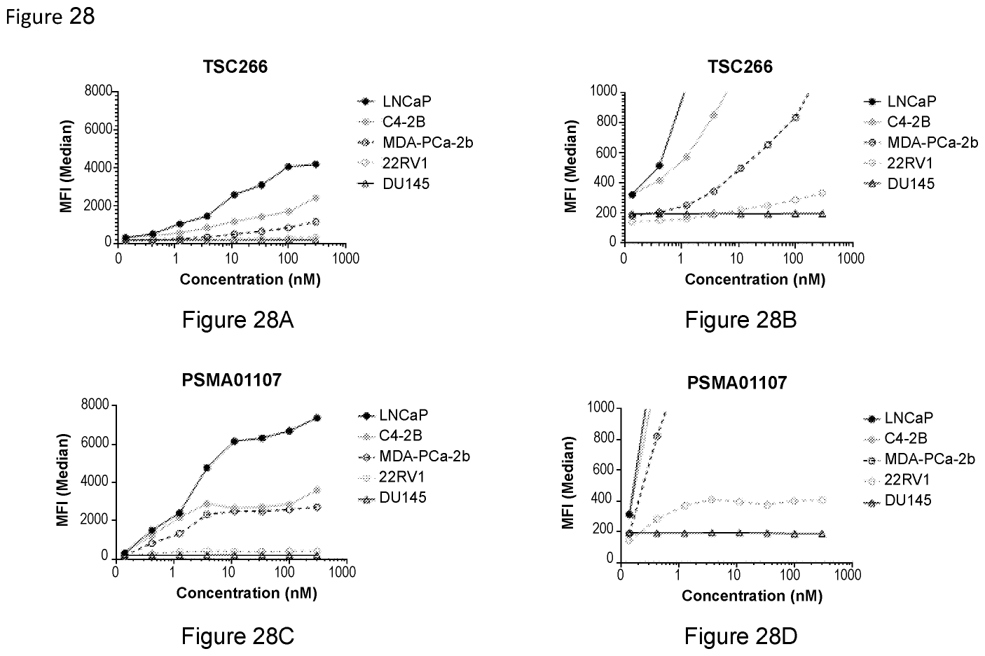

FIG. 28 shows the binding curves of the anti-PSMA×anti-CD3ε constructs on various PSMA-expressing tumor cells. In FIG. 28A, the relative binding of a previously disclosed construct, TSC266 corresponds to the level of PSMA expression for each cell line tested. High PSMA-expressing LNCaP cells have the highest maximum MFI values while low PSMA expressing cell line 22RV1 has a very low maximum MFI value. PSMA negative cell line DU145 exhibits no binding to TSC266. A similar pattern is observed with PSMA01107 (FIG. 28C), in that, the relative binding of the anti-CD3 monovalent construct corresponds to the level of PSMA expression for each cell line tested. However, maximal MFI values measured with the LNCaP, C4-2B and MDA-PCa-2b cells compared to those of the TSC266 construct due to incorporation of a higher affinity anti-PSMA binding domain. Constructs PMSA01108 and PSMA01110 with the same anti-PSMA binding domain as PSMA01107 gave indistinguishable results (data not shown).

Next, the anti-PSMA×CD3ε constructs were evaluated to determine whether they were capable of inducing target-dependent T-cell activation when used with tumor cell lines expressing different levels of PSMA.

For activation assays, PBMC were plated with tumor cells to achieve approximate T-cell to tumor cell ratios of 3:1. Serial dilutions of test molecules at concentrations ranging from 0.02 to 2,000 μM were added to the co-culture. After 24 hours, cells were labeled for flow cytometric analysis as previously described.

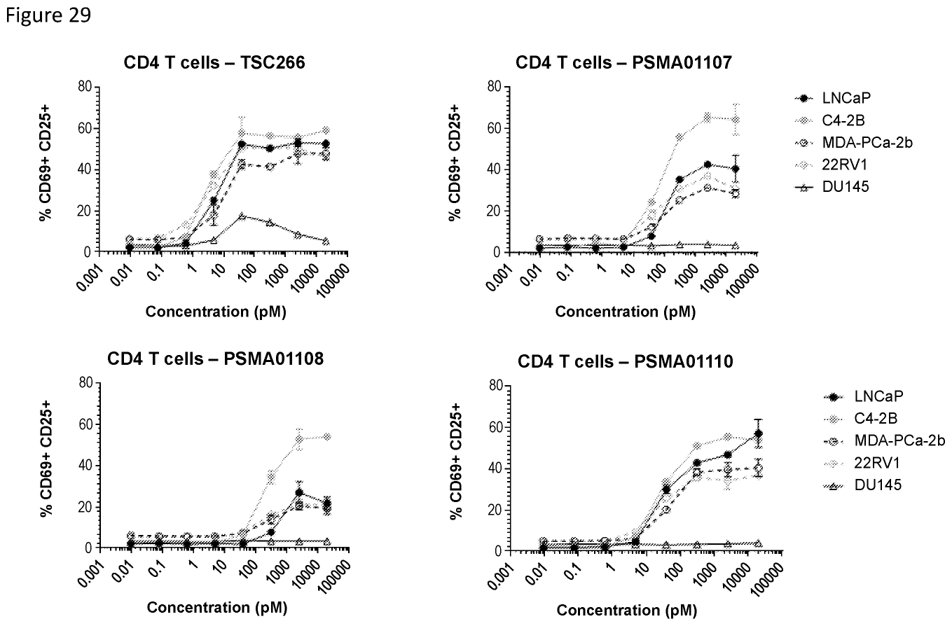

CD4⁺ T-cell activation induced by the anti-PSMA×anti-CD3 constructs was assessed by the upregulation of CD69 and CD25. In the presence of various PSMA-expressing tumor target cells (FIG. 29), all constructs induced robust CD4⁺ T-cell activation with a range of potencies even with low PSMA-expressing tumor cells. Unexpectedly, the level of CD4⁺ T-cell activation did not directly correlate with the level of PSMA expression. In general, TSC266 stimulated strong CD4⁺ T-cell activation with all PSMA cell lines. We also observed a low level of CD4⁺ T-cell activation with the PSMA negative cell line DU-145. In contrast, we saw more separation of CD4⁺ T-cell activation levels with PSMA01107, PSMA01108 and PSMA01110. The high PSMA-expressing cell line C4-2B stimulated the strongest CD4+ T-cell activation with constructs PSMA01107, PSMA01108 and PSMA01110, followed by LNCaP, 22RV1 and MDA-PCa-2b target cells, respectively. Notably, co-cultures containing the PSMA negative cell line DU-145, did not result in CD4⁺ T-cell activation with PSMA01107, PSMA01108 or PSMA01110.

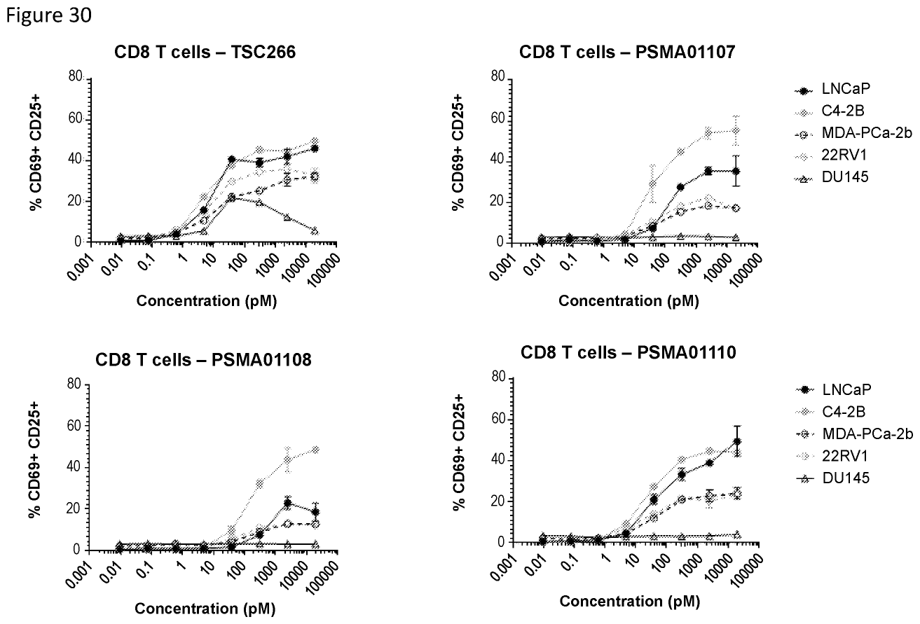

In the presence of various PSMA expressing target cells (FIG. 30), all constructs induced robust CD8⁺ T-cell activation with a diversity of potencies. Similar to CD4⁺ T cell activation, the level of CD8⁺ T-cell activation did not correlate directly with the expression level of PSMA on the target cell lines. In general, TSC266 stimulated strong CD8⁺ T-cell activation with all tumor cell lines, despite the level of PSMA-expression, including the PSMA negative cell line DU-145. In contrast, CD8⁺ T-cell activation levels with the PSMA01107, PSMA01108 and PSMA01110 were tumor cell line-dependent. Co-cultures with the PSMA high expressing cell line C4-2B, resulted in the strongest CD8⁺ T-cell activation with constructs PSMA01107, PSMA01108 and PSMA01110, followed by LNCaP, 22RV1 and MDA-PCa-2b target cells, respectively. Importantly, cultures containing the PSMA negative cell line DU-145 and PSMA01107, PSMA01108 and PSMA01110 did not activate CD8⁺ T cells.

To assess target-cell cytotoxicity, the viability of C4-2B (PSMA high) and MDA-PCa-2b (PSMA low) target cells were measured following co-culture with PBMC and a dilution of bispecific constructs. C4-2B and MDA-PCa-2b cells were transduced to express firefly luciferase using RediFect™ Red-FLuc-Puromycin Lentiviral Particles (PerkinElmer). Cultures were assessed at 72 and 96 hours for luciferase expression by tumor cells as described previously.

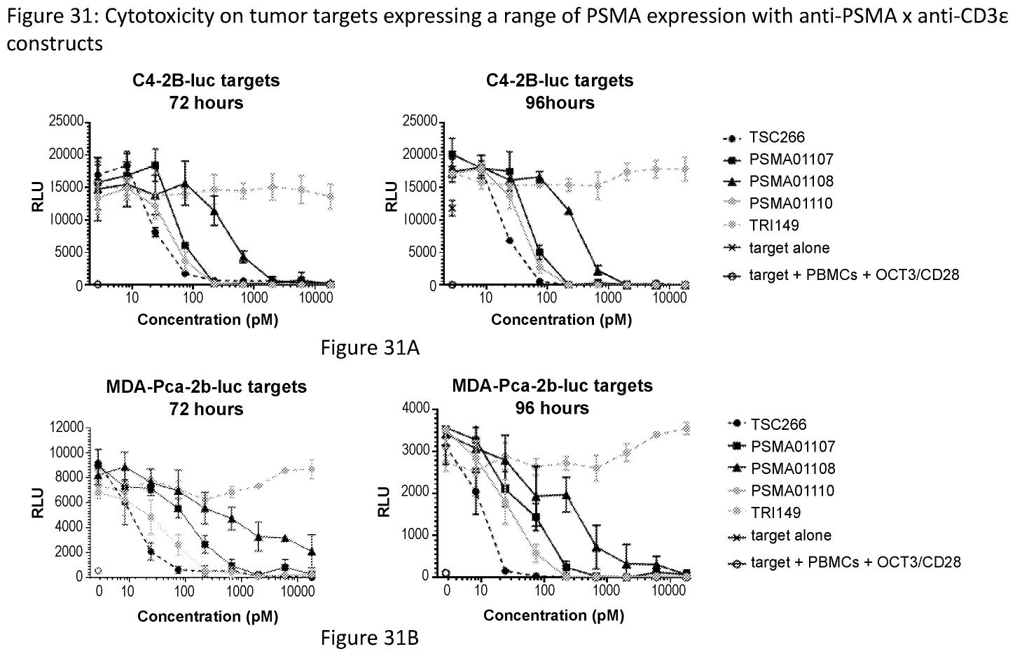

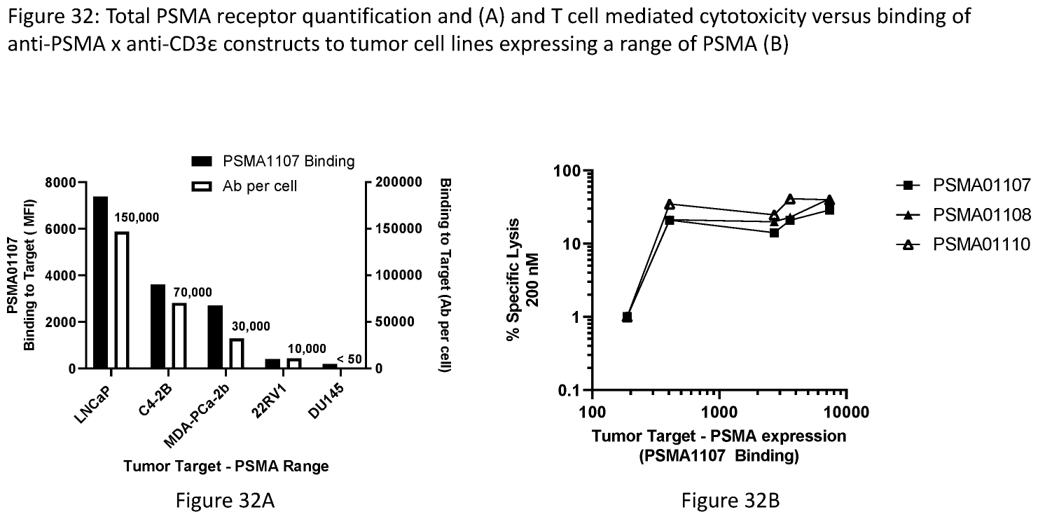

Cytotoxicity assays using C4-2B (PSMA high; FIG. 31A) and MDA-PCa-2b (PSMA low; FIG. 31B) target cells demonstrated that the valency and affinity of the anti-CD3 binding domain impacts the cytotoxic potential of PBMC. TSC266 showed the most robust T-cell redirected cytotoxicity, whereas the PSMA01108 construct was the least efficacious, correlating with the potency in the T-cell activation assays. Despite having low affinity binding to CD3, PSMA01108 was able to show significant antitumor activity over the dose range tested and achieved complete lysis of the PSMA high expressing cell line C4-2B by 72 hours (FIG. 31A). In contrast, on the PSMA low expressing cell line MDA-PCa-2b, PSMA01108 was unable to achieve total lysis until the 96 hour timepoint (FIG. 31B). The negative control ADAPTIR™ TRI149 did not promote T-cell lysis of C4-2B or MDA-PCa-2b tumor cells. Target expression was evaluated by both quantification of antibodies bound per cell and direct binding of the PSMA to the anti-PSMA×anti-CD3ε construct PSMA01107. Binding of PSMA01107 correlated with the amount of PSMA detected by antibody quantification, demonstrating the ability of PSMA01107 to bind tumor targets in a manner directly related to PSMA expression. (FIG. 32A). An evaluation of the cytotoxicity of PSMA01107 demonstrated that PSMA01107 induced specific lysis in a manner directly correlated to PSMA expression, and this response could occur at similar levels even when the tumor target expressed lower levels (FIG. 32B).

In conclusion the anti-PSMA×anti-CD3ε constructs: 1) are binding on differentially expressing PSMA tumor cell lines correlates with the level of PSMA expression; 2) are able to promote T-cell activation equivalently on low or high expressing PSMA tumor cell lines; and 3) because of a weaker (i.e., lower binding affinity) CD3 binding domain (PSMA01107 and PSMA01108) require longer incubation to achieve complete target cell lysis.

### Example 30: In Vitro Evaluation of the Sequence-Modified Anti-PSMA×Anti-CD3 Bispecific Construct, PSMA01116

We evaluated the sequence-modified anti-PSMA×anti-CD3ε construct PSMA01116 for its ability to bind, induce target-dependent T-cell activation, and mediate T-cell redirected tumor lysis as compared to the parental sequence construct PSMA01107.

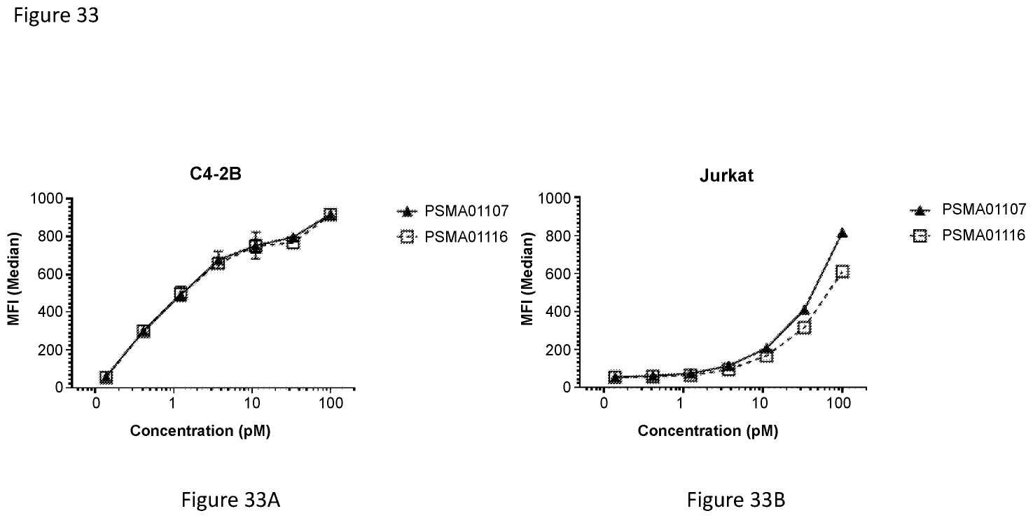

FIG. 33 shows the binding curves of the anti-PSMA×anti-CD3ε constructs on C4-2B and Jurkat cells. In FIG. 33A the relative binding of PSMA01107 and PSMA01116 on PSMA-expressing C4-2B cells are nearly indistinguishable. Similarly, binding of PSMA01107 and PSMA01116 on CD3-expressing Jurkat cells was comparable (FIG. 33B).

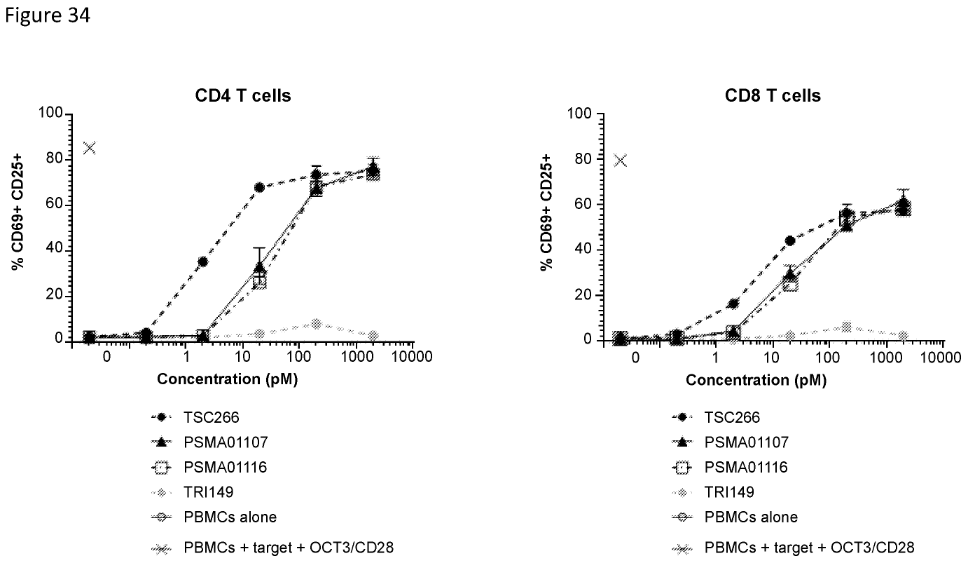

CD4⁺ and CD8⁺ T-cell activation induced by the anti-PSMA×anti-CD3 constructs were assessed at 24 hours, as defined by the upregulation of CD69 and CD25. In the presence of C4-2B target cells (FIG. 34), all constructs induced robust T-cell activation with a range of potencies. TSC266 showed the highest potency (lowest EC50), whereas the PSMA01107 and PSMA01116 constructs showed equivalent, but reduced, potency in comparison.

In conclusion: The sequence modifications made to PSMA01107 (PSMA01116) did not impact the binding to PSMA- or CD3-expressing cells nor the T-cell agonist activity.

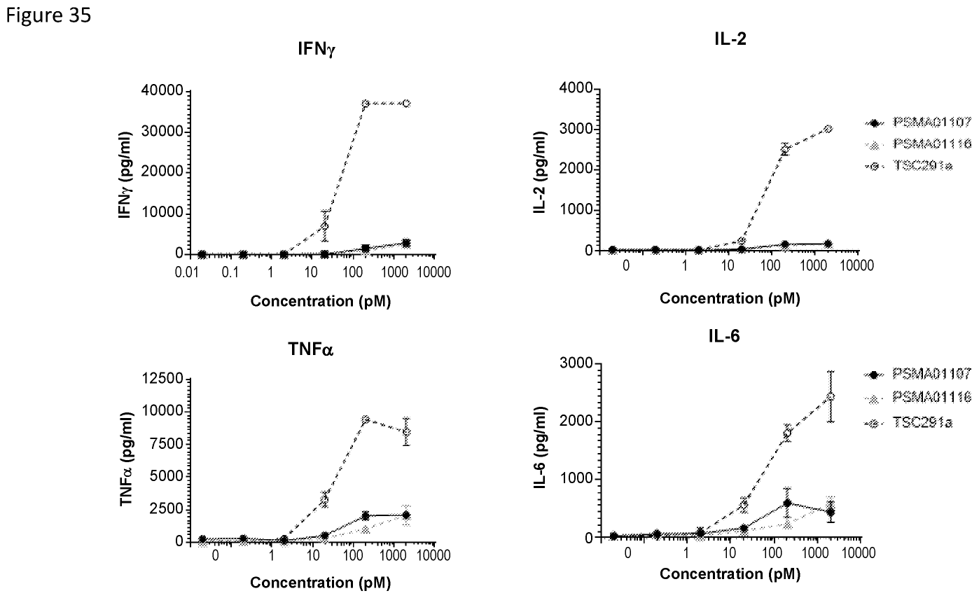

Next, the level of cytokine secretion in the culture supernatant from the T-cell activation assay (described above) was quantified. As expected, TSC291a induced T cells to secrete abundant IFN-γ, IL-2, TNF-α and IL-6 at significantly higher levels than PSMA01107 or PSMA01116 (FIG. 35). Similar to the level of T-cell activation, both PSMA01107 and PSM01116 mediated indistinguishable cytokine levels.

### Example 31. Cytotoxicity of PSMA01107 Compared to PSMA01116

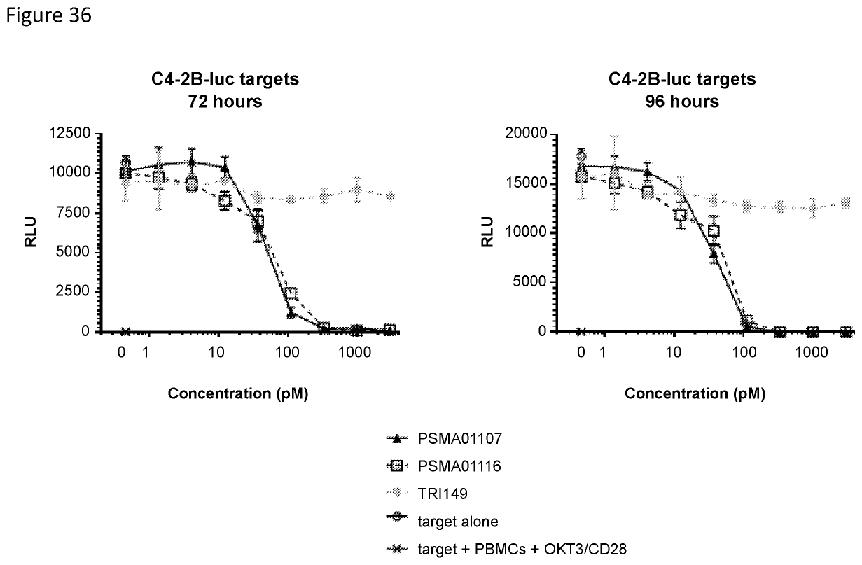

In T-cell redirected cytotoxicity assays using C4-2B (PSMA high) target cells, the sequence changes in PSMA01116 did not impact the induction of cytotoxic potential on PBMCs. As expected, both PSMA01107 and PSMA01116 promoted equivalent tumor lysis, correlating with the potency in the T-cell activation and cytokine secretion assays. Despite having lower affinity binding to CD3, PSMA01107 and PSMA01116 were both able to induce significant anti-tumor activity over the dose range tested and achieved complete tumor lysis by 72 hours (FIG. 36). The TRI149 control did not impact C4-2B tumor cells' growth.

### Example 32: Anti-Tumor Efficacy in Response to Optimized Anti-PSMA×Anti-CD3, Bispecific Protein Treatment

The function and potency of the optimized anti-PSMA×CD3R constructs were assessed in vivo in a prophylactic xenograft tumor model using human effector T cells.

As described previously, NOD/scid mice were challenged with 2×10⁶ C4-2B-luc cancer cells mixed with 1×10⁶ human leukopak T cells delivered subcutaneously on their flank. Two hours later, mice were administered intravenously with either vehicle (PBS), PSMA01110, PSMA01107 or PSMA01108 at dosages of 100, 30, 3 or 0.3 μg/mouse (n=8/group) on days 0, 4 and 8. Tumor growth was monitored by BLI two times/week. Tumor growth was monitored by bioluminescent imaging (BLI) using an IVIS® Spectrum imager (PerkinElmer). Caliper measurements, tumor bioluminescence calculations, and statistical analyses were done as previously described.

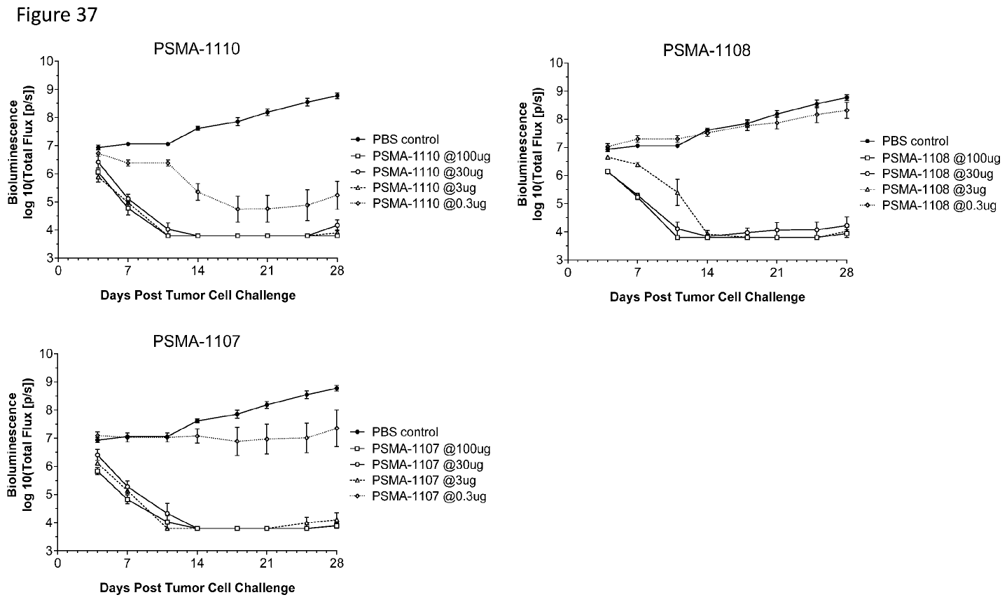

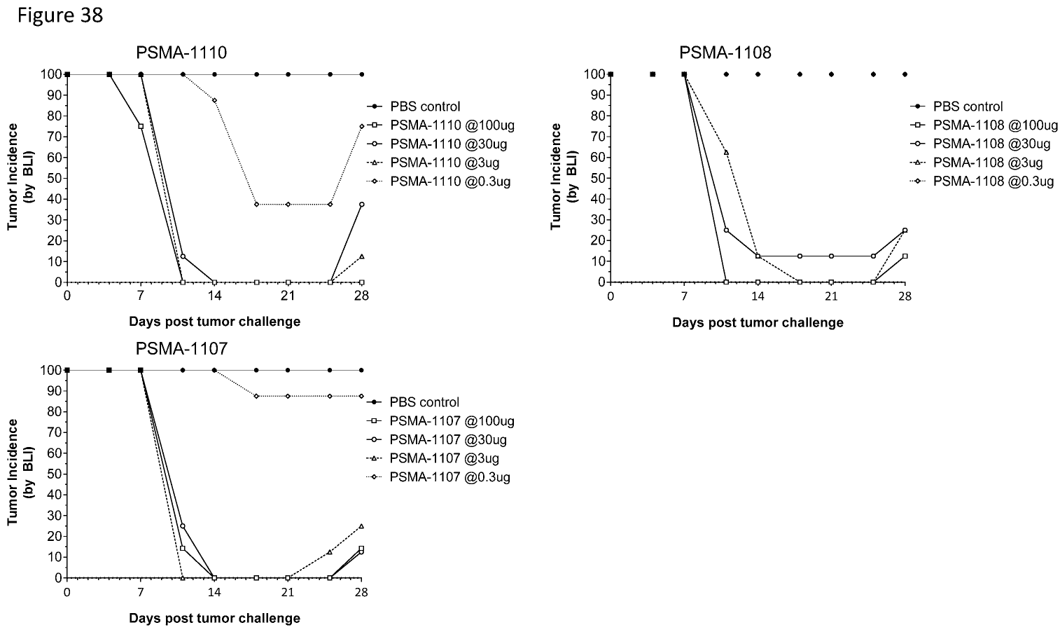

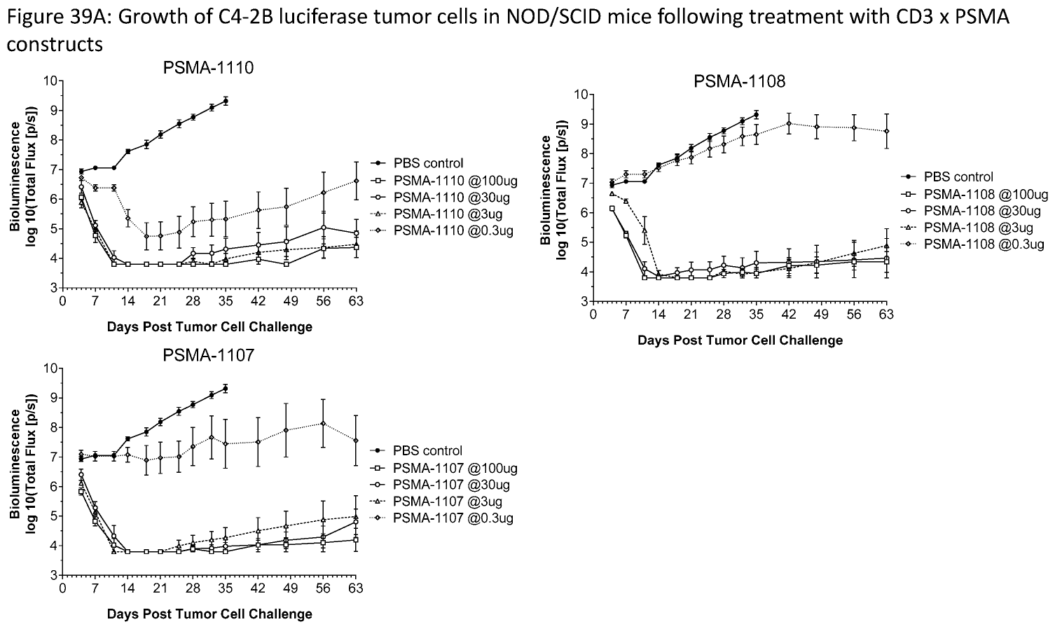

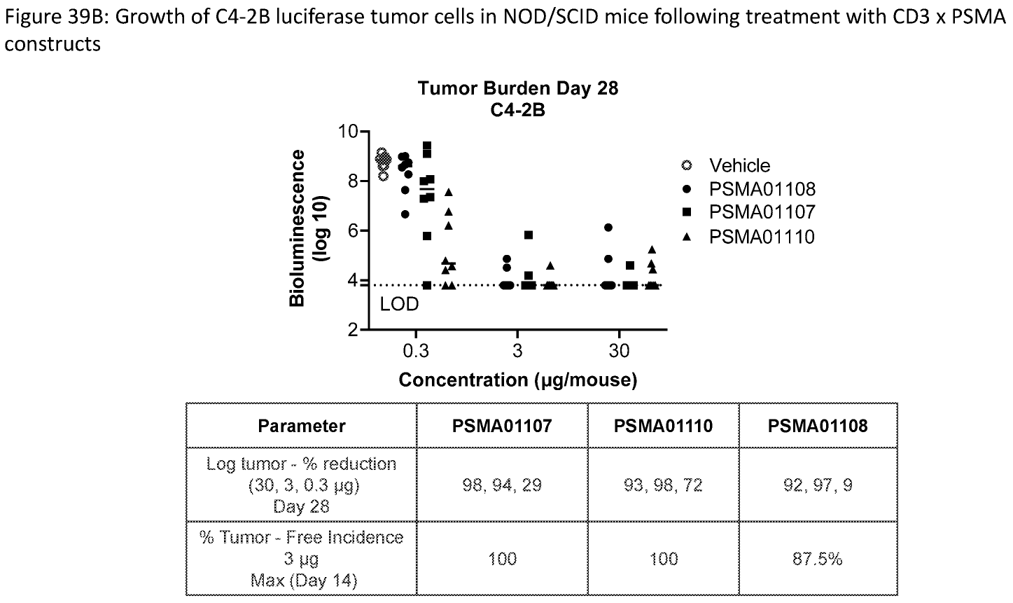

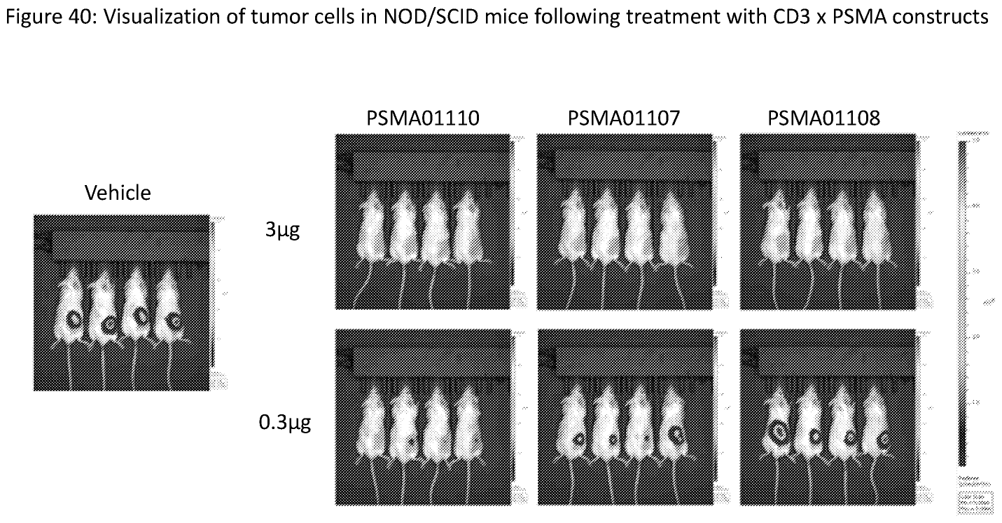

Treatment with all optimized anti-PSMA×anti-CD3ε ADAPTIR™ molecules resulted in a statistically significant reduction of C4-2B-luc tumor growth as determined by bioluminescence in NOD/scid mice (FIGS. 37 and 39; and Table 16). The reduction in tumor bioluminescence was observed at the first imaging time point on day 4 after receiving only a single injection. Further reduction in tumor bioluminescence was observed over the course of the treatment. All constructs resulted in significant reduction of tumor bioluminescent signal at dosages of 3 mg/mouse and above. Only PSMA01110 treatment at the lowest dose of 0.3 mg/mouse resulted in significant tumor bioluminescent signal reduction as observed by bioluminescent imaging at day 14 (FIG. 40). Overall, treatment with all optimized PSMA×CD3 constructs resulted in a statistically significant reduction in tumor volume and prevented the outgrowth of tumors in C4-2B-luc challenged mice. (FIGS. 37 and 38; Table 16). The prevention of tumors by PSMA01107, PSMA01108, and PSMA01110 remained present in all groups up to the termination of the study at day 63. (FIG. 39A). Log % depletion and percent of tumor free incidence were calculated at days 28 and 14, respectively (FIG. 39B). Here, PSMA01107 showed comparable activity to PSMA01110, while PSMA01108 showed overall lower anti-tumor responses (FIG. 39B).

Differences in mean tumor bioluminescence from day 4 through day 25 for the study groups were determined using JMP repeated measures analysis with Tukey multiple comparison test. Values of p<0.05 were considered significant.

TABLE 16
Statistical Comparison of Mean log10 Tumor Bioluminescence
through Day 25
JMP One-way ANOVA Analysis with Tukey-Kramer HSD Method

| Treatment | p-Value |
|---|---|
| PBS Control vs. PSMA01110 100 μg | <0.0001 |
| PBS Control vs. PSMA01110 30 μg | <0.0001 |
| PBS Control vs. PSMA01110 3 μg | <0.0001 |
| PBS Control vs. PSMA01110 0.3 μg | <0.0001 |
| PBS Control vs. PSMA01107 100 μg | <0.0001 |
| PBS Control vs. PSMA01107 30 μg | <0.0001 |
| PBS Control vs. PSMA01107 3 μg | <0.0001 |
| PBS Control vs. PSMA01107 0.3 μg | 0.0534 |
| PBS Control vs. PSMA01108 100 μg | <0.0001 |
| PBS Control vs. PSMA01108 30 μg | <0.0001 |
| PBS Control vs. PSMA01108 3 μg | <0.0001 |
| PBS Control vs. PSMA01108 0.3 μg | 1.000 |

### Example 33: Use of the Technology to Target More than One Tumor-Associated Antigen (TAAs)

In addition to the use of the technology to target PSMA expressing tumors, proteins could be generated that contain one binding domain to CD3, and one or more binding domains to two different TAAs. This would enable a protein therapeutic to target two different tumor antigens on the same tumor type, or possible use the same drug to target two different types of tumor. The affinity of the binding domain to each TAA could be adjusted to enable higher selectivity and specificity, significantly lowering the risk of off-tissue activity. This would allow drugs to overcome low level expression on normal healthy tissues by requiring both TAAs to be present on the tumor cell for the drug to be able to signal and activate T cells.

### Example 34: Differential Effects of Anti-CD3F Binding on NFκB, NFAT and ERK Downstream Signaling Pathways

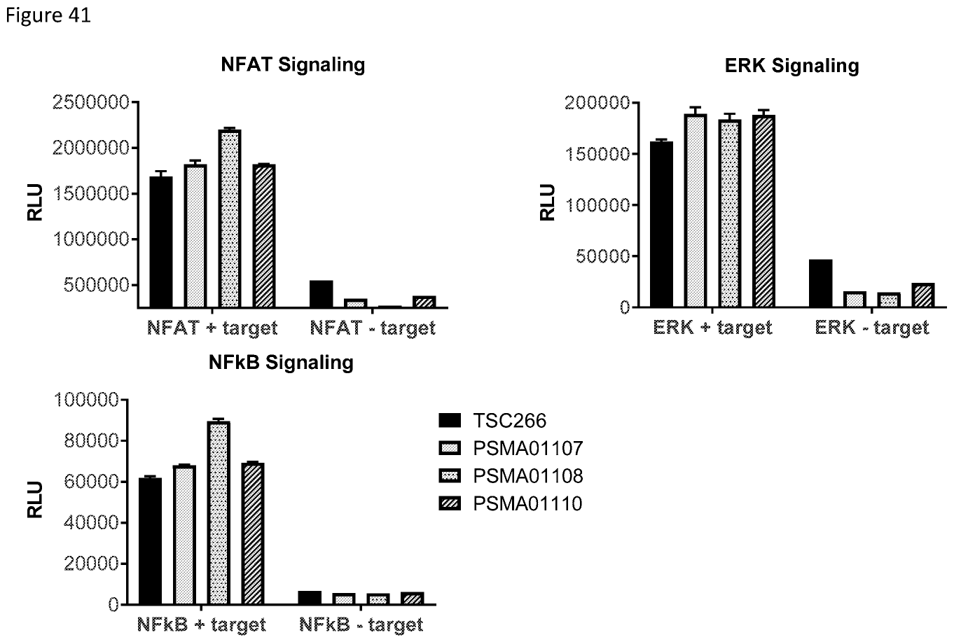

Reporter assays were utilized to assess the strength and duration of downstream CD3 signaling pathways via Nuclear Factor K-light-chain-enhancer of activated B cells (NFκB), Nuclear Factor of Activated T-cells (NFAT), and Extracellular-signal-Regulated Kinase (ERK) following anti-CD3ε stimulation by anti-PSMA×anti-CD3ε ADAPTIR™ constructs. C4-2B target cells were treated with 20 nM of TSC266, PSMA01107, PSMA01108, and PSMA01110. After 24 hours, strong downstream signaling was measured in NFκB, NFAT, and ERK with all constructs in the presence of C4-2B target cells expressing PSMA (FIG. 41). To demonstrate the requirement for PSMA crosslinking of anti-PSMA×anti-CD3ε ADAPTIR™ constructs, the assay was run in the absence of C4-2B target cells. There was a significant reduction in activity without PSMA crosslinking when directly compared to PSMA-crosslinked ADAPTIR™ (FIG. 41). The unoptimized parent construct, TSC266, had higher background signaling when treated in the absence of C4-2B cells as compared to optimized anti-CD3 binding domains in PSMA01107, PSMA01108 or PSMA01110 constructs.

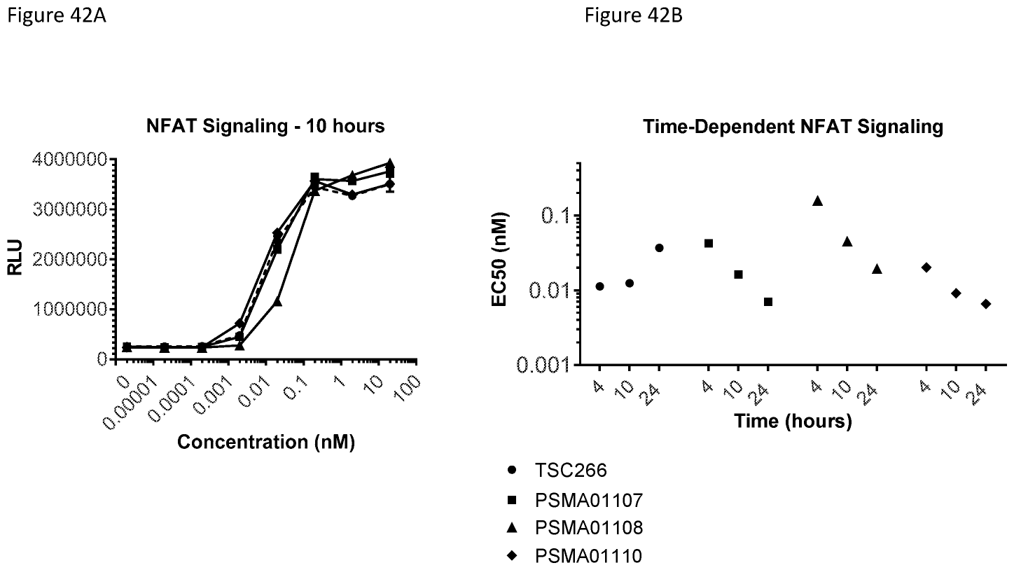

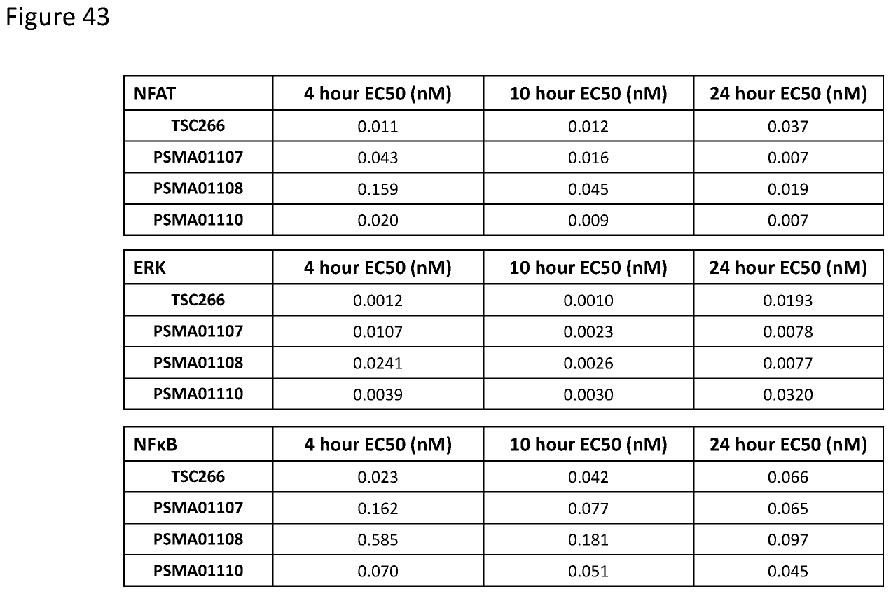

The NFAT reporter assay was performed at 4, 10, and 24 hours to determine if the EC50 values of PSMA01107, PSMA01108, and PSMA01110 constructs were dependent on when CD3 was signaling. The downstream NFAT activity is dependent on the concentration of ADAPTIR™ (FIG. 42A) and the avidity of anti-CD3 binding (FIG. 42B), as PSMA01108 has a reduced potency with slightly higher EC50 at all timepoints. TSC266 has a lower EC50 (stronger potency) at 4 hours but overall lower potency with an increase in EC50 by 24 hours. In contrast, PSMA01107, PSMA1108, and PSMA01110 have slightly higher EC50s at 4 hours, but continue to decrease through the 24 hours time point, demonstrating that the downstream CD3 signaling is sustained for longer time than with TSC266. FIG. 43 provides the EC50 values for TSC266, PSMA01107, PSMA01108, and PSMA01110 constructs at 4, 10, and 24 hours for NFκB, NFAT, and ERK. FIG. 43 demonstrates that PSMA01107, PSMA01108, and PSMA01110 constructs have decreased EC50s from 4 to 24 hours for NFκB, NFAT, and ERK as compared to TSC266.

### Example 35: Effect of CD3 Affinity on T Cell Memory Phenotype

The anti-PSMA×anti-CD3ε constructs (PSMA01107 and PSMA01110) were evaluated to determine their impact on the memory phenotype of CD8⁺ T cells. For phenotyping assays, PBMC were plated with C4-2B tumor cells to achieve approximate T-cell to tumor cell ratios of 3:1. Serial dilutions of test molecules at concentrations ranging from 0.02 to 2,000 μM were added to the co-culture. After 72 hours, cells were labeled for flow cytometric analysis as previously described.

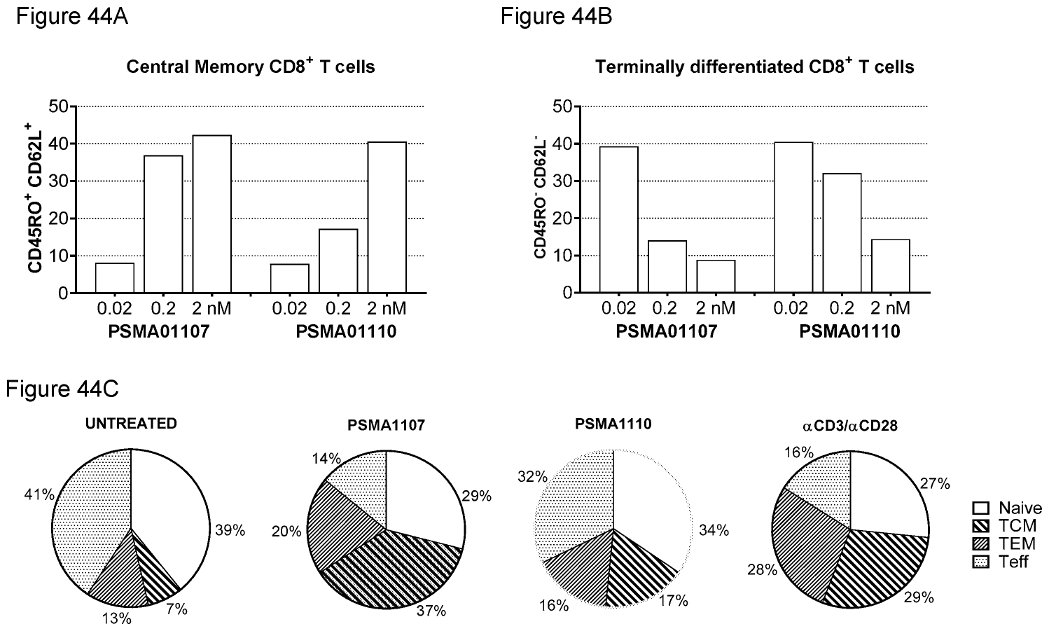

Development of the CD8⁺ T cell memory phenotype was influenced by the anti-PSMA×anti-CD3ε constructs and was assessed by the surface expression of CD45RO and CD62L. In the presence of PSMA-expressing tumor target cells, all constructs induced a dose-dependent change in the memory phenotype of CD8⁺ T cells that was inversely correlated between the number of central memory cells (FIG. 44A) and the number of terminally differentiated cells (FIG. 44B). Constructs given at 0.2 nM induced the greatest difference in ratio of naïve, central memory, effector memory, and terminally differentiated cells between PSMA01107 and PSMA01110 (FIG. 44C). These results suggest that the lower CD3 affinity designed into the ADAPTIR™ can alter the memory phenotype and result in a population of CD8⁺ T cells that may be potent tumor killers that maintain a long-lived memory response.

The disclosure is not to be limited in scope by the specific aspects described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other aspects are within the following claims.

SEQUENCES

| | | | DNA | | AA |
|---|---|---|---|---|---|
| Protein | | | SEQ ID | | SEQ ID |
| Name | DNA Sequence | | NO: | AA Sequence | NO: |
| Avi-His- | catcatcaccatcatcatcatcatcaccatggtct | | 1 | HHHHHHHHHHGLNDIFEAQ | 2 |
| Human | gaatgacatcttcgaggctcagaaaatcgaatggc | | | KIEWHEHKSSNEATNITPKH | |
| PSMA | acgaacataaatcctccaatgaagctactaacatt | | | NMKAFLDELKAENIKKFLYNF | |
| ECD | actccaaagcataatatgaaagcatttttggatga | | | TQIPHLAGTEQNFQLAKQIQ | |
| | attgaaagctgagaacatcaagaagttcttatata | | | SQWKEFGLDSVELAHYDVLL | |
| | attttacacagataccacatttagcaggaacagaa | | | SYPNKTHPNYISIINEDGNEIF | |
| | caaaactttcagcttgcaaagcaaattcaatccca | | | NTSLFEPPPPGYENVSDIVPP | |
| | gtggaaagaatttggcctggattctgttgagctag | | | FSAFSPQGMPEGDLVYVNY | |
| | cacattatgatgtcctgttgtcctacccaaataag | | | ARTEDFFKLERDMKINCSGKI | |
| | actcatcccaactacatctcaataattaatgaaga | | | VIARYGKVFRGNKVKNAQLA | |
| | tggaaatgagattttcaacacatcattatttgaac | | | GAKGVILYSDPADYFAPGVK | |
| | cacctcctccaggatatgaaaatgtttcggatatt | | | SYPDGWNLPGGGVQRGNIL | |
| | gtaccacctttcagtgctttctctcctcaaggaat | | | NLNGAGDPLTPGYPANEYAY | |
| | gccagagggcgatctagtgtatgttaactatgcac | | | RRGIAEAVGLPSIPVHPIGYY | |
| | gaactgaagacttctttaaattggaacgggacatg | | | DAQKLLEKMGGSAPPDSSW | |
| | aaaatcaattgctctgggaaaattgtaattgccag | | | RGSLKVPYNVGPGFTGNFST | |
| | atatgggaaagttttcagaggaaataaggttaaaa | | | QKVKMHIHSTNEVTRIYNVI | |
| | atgcccagctggcaggggccaaaggagtcattctc | | | GTLRGAVEPDRYVILGGHRD | |
| | tactccgaccctgctgactactttgctcctggggt | | | SWVFGGIDPQSGAAVVHEIV | |
| | gaagtcctatccagatggttggaatcttcctggag | | | RSFGTLKKEGWRPRRTILFAS | |
| | gtggtgtccagcgtggaaatatcctaaatctgaat | | | WDAEEFGLLGSTEWAEENS | |
| | ggtgcaggagaccctctcacaccaggttacccagc | | | RLLQERGVAYINADSSIEGNY | |
| | aaatgaatatgcttataggcgtggaattgcagagg | | | TLRVDCTPLMYSLVHNLTKEL | |
| | ctgttggtcttccaagtattcctgttcatccaatt | | | KSPDEGFEGKSLYESWTKKSP | |
| | ggatactatgatgcacagaagctcctagaaaaaat | | | SPEFSGMPRISKLGSGNDFE | |
| | gggtggctcagcaccaccagatagcagctggagag | | | VFFQRLGIASGRARYTKNWE | |
| | gaagtctcaaagtgccctacaatgttggacctggc | | | TNKFSGYPLYHSVYETYELVE | |
| | tttactggaaacttttctacacaaaaagtcaagat | | | KFYDPMFKYHLTVAQVRGG | |
| | gcacatccactctaccaatgaagtgacaagaattt | | | MVFELANSIVLPFDCRDYAV | |
| | acaatgtgataggtactctcagaggagcagtggaa | | | VLRKYADKIYSISMKHPQEM | |
| | ccagacagatatgtcattctgggaggtcaccggga | | | KTYSVSFDSLFSAVKNFTEIAS | |
| | ctcatgggtgtttggtggtattgaccctcagagtg | | | KFSERLQDFDKSNPIVLRMM | |
| | gagcagctgttgttcatgaaattgtgaggagcttt | | | NDQLMFLERAFIDPLGLPDR | |
| | ggaacactgaaaaaggaagggtggagacctagaag | | | PFYRHVIYAPSSHNKYAGESF | |
| | aacaattttgtttgcaagctgggatgcagaagaat | | | PGIYDALFDIESKVDPSKAW | |
| | ttggtcttcttggttctactgagtgggcagaggag | | | GEVKRQIYVAAFTVQAAAET | |
| | aactcaagactccttcaagagcgtggcgtggctta | | | LSEVA | |
| | tattaatgctgactcatctatagaaggaaactaca | | | | |
| | ctctgagagttgattgtacaccgctgatgtacagc | | | | |
| | ttggtacacaacctaacaaaagagctgaaaagccc | | | | |
| | tgatgaaggctttgaaggcaaatctctttatgaaa | | | | |
| | gttggactaaaaaaagtccttccccagagttcagt | | | | |
| | ggcatgcccaggataagcaaattgggatctggaaa | | | | |
| | tgattttgaggtgttcttccaacgacttggaattg | | | | |
| | cttcaggcagagcacggtatactaaaaattgggaa | | | | |
| | acaaacaaattcagcggctatccactgtatcacag | | | | |
| | tgtctatgaaacatatgagttggtggaaaagtttt | | | | |
| | atgatccaatgtttaaatatcacctcactgtggcc | | | | |
| | caggttcgaggagggatggtgtttgagctagccaa | | | | |
| | ttccatagtgctcccttttgattgtcgagattatg | | | | |
| | ctgtagttttaagaaagtatgctgacaaaatctac | | | | |
| | agtatttctatgaaacatccacaggaaatgaagac | | | | |
| | atacagtgtatcatttgattcacttttttctgcag | | | | |
| | taaagaattttacagaaattgcttccaagttcagt | | | | |
| | gagagactccaggactttgacaaaagcaacccaat | | | | |
| | agtattaagaatgatgaatgatcaactcatgtttc | | | | |
| | tggaaagagcatttattgatccattagggttacca | | | | |
| | gacaggcctttttataggcatgtcatctatgctcc | | | | |
| | aagcagccacaacaagtatgcaggggagtcattcc | | | | |
| | caggaatttatgatgctctgtttgatattgaaagc | | | | |
| | aaagtggacccttccaaggcctggggagaagtgaa | | | | |
| | gagacagatttatgttgcagccttcacagtgcagg | | | | |
| | cagctgcagagactttgagtgaagtagcc | | | | |
| Human | atgtggaatctccttcacgaaaccgactcggctgt | | 3 | MWNLLHETDSAVATARRPR | 4 |
| full | ggccaccgcgcgccgcccgcgctggctgtgcgctg | | | WLCAGALVLAGGFFLLGFLF | |
| length | gggcgctggtgctggcgggtggcttctttctcctc | | | GWFIKSSNEATNITPKHNMK | |
| PSMA | ggcttcctcttcgggtggtttataaaatcctccaa | | | AFLDELKAENIKKFLYNFTQIP | |
| | tgaagctactaacattactccaaagcataatatga | | | HLAGTEQNFQLAKQIQSQW | |
| | aagcatttttggatgaattgaaagctgagaacatc | | | KEFGLDSVELAHYDVLLSYPN | |
| | aagaagttcttatataattttacacagataccaca | | | KTHPNYISIINEDGNEIFNTSL | |
| | tttagcaggaacagaacaaaactttcagcttgcaa | | | FEPPPPGYENVSDIVPPFSAF | |
| | agcaaattcaatcccagtggaaagaatttggcctg | | | SPQGMPEGDLVYVNYARTE | |
| | gattctgttgagctagcacattatgatgtcctgtt | | | DFFKLERDMKINCSGKIVIAR | |
| | gtcctacccaaataagactcatcccaactacatct | | | YGKVFRGNKVKNAQLAGAK | |
| | caataattaatgaagatggaaatgagattttcaac | | | GVILYSDPADYFAPGVKSYPD | |
| | acatcattatttgaaccacctcctccaggatatga | | | GWNLPGGGVQRGNILNLNG | |
| | aaatgtttcggatattgtaccacctttcagtgctt | | | AGDPLTPGYPANEYAYRRGI | |
| | tctctcctcaaggaatgccagagggcgatctagtg | | | AEAVGLPSIPVHPIGYYDAQK | |
| | tatgttaactatgcacgaactgaagacttctttaa | | | LLEKMGGSAPPDSSWRGSLK | |
| | attggaacgggacatgaaaatcaattgctctggga | | | VPYNVGPGFTGNFSTQKVK | |
| | aaattgtaattgccagatatgggaaagttttcaga | | | MHIHSTNEVTRIYNVIGTLRG | |
| | ggaaataaggttaaaaatgcccagctggcaggggc | | | AVEPDRYVILGGHRDSWVF | |
| | caaaggagtcattctctactccgaccctgctgact | | | GGIDPQSGAAVVHEIVRSFG | |
| | actttgctcctggggtgaagtcctatccagatggt | | | TLKKEGWRPRRTILFASWDA | |
| | tggaatcttcctggaggtggtgtccagcgtggaaa | | | EEFGLLGSTEWAEENSRLLQE | |
| | tatcctaaatctgaatggtgcaggagaccctctca | | | RGVAYINADSSIEGNYTLRVD | |
| | caccaggttacccagcaaatgaatatgcttatagg | | | CTPLMYSLVHNLTKELKSPDE | |
| | cgtggaattgcagaggctgttggtcttccaagtat | | | GFEGKSLYESWTKKSPSPEFS | |
| | tcctgttcatccaattggatactatgatgcacaga | | | GMPRISKLGSGNDFEVFFQR | |
| | agctcctagaaaaaatgggggctcagcaccacca | | | LGIASGRARYTKNWETNKFS | |
| | gatagcagctggagaggaagtctcaaagtgcccta | | | GYPLYHSVYETYELVEKFYDP | |
| | caatgttggacctggctttactggaaacttttcta | | | MFKYHLTVAQVRGGMVFEL | |
| | cacaaaaagtcaagatgcacatccactctaccaat | | | ANSIVLPFDCRDYAVVLRKYA | |
| | gaagtgacaagaatttacaatgtgataggtactct | | | DKIYSISMKHPQEMKTYSVSF | |
| | cagaggagcagtggaaccagacagatatgtcattc | | | DSLFSAVKNFTEIASKFSERLQ | |
| | tgggaggtcaccgggactcatgggtgtttggtggt | | | DFDKSNPIVLRMMNDQLMF | |
| | attgaccctcagagtggagcagctgttgttcatga | | | LERAFIDPLGLPDRPFYRHVIY | |
| | aattgtgaggagctttggaacactgaaaaaggaag | | | APSSHNKYAGESFPGIYDALF | |
| | ggtggagacctagaagaacaattttgtttgcaagc | | | DIESKVDPSKAWGEVKRQIY | |
| | tgggatgcagaagaatttggtcttcttggttctac | | | VAAFTVQAAAETLSEVA | |
| | tgagtgggcagaggagaactcaagactccttcaag | | | | |
| | agcgtggcgtggcttatattaatgctgactcatct | | | | |
| | atagaaggaaactacactctgagagttgattgtac | | | | |
| | accgctgatgtacagcttggtacacaacctaacaa | | | | |
| | aagagctgaaaagccctgatgaaggctttgaaggc | | | | |
| | aaatctctttatgaaagttggactaaaaaaagtcc | | | | |
| | ttccccagagttcagtggcatgcccaggataagca | | | | |
| | aattgggatctggaaatgattttgaggtgttcttc | | | | |
| | caacgacttggaattgcttcaggcagagcacggta | | | | |
| | tactaaaaattgggaaacaaacaaattcagcggct | | | | |
| | atccactgtatcacagtgtctatgaaacatatgag | | | | |
| | ttggtggaaaagttttatgatccaatgtttaaata | | | | |
| | tcacctcactgtggcccaggttcgaggagggatgg | | | | |
| | tgtttgagctagccaattccatagtgctccctttt | | | | |
| | gattgtcgagattatgctgtagttttaagaaagta | | | | |
| | tgctgacaaaatctacagtatttctatgaaacatc | | | | |
| | cacaggaaatgaagacatacagtgtatcatttgat | | | | |
| | tcacttttttctgcagtaaagaattttacagaaat | | | | |
| | tgcttccaagttcagtgagagactccaggactttg | | | | |
| | acaaaagcaacccaatagtattaagaatgatgaat | | | | |
| | gatcaactcatgtttctggaaagagcatttattga | | | | |
| | tccattagggttaccagacaggcctttttataggc | | | | |
| | atgtcatctatgctccaagcagccacaacaagtat | | | | |
| | gcaggggagtcattcccaggaatttatgatgctct | | | | |
| | gtttgatattgaaagcaaagtggacccttccaagg | | | | |
| | cctggggagaagtgaagagacagatttatgttgca | | | | |
| | gccttcacagtgcaggcagctgcagagactttgag | | | | |
| | tgaagtagcc | | | | |
| Cyno | atgtggaatctcctgcacgaaaccgactcggctgt | | 5 | MWNLLHETDSAVATARRPR | 6 |
| Full | ggccaccgcgcgccgcccgcgctggctgtgcgctg | | | WLCAGALVLAGGFFLLGFLF | |
| length | gggcactggtgctggcgggtggcttctttctcctc | | | GWFIKSSSEATNITPKHNMK | |
| PSMA | ggcttcctcttcggatggtttataaaatcctccag | | | AFLDELKAENIKKFLHNFTQIP | |
| | tgaagctactaacattactccaaagcataatatga | | | HLAGTEQNFQLAKQIQSQW | |
| | aagcatttttggatgaactgaaagctgagaacatc | | | KEFGLDSVELTHYDVLLSYPN | |
| | aagaagttcttacataattttacacagataccaca | | | KTHPNYISIINEDGNEIFNTSL | |
| | tttagcaggaacagaacaaaactttcaacttgcaa | | | FEPPPAGYENVSDIVPPFSAF | |
| | agcaaattcaatcccagtggaaagaatttggcctg | | | SPQGMPEGDLVYVNYARTE | |
| | gattctgttgagctaactcattatgatgtcctgtt | | | DFFKLERDMKINCSGKIVIAR | |
| | gtcctacccaaataagactcatcccaactacatct | | | YGKVFRGNKVKNAQLAGAT | |
| | caataattaatgaagatggaaatgagattttcaac | | | GVILYSDPADYFAPGVKSYPD | |
| | acatcattatttgaaccacctcctgcaggatatga | | | GWNLPGGGVQRGNILNLNG | |
| | aaatgtttcggatattgtaccacctttcagtgctt | | | AGDPLTPGYPANEYAYRRG | |
| | tctctcctcaaggaatgccagagggcgatctagtg | | | MAEAVGLPSIPVHPIGYYDA | |
| | tatgttaactatgcacgaactgaagacttctttaa | | | QKLLEKMGGSASPDSSWRG | |
| | attggaacgggacatgaaaatcaattgctctggga | | | SLKVPYNVGPGFTGNFSTQK | |
| | aaattgtaattgccagatatgggaaagttttcaga | | | VKMHIHSTSEVTRIYNVIGTL | |
| | ggaaataaggttaaaaatgcccagctggcaggggc | | | RGAVEPDRYVILGGHRDSW | |
| | cacaggagtcattctctactcagaccctgctgact | | | VFGGIDPQSGAAVVHEIVRS | |
| | actttgctcctggggtaaagtcttatccagatggt | | | FGTLKKEGWRPRRTILFASW | |
| | tggaatcttcctggaggtggtgtccagcgtggaaa | | | DAEEFGLLGSTEWAEENSRL | |
| | tatcctaaatctgaatggtgcaggagaccctctca | | | LQERGVAYINADSSIEGNYTL | |
| | caccaggttacccagcaaatgaatatgcttatagg | | | RVDCTPLMYSLVYNLTKELES | |
| | cgtggaatggcagaggctgttggtcttccaagtat | | | PDEGFEGKSLYESWTKKSPSP | |
| | tcccgttcatccaattgggtactatgatgcacaga | | | EFSGMPRISKLGSGNDFEVFF | |
| | agctcctagaaaaaatgggtggctcagcatcacca | | | QRLGIASGRARYTKNWETNK | |
| | gatagcagctggagaggaagtctcaaagtgcccta | | | FSSYPLYHSVYETYELVEKFYD | |
| | caatgttggacctggctttactggaaacttttcta | | | PMFKYHLTVAQVRGGMVFE | |
| | cacaaaaagtcaagatgcacatccactctaccagt | | | LANSVVLPFDCRDYAVVLRK | |
| | gaagtgacaagaatttacaatgtgataggtactct | | | YADKIYNISMKHPQEMKTYS | |
| | cagaggagcagtggaaccagacagatacgtcattc | | | VSFDSLFSAVKNFTEIASKFSE | |
| | tgggaggtcaccgggactcatgggtgtttggtggt | | | RLRDFDKSNPILLRMMNDQL | |
| | attgaccctcagagtggagcagctgttgttcatga | | | MFLERAFIDPLGLPDRPFYRH | |
| | aattgtgaggagctttggaacgctgaaaaaggaag | | | VIYAPSSHNKYAGESFPGIYD | |
| | ggtggagacctagaagaacaattttgtttgcaagc | | | ALFDIESKVDPSQAWGEVKR | |
| | tgggatgcagaagaatttggtcttcttggttctac | | | QISIATFTVQAAAETLSEVA | |
| | tgaatgggcagaggagaactcaagactccttcaag | | | | |
| | agcgtggcgtggcttatattaatgctgattcgtct | | | | |
| | atagaaggaaactacactctgagagttgattgtac | | | | |
| | accactgatgtacagcttggtatacaacctaacaa | | | | |
| | aagagctggaaagccctgatgaaggctttgaaggc | | | | |
| | aaatctctttatgaaagttggactaaaaaaagtcc | | | | |
| | ttcccccgagttcagtggcatgcccaggataagca | | | | |
| | aattgggatctggaaatgattttgaggtgttcttc | | | | |
| | caacgacttggaattgcctcaggcagagcacggta | | | | |
| | tactaaaaattgggaaacaaacaaattcagcagct | | | | |
| | atccactgtatcacagtgtctatgagacatatgag | | | | |
| | ttggtggaaaagttttatgatccaatgtttaaata | | | | |
| | tcacctcactgtggcccaggttcgaggagggatgg | | | | |
| | tgtttgaactagccaattccgtagtgctccctttt | | | | |
| | gattgtcgagattatgctgtagttttaagaaagta | | | | |
| | tgctgacaaaatctacaatatttctatgaaacatc | | | | |
| | cacaggaaatgaagacatacagtgtatcatttgat | | | | |
| | tcacttttttctgcagtaaagaattttacagaaat | | | | |
| | tgcttccaagttcagtgagagactccgggactttg | | | | |
| | acaaaagcaacccaatattattaagaatgatgaat | | | | |
| | gatcaactcatgtttctggaaagagcatttattga | | | | |
| | tccattagggttaccagacagacctttttataggc | | | | |
| | atgtcatctatgctccaagcagccacaacaagtat | | | | |
| | gcaggggagtcattcccaggaatttatgatgctct | | | | |
| | gtttgatatcgaaagcaaagtggacccttcccagg | | | | |
| | cctggggagaagtgaagagacagatttctattgca | | | | |
| | accttcacagtgcaagcagctgcagagactttgag | | | | |
| | tgaagtggcc | | | | |
| 107-1A4 | gagatccagctgcaacagtctggacctgagctggt | | 117 | EIQLQQSGPELVKPGASVKM | 118 |
| VH | gaagcctggggcttcagtgaagatgtcctgcaagg | | | SCKASGYTFTDYYMHWVKQ | |
| domain | cttctggatacacattcactgactactacatgcac | | | NNGESLEWIGYFNPYNDYTR | |
| | tgggtgaagcagaacaatggagagagccttgagtg | | | YNQNFNGKATLTVDKSSSTA | |
| | gattggatattttaatccttataatgattatacta | | | YMQLNSLTSEDSAFYYCARS | |
| | gatacaaccagaatttcaatggcaaggccacattg | | | DGYYDAMDYWGQGTSVTV | |
| | actgtagacaagtcctccagcacagcctacatgca | | | SS | |
| | gctcaacagcctgacatctgaggactctgcattct | | | | |
| | attactgtgcaagatcggatggttactacgatgct | | | | |
| | atggactactggggtcaaggaacctcagtcaccgt | | | | |
| | ctcctcg | | | | |
| 107-1A4 | gatgtccagataacccagtctccatcttatcttgc | | 119 | DVQITQSPSYLAASPGETITIN | 120 |
| VL | tgcatctcctggagaaaccattactattaattgca | | | CRASKSISKYLAWYQEKPGK | |
| domain | gggcaagtaagagcattagcaaatatttagcctgg | | | ANKLLIHSGSTLQSGIPSRFSG | |
| | tatcaagagaaacctgggaaagctaataagctact | | | SGSGTDFTLTISSLEPEDFAM | |
| | tatccattctggatccactttgcaatctggaatac | | | YYCQQHIEYPWTFGGGTKLE | |
| | catcaaggttcagtggcagtggatctggtacagat | | | IK | |
| | ttcactctcaccatcagtagcctggagcctgaaga | | | | |
| | ttttgcaatgtattactgtcaacagcatattgaat | | | | |
| | acccgtggacgttcggtggtggcaccaaactggaa | | | | |
| | attaaacgggcc | | | | |
| CRIS-7 | | | | QVQLQQSGAELARPGASVK | 122 |
| VH | | | | MSCKASGYTFTRSTMHWVK | |
| domain | | | | QRPGQGLEWIGYINPSSAYT | |
| | | | | NYNQKFKDKATLTADKSSST | |
| | | | | AYMQLSSLTSEDSAVYYCAS | |
| | | | | PQVHYDYNGFPYWGQGT | |
| CRIS-7 | | | | QVVLTQSPAIMSAFPGEKVT | 124 |
| VL | | | | MTCSASSSVSYMNWYQQKS | |
| domain | | | | GTSPKRWIYDSSKLASGVPA | |
| | | | | RFSGSGSGTSYSLTISSMETE | |
| | | | | DAATYYCQQWSRNPPTFGG | |
| | | | | GTKLQITR | |
| TSC266 | gatatccagatgacccagtctccatccgccatgtc | | 7 | DIQMTQSPSAMSASVGDRV | 8 |
| Anti | tgcatctgtaggagacagagtcaccatcacttgcc | | | TITCRASKSISKYLAWFQQKP | |
| PSMA | gggcgagtaagagcattagcaaatatttagcctgg | | | GKVPKLRIHSGSTLQSGVPSR | |
| scFv x | tttcagcagaaaccagggaaagttcctaagctccg | | | FSGSGSGTEFTLTISSLQPEDF | |
| Anti | catccattctggatctactttgcaatcaggggtcc | | | ATYYCQQHIEYPWTFGQGTK | |
| CD3 scFv | catctcggttcagtggcagtggatctgggacagaa | | | VEIKRGGGGSGGGGSGGGG | |
| ADAPTIR ^{™} | tttactctcaccatcagcagcctgcagcctgaaga | | | SQVQLVQSGAEVKKPGASVK | |
| | ttttgcaacttattactgtcaacagcatattgaat | | | VSCKASGYTFTDYYMHWVR | |
| | acccgtggacgttcggccaagggaccaaggtggaa | | | QAPGQGLEWMGYFNPYND | |
| | atcaaacgaggtggcggagggtctgggggtggcgg | | | YTRYAQKFQGRVTMTRDTSI | |
| | atccggaggtggtggctctcaggtccagctggtac | | | STAYMELSSLRSDDTAVYYCA | |
| | agtctggggctgaggtgaagaagcctggggcttca | | | RSDGYYDAMDYWGQGTTV | |
| | gtgaaggtctcctgcaaggcttctggatacacatt | | | TVSSSEPKSSDKTHTCPPCPA | |
| | cactgactactacatgcactgggtgcgacaggccc | | | PEAAGAPSVFLFPPKPKDTL | |
| | ctggacaagggcttgagtggatgggatattttaat | | | MISRTPEVTCVVVDVSHEDP | |
| | ccttataatgattatactagatacgcacagaagtt | | | EVKFNWYVDGVEVHNAKTK | |
| | ccagggcagagtcaccatgaccagggacacgtcta | | | PREEQYNSTYRVVSVLTVLH | |
| | tcagcacagcctacatggagctgagcagcctgaga | | | QDWLNGKAYACAVSNKALP | |
| | tctgacgacacggccgtgtattactgtgcaagatc | | | APIEKTISKAKGQPREPQVYT | |
| | ggatggttactacgatgctatggactactggggtc | | | LPPSRDELTKNQVSLTCLVKG | |
| | aaggaaccacagtcaccgtctcctcgagtgagccc | | | FYPSDIAVEWESNGQPENNY | |
| | aaatcttctgacaaaactcacacatgcccaccgtg | | | KTTPPVLDSDGSFFLYSKLTV | |
| | cccagcacctgaagccgcgggtgcaccgtcagtct | | | DKSRWQQGNVFSCSVMHE | |
| | tcctcttccccccaaaacccaaggacaccctcatg | | | ALHNHYTQKSLSLSPGQRHN | |
| | atctcccggacccctgaggtcacatgcgtggtggt | | | NSSLNTGTQMAGHSPNSQV | |
| | ggacgtgagccacgaagaccctgaggtcaagttca | | | QLVESGGGVVQPGRSLRLSC | |
| | actggtacgtggacggcgtggaggtgcataatgcc | | | KASGYTFTRSTMHWVRQAP | |
| | aagacaaagccgcgggaggagcagtacaacagcac | | | GQGLEWIGYINPSSAYTNYN | |
| | gtaccgtgtggtcagcgtcctcaccgtcctgcacc | | | QKFKDRFTISADKSKSTAFLQ | |
| | aggactggctgaatggcaaggcgtacgcgtgcgcg | | | MDSLRPEDTGVYFCARPQV | |
| | gtctccaacaaagccctcccagcccccatcgagaa | | | HYDYNGFPYWGQGTPVTVS | |
| | aaccatctccaaagccaaagggcagccccgagaac | | | SGGGGSGGGGSGGGGSAQ | |
| | cacaggtgtacaccctgcccccatcccgggatgag | | | DIQMTQSPSSLSASVGDRVT | |
| | ctgaccaagaaccaggtcagcctgacctgcctggt | | | MTCSASSSVSYMNWYQQKP | |
| | caaaggcttctatccaagcgacatcgccgtggagt | | | GKAPKRWIYDSSKLASGVPA | |
| | gggagagcaatgggcagccggagaacaactacaag | | | RFSGSGSGTDYTLTISSLQPE | |
| | accacgcctcccgtgctggactccgacggctcctt | | | DFATYYCQQWSRNPPTFGG | |
| | cttcctctacagcaagctcaccgtggacaagagca | | | GTKLQITSSS | |
| | ggtggcagcaggggaacgtcttctcatgctccgtg | | | | |
| | atgcatgaggctctgcacaaccactacacgcagaa | | | | |
| | gagcctctccctgtctccgggtcagaggcacaaca | | | | |
| | attcttccctgaatacaggaactcagatggcaggt | | | | |
| | cattctccgaattctcaggtccagctggtggagtc | | | | |
| | tgggggcggagtggtgcagcctgggcggtcactga | | | | |
| | ggctgtcctgcaaggcttctggctacacctttact | | | | |
| | agatctacgatgcactgggtaaggcaggcccctgg | | | | |
| | acaaggtctggaatggattggatacattaatccta | | | | |
| | gcagtgcttatactaattacaatcagaaattcaag | | | | |
| | gacaggttcacaatcagcgcagacaaatccaagag | | | | |
| | cacagccttcctgcagatggacagcctgaggcccg | | | | |
| | aggacaccggcgtctatttctgtgcacggccccaa | | | | |
| | gtccactatgattacaacgggtttccttactgggg | | | | |
| | ccaagggactcccgtcactgtctctagcggtggcg | | | | |
| | gagggtctgggggggcggatccggaggtggtggc | | | | |
| | tctgcacaagacatccagatgacccagtctccaag | | | | |
| | cagcctgtctgcaagcgtgggggacagggtcacca | | | | |
| | tgacctgcagtgccagctcaagtgtaagttacatg | | | | |
| | aactggtaccagcagaagccgggcaaggcccccaa | | | | |
| | aagatggatttatgactcatccaaactggcttctg | | | | |
| | gagtccctgctcgcttcagtggcagtgggtctggg | | | | |
| | accgactataccctcacaatcagcagcctgcagcc | | | | |
| | cgaagatttcgccacttattactgccagcagtgga | | | | |
| | gtcgtaacccacccacgttcggaggggggaccaag | | | | |
| | ctacaaattacatcctccagc | | | | |
| DRA222 | | | | QVQLVESGGGVVQPGRSLR | 126 |
| VH | | | | LSCKASGYTFTRSTMHWVR | |
| domain | | | | QAPGQGLEWIGYINPSSAYT | |
| (TSC266 | | | | NYNQKFKDRFTISADKSKSTA | |
| CD3 VH | | | | FLQMDSLRPEDTGVYFCARP | |
| domain) | | | | QVHYDYNGFPYWGQGTPV | |
| | | | | TVSS | |
| DRA222 | | | | DIQMTQSPSSLSASVGDRVT | 128 |
| VL | | | | MTCSASSSVSYMNWYQQKP | |
| domain | | | | GKAPKRWIYDSSKLASGVPA | |
| (TSC266 | | | | RFSGSGSGTDYTLTISSLQPE | |
| CD3 VL | | | | DFATYYCQQWSRNPPTFGG | |
| domain) | | | | GTKLQIT | |
| TSC456 | | | | QVQLVQSGPEVKKPGSSVKV | 130 |
| VH | | | | SCKASGYTFSRSTMHWVRQ | |
| domain | | | | APGQGLEWIGYINPSSAYTN | |
| | | | | YNQKFKDRVTITADKSTSTAY | |
| | | | | MELSSLRSEDTAVYYCARPQ | |
| | | | | VHYDYNGFPYWGQGTLVTV | |
| | | | | SS | |
| TSC456 | | | | DIQMTQSPSTLSASVGDRVT | 132 |
| VL | | | | MTCSASSSVSYMNWYQQKP | |
| domain | | | | GKAPKRWIYDSSKLASGVPS | |
| | | | | RFSGSGSGTDYTLTISSLQPD | |
| | | | | DFATYYCQQWSRNPPTFGG | |
| | | | | GTKVEIK | |
| CRIS7H | | | | QVQLVQSGAEVKKPGASVK | 134 |
| 14 VH | | | | VSCKASGYTFTRSTMHWVR | |
| domain | | | | QAPGQGLEWIGYINPSSAYT | |
| | | | | NYAQKFQGRVTLTADKSTST | |
| | | | | AYMELSSLRSEDTAVYYCASP | |
| | | | | QVHYDYNGFPYWGQGTLVT | |
| | | | | VSS | |
| CRIS7H | | | | DVQLTQSPSTLSASVGDRVTI | 136 |
| 14 VL | | | | TCSASSSVSYMNWYQQKPG | |
| domain | | | | KAPKRWIYDSSKLASGVPAR | |
| | | | | FSGSGSGTLTISSLQPDDFAT | |
| | | | | YYCQQWSRMPPTFGQGTK | |
| | | | | VEVK | |
| CRIS7H | | | | QVQLVQSGAEVKKPGASVK | 138 |
| 15 VH | | | | VSCKASGYTFTRSTMHWVR | |
| domain | | | | QAPGQGLEWIGYINPSSAYT | |
| | | | | NYNQKRFQGRVTLTADKSTS | |
| | | | | TAYMELSSLRSEDTAVYYCAS | |
| | | | | PQVHYDYNGFPYWGQGTLV | |
| | | | | TVSS | |
| CRIS7H | | | | DVQLTQSPSTLSASVGDRVTI | 140 |
| 15 VL | | | | TCSASSSVSYMNWYQQKPG | |
| domain | | | | KAPKRWIYDSSKLASGVPAR | |
| | | | | FSGSGSGTEYTLTISSLQPDDF | |
| | | | | ATYYCQQWSRNPPTFGQGT | |
| | | | | KVEVK | |
| CRIS7H | | | | QVQLVQSGAEVKKPGASVK | 142 |
| 16 VH | | | | VSCKASGYTFTRSTMHWVR | |
| domain | | | | QAPGQGLEWIGYINPSSAYT | |
| | | | | NYAQKFQGRVTLTADKSTST | |
| | | | | AYMELSSLRSEDTAVYYCASP | |
| | | | | QVHYDYNGFPYWGQGTLVT | |
| | | | | VSS | |
| CRIS7H | | | | DIQMTQSPSSLSASVGDRVTI | 144 |
| 16 VL | | | | TCRASSSVSYMNWYQQKPG | |
| domain | | | | KAPKRWIYDSSKLASGVPSRF | |
| | | | | SGSGSGTDFTLTISSLQPEDF | |
| | | | | ATYYCQQWSRNPPTFGQGT | |
| | | | | KVEIK | |
| TSC291 | caggtgcagctggtcgagtctggcggcggactggt | | 9 | QVQLVESGGGLVKPGESLRL | 10 |
| Anti | caagcctggcgagtccctgagactgtcttgcgctg | | | SCAASGFTFSDYYMYWVRQ | |
| PSMA | cctccggcttcaccttctccgactactacatgtac | | | APGKGLEWVAIISDGGYYTY | |
| scFv- | tgggtccgccaggctcctggcaagggactggaatg | | | YSDIIKGRFTISRDNAKNSLYL | |
| linker | ggtggccatcatctccgacggcggctactacacct | | | QMNSLKAEDTAVYYCARGF | |
| Anti CD3 | actactccgacatcatcaagggccggttcaccatc | | | PLLRHGAMDYWGQGTLVTV | |
| scFv-His | tccagggacaacgccaagaactccctgtacctgca | | | SSGGGGSGGGGSGGGGSDI | |
| | gatgaactccctgaaggccgaggacaccgccgtgt | | | QMTQSPSSLSASVGDRVTIT | |
| | actactgcgccaggggcttcccactgctgagacac | | | CKASQNVDTNVAWYQQKP | |
| | ggcgccatggattactggggccagggcaccctggt | | | GQAPKSLIYSASYRYSDVPSR | |
| | cacagtgtcctctggcggaggcggaagtggaggcg | | | FSGSASGTDFTLTISSVQSEDF | |
| | gaggaagcggaggcggcggatccgacatccagatg | | | ATYYCQQYDSYPYTFGGGTK | |
| | acccagtccccatcctccctgtctgcctccgtggg | | | LEIKSGGGGSEVQLVESGGG | |
| | cgacagagtgaccatcacatgcaaggcctcccaga | | | LVQPGGSLKLSCAASGFTFN | |
| | acgtggacaccaacgtggcatggtatcagcagaag | | | KYAMNWVRQAPGKGLEWV | |
| | ccaggccaggcccctaagtccctgatctactctgc | | | ARIRSKYNNYATYYADSVKD | |
| | ctcctaccggtactccgacgtgccctccaggttct | | | RFTISRDDSKNTAYLQMNNL | |
| | ctggctccgcctctggcaccgacttcaccctgacc | | | KTEDTAVYYCVRHGNFGNSY | |
| | atctcttccgtgcagtccgaggacttcgctaccta | | | ISYWAYWGQGTLVTVSSGG | |
| | ctactgccagcagtacgactcctacccttacacct | | | GGSGGGGSGGGGSQTVVT | |
| | tcggcggaggcaccaagctggaaatcaagtccggc | | | QEPSLTVSPGGTVTLTCGSST | |
| | ggagggggctctgaagtgcagctggtggaaagcgg | | | GAVTSGNYPNWVQQKPGQ | |
| | agggggactggtgcagcccgggggaagtctgaagc | | | APRGLIGGTKFLAPGTPARFS | |
| | tgtcctgtgccgccagcggctttaccttcaacaag | | | GSLLGGKAALTLSGVQPEDE | |
| | tacgccatgaattgggtccgacaggccccagggaa | | | AEYYCVLWYSNRWVFGGGT | |
| | aggcctggaatgggtggcacggatccggtccaagt | | | KLTVLHHHHHHHHHH | |
| | acaacaactacgccacctactacgctgactccgtg | | | | |
| | aaggacagattcaccatcagccgggacgactctaa | | | | |
| | gaacaccgcctatctgcagatgaacaacctgaaaa | | | | |
| | ccgaggatacagctgtgtactattgtgtgcggcac | | | | |
| | ggcaacttcggcaactcctacatctcctactgggc | | | | |
| | ctattggggacagggaacactggtcaccgtgtcta | | | | |
| | gcggaggtggcggaagtgggggaggcggatctggc | | | | |
| | ggtggcggatcccagaccgtggtcacccaggaacc | | | | |
| | ttccctgaccgtctccccaggcggcaccgtgaccc | | | | |
| | tgacctgtggctcctctaccggcgctgtgacctcc | | | | |
| | ggcaactaccctaactgggtgcagcagaaacccgg | | | | |
| | acaggctcctagaggcctgatcggcggcaccaagt | | | | |
| | ttctggcccctggcacccctgccagattctccggc | | | | |
| | tccctgctgggaggcaaggccgctctgaccctgtc | | | | |
| | tggcgtgcagcctgaggacgaggccgagtactact | | | | |
| | gtgtgctgtggtactccaacagatgggtgttcgga | | | | |
| | ggcggcacaaagctgaccgtgctgcatcatcatca | | | | |
| | tcatcaccatcatcatcac | | | | |
| TRI130 | gacatcgtgatgacccagtctccagactccctggc | | 11 | DIVMTQSPDSLAVSLGERATI | 12 |
| Anti TA | tgtgtctctgggcgagagggccaccatcaactgca | | | NCKSSHSVLYSSNNKNYLAW | |
| scFv x | agtccagccacagtgttttatacagctccaacaat | | | YQQKPGQPPKLLIYWASTRE | |
| Anti CD3 | aagaactacttagcttggtaccagcagaaaccagg | | | SGVPDRFSGSGSGTDFTLTIS | |
| scFv | acagcctcctaagctgctcatttactgggcatcta | | | SLQAEDVAVYYCQQYYSTPP | |
| ADAPTIR ^{™} | cccgggaatccggggtccctgaccgattcagtggc | | | TTFGGGTKVEIKGGGGSGGG | |
| | agcgggtctgggacagatttcactctcaccatcag | | | GSGGGGSGGGGSEVQLLES | |
| | cagcctgcaggctgaagatgtggcagtttattact | | | GGGLVQPGGSLRLSCAASGF | |
| | gtcagcaatattatagtactcctccgaccactttc | | | TFSSYGMSWVRQAPGKGLE | |
| | ggcggagggaccaaggtggagatcaaaggtggagg | | | GVSAISGSGGSTYYADSVKG | |
| | cggttcaggcggaggtggatccggcggtggcggct | | | RFTISRDNSKNTLYLQMNSLR | |
| | ccggtggcggcggatctgaggtgcagctgttggag | | | AEDTAVYYCAKEKLRYFDWL | |
| | tctgggggaggcttggtacagcctggggggtccct | | | SDAFDIWGQGTMVTVSSSE | |
| | gagactctcctgtgcagcctctggattcaccttta | | | PKSSDKTHTCPPCPAPEAAG | |
| | gcagctatggcatgagctgggtccgccaggctcca | | | APSVFLFPPKPKDTLMISRTP | |
| | gggaaggggctggagggggtctcagctattagtgg | | | EVTCVVVDVSHEDPEVKFN | |
| | tagtggtggtagcacatactacgcagactccgtga | | | WYVDGVEVHNAKTKPREEQ | |
| | agggccggttcaccatctccagagacaattccaag | | | YNSTYRVVSVLTVLHQDWLN | |
| | aacacgctgtatctgcaaatgaacagcctgagagc | | | GKEYKCAVSNKALPAPIEKTIS | |
| | cgaggacacggccgtatattactgtgcgaaagaaa | | | KAKGQPREPQVYTLPPSRDE | |
| | agttacgatattttgactggttatccgatgctttt | | | LTKNQVSLTCLVKGFYPSDIA | |
| | gatatctggggccaagggacaatggtcaccgtctc | | | VEWESNGQPENNYKTTPPV | |
| | ctcgagtgagcccaaatcttctgacaaaactcaca | | | LDSDGSFFLYSKLTVDKSRW | |
| | catgcccaccgtgcccagcacctgaagccgcgggt | | | QQGNVFSCSVMHEALHNHY | |
| | gcaccgtcagtcttcctcttccccccaaaacccaa | | | TQKSLSLSPGSGGGGSGGGG | |
| | ggacaccctcatgatctcccggacccctgaggtca | | | SGGGGSPSQVQLVQSGPEV | |
| | catgcgtggtggtggacgtgagccacgaagaccct | | | KKPGSSVKVSCKASGYTFSRS | |
| | gaggtcaagttcaactggtacgtggacggcgtgga | | | TMHWVRQAPGQGLEWIGY | |
| | ggtgcataatgccaagacaaagccgcgggaggagc | | | INPSSAYTNYNQKFKDRVTIT | |
| | agtacaacagcacgtaccgtgtggtcagcgtcctc | | | ADKSTSTAYMELSSLRSEDTA | |
| | accgtcctgcaccaggactggctgaatggcaagga | | | VYYCARPQVHYDYNGFPYW | |
| | atacaagtgcgcggtctccaacaaagccctcccag | | | GQGTLVTVSSGGGGSGGGG | |
| | cccccatcgagaaaaccatctccaaagccaaaggg | | | SGGGGSGGGGSDIQMTQSP | |
| | cagccccgagaaccacaggtgtacaccctgccccc | | | STLSASVGDRVTMTCSASSS | |
| | atcccgggatgagctgaccaagaaccaggtcagcc | | | VSYMNWYQQKPGKAPKRW | |
| | tgacctgcctggtcaaaggcttctatccaagcgac | | | IYDSSKLASGVPSRFSGSGSG | |
| | atcgccgtggagtgggagagcaatgggcagccgga | | | TDYTLTISSLQPDDFATYYCQ | |
| | gaacaactacaagaccacgcctcccgtgctggact | | | QWSRNPPTFGGGTKVEIKRS | |
| | ccgacggctccttcttcctctacagcaagctcacc | | | | |
| | gtggacaagagcaggtggcagcaggggaacgtctt | | | | |
| | ctcatgctccgtgatgcatgaggctctgcacaacc | | | | |
| | actacacgcagaagagcctctccctgtctccgggt | | | | |
| | tccggaggagggggttcaggtgggggaggttctgg | | | | |
| | cggcgggggaagcccttcacaggtgcaactggtgc | | | | |
| | agagtggacccgaggttaaaaaaccagggtcctcc | | | | |
| | gttaaggttagctgcaaagcctctggctacacatt | | | | |
| | ttccaggagtacaatgcactgggtgaggcaggctc | | | | |
| | ctggacagggactcgagtggatcgggtatatcaac | | | | |
| | ccatctagcgcctataccaattacaaccaaaagtt | | | | |
| | taaggaccgagttaccattaccgctgacaaatcca | | | | |
| | ccagtacagcttatatggagctgtcatctcttagg | | | | |
| | tccgaggacactgctgtttattactgcgctcgtcc | | | | |
| | tcaggttcactatgactataatggttttccctact | | | | |
| | ggggtcagggaaccctggtgactgtctcttctggc | | | | |
| | ggtggaggcagcggtgggggtgggtctggaggcgg | | | | |
| | tggcagtggcggcggaggctctgatattcagatga | | | | |
| | ctcagtctcctagcactctcagcgccagcgtgggg | | | | |
| | gatcgtgtgacaatgacttgctccgctagcagtag | | | | |
| | tgtgtcttacatgaattggtatcagcagaagcccg | | | | |
| | ggaaagcacctaagcgctggatctatgactcttcc | | | | |
| | aagctggcaagtggtgtcccctcacggttctctgg | | | | |
| | ctcaggttctggtactgactatactttgactatct | | | | |
| | cctccctccagcccgatgatttcgctacctattat | | | | |
| | tgtcagcagtggagccgtaacccacccactttcgg | | | | |
| | aggcggtaccaaagtggagatcaagaggtca | | | | |
| TRI149 | gaggtgcagctgttggagtctgggggaggcttggt | | 13 | EVQLLESGGGLVQPGGSLRL | 14 |
| Anti TA | acagcctggggggtccctgagactctcctgtgcag | | | SCAASGFTFSSYAMSWVRQ | |
| scFv x | cctctggattcacctttagcagctatgccatgagc | | | APGKGLEWVSAISGSGGSTY | |
| Anti | tgggtccgccaggctccagggaaggggctggagtg | | | YADSVKGRFTISRDNSKNTLY | |
| CD3 scFv | ggtctcagctattagtggtagtggtggtagcacat | | | LQMNSLRAEDTAVYYCAKEK | |
| ADAPTIR ^{™} | actacgcagactccgtgaagggccggttcaccatc | | | LRYFDWLSDAFDIWGQGTM | |
| | tccagagacaattccaagaacacgctgtatctgca | | | VTVSSGGGGSGGGGSGGGG | |
| | aatgaacagcctgagagccgaggacacggccgtat | | | SGGGGSDIVMTQSPDSLAVS | |
| | attactgtgcgaaagaaaagttacgatattttgac | | | LGERATINCKSSQSVLYSSNN | |
| | tggttatccgatgcttttgatatctggggccaagg | | | KNYLAWYQQKPGQPPKLLIY | |
| | gacaatggtcaccgtctcttcaggtggaggcggtt | | | WASTRESGVPDRFSGSGSGT | |
| | caggcggaggtggatccggcggtggcggctccggt | | | DFTLTISSLQAEDVAVYYCQQ | |
| | ggcggcggatctgacatcgtgatgacccagtctcc | | | YYSTPPTTFGGGTKVEIKSSSE | |
| | agactccctggctgtgtctctgggcgagagggcca | | | PKSSDKTHTCPPCPAPEAAG | |
| | ccatcaactgcaagtccagccaaagtgttttatac | | | APSVFLFPPKPKDTLMISRTP | |
| | agctccaacaataagaactacttagcttggtacca | | | EVTCVVVDVSHEDPEVKFN | |
| | gcagaaaccaggacagcctcctaagctgctcattt | | | WYVDGVEVHNAKTKPREEQ | |
| | actgggcatctacccgggaatccggggtccctgac | | | YNSTYRVVSVLTVLHQDWLN | |
| | cgattcagtggcagcgggtctgggacagatttcac | | | GKEYKCAVSNKALPAPIEKTIS | |
| | tctcaccatcagcagcctgcaggctgaagatgtgg | | | KAKGQPREPQVYTLPPSRDE | |
| | cagtttattactgtcagcaatattatagtactcct | | | LTKNQVSLTCLVKGFYPSDIA | |
| | ccgaccactttcggcggagggaccaaggtggagat | | | VEWESNGQPENNYKTTPPV | |
| | caaatcctcgagtgagcccaaatcttctgacaaaa | | | LDSDGSFFLYSKLTVDKSRW | |
| | ctcacacatgcccaccgtgcccagcacctgaagcc | | | QQGNVFSCSVMHEALHNHY | |
| | gcgggtgcaccgtcagtcttcctcttccccccaaa | | | TQKSLSLSPGSGGGGSGGGG | |
| | acccaaggacaccctcatgatctcccggacccctg | | | SGGGGSPSQVQLVQSGPEV | |
| | aggtcacatgcgtggtggtggacgtgagccacgaa | | | KKPGSSVKVSCKASGYTFSRS | |
| | gaccctgaggtcaagttcaactggtacgtggacgg | | | TMHWVRQAPGQGLEWIGY | |
| | cgtggaggtgcataatgccaagacaaagccgcggg | | | INPSSAYTNYNQKFKDRVTIT | |
| | aggagcagtacaacagcacgtaccgtgtggtcagc | | | ADKSTSTAYMELSSLRSEDTA | |
| | gtcctcaccgtcctgcaccaggactggctgaatgg | | | VYYCARPQVHYDYNGFPYW | |
| | caaggaatacaagtgcgcggtctccaacaaagccc | | | GQGTLVTVSSGGGGSGGGG | |
| | tcccagcccccatcgagaaaaccatctccaaagcc | | | SGGGGSGGGGSDIQMTQSP | |
| | aaagggcagccccgagaaccacaggtgtacaccct | | | STLSASVGDRVTMTCSASSS | |
| | gcccccatcccgggatgagctgaccaagaaccagg | | | VSYMNWYQQKPGKAPKRW | |
| | tcagcctgacctgcctggtcaaaggcttctatcca | | | IYDSSKLASGVPSRFSGSGSG | |
| | agcgacatcgccgtggagtgggagagcaatgggca | | | TDYTLTISSLQPDDFATYYCQ | |
| | gccggagaacaactacaagaccacgcctcccgtgc | | | QWSRNPPTFGGGTKVEIKRS | |
| | tggactccgacggctccttcttcctctacagcaag | | | | |
| | ctcaccgtggacaagagcaggtggcagcaggggaa | | | | |
| | cgtcttctcatgctccgtgatgcatgaggctctgc | | | | |
| | acaaccactacacgcagaagagcctctccctgtct | | | | |
| | ccgggttccggaggagggggttcaggtgggggagg | | | | |
| | ttctggcggcgggggaagcccttcacaggtgcaac | | | | |
| | tggtgcagagtggacccgaggttaaaaaaccaggg | | | | |
| | tcctccgttaaggttagctgcaaagcctctggcta | | | | |
| | cacattttccaggagtacaatgcactgggtgaggc | | | | |
| | aggctcctggacagggactcgagtggatcgggtat | | | | |
| | atcaacccatctagcgcctataccaattacaacca | | | | |
| | aaagtttaaggaccgagttaccattaccgctgaca | | | | |
| | aatccaccagtacagcttatatggagctgtcatct | | | | |
| | cttaggtccgaggacactgctgtttattactgcgc | | | | |
| | tcgtcctcaggttcactatgactataatggttttc | | | | |
| | cctactggggtcagggaaccctggtgactgtctct | | | | |
| | tctggcggtggaggcagcggtgggggtgggtctgg | | | | |
| | aggcggtggcagtggcggcggaggctctgatattc | | | | |
| | agatgactcagtctcctagcactctcagcgccagc | | | | |
| | gtgggggatcgtgtgacaatgacttgctccgctag | | | | |
| | cagtagtgtgtcttacatgaattggtatcagcaga | | | | |
| | agcccgggaaagcacctaagcgctggatctatgac | | | | |
| | tcttccaagctggcaagtggtgtcccctcacggtt | | | | |
| | ctctggctcaggttctggtactgactatactttga | | | | |
| | ctatctcctccctccagcccgatgatttcgctacc | | | | |
| | tattattgtcagcagtggagccgtaacccacccac | | | | |
| | tttcggaggcggtaccaaagtggagatcaagaggt | | | | |
| | ca | | | | |
| TRI | gacatcgtgatgacccagtctccagactccctggc | | 15 | DIVMTQSPDSLAVSLGERATI | 16 |
| 01043 | tgtgtctctgggcgagagggccaccatcaactgca | | | NCKSSHSVLYSSNNKNYLAW | |
| Anti TA | agtccagccacagtgttttatacagctccaacaat | | | YQQKPGQPPKLLIYWASTRE | |
| scFv x | aagaactacttagcttggtaccagcagaaaccagg | | | SGVPDRFSGSGSGTDFTLTIS | |
| Anti | acagcctcctaagctgctcatttactgggcatcta | | | SLQAEDVAVYYCQQYYSTPP | |
| CD3 scFv | cccgggaatccggggtccctgaccgattcagtggc | | | TTFGGGTKVEIKGGGGSGGG | |
| ADAPTIR ^{™} | agcgggtctgggacagatttcactctcaccatcag | | | GSGGGGSGGGGSEVQLLES | |
| | cagcctgcaggctgaagatgtggcagtttattact | | | GGGLVQPGGSLRLSCAASGF | |
| | gtcagcaatattatagtactcctccgaccactttc | | | TFSSYGMSWVRQAPGKGLE | |
| | ggcggagggaccaaggtggagatcaaaggtggagg | | | GVSAISGSGGSTYYADSVKG | |
| | cggttcaggcggaggtggatccggcggtggcggct | | | RFTISRDNSKNTLYLQMNSLR | |
| | ccggtggcggcggatctgaggtgcagctgttggag | | | AEDTAVYYCAKEKLRYFDWL | |
| | tctgggggaggcttggtacagcctggggggtccct | | | SDAFDIWGQGTMVTVSSSE | |
| | gagactctcctgtgcagcctctggattcaccttta | | | PKSSDKTHTCPPCPAPPAAA | |
| | gcagctatggcatgagctgggtccgccaggctcca | | | PSVFLFPPKPKDTLMISRTPE | |
| | gggaaggggctggagggggtctcagctattagtgg | | | VTCVVVDVSHEDPEVKFNW | |
| | tagtggtggtagcacatactacgcagactccgtga | | | YVDGVEVHNAKTKPREEQY | |
| | agggccggttcaccatctccagagacaattccaag | | | NSTYRVVSVLTVLHQDWLN | |
| | aacacgctgtatctgcaaatgaacagcctgagagc | | | GKEYKCAVSNKALPAPIEKTIS | |
| | cgaggacacggccgtatattactgtgcgaaagaaa | | | KAKGQPREPQVYTLPPSRDE | |
| | agttacgatattttgactggttatccgatgctttt | | | LTKNQVSLTCLVKGFYPSDIA | |
| | gatatctggggccaagggacaatggtcaccgtctc | | | VEWESNGQPENNYKTTPPV | |
| | ctcgagtgagcccaaatcttctgacaaaactcaca | | | LDSDGSFFLYSKLTVDKSRW | |
| | catgcccaccgtgcccagcacctccagccgctgca | | | QQGNVFSCSVMHEALHNHY | |
| | ccgtcagtcttcctcttccccccaaaacccaagga | | | TQKSLSLSPGGGGSPSQVQL | |
| | caccctcatgatctcccggacccctgaggtcacat | | | VQSGAEVKKPGASVKVSCKA | |
| | gcgtggtggtggacgtgagccacgaagaccctgag | | | SGYTFTRSTMHWVRQAPGQ | |
| | gtcaagttcaactggtacgtggacggcgtggaggt | | | GLEWIGYINPSSAYTNYNQK | |
| | gcataatgccaagacaaagccgcgggaggagcagt | | | FQGRVTLTADKSTSTAYMEL | |
| | acaacagcacgtaccgtgtggtcagcgtcctcacc | | | SSLRSEDTAVYYCASPQVHY | |
| | gtcctgcaccaggactggctgaatggcaaggaata | | | DYNGFPYWGQGTLVTVSSG | |
| | caagtgcgcggtctccaacaaagccctcccagccc | | | GGGSGGGGSGGGGSGGGG | |
| | ccatcgagaaaaccatctccaaagccaaagggcag | | | SDVQLTQSPSTLSASVGDRV | |
| | ccccgagaaccacaggtgtacaccctgcccccatc | | | TITCSASSSVSYMNWYQQKP | |
| | ccgggatgagctgaccaagaaccaggtcagcctga | | | GKAPKRWIYDSSKLASGVPA | |
| | cctgcctggtcaaaggcttctatccaagcgacatc | | | RFSGSGSGTEYTLTISSLQPD | |
| | gccgtggagtgggagagcaatgggcagccggagaa | | | DFATYYCQQWSRNPPTFGQ | |
| | caactacaagaccacgcctcccgtgctggactccg | | | GTKVEVKRS | |
| | acggctccttcttcctctacagcaagctcaccgtg | | | | |
| | gacaagagcaggtggcagcaggggaacgtcttctc | | | | |
| | atgctccgtgatgcatgaggctctgcacaaccact | | | | |
| | acacgcagaagagcctctccctgtctccgggcggc | | | | |
| | gggggatccccgtcacaagtacaactcgttcaaag | | | | |
| | tggcgcagaagtaaagaagccaggcgccagtgtta | | | | |
| | aggtgagctgcaaggcaagcgggtacaccttcacc | | | | |
| | cggtctacaatgcactgggtaagacaagcaccagg | | | | |
| | gcaaggactcgaatggattggttacatcaaccctt | | | | |
| | cctctgcatacaccaactacaatcaaaagttccag | | | | |
| | ggccgcgttactttgacagcggataaatctacatc | | | | |
| | cacggcctatatggaactgtcaagcctcaggagcg | | | | |
| | aggacacagcggtatattactgtgcatctccccag | | | | |
| | gtccattatgactacaacgggtttccgtactgggg | | | | |
| | acaaggaactctggttacagtcagtagcggtggtg | | | | |
| | gaggtagtggtggaggcggatctggcggcggcggt | | | | |
| | tcaggaggtggtggatccgatgtccagcttaccca | | | | |
| | aagtccgagcacgttgagtgcaagtgtaggagacc | | | | |
| | gcgtaacgattacttgctctgcttcaagttccgta | | | | |
| | tcctacatgaattggtatcagcaaaagcctggaaa | | | | |
| | agcccctaagcgctggatatacgattcaagtaagt | | | | |
| | tggcttctggcgtcccagcacggttttctggttca | | | | |
| | ggttccggtacagaatatacgctgacaatcagctc | | | | |
| | tctccaaccggatgatttcgcaacctattactgtc | | | | |
| | aacaatggtcaagaaatccgccgacattcgggcag | | | | |
| | ggaacaaaagtcgaggtaaaaaggtca | | | | |
| TRI | gacatcgtgatgacccagtctccagactccctggc | | 17 | DIVMTQSPDSLAVSLGERATI | 18 |
| 01046 | tgtgtctctgggcgagagggccaccatcaactgca | | | NCKSSHSVLYSSNNKNYLAW | |
| Anti TA | agtccagccacagtgttttatacagctccaacaat | | | YQQKPGQPPKLLIYWASTRE | |
| scFv x | aagaactacttagcttggtaccagcagaaaccagg | | | SGVPDRFSGSGSGTDFTLTIS | |
| Anti | acagcctcctaagctgctcatttactgggcatcta | | | SLQAEDVAVYYCQQYYSTPP | |
| CD3 scFv | cccgggaatccggggtccctgaccgattcagtggc | | | TTFGGGTKVEIKGGGGSGGG | |
| ADAPTIR ^{™} | agcgggtctgggacagatttcactctcaccatcag | | | GSGGGGSGGGGSEVQLLES | |
| | cagcctgcaggctgaagatgtggcagtttattact | | | GGGLVQPGGSLRLSCAASGF | |
| | gtcagcaatattatagtactcctccgaccactttc | | | TFSSYGMSWVRQAPGKGLE | |
| | ggcggagggaccaaggtggagatcaaaggtggagg | | | GVSAISGSGGSTYYADSVKG | |
| | cggttcaggcggaggtggatccggcggtggcggct | | | RFTISRDNSKNTLYLQMNSLR | |
| | ccggtggcggcggatctgaggtgcagctgttggag | | | AEDTAVYYCAKEKLRYFDWL | |
| | tctgggggaggcttggtacagcctggggggtccct | | | SDAFDIWGQGTMVTVSSSE | |
| | gagactctcctgtgcagcctctggattcaccttta | | | PKSSDKTHTCPPCPAPPAAA | |
| | gcagctatggcatgagctgggtccgccaggctcca | | | PSVFLFPPKPKDTLMISRTPE | |
| | gggaaggggctggagggggtctcagctattagtgg | | | VTCVVVDVSHEDPEVKFNW | |
| | tagtggtggtagcacatactacgcagactccgtga | | | YVDGVEVHNAKTKPREEQY | |
| | agggccggttcaccatctccagagacaattccaag | | | NSTYRVVSVLTVLHQDWLN | |
| | aacacgctgtatctgcaaatgaacagcctgagagc | | | GKEYKCAVSNKALPAPIEKTIS | |
| | cgaggacacggccgtatattactgtgcgaaagaaa | | | KAKGQPREPQVYTLPPSRDE | |
| | agttacgatattttgactggttatccgatgctttt | | | LTKNQVSLTCLVKGFYPSDIA | |
| | gatatctggggccaagggacaatggtcaccgtctc | | | VEWESNGQPENNYKTTPPV | |
| | ctcgagtgagcccaaatcttctgacaaaactcaca | | | LDSDGSFFLYSKLTVDKSRW | |
| | catgcccaccgtgcccagcacctccagccgctgca | | | QQGNVFSCSVMHEALHNHY | |
| | ccgtcagtcttcctcttccccccaaaacccaagga | | | TQKSLSLSPGGGGSPSQVQL | |
| | caccctcatgatctcccggacccctgaggtcacat | | | VQSGAEVKKPGASVKVSCKA | |
| | gcgtggtggtggacgtgagccacgaagaccctgag | | | SGYTFTRSTMHWVRQAPGQ | |
| | gtcaagttcaactggtacgtggacggcgtggaggt | | | GLEWIGYINPSSAYTNYAQKF | |
| | gcataatgccaagacaaagccgcgggaggagcagt | | | QGRVTLTADKSTSTAYMELS | |
| | acaacagcacgtaccgtgtggtcagcgtcctcacc | | | SLRSEDTAVYYCASPQVHYD | |
| | gtcctgcaccaggactggctgaatggcaaggaata | | | YNGFPYWGQGTLVTVSSGG | |
| | caagtgcgcggtctccaacaaagccctcccagccc | | | GGSGGGGSGGGGSGGGGS | |
| | ccatcgagaaaaccatctccaaagccaaagggcag | | | DVQLTQSPSTLSASVGDRVTI | |
| | ccccgagaaccacaggtgtacaccctgcccccatc | | | TCSASSSVSYMNWYQQKPG | |
| | ccgggatgagctgaccaagaaccaggtcagcctga | | | KAPKRWIYDSSKLASGVPAR | |
| | cctgcctggtcaaaggcttctatccaagcgacatc | | | FSGSGSGTEYTLTISSLQPDDF | |
| | gccgtggagtgggagagcaatgggcagccggagaa | | | ATYYCQQWSRNPPTFGQGT | |
| | caactacaagaccacgcctcccgtgctggactccg | | | KVEVKRS | |
| | acggctccttcttcctctacagcaagctcaccgtg | | | | |
| | gacaagagcaggtggcagcaggggaacgtcttctc | | | | |
| | atgctccgtgatgcatgaggctctgcacaaccact | | | | |
| | acacgcagaagagcctctccctgtctccgggcggc | | | | |
| | gggggatccccgtcacaagtacaactcgttcaaag | | | | |
| | tggcgcagaagtaaagaagccaggcgccagtgtta | | | | |
| | aggtgagctgcaaggcaagcgggtacaccttcacc | | | | |
| | cggtctacaatgcactgggtaagacaagcaccagg | | | | |
| | gcaaggactcgaatggattggttacatcaaccctt | | | | |
| | cctctgcatacaccaactacgctcaaaagttccag | | | | |
| | ggccgcgttactttgacagcggataaatctacatc | | | | |
| | cacggcctatatggaactgtcaagcctcaggagcg | | | | |
| | aggacacagcggtatattactgtgcatctccccag | | | | |
| | gtccattatgactacaacgggtttccgtactgggg | | | | |
| | acaaggaactctggttacagtcagtagcggtggtg | | | | |
| | gaggtagtggtggaggcggatctggcggcggcggt | | | | |
| | tcaggaggtggtggatccgatgtccagcttaccca | | | | |
| | aagtccgagcacgttgagtgcaagtgtaggagacc | | | | |
| | gcgtaacgattacttgctctgcttcaagttccgta | | | | |
| | tcctacatgaattggtatcagcaaaagcctggaaa | | | | |
| | agcccctaagcgctggatatacgattcaagtaagt | | | | |
| | tggcttctggcgtcccagcacggttttctggttca | | | | |
| | ggttccggtacagaatatacgctgacaatcagctc | | | | |
| | tctccaaccggatgatttcgcaacctattactgtc | | | | |
| | aacaatggtcaagaaatccgccgacattcgggcag | | | | |
| | ggaacaaaagtcgaggtaaaaaggtca | | | | |
| PSMA | gatatccagatgacccagtctccatccgccatgtc | | 19 | DIQMTQSPSAMSASVGDRV | 20 |
| 01012 | tgcatctgtaggagacagagtcaccatcacttgcc | | | TITCRASKSISKYLAWFQQKP | |
| Anti- | gggcgagtaagagcattagcaaatatttagcctgg | | | GKVPKLRIHSGSTLQSGVPSR | |
| PSMA | tttcagcagaaaccagggaaagttcctaagctccg | | | FSGSGSGTEFTLTISSLQPEDF | |
| scFv-Fc | catccattctggatctactttgcaatcaggggtcc | | | ATYYCQQHIEYPWTFGQGTK | |
| | catctcggttcagtggcagtggatctgggacagaa | | | VEIKRGGGGSGGGGSGGGG | |
| | tttactctcaccatcagcagcctgcagcctgaaga | | | SQVQLVQSGAEVKKPGASVK | |
| | ttttgcaacttattactgtcaacagcatattgaat | | | VSCKASGYTFTDYYMHWVR | |
| | acccgtggacgttcggccaagggaccaaggtggaa | | | QAPGQGLEWMGYFNPYND | |
| | atcaaacgaggtggcggagggtctgggggtggcgg | | | YTRYAQKFQGRVTMTRDTSI | |
| | atccggaggtggtggctctcaggtccagctggtac | | | STAYMELSSLRSDDTAVYYCA | |
| | agtctggggctgaggtgaagaagcctggggcttca | | | RSDGYYDAMDYWGQGTTV | |
| | gtgaaggtctcctgcaaggcttctggatacacatt | | | TVSSSEPKSSDKTHTCPPCPA | |
| | cactgactactacatgcactgggtgcgacaggccc | | | PEAAGAPSVFLFPPKPKDTL | |
| | ctggacaagggcttgagtggatgggatattttaat | | | MISRTPEVTCVVVDVSHEDP | |
| | ccttataatgattatactagatacgcacagaagtt | | | EVKFNWYVDGVEVHNAKTK | |
| | ccagggcagagtcaccatgaccagggacacgtcta | | | PREEQYNSTYRVVSVLTVLH | |
| | tcagcacagcctacatggagctgagcagcctgaga | | | QDWLNGKEYKCAVSNKALP | |
| | tctgacgacacggccgtgtattactgtgcaagatc | | | APIEKTISKAKGQPREPQVYT | |
| | ggatggttactacgatgctatggactactggggtc | | | LPPSRDELTKNQVSLTCLVKG | |
| | aaggaaccacagtcaccgtctcctcgagtgagccc | | | FYPSDIAVEWESNGQPENNY | |
| | aaatcttctgacaaaactcacacatgcccaccgtg | | | KTTPPVLDSDGSFFLYSKLTV | |
| | cccagcacctgaagccgcgggtgcaccgtcagtct | | | DKSRWQQGNVFSCSVMHE | |
| | tcctcttccccccaaaacccaaggacaccctcatg | | | ALHNHYTQKSLSLSPGK | |
| | atctcccggacccctgaggtcacatgcgtggtggt | | | | |
| | ggacgtgagccacgaagaccctgaggtcaagttca | | | | |
| | actggtacgtggacggcgtggaggtgcataatgcc | | | | |
| | aagacaaagccgcgggaggagcagtacaacagcac | | | | |
| | gtaccgtgtggtcagcgtcctcaccgtcctgcacc | | | | |
| | aggactggctgaatggcaaggaatacaagtgcgcg | | | | |
| | gtctccaacaaagccctcccagcccccatcgagaa | | | | |
| | aaccatctccaaagccaaagggcagccccgagaac | | | | |
| | cacaggtgtacaccctgcccccatcccgggatgag | | | | |
| | ctgaccaagaaccaggtcagcctgacctgcctggt | | | | |
| | caaaggcttctatccaagcgacatcgccgtggagt | | | | |
| | gggagagcaatgggcagccggagaacaactacaag | | | | |
| | accacgcctcccgtgctggactccgacggctcctt | | | | |
| | cttcctctacagcaagctcaccgtggacaagagca | | | | |
| | ggtggcagcaggggaacgtcttctcatgctccgtg | | | | |
| | atgcatgaggctctgcacaaccactacacgcagaa | | | | |
| | gagcctctccctgtctccgggtaaa | | | | |
| PSMA01 | caggtccagctggtacagtctggggctgaggtgaa | | 113 | QVQLVQSGAEVKKPGASVK | 114 |
| 012 | gaagcctggggcttcagtgaaggtctcctgcaagg | | | VSCKASGYTFTDYYMHWVR | |
| VH | cttctggatacacattcactgactactacatgcac | | | QAPGQGLEWMGYFNPYND | |
| domain | tgggtgcgacaggcccctggacaagggcttgagtg | | | YTRYAQKFQGRVTMTRDTSI | |
| | gatgggatattttaatccttataatgattatacta | | | STAYMELSSLRSDDTAVYYCA | |
| | gatacgcacagaagttccagggcagagtcaccatg | | | RSDGYYDAMDYWGQGTTV | |
| | accagggacacgtctatcagcacagcctacatgga | | | TVSS | |
| | gctgagcagcctgagatctgacgacacggccgtgt | | | | |
| | attactgtgcaagatcggatggttactacgatgct | | | | |
| | atggactactggggtcaaggaaccacagtcaccgt | | | | |
| | ctcctcg | | | | |
| PSMA01 | gatatccagatgacccagtctccatccgccatgtc | | 115 | DIQMTQSPSAMSASVGDRV | 116 |
| 012 | tgcatctgtaggagacagagtcaccatcacttgcc | | | TITCRASKSISKYLAWFQQKP | |
| VL | gggcgagtaagagcattagcaaatatttagcctgg | | | GKVPKLRIHSGSTLQSGVPSR | |
| domain | tttcagcagaaaccagggaaagttcctaagctccg | | | FSGSGSGTEFTLTISSLQPEDF | |
| | catccattctggatctactttgcaatcaggggtcc | | | ATYYCQQHIEYPWTFGQGTK | |
| | catctcggttcagtggcagtggatctgggacagaa | | | VEIKR | |
| | tttactctcaccatcagcagcctgcagcctgaaga | | | | |
| | ttttgcaacttattactgtcaacagcatattgaat | | | | |
| | acccgtggacgttcggccaagggaccaaggtggaa | | | | |
| | atcaaacga | | | | |
| PSMA | gacgtacagattactcaaagcccttcaaccctgtc | | 21 | DVQITQSPSTLSASVGDRVTI | 22 |
| 01019 | cgcaagtgtcggggacagagttactataacgtgca | | | TCRASKSISKYLAWYQQKPG | |
| Anti- | gggcaagtaagagtataagtaaatatctggcctgg | | | KAPKLLIHSGSSLESGVPSRFS | |
| PSMA | tatcaacagaaacccgggaaagcccctaaactcct | | | GSGSGTEFTLTISSLQPDDFA | |
| scFv-Fc | catacattcaggatctagccttgagtcaggtgttc | | | TYYCQQHIEYPWTFGQGTKV | |
| | cgtcaagattctctggttccggaagcgggaccgag | | | EIKGGGGSGGGGSGGGGSG | |
| | ttcaccctgacaatcagttcactccagcctgacga | | | GGGSEVQLLESGGGLVQPG | |
| | cttcgctacttactactgccagcagcacatagagt | | | GSLRLSCAASGYTFTDYYIHW | |
| | acccctggacattcggacaaggaacgaaagtcgaa | | | VRQAPGKGLEWIGSFNPYN | |
| | atcaagggtggtggaggtagtggtggaggcggatc | | | DYTSYADSVKGRATLSVDKS | |
| | tggcggcggcggttcaggaggtggtggatccgagg | | | KNTAYLQMNSLRAEDTAVYY | |
| | tgcaacttctcgaatccggcggtgggctggtacaa | | | CARSDGYYDAMDYWGQGT | |
| | ccgggaggatcccttcgcctctcttgtgctgcctc | | | LVTVSSSEPKSSDKTHTCPPC | |
| | cgggtataccttcacggactactatattcactggg | | | PAPEAAGAPSVFLFPPKPKDT | |
| | tgcgccaggcgccaggaaagggattggaatggata | | | LMISRTPEVTCVVVDVSHED | |
| | gggagctttaacccctacaacgactacacttctta | | | PEVKFNWYVDGVEVHNAKT | |
| | cgccgactctgtaaaggggagagctactctgtccg | | | KPREEQYNSTYRVVSVLTVLH | |
| | tcgataaatcaaagaatacggcataccttcaaatg | | | QDWLNGKEYKCAVSNKALP | |
| | aactccctgagggctgaggataccgcagtgtacta | | | APIEKTISKAKGQPREPQVYT | |
| | ttgtgcaaggagtgatggttactacgacgcgatgg | | | LPPSRDELTKNQVSLTCLVKG | |
| | actactggggccaaggcaccctggtcacagtctcc | | | FYPSDIAVEWESNGQPENNY | |
| | tcgagtgagcccaaatcttctgacaaaactcacac | | | KTTPPVLDSDGSFFLYSKLTV | |
| | atgcccaccgtgcccagcacctgaagccgcgggtg | | | DKSRWQQGNVFSCSVMHE | |
| | caccgtcagtcttcctcttccccccaaaacccaag | | | ALHNHYTQKSLSLSPGK | |
| | gacaccctcatgatctcccggacccctgaggtcac | | | | |
| | atgcgtggtggtggacgtgagccacgaagaccctg | | | | |
| | aggtcaagttcaactggtacgtggacggcgtggag | | | | |
| | gtgcataatgccaagacaaagccgcgggaggagca | | | | |
| | gtacaacagcacgtaccgtgtggtcagcgtcctca | | | | |
| | ccgtcctgcaccaggactggctgaatggcaaggaa | | | | |
| | tacaagtgcgcggtctccaacaaagccctcccagc | | | | |
| | ccccatcgagaaaaccatctccaaagccaaagggc | | | | |
| | agccccgagaaccacaggtgtacaccctgccccca | | | | |
| | tcccgggatgagctgaccaagaaccaggtcagcct | | | | |
| | gacctgcctggtcaaaggcttctatccaagcgaca | | | | |
| | tcgccgtggagtgggagagcaatgggcagccggag | | | | |
| | aacaactacaagaccacgcctcccgtgctggactc | | | | |
| | cgacggctccttcttcctctacagcaagctcaccg | | | | |
| | tggacaagagcaggtggcagcaggggaacgtcttc | | | | |
| | tcatgctccgtgatgcatgaggctctgcacaacca | | | | |
| | ctacacgcagaagagcctctccctgtctccgggta | | | | |
| | aa | | | | |
| PSMA | gacatccagatgactcaaagcccttcaaccctgtc | | 23 | DIQMTQSPSTLSASVGDRVTI | 24 |
| 01020 | cgcaagtgtcggggacagagttactataacgtgca | | | TCRASKSISKYLAWYQQKPG | |
| Anti- | gggcaagtaagagtataagtaaatatctggcctgg | | | KAPKLLIHSGSSLESGVPSRFS | |
| PSMA | tatcaacagaaacccgggaaagcccctaaactcct | | | GSGSGTEFTLTISSLQPDDFA | |
| scFv-Fc | catacattcaggatctagccttgagtcaggtgttc | | | TYYCQQHIEYPWTFGQGTKV | |
| | cgtcaagattctctggttccggaagcgggaccgag | | | EIKGGGGSGGGGSGGGGSG | |
| | ttcaccctgacaatcagttcactccagcctgacga | | | GGGSEVQLLESGGGLVQPG | |
| | cttcgctacttactactgccagcagcacatagagt | | | GSLRLSCAASGYTFTDYYIHW | |
| | acccctggacattcggacaaggaacgaaagtcgaa | | | VRQAPGKGLEWIGSFNPYN | |
| | atcaagggtggtggaggtagtggtggaggcggatc | | | DYTSYADSVKGRATLSVDKS | |
| | tggcggcggcggttcaggaggtggtggatccgagg | | | KNTAYLQMNSLRAEDTAVYY | |
| | tgcaacttctcgaatccggcggtgggctggtacaa | | | CARSDGYYDAMDYWGQGT | |
| | ccgggaggatcccttcgcctctcttgtgctgcctc | | | LVTVSSSEPKSSDKTHTCPPC | |
| | cgggtataccttcacggactactatattcactggg | | | PAPEAAGAPSVFLFPPKPKDT | |
| | tgcgccaggcgccaggaaagggattggaatggata | | | LMISRTPEVTCVVVDVSHED | |
| | gggagctttaacccctacaacgactacacttctta | | | PEVKFNWYVDGVEVHNAKT | |
| | cgccgactctgtaaaggggagagctactctgtccg | | | KPREEQYNSTYRVVSVLTVLH | |
| | tcgataaatcaaagaatacggcataccttcaaatg | | | QDWLNGKEYKCAVSNKALP | |
| | aactccctgagggctgaggataccgcagtgtacta | | | APIEKTISKAKGQPREPQVYT | |
| | ttgtgcaaggagtgatggttactacgacgcgatgg | | | LPPSRDELTKNQVSLTCLVKG | |
| | actactggggccaaggcaccctggtcacagtctcc | | | FYPSDIAVEWESNGQPENNY | |
| | tcgagtgagcccaaatcttctgacaaaactcacac | | | KTTPPVLDSDGSFFLYSKLTV | |
| | atgcccaccgtgcccagcacctgaagccgcgggtg | | | DKSRWQQGNVFSCSVMHE | |
| | caccgtcagtcttcctcttccccccaaaacccaag | | | ALHNHYTQKSLSLSPGK | |
| | gacaccctcatgatctcccggacccctgaggtcac | | | | |
| | atgcgtggtggtggacgtgagccacgaagaccctg | | | | |
| | aggtcaagttcaactggtacgtggacggcgtggag | | | | |
| | gtgcataatgccaagacaaagccgcgggaggagca | | | | |
| | gtacaacagcacgtaccgtgtggtcagcgtcctca | | | | |
| | ccgtcctgcaccaggactggctgaatggcaaggaa | | | | |
| | tacaagtgcgcggtctccaacaaagccctcccagc | | | | |
| | ccccatcgagaaaaccatctccaaagccaaagggc | | | | |
| | agccccgagaaccacaggtgtacaccctgccccca | | | | |
| | tcccgggatgagctgaccaagaaccaggtcagcct | | | | |
| | gacctgcctggtcaaaggcttctatccaagcgaca | | | | |
| | tcgccgtggagtgggagagcaatgggcagccggag | | | | |
| | aacaactacaagaccacgcctcccgtgctggactc | | | | |
| | cgacggctccttcttcctctacagcaagctcaccg | | | | |
| | tggacaagagcaggtggcagcaggggaacgtcttc | | | | |
| | tcatgctccgtgatgcatgaggctctgcacaacca | | | | |
| | ctacacgcagaagagcctctccctgtctccgggta | | | | |
| | aa | | | | |
| PSMA | gacgtacagattactcaaagcccttcaaccctgtc | | 25 | DVQITQSPSTLSASVGDRVTI | 26 |
| 01021 | cgcaagtgtcggggacagagttactataacgtgca | | | TCRASKSISKYLAWYQQKPG | |
| Anti- | gggcaagtaagagtataagtaaatatctggcctgg | | | KAPKLLIHSGSSLESGVPSRFS | |
| PSMA | tatcaacagaaacccgggaaagcccctaaactcct | | | GSGSGTEFTLTISSLQPDDFA | |
| scFv-Fc | catacattcaggatctagccttgagtcaggtgttc | | | TYYCQQHIEYPWTFGQGTKV | |
| | cgtcaagattctctggttccggaagcgggaccgag | | | EIKGGGGSGGGGSGGGGSG | |
| | ttcaccctgacaatcagttcactccagcctgacga | | | GGGSQVQLVQSGAEVKKPG | |
| | cttcgctacttactactgccagcagcacatagagt | | | ASVKVSCKASGYTFTDYYMH | |
| | acccctggacattcggacaaggaacgaaagtcgaa | | | WVRQAPGQGLEWMGYFN | |
| | atcaagggtggtggaggtagtggtggaggcggatc | | | PYNDYTRYAQKFQGRVTMT | |
| | tggcggcggcggttcaggaggtggtggatcccagg | | | RDTSISTAYMELSSLRSDDTA | |
| | tccagctggtacagtctggggctgaggtgaagaag | | | VYYCARSDGYYDAMDYWG | |
| | cctggggcttcagtgaaggtctcctgcaaggcttc | | | QGTTVTVSSSEPKSSDKTHTC | |
| | tggatacacattcactgactactacatgcactggg | | | PPCPAPEAAGAPSVFLFPPKP | |
| | tgcgacaggcccctggacaagggcttgagtggatg | | | KDTLMISRTPEVTCVVVDVS | |
| | ggatattttaatccttataatgattatactagata | | | HEDPEVKFNWYVDGVEVHN | |
| | cgcacagaagttccagggcagagtcaccatgacca | | | AKTKPREEQYNSTYRVVSVLT | |
| | gggacacgtctatcagcacagcctacatggagctg | | | VLHQDWLNGKEYKCAVSNK | |
| | agcagcctgagatctgacgacacggccgtgtatta | | | ALPAPIEKTISKAKGQPREPQ | |
| | ctgtgcaagatcggatggttactacgatgctatgg | | | VYTLPPSRDELTKNQVSLTCL | |
| | actactggggtcaaggaaccacagtcaccgtctcc | | | VKGFYPSDIAVEWESNGQPE | |
| | tcgagtgagcccaaatcttctgacaaaactcacac | | | NNYKTTPPVLDSDGSFFLYSK | |
| | atgcccaccgtgcccagcacctgaagccgcgggtg | | | LTVDKSRWQQGNVFSCSVM | |
| | caccgtcagtcttcctcttccccccaaaacccaag | | | HEALHNHYTQKSLSLSPGK | |
| | gacaccctcatgatctcccggacccctgaggtcac | | | | |
| | atgcgtggtggtggacgtgagccacgaagaccctg | | | | |
| | aggtcaagttcaactggtacgtggacggcgtggag | | | | |
| | gtgcataatgccaagacaaagccgcgggaggagca | | | | |
| | gtacaacagcacgtaccgtgtggtcagcgtcctca | | | | |
| | ccgtcctgcaccaggactggctgaatggcaaggaa | | | | |
| | tacaagtgcgcggtctccaacaaagccctcccagc | | | | |
| | ccccatcgagaaaaccatctccaaagccaaagggc | | | | |
| | agccccgagaaccacaggtgtacaccctgccccca | | | | |
| | tcccgggatgagctgaccaagaaccaggtcagcct | | | | |
| | gacctgcctggtcaaaggcttctatccaagcgaca | | | | |
| | tcgccgtggagtgggagagcaatgggcagccggag | | | | |
| | aacaactacaagaccacgcctcccgtgctggactc | | | | |
| | cgacggctccttcttcctctacagcaagctcaccg | | | | |
| | tggacaagagcaggtggcagcaggggaacgtcttc | | | | |
| | tcatgctccgtgatgcatgaggctctgcacaacca | | | | |
| | ctacacgcagaagagcctctccctgtctccgggta | | | | |
| | aa | | | | |
| PSMA | gacgtacagattactcaaagcccttcaaccctgtc | | 27 | DVQITQSPSTLSASVGDRVTI | 28 |
| 01022 | cgcaagtgtcggggacagagttactataacgtgca | | | TCRASKSISKYLAWYQQKPG | |
| Anti | gggcaagtaagagtataagtaaatatctggcctgg | | | KAPKLLIHSGSSLESGVPSRFS | |
| PSMA | tatcaacagaaacccgggaaagcccctaaactcct | | | GSGSGTEFTLTISSLQPDDFA | |
| scFv-Fc | catacattcaggatctagccttgagtcaggtgttc | | | TYYCQQHIEYPWTFGQGTKV | |
| | cgtcaagattctctggttccggaagcgggaccgag | | | EIKGGGGSGGGGSGGGGSG | |
| | ttcaccctgacaatcagttcactccagcctgacga | | | GGGSQVQLVQSGAEVKKPG | |
| | cttcgctacttactactgccagcagcacatagagt | | | ASVKVSCKASGYTFTDYYMH | |
| | acccctggacattcggacaaggaacgaaagtcgaa | | | WVRQAPGQGLEWMGYFN | |
| | atcaagggtggtggaggtagtggtggaggcggatc | | | PYNDYTRYAQKFQGRVTMT | |
| | tggcggcggcggttcaggaggtggtggatcccagg | | | RDTSTSTVYMELSSLRSEDTA | |
| | tccagctggtacagtctggggctgaggtgaagaag | | | VYYCARSDGYYDAMDYWG | |
| | cctggggcttcagtgaaggtctcctgcaaggcttc | | | QGTTVTVSSSEPKSSDKTHTC | |
| | tggatacacattcactgactactacatgcactggg | | | PPCPAPEAAGAPSVFLFPPKP | |
| | tgcgacaggcccctggacaagggcttgagtggatg | | | KDTLMISRTPEVTCVVVDVS | |
| | ggatattttaatccttataatgattatactagata | | | HEDPEVKFNWYVDGVEVHN | |
| | cgcacagaagttccagggcagagtcaccatgacca | | | AKTKPREEQYNSTYRVVSVLT | |
| | gggacacgtctaccagcacagtgtacatggagctg | | | VLHQDWLNGKEYKCAVSNK | |
| | agcagcctgagatctgaggacacggccgtgtatta | | | ALPAPIEKTISKAKGQPREPQ | |
| | ctgtgcaagatcggatggttactacgatgctatgg | | | VYTLPPSRDELTKNQVSLTCL | |
| | actactggggtcaaggaaccacagtcaccgtctcc | | | VKGFYPSDIAVEWESNGQPE | |
| | tcgagtgagcccaaatcttctgacaaaactcacac | | | NNYKTTPPVLDSDGSFFLYSK | |
| | atgcccaccgtgcccagcacctgaagccgcgggtg | | | LTVDKSRWQQGNVFSCSVM | |
| | caccgtcagtcttcctcttccccccaaaacccaag | | | HEALHNHYTQKSLSLSPGK | |
| | gacaccctcatgatctcccggacccctgaggtcac | | | | |
| | atgcgtggtggtggacgtgagccacgaagaccctg | | | | |
| | aggtcaagttcaactggtacgtggacggcgtggag | | | | |
| | gtgcataatgccaagacaaagccgcgggaggagca | | | | |
| | gtacaacagcacgtaccgtgtggtcagcgtcctca | | | | |
| | ccgtcctgcaccaggactggctgaatggcaaggaa | | | | |
| | tacaagtgcgcggtctccaacaaagccctcccagc | | | | |
| | ccccatcgagaaaaccatctccaaagccaaagggc | | | | |
| | agccccgagaaccacaggtgtacaccctgccccca | | | | |
| | tcccgggatgagctgaccaagaaccaggtcagcct | | | | |
| | gacctgcctggtcaaaggcttctatccaagcgaca | | | | |
| | tcgccgtggagtgggagagcaatgggcagccggag | | | | |
| | aacaactacaagaccacgcctcccgtgctggactc | | | | |
| | cgacggctccttcttcctctacagcaagctcaccg | | | | |
| | tggacaagagcaggtggcagcaggggaacgtcttc | | | | |
| | tcatgctccgtgatgcatgaggctctgcacaacca | | | | |
| | ctacacgcagaagagcctctccctgtctccgggta | | | | |
| | aa | | | | |
| PSMA | gacatccagatgactcaaagcccttcaaccctgtc | | 29 | DIQMTQSPSTLSASVGDRVTI | 30 |
| 01023 | cgcaagtgtcggggacagagttactataacgtgca | | | TCRASKSISKYLAWYQQKPG | |
| Anti | gggcaagtaagagtataagtaaatatctggcctgg | | | KAPKLLIHSGSSLESGVPSRFS | |
| PSMA | tatcaacagaaacccgggaaagcccctaaactcct | | | GSGSGTEFTLTISSLQPDDFA | |
| scFv-Fc | catacattcaggatctagccttgagtcaggtgttc | | | TYYCQQHIEYPWTFGQGTKV | |
| | cgtcaagattctctggttccggaagcgggaccgag | | | EIKGGGGSGGGGSGGGGSG | |
| | ttcaccctgacaatcagttcactccagcctgacga | | | GGGSQVQLVQSGAEVKKPG | |
| | cttcgctacttactactgccagcagcacatagagt | | | ASVKVSCKASGYTFTDYYMH | |
| | acccctggacattcggacaaggaacgaaagtcgaa | | | WVRQAPGQGLEWMGYFN | |
| | atcaagggtggtggaggtagtggtggaggcggatc | | | PYNDYTRYAQKFQGRVTMT | |
| | tggcggcggcggttcaggaggtggtggatcccagg | | | RDTSTSTVYMELSSLRSEDTA | |
| | tccagctggtacagtctggggctgaggtgaagaag | | | VYYCARSDGYYDAMDYWG | |
| | cctggggcttcagtgaaggtctcctgcaaggcttc | | | QGTTVTVSSSEPKSSDKTHTC | |
| | tggatacacattcactgactactacatgcactggg | | | PPCPAPEAAGAPSVFLFPPKP | |
| | tgcgacaggcccctggacaagggcttgagtggatg | | | KDTLMISRTPEVTCVVVDVS | |
| | ggatattttaatccttataatgattatactagata | | | HEDPEVKFNWYVDGVEVHN | |
| | cgcacagaagttccagggcagagtcaccatgacca | | | AKTKPREEQYNSTYRVVSVLT | |
| | gggacacgtctaccagcacagtgtacatggagctg | | | VLHQDWLNGKEYKCAVSNK | |
| | agcagcctgagatctgaggacacggccgtgtatta | | | ALPAPIEKTISKAKGQPREPQ | |
| | ctgtgcaagatcggatggttactacgatgctatgg | | | VYTLPPSRDELTKNQVSLTCL | |
| | actactggggtcaaggaaccacagtcaccgtctcc | | | VKGFYPSDIAVEWESNGQPE | |
| | tcgagtgagcccaaatcttctgacaaaactcacac | | | NNYKTTPPVLDSDGSFFLYSK | |
| | atgcccaccgtgcccagcacctgaagccgcgggtg | | | LTVDKSRWQQGNVFSCSVM | |
| | caccgtcagtcttcctcttccccccaaaacccaag | | | HEALHNHYTQKSLSLSPGK | |
| | gacaccctcatgatctcccggacccctgaggtcac | | | | |
| | atgcgtggtggtggacgtgagccacgaagaccctg | | | | |
| | aggtcaagttcaactggtacgtggacggcgtggag | | | | |
| | gtgcataatgccaagacaaagccgcgggaggagca | | | | |
| | gtacaacagcacgtaccgtgtggtcagcgtcctca | | | | |
| | ccgtcctgcaccaggactggctgaatggcaaggaa | | | | |
| | tacaagtgcgcggtctccaacaaagccctcccagc | | | | |
| | ccccatcgagaaaaccatctccaaagccaaagggc | | | | |
| | agccccgagaaccacaggtgtacaccctgccccca | | | | |
| | tcccgggatgagctgaccaagaaccaggtcagcct | | | | |
| | gacctgcctggtcaaaggcttctatccaagcgaca | | | | |
| | tcgccgtggagtgggagagcaatgggcagccggag | | | | |
| | aacaactacaagaccacgcctcccgtgctggactc | | | | |
| | cgacggctccttcttcctctacagcaagctcaccg | | | | |
| | tggacaagagcaggtggcagcaggggaacgtcttc | | | | |
| | tcatgctccgtgatgcatgaggctctgcacaacca | | | | |
| | ctacacgcagaagagcctctccctgtctccgggta | | | | |
| | aa | | | | |
| PSMA | gacgtacagattactcaaagcccttcaaccctgtc | | 31 | DVQITQSPSTLSASVGDRVTI | 32 |
| 01024 | cgcaagtgtcggggacagagttactataacgtgca | | | TCRASKSISKYLAWYQQKPG | |
| Anti- | gggcaagtaagagtataagtaaatatctggcctgg | | | KAPKLLIHSGSSLESGVPSRFS | |
| PSMA | tatcaacagaaacccgggaaagcccctaaactcct | | | GSGSGTEFTLTISSLQPDDFA | |
| scFv-Fc | catacattcaggatctagccttgagtcaggtgttc | | | TYYCQQHIEYPWTFGQGTKV | |
| | cgtcaagattctctggttccggaagcgggaccgag | | | EIKGGGGGGGGSGGGGSG | |
| | ttcaccctgacaatcagttcactccagcctgacga | | | GGGSQVQLVQSGAEVKKPG | |
| | cttcgctacttactactgccagcagcacatagagt | | | ASVKVSCKASGYTFTDYYMH | |
| | acccctggacattcggacaaggaacgaaagtcgaa | | | WVRQAPGQGLEWMGIFNP | |
| | atcaagggtggtggaggtagtggtggaggcggatc | | | YNDYTSYAQKFQGRVTMTR | |
| | tggcggcggcggttcaggaggtggtggatcccagg | | | DTSTSTVYMELSSLRSEDTAV | |
| | tccagctggtacagtctggggctgaggtgaagaag | | | YYCARSDGYYDAMDYWGQ | |
| | cctggggcttcagtgaaggtctcctgcaaggcttc | | | GTTVTVSSSEPKSSDKTHTCP | |
| | tggatacacattcactgactactacatgcactggg | | | PCPAPEAAGAPSVFLFPPKPK | |
| | tgcgacaggcccctggacaagggcttgagtggatg | | | DTLMISRTPEVTCVVVDVSH | |
| | ggaatttttaatccttataatgattatactagtta | | | EDPEVKFNWYVDGVEVHNA | |
| | cgcacagaagttccagggcagagtcaccatgacca | | | KTKPREEQYNSTYRVVSVLTV | |
| | gggacacgtctaccagcacagtgtacatggagctg | | | LHQDWLNGKEYKCAVSNKA | |
| | agcagcctgagatctgaggacacggccgtgtatta | | | LPAPIEKTISKAKGQPREPQV | |
| | ctgtgcaagatcggatggttactacgatgctatgg | | | YTLPPSRDELTKNQVSLTCLV | |
| | actactggggtcaaggaaccacagtcaccgtctcc | | | KGFYPSDIAVEWESNGQPEN | |
| | tcgagtgagcccaaatcttctgacaaaactcacac | | | NYKTTPPVLDSDGSFFLYSKL | |
| | atgcccaccgtgcccagcacctgaagccgcgggtg | | | TVDKSRWQQGNVFSCSVM | |
| | caccgtcagtcttcctcttccccccaaaacccaag | | | HEALHNHYTQKSLSLSPGK | |
| | gacaccctcatgatctcccggacccctgaggtcac | | | | |
| | atgcgtggtggtggacgtgagccacgaagaccctg | | | | |
| | aggtcaagttcaactggtacgtggacggcgtggag | | | | |
| | gtgcataatgccaagacaaagccgcgggaggagca | | | | |
| | gtacaacagcacgtaccgtgtggtcagcgtcctca | | | | |
| | ccgtcctgcaccaggactggctgaatggcaaggaa | | | | |
| | tacaagtgcgcggtctccaacaaagccctcccagc | | | | |
| | ccccatcgagaaaaccatctccaaagccaaagggc | | | | |
| | agccccgagaaccacaggtgtacaccctgccccca | | | | |
| | tcccgggatgagctgaccaagaaccaggtcagcct | | | | |
| | gacctgcctggtcaaaggcttctatccaagcgaca | | | | |
| | tcgccgtggagtgggagagcaatgggcagccggag | | | | |
| | aacaactacaagaccacgcctcccgtgctggactc | | | | |
| | cgacggctccttcttcctctacagcaagctcaccg | | | | |
| | tggacaagagcaggtggcagcaggggaacgtcttc | | | | |
| | tcatgctccgtgatgcatgaggctctgcacaacca | | | | |
| | ctacacgcagaagagcctctccctgtctccgggta | | | | |
| | aa | | | | |
| PSMA | gacatccagatgactcaaagcccttcaaccctgtc | | 33 | DIQMTQSPSTLSASVGDRVTI | 34 |
| 01025 | cgcaagtgtcggggacagagttactataacgtgca | | | TCRASKSISKYLAWYQQKPG | |
| Anti- | gggcaagtaagagtataagtaaatatctggcctgg | | | KAPKLLIHSGSSLESGVPSRFS | |
| PSMA | tatcaacagaaacccgggaaagcccctaaactcct | | | GSGSGTEFTLTISSLQPDDFA | |
| scFv-Fc | catacattcaggatctagccttgagtcaggtgttc | | | TYYCQQHIEYPWTFGQGTKV | |
| | cgtcaagattctctggttccggaagCgggaccgag | | | EIKGGGGSGGGGSGGGGSG | |
| | ttcaccctgacaatcagttcactccagcctgacga | | | GGGSQVQLVQSGAEVKKPG | |
| | cttcgctacttactactgccagcagcacatagagt | | | ASVKVSCKASGYTFTDYYMH | |
| | acccctggacattcggacaaggaacgaaagtcgaa | | | WVRQAPGQGLEWMGIFNP | |
| | atcaagggtggtggaggtagtggtggaggcggatc | | | YNDYTSYAQKFQGRVTMTR | |
| | tggcggcggcggttcaggaggtggtggatcccagg | | | DTSTSTVYMELSSLRSEDTAV | |
| | tccagctggtacagtctggggctgaggtgaagaag | | | YYCARSDGYYDAMDYWGQ | |
| | cctggggcttcagtgaaggtctcctgcaaggcttc | | | GTTVTVSSSEPKSSDKTHTCP | |
| | tggatacacattcactgactactacatgcactggg | | | PCPAPEAAGAPSVFLFPPKPK | |
| | tgcgacaggcccctggacaagggcttgagtggatg | | | DTLMISRTPEVTCVVVDVSH | |
| | ggaatttttaatccttataatgattatactagtta | | | EDPEVKFNWYVDGVEVHNA | |
| | cgcacagaagttccagggcagagtcaccatgacca | | | KTKPREEQYNSTYRVVSVLTV | |
| | gggacacgtctaccagcacagtgtacatggagctg | | | LHQDWLNGKEYKCAVSNKA | |
| | agcagcctgagatctgaggacacggccgtgtatta | | | LPAPIEKTISKAKGQPREPQV | |
| | ctgtgcaagatcggatggttactacgatgctatgg | | | YTLPPSRDELTKNQVSLTCLV | |
| | actactggggtcaaggaaccacagtcaccgtctcc | | | KGFYPSDIAVEWESNGQPEN | |
| | tcgagtgagcccaaatcttctgacaaaactcacac | | | NYKTTPPVLDSDGSFFLYSKL | |
| | atgcccaccgtgcccagcacctgaagccgcgggtg | | | TVDKSRWQQGNVFSCSVM | |
| | caccgtcagtcttcctcttccccccaaaacccaag | | | HEALHNHYTQKSLSLSPGK | |
| | gacaccctcatgatctcccggacccctgaggtcac | | | | |
| | atgcgtggtggtggacgtgagccacgaagaccctg | | | | |
| | aggtcaagttcaactggtacgtggacggcgtggag | | | | |
| | gtgcataatgccaagacaaagccgcgggaggagca | | | | |
| | gtacaacagcacgtaccgtgtggtcagcgtcctca | | | | |
| | ccgtcctgcaccaggactggctgaatggcaaggaa | | | | |
| | tacaagtgcgcggtctccaacaaagccctcccagc | | | | |
| | ccccatcgagaaaaccatctccaaagccaaagggc | | | | |
| | agccccgagaaccacaggtgtacaccctgccccca | | | | |
| | tcccgggatgagctgaccaagaaccaggtcagcct | | | | |
| | gacctgcctggtcaaaggcttctatccaagcgaca | | | | |
| | tcgccgtggagtgggagagcaatgggcagccggag | | | | |
| | aacaactacaagaccacgcctcccgtgctggactc | | | | |
| | cgacggctccttcttcctctacagcaagctcaccg | | | | |
| | tggacaagagcaggtggcagcaggggaacgtcttc | | | | |
| | tcatgctccgtgatgcatgaggctctgcacaacca | | | | |
| | ctacacgcagaagagcctctccctgtctccgggta | | | | |
| | aa | | | | |
| PSMA | gacatccagatgacccagtctccttccaccctgtc | | 35 | DIQMTQSPSTLSASVGDRVTI | 36 |
| 01036 | tgcatctgtaggagacagagtcaccatcacttgcc | | | TCRASKSISKYLAWYQQKPG | |
| Anti- | gggccagtaagagtattagtaagtacttggcctgg | | | KAPKLLIHSGSSLESGVPSRFS | |
| PSMA | tatcagcagaaaccagggaaagcccctaagctcct | | | GSGSGTEFTLTISSLQPDDFA | |
| scFv-Fc | gatccattctggctccagtttggaaagtggggtcc | | | TYYCQQHIEYPWTFGQGTKV | |
| | catcaaggttcagcggcagtggatctgggacagaa | | | EIKGGGGSGGGGSGGGGSG | |
| | ttcactctcaccatcagcagcctgcagcctgatga | | | GGGSQVQLVQSGAEVKKPG | |
| | ttttgcaacttattactgccaacagcatattgaat | | | ASVKVSCKASGYTFTDYYMH | |
| | atccttggacgttcggccaagggaccaaggtggaa | | | WVRQAPGQGLEWMGYFN | |
| | atcaaaggtggtggaggtagtggtggaggcggatc | | | PYNDYTRYAQKFQGRVTMT | |
| | tggcggcggcggttcaggaggtggtggatcccagg | | | RDTSTSTVYMELSSLRSEDTA | |
| | tgcagctggtgcagtctggggctgaggtgaagaag | | | VYYCARSDGYYDAMDYWG | |
| | cctggggcctcagtgaaggtttcctgcaaggcatc | | | QGTTVTVSSSEPKSSDKTHTC | |
| | tggatacaccttcaccgactactatatgcactggg | | | PPCPAPPAAAPSVFLFPPKPK | |
| | tgcgacaggcccctggacaagggcttgagtggatg | | | DTLMISRTPEVTCVVVDVSH | |
| | ggatatttcaacccttataatgattacacacgcta | | | EDPEVKFNWYVDGVEVHNA | |
| | cgcacagaagttccagggcagagtcaccatgacca | | | KTKPREEQYNSTYRVVSVLTV | |
| | gggacacgtccacgagcacagtctacatggagctg | | | LHQDWLNGKEYKCAVSNKA | |
| | agcagcctgagatctgaggacacggccgtgtatta | | | LPAPIEKTISKAKGQPREPQV | |
| | ctgtgcgagatctgacggctactacgacgctatgg | | | YTLPPSRDELTKNQVSLTCLV | |
| | actactgggggcaagggaccacggtcaccgtctcc | | | KGFYPSDIAVEWESNGQPEN | |
| | tcgagtgagcccaaatcttctgacaaaactcacac | | | NYKTTPPVLDSDGSFFLYSKL | |
| | atgcccaccgtgcccagcacctccagccgctgcac | | | TVDKSRWQQGNVFSCSVM | |
| | cgtcagtcttcctcttccccccaaaacccaaggac | | | HEALHNHYTQKSLSLSPGK | |
| | accctcatgatctcccggacccctgaggtcacatg | | | | |
| | cgtggtggtggacgtgagccacgaagaccctgagg | | | | |
| | tcaagttcaactggtacgtggacggcgtggaggtg | | | | |
| | cataatgccaagacaaagccgcgggaggagcagta | | | | |
| | caacagcacgtaccgtgtggtcagcgtcctcaccg | | | | |
| | tcctgcaccaggactggctgaatggcaaggaatac | | | | |
| | aagtgcgcggtctccaacaaagccctcccagcccc | | | | |
| | catcgagaaaaccatctccaaagccaaagggcagc | | | | |
| | cccgagaaccacaggtgtacaccctgcccccatcc | | | | |
| | cgggatgagctgaccaagaaccaggtcagcctgac | | | | |
| | ctgcctggtcaaaggcttctatccaagcgacatcg | | | | |
| | ccgtggagtgggagagcaatgggcagccggagaac | | | | |
| | aactacaagaccacgcctcccgtgctggactccga | | | | |
| | cggctccttcttcctctacagcaagctcaccgtgg | | | | |
| | acaagagcaggtggcagcaggggaacgtcttctca | | | | |
| | tgctccgtgatgcatgaggctctgcacaaccacta | | | | |
| | cacgcagaagagcctctccctgtctccgggtaaa | | | | |
| PSMA | caggtgcagctggtgcagtctggggctgaggtgaa | | 37 | QVQLVQSGAEVKKPGASVK | 38 |
| 01037 | gaagcctggggcctcagtgaaggtttcctgcaagg | | | VSCKASGYTFTDYYMHWVR | |
| Anti- | catctggatacaccttcaccgactactatatgcac | | | QAPGQGLEWMGYFNPYND | |
| PSMA | tgggtgcgacaggcccctggacaagggcttgagtg | | | YTRYAQKFQGRVTMTRDTS | |
| scFv-Fc | gatgggatatttcaacccttataatgattacacac | | | TSTVYMELSSLRSEDTAVYYC | |
| | gctacgcacagaagttccagggcagagtcaccatg | | | ARSDGYYDAMDYWGQGTT | |
| | accagggacacgtccacgagcacagtctacatgga | | | VTVSSGGGGGGGGSGGGG | |
| | gctgagcagcctgagatctgaggacacggccgtgt | | | SGGGGSDIQMTQSPSTLSAS | |
| | attactgtgcgagatctgacggctactacgacgct | | | VGDRVTITCRASKSISKYLAW | |
| | atggactactgggggcaagggaccacggtcaccgt | | | YQQKPGKAPKLLIHSGSSLES | |
| | ctcctcgggtggtggaggtagtggtggaggcggat | | | GVPSRFSGSGSGTEFTLTISSL | |
| | ctggcggcggcggttcaggaggtggtggatccgac | | | QPDDFATYYCQQHIEYPWTF | |
| | atccagatgacccagtctccttccaccctgtctgc | | | GQGTKVEIKSSSEPKSSDKTH | |
| | atctgtaggagacagagtcaccatcacttgccggg | | | TCPPCPAPPAAAPSVFLFPPK | |
| | ccagtaagagtattagtaagtacttggcctggtat | | | PKDTLMISRTPEVTCVVVDV | |
| | cagcagaaaccagggaaagcccctaagctcctgat | | | SHEDPEVKFNWYVDGVEVH | |
| | ccattctggctccagtttggaaagtggggtcccat | | | NAKTKPREEQYNSTYRVVSV | |
| | caaggttcagcggcagtggatctgggacagaattc | | | LTVLHQDWLNGKEYKCAVS | |
| | actctcaccatcagcagcctgcagcctgatgattt | | | NKALPAPIEKTISKAKGQPRE | |
| | tgcaacttattactgccaacagcatattgaatatc | | | PQVYTLPPSRDELTKNQVSLT | |
| | cttggacgttcggccaagggaccaaggtggaaatc | | | CLVKGFYPSDIAVEWESNGQ | |
| | aaatcctcgagtgagcccaaatcttctgacaaaac | | | PENNYKTTPPVLDSDGSFFLY | |
| | tcacacatgcccaccgtgcccagcacctccagccg | | | SKLTVDKSRWQQGNVFSCS | |
| | ctgcaccgtcagtcttcctcttccccccaaaaccc | | | VMHEALHNHYTQKSLSLSPG | |
| | aaggacaccctcatgatctcccggacccctgaggt | | | K | |
| | cacatgcgtggtggtggacgtgagccacgaagacc | | | | |
| | ctgaggtcaagttcaactggtacgtggacggcgtg | | | | |
| | gaggtgcataatgccaagacaaagccgcgggagga | | | | |
| | gcagtacaacagcacgtaccgtgtggtcagcgtcc | | | | |
| | tcaccgtcctgcaccaggactggctgaatggcaag | | | | |
| | gaatacaagtgcgcggtctccaacaaagccctccc | | | | |
| | agcccccatcgagaaaaccatctccaaagccaaag | | | | |
| | ggcagccccgagaaccacaggtgtacaccctgccc | | | | |
| | ccatcccgggatgagctgaccaagaaccaggtcag | | | | |
| | cctgacctgcctggtcaaaggcttctatccaagcg | | | | |
| | acatcgccgtggagtgggagagcaatgggcagccg | | | | |
| | gagaacaactacaagaccacgcctcccgtgctgga | | | | |
| | ctccgacggctccttcttcctctacagcaagctca | | | | |
| | ccgtggacaagagcaggtggcagcaggggaacgtc | | | | |
| | ttctcatgctccgtgatgcatgaggctctgcacaa | | | | |
| | ccactacacgcagaagagcctctccctgtctccgg | | | | |
| | gtaaa | | | | |
| PSMA | tcctcggagcccaaatcttctgacaaaactcacac | | 39 | SSEPKSSDKTHTCPPCPAPPA | 40 |
| 01040 | atgcccaccgtgcccagcacctccagccgctgcac | | | AAPSVFLFPPKPKDTLMISRT | |
| Fc-linker | cgtcagtcttcctcttccccccaaaacccaaggac | | | PEVTCVVVDVSHEDPEVKFN | |
| anti PSMA | accctcatgatctcccggacccctgaggtcacatg | | | WYVDGVEVHNAKTKPREEQ | |
| scFv | cgtggtggtggacgtgagccacgaagaccctgagg | | | YNSTYRVVSVLTVLHQDWLN | |
| | tcaagttcaactggtacgtggacggcgtggaggtg | | | GKEYKCAVSNKALPAPIEKTIS | |
| | cataatgccaagacaaagccgcgggaggagcagta | | | KAKGQPREPQVYTLPPSRDE | |
| | caacagcacgtaccgtgtggtcagcgtcctcaccg | | | LTKNQVSLTCLVKGFYPSDIA | |
| | tcctgcaccaggactggctgaatggcaaggaatac | | | VEWESNGQPENNYKTTPPV | |
| | aagtgcgcggtctccaacaaagccctcccagcccc | | | LDSDGSFFLYSKLTVDKSRW | |
| | catcgagaaaaccatctccaaagccaaagggcagc | | | QQGNVFSCSVMHEALHNHY | |
| | cccgagaaccacaggtgtacaccctgcccccatcc | | | TQKSLSLSPGGGGSPSDIQM | |
| | cgggatgagctgaccaagaaccaggtcagcctgac | | | TQSPSTLSASVGDRVTITCRA | |
| | ctgcctggtcaaaggcttctatccaagcgacatcg | | | SKSISKYLAWYQQKPGKAPK | |
| | ccgtggagtgggagagcaatgggcagccggagaac | | | LLIHSGSSLESGVPSRFSGSGS | |
| | aactacaagaccacgcctcccgtgctggactccga | | | GTEFTLTISSLQPDDFATYYC | |
| | cggctccttcttcctctacagcaagctcaccgtgg | | | QQHIEYPWTFGQGTKVEIKG | |
| | acaagagcaggtggcagcaggggaacgtcttctca | | | GGGSGGGGGGGGSGGGG | |
| | tgctccgtgatgcatgaggctctgcacaaccacta | | | SQVQLVQSGAEVKKPGASVK | |
| | cacgcagaagagcctctccctgtctccgggcggcg | | | VSCKASGYTFTDYYMHWVR | |
| | ggggatccccgtcagacatccagatgacccagtct | | | QAPGQGLEWMGYFNPYND | |
| | ccttccaccctgtctgcatctgtaggagacagagt | | | YTRYAQKFQGRVTMTRDTS | |
| | caccatcacttgccgggccagtaagagtattagta | | | TSTVYMELSSLRSEDTAVYYC | |
| | agtacttggcctggtatcagcagaaaccagggaaa | | | ARSDGYYDAMDYWGQGTT | |
| | gcccctaagctcctgatccattctggctccagttt | | | VTVSSR | |
| | ggaaagtggggtcccatcaaggttcagcggcagtg | | | | |
| | gatctgggacagaattcactctcaccatcagcagc | | | | |
| | ctgcagcctgatgattttgcaacttattactgcca | | | | |
| | acagcatattgaatatccttggacgttcggccaag | | | | |
| | ggaccaaggtggaaatcaaaggtggtggaggtagt | | | | |
| | ggtggaggcggatctggcggcggcggttcaggagg | | | | |
| | tggtggatcccaggtgcagctggtgcagtctgggg | | | | |
| | ctgaggtgaagaagcctggggcctcagtgaaggtt | | | | |
| | tcctgcaaggcatctggatacaccttcaccgacta | | | | |
| | ctatatgcactgggtgcgacaggcccctggacaag | | | | |
| | ggcttgagtggatgggatatttcaacccttataat | | | | |
| | gattacacacgctacgcacagaagttccagggcag | | | | |
| | agtcaccatgaccagggacacgtccacgagcacag | | | | |
| | tctacatggagctgagcagcctgagatctgaggac | | | | |
| | acggccgtgtattactgtgcgagatctgacggcta | | | | |
| | ctacgacgctatggactactgggggcaagggacca | | | | |
| | cggtcaccgtctcctcgcgc | | | | |
| PSMA | tcctcggagcccaaatcttctgacaaaactcacac | | 41 | SSEPKSSDKTHTCPPCPAPPA | 42 |
| 01041 | atgcccaccgtgcccagcacctccagccgctgcac | | | AAPSVFLFPPKPKDTLMISRT | |
| Fc-linker- | cgtcagtcttcctcttccccccaaaacccaaggac | | | PEVTCVVVDVSHEDPEVKFN | |
| anti PSMA | accctcatgatctcccggacccctgaggtcacatg | | | WYVDGVEVHNAKTKPREEQ | |
| scFv | cgtggtggtggacgtgagccacgaagaccctgagg | | | YNSTYRVVSVLTVLHQDWLN | |
| | tcaagttcaactggtacgtggacggcgtggaggtg | | | GKEYKCAVSNKALPAPIEKTIS | |
| | cataatgccaagacaaagccgcgggaggagcagta | | | KAKGQPREPQVYTLPPSRDE | |
| | caacagcacgtaccgtgtggtcagcgtcctcaccg | | | LTKNQVSLTCLVKGFYPSDIA | |
| | tcctgcaccaggactggctgaatggcaaggaatac | | | VEWESNGQPENNYKTTPPV | |
| | aagtgcgcggtctccaacaaagccctcccagcccc | | | LDSDGSFFLYSKLTVDKSRW | |
| | catcgagaaaaccatctccaaagccaaagggcagc | | | QQGNVFSCSVMHEALHNHY | |
| | cccgagaaccacaggtgtacaccctgcccccatcc | | | TQKSLSLSPGGGGSPSQVQL | |
| | cgggatgagctgaccaagaaccaggtcagcctgac | | | VQSGAEVKKPGASVKVSCKA | |
| | ctgcctggtcaaaggcttctatccaagcgacatcg | | | SGYTFTDYYMHWVRQAPG | |
| | ccgtggagtgggagagcaatgggcagccggagaac | | | QGLEWMGYFNPYNDYTRYA | |
| | aactacaagaccacgcctcccgtgctggactccga | | | QKFQGRVTMTRDTSTSTVY | |
| | cggctccttcttcctctacagcaagctcaccgtgg | | | MELSSLRSEDTAVYYCARSD | |
| | acaagagcaggtggcagcaggggaacgtcttctca | | | GYYDAMDYWGQGTTVTVS | |
| | tgctccgtgatgcatgaggctctgcacaaccacta | | | SGGGGSGGGGSGGGGSGG | |
| | cacgcagaagagcctctccctgtctccgggcggcg | | | GGSDIQMTQSPSTLSASVGD | |
| | ggggatccccgtcacaggtgcagctggtgcagtct | | | RVTITCRASKSISKYLAWYQQ | |
| | ggggctgaggtgaagaagcctggggcctcagtgaa | | | KPGKAPKLLIHSGSSLESGVPS | |
| | ggtttcctgcaaggcatctggatacaccttcaccg | | | RFSGSGSGTEFTLTISSLQPD | |
| | actactatatgcactgggtgcgacaggcccctgga | | | DFATYYCQQHIEYPWTFGQ | |
| | caagggcttgagtggatgggatatttcaaccctta | | | GTKVEIKR | |
| | taatgattacacacgctacgcacagaagttccagg | | | | |
| | gcagagtcaccatgaccagggacacgtccacgagc | | | | |
| | acagtctacatggagctgagcagcctgagatctga | | | | |
| | ggacacggccgtgtattactgtgcgagatctgacg | | | | |
| | gctactacgacgctatggactactgggggcaaggg | | | | |
| | accacggtcaccgtctcctcgggtggtggaggtag | | | | |
| | tggtggaggcggatctggcggcggcggttcaggag | | | | |
| | gtggtggatccgacatccagatgacccagtctcct | | | | |
| | tccaccctgtctgcatctgtaggagacagagtcac | | | | |
| | catcacttgccgggccagtaagagtattagtaagt | | | | |
| | acttggcctggtatcagcagaaaccagggaaagcc | | | | |
| | cctaagctcctgatccattctggctccagtttgga | | | | |
| | aagtggggtcccatcaaggttcagcggcagtggat | | | | |
| | ctgggacagaattcactctcaccatcagcagcctg | | | | |
| | cagcctgatgattttgcaacttattactgccaaca | | | | |
| | gcatattgaatatccttggacgttcggccaaggga | | | | |
| | ccaaggtggaaatcaaacgc | | | | |
| PSMA | caggtgcagctggtgcagtctggggctgaggtgaa | | 43 | QVQLVQSGAEVKKPGASVK | 44 |
| 01072 | gaagcctggggcctcagtgaaggtttcctgcaagg | | | VSCKASGYTFTDYYMHWVR | |
| Anti | catctggatacaccttcaccgactactatatgcac | | | QAPGQGLEWMGYFNPYND | |
| PSMA | tgggtgcgacaggcccctggacaagggcttgagtg | | | YTRYAQKFQGRVTMTRDTS | |
| scFv x | gatgggatatttcaacccttataatgattacacac | | | TSTVYMELSSLRSEDTAVYYC | |
| Anti | gctacgcacagaagttccagggcagagtcaccatg | | | ARSDGYYDAMDYWGQGTT | |
| CD3 scFv | accagggacacgtccacgagcacagtctacatgga | | | VTVSSGGGGSGGGGSGGGG | |
| ADAPTIR ^{™} | gctgagcagcctgagatctgaggacacggccgtgt | | | SGGGGSDIQMTQSPSSLSAS | |
| | attactgtgcgagatctgacggctactacgacgct | | | VGDRVTITCRASKSISKYLAW | |
| | atggactactgggggcaagggaccacggtcaccgt | | | YQQKPGKAPKLLIHSGSSLES | |
| | ctcctcgggtggtggaggtagtggtggaggcggat | | | GVPSRFSGSGSGTEFTLTISSL | |
| | ctggcggcggcggttcaggaggtggtggatccgac | | | QPDDFATYYCQQHIEYPWTF | |
| | atccagatgacccagtctccttcctccctgtctgc | | | GQGTKVEIKEPKSSDKTHTCP | |
| | atctgtaggagacagagtcaccatcacttgccggg | | | PCPAPPAAAPSVFLFPPKPKD | |
| | ccagtaagagtattagtaagtacttggcctggtat | | | TLMISRTPEVTCVVVDVSHE | |
| | cagcagaaaccagggaaagcccctaagctcctgat | | | DPEVKFNWYVDGVEVHNAK | |
| | ccattctggctccagtttggaaagtggggtcccat | | | TKPREEQYNSTYRVVSVLTVL | |
| | caaggttcagcggcagtggatctgggacagaattc | | | HQDWLNGKEYKCAVSNKAL | |
| | actctcaccatcagcagcctgcagcctgatgattt | | | PAPIEKTISKAKGQPREPQVY | |
| | tgcaacttattactgccaacagcatattgaatatc | | | TLPPSRDELTKNQVSLTCLVK | |
| | cttggacgttcggccaagggaccaaggtggaaatc | | | GFYPSDIAVEWESNGQPEN | |
| | aaagagcccaaatcttctgacaaaactcacacatg | | | NYKTTPPVLDSDGSFFLYSKL | |
| | cccaccgtgcccagcacctccagccgctgcaccgt | | | TVDKSRWQQGNVFSCSVM | |
| | cagtcttcctcttccccccaaaacccaaggacacc | | | HEALHNHYTQKSLSLSPGGG | |
| | ctcatgatctcccggacccctgaggtcacatgcgt | | | GSPSQVQLVQSGAEVKKPG | |
| | ggtggtggacgtgagccacgaagaccctgaggtca | | | ASVKVSCKASGYTFTRSTMH | |
| | agttcaactggtacgtggacggcgtggaggtgcat | | | WVRQAPGQGLEWIGYINPS | |
| | aatgccaagacaaagccgcgggaggagcagtacaa | | | SAYTNYAQKFQGRVTLTADK | |
| | cagcacgtaccgtgtggtcagcgtcctcaccgtcc | | | STSTAYMELSSLRSEDTAVYY | |
| | tgcaccaggactggctgaatggcaaggaatacaag | | | CASPQVHYDYNGFPYWGQ | |
| | tgcgcggtctccaacaaagccctcccagcccccat | | | GTLVTVSSGGGGGGGGSG | |
| | cgagaaaaccatctccaaagccaaagggcagcccc | | | GGGSGGGGSDIQMTQSPSS | |
| | gagaaccacaggtgtacaccctgcccccatcccgg | | | LSASVGDRVTITCRASSSVSY | |
| | gatgagctgaccaagaaccaggtcagcctgacctg | | | MNWYQQKPGKAPKRWIYD | |
| | cctggtcaaaggcttctatccaagcgacatcgccg | | | SSKLASGVPSRFSGSGSGTDF | |
| | tggagtgggagagcaatgggcagccggagaacaac | | | TLTISSLQPEDFATYYCQQWS | |
| | tacaagaccacgcctcccgtgctggactccgacgg | | | RNPPTFGQGTKVEIKRS | |
| | ctccttcttcctctacagcaagctcaccgtggaca | | | | |
| | agagcaggtggcagcaggggaacgtcttctcatgc | | | | |
| | tccgtgatgcatgaggctctgcacaaccactacac | | | | |
| | gcagaagagcctctccctgtctccgggcggcgggg | | | | |
| | gatccccgtcacaagtacaactcgttcaaagtggc | | | | |
| | gcagaagtaaagaagccaggcgccagtgttaaggt | | | | |
| | gagctgcaaggcaagcgggtacaccttcacccggt | | | | |
| | ctacaatgcactgggtaagacaagcaccagggcaa | | | | |
| | ggactcgaatggattggttacatcaacccttcctc | | | | |
| | tgcatacaccaactacgctcaaaagttccagggcc | | | | |
| | gcgttactttgacagcggataaatctacatccacg | | | | |
| | gcctatatggaactgtcaagcctcaggagcgagga | | | | |
| | cacagcggtatattactgtgcatctccccaggtcc | | | | |
| | attatgactacaacgggtttccgtactggggacaa | | | | |
| | ggaactctggttacagtcagtagcggtggtggagg | | | | |
| | tagtggtggaggcggatctggcggcggcggttcag | | | | |
| | gaggtggtggatccgatatccagatgacccaaagt | | | | |
| | ccgagctcgttgagtgcaagtgtaggagaccgcgt | | | | |
| | aacgattacttgcagagcttcaagttccgtatcct | | | | |
| | acatgaattggtatcagcaaaagcctggaaaagcc | | | | |
| | cctaagcgctggatatacgattcaagtaagttggc | | | | |
| | ttctggcgtcccatcacggttttctggttcaggtt | | | | |
| | ccggtacagattttacgctgacaatcagctctctc | | | | |
| | caaccggaagatttcgcaacctattactgtcaaca | | | | |
| | atggtcaagaaatccgccgacattcgggcagggaa | | | | |
| | caaaagtcgagataaaaaggtca | | | | |
| | | DNA | | AA | |
| Construct | Component | Sequence | | Sequence | |
| TRI | Anti TA | gacatcgtgatgacccagtctcca | 45 | DIVMTQSPDSLAVSLGERATI | 46 |
| 01044 | scFv x Fc | gactccctggctgtgtctctgggc | | NCKSSHSVLYSSNNKNYLAW | |
| Anti TA | Knob | gagagggccaccatcaactgcaag | | YQQKPGQPPKLLIYWASTRE | |
| scFv x Fc; | (Chain 1) | tccagccacagtgttttatacagc | | SGVPDRFSGSGSGTDFTLTIS | |
| Anti TA | | tccaacaataagaactacttagct | | SLQAEDVAVYYCQQYYSTPP | |
| scFv x Fc | | tggtaccagcagaaaccaggacag | | TTFGGGTKVEIKGGGGSGGG | |
| x linker | | cctgaccgattcagtggcagcggg | | GSGGGGSGGGGSEVQLLES | |
| x Anti | | gcatctacccgggaatccggggtc | | GGGLVQPGGSLRLSCAASGF | |
| CD3 scFv | | cctcctaagctgctcatttactgg | | TFSSYGMSWVRQAPGKGLE | |
| | | tctgggacagatttcactctcacc | | GVSAISGSGGSTYYADSVKG | |
| | | atcagcagcctgcaggctgaagat | | RFTISRDNSKNTLYLQMNSLR | |
| | | gtggcagtttattactgtcagcaa | | AEDTAVYYCAKEKLRYFDWL | |
| | | tattatagtactcctccgaccact | | SDAFDIWGQGTMVTVSSSE | |
| | | ttcggcggagggaccaaggtggag | | PKSSDKTHTCPPCPAPPAAA | |
| | | atcaaaggtggaggcggttcaggc | | PSVFLFPPKPKDTLMISRTPE | |
| | | ggaggtggatccggcggtggcggc | | VTCVVVDVSHEDPEVKFNW | |
| | | tccggtggcggcggatctgaggtg | | YVDGVEVHNAKTKPREEQY | |
| | | cagctgttggagtctgggggaggc | | NSTYRVVSVLTVLHQDWLN | |
| | | ttggtacagcctggggggtccctg | | GKEYKCAVSNKALPAPIEKTIS | |
| | | agactctcctgtgcagcctctgga | | KAKGQPREPQVYTLPPSRDE | |
| | | ttcacctttagcagctatggcatg | | LTKNQVSLWCLVKGFYPSDI | |
| | | agctgggtccgccaggctccaggg | | AVEWESNGQPENNYKTTPP | |
| | | aaggggctggagggggtctcagct | | VLDSDGSFFLYSKLTVDKSR | |
| | | attagtggtagtggtggtagcaca | | WQQGNVFSCSVMHEALHN | |
| | | tactacgcagactccgtgaagggc | | HYTQKSLSLSPGK | |
| | | cggttcaccatctccagagacaat | | | |
| | | tccaagaacacgctgtatctgcaa | | | |
| | | atgaacagcctgagagccgaggac | | | |
| | | acggccgtatattactgtgcgaaa | | | |
| | | gaaaagttacgatattttgactgg | | | |
| | | ttatccgatgcttttgatatctgg | | | |
| | | ggccaagggacaatggtcaccgtc | | | |
| | | tcctcgagtgagcccaaatcttct | | | |
| | | gacaaaactcacacatgcccaccg | | | |
| | | tgcccagcacctccagccgctgca | | | |
| | | ccgtcagtcttcctcttcccccca | | | |
| | | aaacccaaggacaccctcatgatc | | | |
| | | tcccggacccctgaggtcacatgc | | | |
| | | gtggtggtggacgtgagccacgaa | | | |
| | | gaccctgaggtcaagttcaactgg | | | |
| | | tacgtggacggcgtggaggtgcat | | | |
| | | aatgccaagacaaagccgcgggag | | | |
| | | gagcagtacaacagcacgtaccgt | | | |
| | | gtggtcagcgtcctcaccgtcctg | | | |
| | | caccaggactggctgaatggcaag | | | |
| | | gaatacaagtgcgcggtctccaac | | | |
| | | aaagccctcccagcccccatcgag | | | |
| | | aaaaccatctccaaagccaaaggg | | | |
| | | cagccccgagaaccacaggtgtac | | | |
| | | accctgcccccatcccgggatgag | | | |
| | | ctgaccaagaaccaggtcagcctg | | | |
| | | tggtgcctggtcaaaggcttctat | | | |
| | | ccaagcgacatcgccgtggagtgg | | | |
| | | gagagcaatgggcagccggagaac | | | |
| | | aactacaagaccacgcctcccgtg | | | |
| | | ctggactccgacggctccttcttc | | | |
| | | ctctacagcaagctcaccgtggac | | | |
| | | aagagcaggtggcagcaggggaac | | | |
| | | gtcttctcatgctccgtgatgcat | | | |
| | | gaggctctgcacaaccactacacg | | | |
| | | cagaagagcctctccctgtctccg | | | |
| | | ggtaaa | | | |
| | Anti TA | gacatcgtgatgacccagtctcca | 47 | DIVMTQSPDSLAVSLGERATI | 48 |
| | scFv x Fc | gactccctggctgtgtctctgggc | | NCKSSHSVLYSSNNKNYLAW | |
| | Hole x | gagagggccaccatcaactgcaag | | YQQKPGQPPKLLIYWASTRE | |
| | Anti | tccagccacagtgttttatacagc | | SGVPDRFSGSGSGTDFTLTIS | |
| | CD3 scFv | tccaacaataagaactacttagct | | SLQAEDVAVYYCQQYYSTPP | |
| | (Chain 2) | tggtaccagcagaaaccaggacag | | TTFGGGTKVEIKGGGGSGGG | |
| | | cctcctaagctgctcatttactgg | | GSGGGGSGGGGSEVQLLES | |
| | | gcatctacccgggaatccggggtc | | GGGLVQPGGSLRLSCAASGF | |
| | | cctgaccgattcagtggcagcggg | | TFSSYGMSWVRQAPGKGLE | |
| | | tctgggacagatttcactctcacc | | GVSAISGSGGSTYYADSVKG | |
| | | atcagcagcctgcaggctgaagat | | RFTISRDNSKNTLYLQMNSLR | |
| | | gtggcagtttattactgtcagcaa | | AEDTAVYYCAKEKLRYFDWL | |
| | | tattatagtactcctccgaccact | | SDAFDIWGQGTIVTVSSSEPK | |
| | | ttcggcggagggaccaaggtggag | | SSDKTHTCPPCPAPPAAAPS | |
| | | atcaaaggtggaggcggttcaggc | | VFLFPPKPKDTLMISRTPEVT | |
| | | ggaggtggatccggcggtggcggc | | CVVVDVSHEDPEVKFNWYV | |
| | | tccggtggcggcggatctgaggtg | | DGVEVHNAKTKPREEQYNST | |
| | | cagctgttggagtctgggggaggc | | YRVVSVLTVLHQDWLNGKE | |
| | | ttggtacagcctggggggtccctg | | YKCAVSNKALPAPIEKTISKAK | |
| | | agactctcctgtgcagcctctgga | | GQPREPQVYTLPPSRDELTK | |
| | | ttcacctttagcagctatggcatg | | NQVSLSCAVKGFYPSDIAVE | |
| | | agctgggtccgccaggctccaggg | | WESNGQPENNYKTTPPVLD | |
| | | aaggggctggagggggtctcagct | | SDGSFFLVSKLTVDKSRWQQ | |
| | | attagtggtagtggtggtagcaca | | GNVFSCSVMHEALHNRFTQ | |
| | | tactacgcagactccgtgaagggc | | KSLSLSPGGGGSPSQVQLVQ | |
| | | cggttcaccatctccagagacaat | | SGAEVKKPGASVKVSCKASG | |
| | | tccaagaacacgctgtatctgcaa | | YTFTRSTMHWVRQAPGQGL | |
| | | atgaacagcctgagagccgaggac | | EWIGYINPSSAYTNYAQKFQ | |
| | | acggccgtatattactgtgcgaaa | | GRVTLTADKSTSTAYMELSSL | |
| | | gaaaagttacgatattttgactgg | | RSEDTAVYYCASPQVHYDYN | |
| | | ttatccgatgcttttgatatctgg | | GFPYWGQGTLVTVSSGGGG | |
| | | ggccaagggacaattgtcaccgtc | | SGGGGSGGGGSGGGGSDV | |
| | | tcctcgagtgagcccaaatcttct | | QLTQSPSTLSASVGDRVTITC | |
| | | gacaaaactcacacatgcccaccg | | SASSSVSYMNWYQQKPGKA | |
| | | tgcccagcacctccagccgctgca | | PKRWIYDSSKLASGVPARFS | |
| | | ccgtcagtcttcctcttcccccca | | GSGSGTEYTLTISSLQPDDFA | |
| | | aaacccaaggacaccctcatgatc | | TYYCQQWSRNPPTFGQGTK | |
| | | tcccggacccctgaggtcacatgc | | VEVKRS | |
| | | gtggtggtggacgtgagccacgaa | | | |
| | | gaccctgaggtcaagttcaactgg | | | |
| | | tacgtggacggcgtggaggtgcat | | | |
| | | aatgccaagacaaagccgcgggag | | | |
| | | gagcagtacaacagcacgtaccgt | | | |
| | | gtggtcagcgtcctcaccgtcctg | | | |
| | | caccaggactggctgaatggcaag | | | |
| | | gaatacaagtgcgcggtctccaac | | | |
| | | aaagccctcccagcccccatcgag | | | |
| | | aaaaccatctccaaagccaaaggg | | | |
| | | cagccccgagaaccacaggtgtac | | | |
| | | accctgcccccatcccgggatgag | | | |
| | | ctgaccaagaaccaggtcagcctg | | | |
| | | tcttgcgctgtcaaaggcttctat | | | |
| | | ccaagcgacatcgccgtggagtgg | | | |
| | | gagagcaatgggcagccggagaac | | | |
| | | aactacaagaccacgcctcccgtg | | | |
| | | ctggactccgacggctccttcttc | | | |
| | | ctcgttagcaagctcaccgtggac | | | |
| | | aagagcaggtggcagcaggggaac | | | |
| | | gtcttctcatgctccgtgatgcat | | | |
| | | gaggctctgcacaaccgtttcacg | | | |
| | | cagaagagcctctccctgtctccg | | | |
| | | ggcggcgggggatccccgtcacaa | | | |
| | | gtacaactcgttcaaagtggcgca | | | |
| | | gaagtaaagaagccaggcgccagt | | | |
| | | gttaaggtgagctgcaaggcaagc | | | |
| | | gggtacaccttcacccggtctaca | | | |
| | | atgcactgggtaagacaagcacca | | | |
| | | gggcaaggactcgaatggattggt | | | |
| | | tacatcaacccttcctctgcatac | | | |
| | | accaactacgctcaaaagttccag | | | |
| | | ggccgcgttactttgacagcggat | | | |
| | | aaatctacatccacggcctatatg | | | |
| | | gaactgtcaagcctcaggagcgag | | | |
| | | gacacagcggtatattactgtgca | | | |
| | | tctccccaggtccattatgactac | | | |
| | | aacgggtttccgtactggggacaa | | | |
| | | ggaactctggttacagtcagtagc | | | |
| | | ggtggtggaggtagtggtggaggc | | | |
| | | ggatctggcggcggcggttcagga | | | |
| | | ggtggtggatccgatgtccagctt | | | |
| | | acccaaagtccgagcacgttgagt | | | |
| | | gcaagtgtaggagaccgcgtaacg | | | |
| | | attacttgctctgcttcaagttcc | | | |
| | | gtatcctacatgaattggtatcag | | | |
| | | caaaagcctggaaaagcccctaag | | | |
| | | cgctggatatacgattcaagtaag | | | |
| | | ttggcttctggcgtcccagcacgg | | | |
| | | ttttctggttcaggttccggtaca | | | |
| | | gaatatacgctgacaatcagctct | | | |
| | | ctccaaccggatgatttcgcaacc | | | |
| | | tattactgtcaacaatggtcaaga | | | |
| | | aatccgccgacattcgggcaggga | | | |
| | | acaaaagtcgaggtaaaaaggtca | | | |
| TRI | Anti TA | Same sequence as Chain 1 | 45 | | |
| 01045 | scFv x Fc | for TRI01044 | | | |
| Anti TA | Knob | | | | |
| scFv x Fc; | (Chain 1) | | | | |
| Anti TA | Anti TA | gacatcgtgatgacccagtctcca | 49 | DIVMTQSPDSLAVSLGERATI | |
| scFv x Fc | scFv x Fc | gactccctggctgtgtctctgggc | | NCKSSHSVLYSSNNKNYLAW | |
| x linker | Hole x | gagagggccaccatcaactgcaag | | YQQKPGQPPKLLIYWASTRE | |
| x Anti | Anti | tccagccacagtgttttatacagc | | SGVPDRFSGSGSGTDFTLTIS | |
| CD3 scFv | CD3 scFv | tccaacaataagaactacttagct | | SLQAEDVAVYYCQQYYSTPP | |
| | (Chain 2) | tggtaccagcagaaaccaggacag | | TTFGGGTKVEIKGGGGSGGG | |
| | | cctcctaagctgctcatttactgg | | GSGGGGSGGGGSEVQLLES | |
| | | gcatctacccgggaatccggggtc | | GGGLVQPGGSLRLSCAASGF | |
| | | cctgaccgattcagtggcagcggg | | TFSSYGMSWVRQAPGKGLE | |
| | | tctgggacagatttcactctcacc | | GVSAISGSGGSTYYADSVKG | |
| | | atcagcagcctgcaggctgaagat | | RFTISRDNSKNTLYLQMNSLR | |
| | | gtggcagtttattactgtcagcaa | | AEDTAVYYCAKEKLRYFDWL | |
| | | tattatagtactcctccgaccact | | SDAFDIWGQGTMVTVSSSE | |
| | | ttcggcggagggaccaaggtggag | | PKSSDKTHTCPPCPAPPAAA | |
| | | atcaaaggtggaggcggttcaggc | | PSVFLFPPKPKDTLMISRTPE | |
| | | ggaggtggatccggcggtggcggc | | VTCVVVDVSHEDPEVKFNW | |
| | | tccggtggcggcggatctgaggtg | | YVDGVEVHNAKTKPREEQY | |
| | | cagctgttggagtctgggggaggc | | NSTYRVVSVLTVLHQDWLN | |
| | | ttggtacagcctggggggtccctg | | GKEYKCAVSNKALPAPIEKTIS | |
| | | agactctcctgtgcagcctctgga | | KAKGQPREPQVYTLPPSRDE | |
| | | ttcacctttagcagctatggcatg | | LTKNQVSLSCAVKGFYPSDIA | |
| | | agctgggtccgccaggctccaggg | | VEWESNGQPENNYKTTPPV | |
| | | aaggggctggagggggtctcagct | | LDSDGSFFLVSKLTVDKSRW | |
| | | attagtggtagtggtggtagcaca | | QQGNVFSCSVMHEALHNRF | |
| | | tactacgcagactccgtgaagggc | | TQKSLSLSPGGGGSPSQVQL | |
| | | cggttcaccatctccagagacaat | | VQSGAEVKKPGASVKVSCKA | |
| | | tccaagaacacgctgtatctgcaa | | SGYTFTRSTMHWVRQAPGQ | |
| | | atgaacagcctgagagccgaggac | | GLEWIGYINPSSAYTNYNQK | |
| | | acggccgtatattactgtgcgaaa | | FQGRVTLTADKSTSTAYMEL | |
| | | gaaaagttacgatattttgactgg | | SSLRSEDTAVYYCASPQVHY | |
| | | ttatccgatgcttttgatatctgg | | DYNGFPYWGQGTLVTVSSG | |
| | | ggccaagggacaatggtcaccgtc | | GGGSGGGGSGGGGSGGGG | |
| | | tcctcgagtgagcccaaatcttct | | SDVQLTQSPSTLSASVGDRV | |
| | | gacaaaactcacacatgcccaccg | | TITCSASSSVSYMNWYQQKP | |
| | | tgcccagcacctccagccgctgca | | GKAPKRWIYDSSKLASGVPA | |
| | | ccgtcagtcttcctcttcccccca | | RFSGSGSGTEYTLTISSLQPD | |
| | | aaacccaaggacaccctcatgatc | | DFATYYCQQWSRNPPTFGQ | |
| | | tcccggacccctgaggtcacatgc | | GTKVEVKRS | |
| | | gtggtggtggacgtgagccacgaa | | | |
| | | gaccctgaggtcaagttcaactgg | | | |
| | | tacgtggacggcgtggaggtgcat | | | |
| | | aatgccaagacaaagccgcgggag | | | |
| | | gagcagtacaacagcacgtaccgt | | | |
| | | gtggtcagcgtcctcaccgtcctg | | | |
| | | caccaggactggctgaatggcaag | | | |
| | | gaatacaagtgcgcggtctccaac | | | |
| | | aaagccctcccagcccccatcgag | | | |
| | | aaaaccatctccaaagccaaaggg | | | |
| | | cagccccgagaaccacaggtgtac | | | |
| | | accctgcccccatcccgggatgag | | | |
| | | ctgaccaagaaccaggtcagcctg | | | |
| | | tcttgcgctgtcaaaggcttctat | | | |
| | | ccaagcgacatcgccgtggagtgg | | | |
| | | gagagcaatgggcagccggagaac | | | |
| | | aactacaagaccacgcctcccgtg | | | |
| | | ctggactccgacggctccttcttc | | | |
| | | ctcgttagcaagctcaccgtggac | | | |
| | | aagagcaggtggcagcaggggaac | | | |
| | | gtcttctcatgctccgtgatgcat | | | |
| | | gaggctctgcacaaccgtttcacg | | | |
| | | cagaagagcctctccctgtctccg | | | |
| | | ggcggcgggggatccccgtcacaa | | | |
| | | gtacaactcgttcaaagtggcgca | | | |
| | | gaagtaaagaagccaggcgccagt | | | |
| | | gttaaggtgagctgcaaggcaagc | | | |
| | | gggtacaccttcacccggtctaca | | | |
| | | atgcactgggtaagacaagcacca | | | |
| | | gggcaaggactcgaatggattggt | | | |
| | | tacatcaacccttcctctgcatac | | | |
| | | accaactacaatcaaaagttccag | | | |
| | | ggccgcgttactttgacagcggat | | | |
| | | aaatctacatccacggcctatatg | | | |
| | | gaactgtcaagcctcaggagcgag | | | |
| | | gacacagcggtatattactgtgca | | | |
| | | tctccccaggtccattatgactac | | | |
| | | aacgggtttccgtactggggacaa | | | |
| | | ggaactctggttacagtcagtagc | | | |
| | | ggtggtggaggtagtggtggaggc | | | |
| | | ggatctggcggcggcggttcagga | | | |
| | | ggtggtggatccgatgtccagctt | | | |
| | | acccaaagtccgagcacgttgagt | | | |
| | | gcaagtgtaggagaccgcgtaacg | | | |
| | | attacttgctctgcttcaagttcc | | | |
| | | gtatcctacatgaattggtatcag | | | |
| | | caaaagcctggaaaagcccctaag | | | |
| | | cgctggatatacgattcaagtaag | | | |
| | | ttggcttctggcgtcccagcacgg | | | |
| | | ttttctggttcaggttccggtaca | | | |
| | | gaatatacgctgacaatcagctct | | | |
| | | ctccaaccggatgatttcgcaacc | | | |
| | | tattactgtcaacaatggtcaaga | | | |
| | | aatccgccgacattcgggcaggga | | | |
| | | acaaaagtcgaggtaaaaaggtca | | | |
| TRI | Anti TA | Same sequence as Chain 1 | 45 | | 46 |
| 01047 | scFv x Fc | for TRI01044 | | | |
| Anti TA | Knob | | | | |
| scFv x Fc; | (Chain 1) | | | | |
| Anti TA | Anti TA | gacatcgtgatgacccagtctcca | 51 | DIVMTQSPDSLAVSLGERATI | |
| scFv x Fc | scFv x Fc | gactccctggctgtgtctctgggc | | NCKSSHSVLYSSNNKNYLAW | |
| x linker | Hole x | gagagggccaccatcaactgcaag | | YQQKPGQPPKLLIYWASTRE | |
| x Anti | Anti | tccagccacagtgttttatacagc | | SGVPDRFSGSGSGTDFTLTIS | |
| CD3 scFv | CD3 scFv | tccaacaataagaactacttagct | | SLQAEDVAVYYCQQYYSTPP | |
| | (Chain 2) | tggtaccagcagaaaccaggacag | | TTFGGGTKVEIKGGGGSGGG | |
| | | cctcctaagctgctcatttactgg | | GSGGGGSGGGGSEVQLLES | |
| | | gcatctacccgggaatccggggtc | | GGGLVQPGGSLRLSCAASGF | |
| | | cctgaccgattcagtggcagcggg | | TFSSYGMSWVRQAPGKGLE | |
| | | tctgggacagatttcactctcacc | | GVSAISGSGGSTYYADSVKG | |
| | | atcagcagcctgcaggctgaagat | | RFTISRDNSKNTLYLQMNSLR | |
| | | gtggcagtttattactgtcagcaa | | AEDTAVYYCAKEKLRYFDWL | |
| | | tattatagtactcctccgaccact | | SDAFDIWGQGTIVTVSSSEPK | |
| | | ttcggcggagggaccaaggtggag | | SSDKTHTCPPCPAPPAAAPS | |
| | | atcaaaggtggaggcggttcaggc | | VFLFPPKPKDTLMISRTPEVT | |
| | | ggaggtggatccggcggtggcggc | | CVVVDVSHEDPEVKFNWYV | |
| | | tccggtggcggcggatctgaggtg | | DGVEVHNAKTKPREEQYNST | |
| | | cagctgttggagtctgggggaggc | | YRVVSVLTVLHQDWLNGKE | |
| | | ttggtacagcctggggggtccctg | | YKCAVSNKALPAPIEKTISKAK | |
| | | agactctcctgtgcagcctctgga | | GQPREPQVYTLPPSRDELTK | |
| | | ttcacctttagcagctatggcatg | | NQVSLSCAVKGFYPSDIAVE | |
| | | agctgggtccgccaggctccaggg | | WESNGQPENNYKTTPPVLD | |
| | | aaggggctggagggggtctcagct | | SDGSFFLVSKLTVDKSRWQQ | |
| | | attagtggtagtggtggtagcaca | | GNVFSCSVMHEALHNRFTQ | |
| | | tactacgcagactccgtgaagggc | | KSLSLSPGGGGSPSQVQLVQ | |
| | | cggttcaccatctccagagacaat | | SGAEVKKPGASVKVSCKASG | |
| | | tccaagaacacgctgtatctgcaa | | YTFTRSTMHWVRQAPGQGL | |
| | | atgaacagcctgagagccgaggac | | EWIGYINPSSAYTNYAQKFQ | |
| | | acggccgtatattactgtgcgaaa | | GRVTLTADKSTSTAYMELSSL | |
| | | gaaaagttacgatattttgactgg | | RSEDTAVYYCASPQVHYDYN | |
| | | ttatccgatgcttttgatatctgg | | GFPYWGQGTLVTVSSGGGG | |
| | | ggccaagggacaattgtcaccgtc | | SGGGGSGGGGSGGGGSDIQ | |
| | | tcctcgagtgagcccaaatcttct | | MTQSPSSLSASVGDRVTITCR | |
| | | gacaaaactcacacatgcccaccg | | ASSSVSYMNWYQQKPGKAP | |
| | | tgcccagcacctccagccgctgca | | KRWIYDSSKLASGVPSRFSGS | |
| | | ccgtcagtcttcctcttcccccca | | GSGTDFTLTISSLQPEDFATYY | |
| | | aaacccaaggacaccctcatgatc | | CQQWSRNPPTFGQGTKVEI | |
| | | tcccggacccctgaggtcacatgc | | KRS | |
| | | gtggtggtggacgtgagccacgaa | | | |
| | | gaccctgaggtcaagttcaactgg | | | |
| | | tacgtggacggcgtggaggtgcat | | | |
| | | aatgccaagacaaagccgcgggag | | | |
| | | gagcagtacaacagcacgtaccgt | | | |
| | | gtggtcagcgtcctcaccgtcctg | | | |
| | | caccaggactggctgaatggcaag | | | |
| | | gaatacaagtgcgcggtctccaac | | | |
| | | aaagccctcccagcccccatcgag | | | |
| | | aaaaccatctccaaagccaaaggg | | | |
| | | cagccccgagaaccacaggtgtac | | | |
| | | accctgcccccatcccgggatgag | | | |
| | | ctgaccaagaaccaggtcagcctg | | | |
| | | tcttgcgctgtcaaaggcttctat | | | |
| | | ccaagcgacatcgccgtggagtgg | | | |
| | | gagagcaatgggcagccggagaac | | | |
| | | aactacaagaccacgcctcccgtg | | | |
| | | ctggactccgacggctccttcttc | | | |
| | | ctcgttagcaagctcaccgtggac | | | |
| | | aagagcaggtggcagcaggggaac | | | |
| | | gtcttctcatgctccgtgatgcat | | | |
| | | gaggctctgcacaaccgtttcacg | | | |
| | | cagaagagcctctccctgtctccg | | | |
| | | ggcggcgggggatccccgtcacaa | | | |
| | | gtacaactcgttcaaagtggcgca | | | |
| | | gaagtaaagaagccaggcgccagt | | | |
| | | gttaaggtgagctgcaaggcaagc | | | |
| | | gggtacaccttcacccggtctaca | | | |
| | | atgcactgggtaagacaagcacca | | | |
| | | gggcaaggactcgaatggattggt | | | |
| | | tacatcaacccttcctctgcatac | | | |
| | | accaactacgctcaaaagttccag | | | |
| | | ggccgcgttactttgacagcggat | | | |
| | | aaatctacatccacggcctatatg | | | |
| | | gaactgtcaagcctcaggagcgag | | | |
| | | gacacagcggtatattactgtgca | | | |
| | | tctccccaggtccattatgactac | | | |
| | | aacgggtttccgtactggggacaa | | | |
| | | ggaactctggttacagtcagtagc | | | |
| | | ggtggtggaggtagtggtggaggc | | | |
| | | ggatctggcggcggcggttcagga | | | |
| | | ggtggtggatccgatatccagatg | | | |
| | | acccaaagtccgagctcgttgagt | | | |
| | | gcaagtgtaggagaccgcgtaacg | | | |
| | | attacttgcagagcttcaagttcc | | | |
| | | gtatcctacatgaattggtatcag | | | |
| | | caaaagcctggaaaagcccctaag | | | |
| | | cgctggatatacgattcaagtaag | | | |
| | | ttggcttctggcgtcccatcacgg | | | |
| | | ttttctggttcaggttccggtaca | | | |
| | | gattttacgctgacaatcagctct | | | |
| | | ctccaaccggaagatttcgcaacc | | | |
| | | tattactgtcaacaatggtcaaga | | | |
| | | aatccgccgacattcgggcaggga | | | |
| | | acaaaagtcgagataaaaaggtca | | | |
| PSMA | Anti-PSMA | gacatccagatgactcaaagccct | 53 | DIQMTQSPSTLSASVGDRVTI | 54 |
| 01026 | scFv x Fc | tcaaccctgtccgcaagtgtcggg | | TCRASKSISKYLAWYQQKPG | |
| Anti | Knob | gacagagttactataacgtgcagg | | KAPKLLIHSGSSLESGVPSRFS | |
| PSMA | (Chain 1) | gcaagtaagagtataagtaaatat | | GSGSGTEFTLTISSLQPDDFA | |
| scFv-Fc; | | ctggcctggtatcaacagaaaccc | | TYYCQQHIEYPWTFGQGTKV | |
| Anti CD3 | | gggaaagcccctaaactcctcata | | EIKGGGGSGGGGSGGGGSG | |
| scFv-Fc | | cattcaggatctagccttgagtca | | GGGSQVQLVQSGAEVKKPG | |
| | | ggtgttccgtcaagattctctggt | | ASVKVSCKASGYTFTDYYMH | |
| | | tccggaagcgggaccgagttcacc | | WVRQAPGQGLEWMGYFN | |
| | | ctgacaatcagttcactccagcct | | PYNDYTRYAQKFQGRVTMT | |
| | | gacgacttcgctacttactactgc | | RDTSTSTVYMELSSLRSEDTA | |
| | | cagcagcacatagagtacccctgg | | VYYCARSDGYYDAMDYWG | |
| | | acattcggacaaggaacgaaagtc | | QGTTVTVEPKSSDKTHTCPP | |
| | | gaaatcaagggtggtggaggtagt | | CPAPPAAAPSVFLFPPKPKDT | |
| | | ggtggaggcggatctggcggcggc | | LMISRTPEVTCVVVDVSHED | |
| | | ggttcaggaggtggtggatcccag | | PEVKFNWYVDGVEVHNAKT | |
| | | gtccagctggtacagtctggggct | | KPREEQYNSTYRVVSVLTVLH | |
| | | gaggtgaagaagcctggggcttca | | QDWLNGKEYKCAVSNKALP | |
| | | gtgaaggtctcctgcaaggcttct | | APIEKTISKAKGQPREPQVYT | |
| | | ggatacacattcactgactactac | | LPPSRDELTKNQVSLWCLVK | |
| | | atgcactgggtgcgacaggcccct | | GFYPSDIAVEWESNGQPEN | |
| | | ggacaagggcttgagtggatggga | | NYKTTPPVLDSDGSFFLYSKL | |
| | | tattttaatccttataatgattat | | TVDKSRWQQGNVFSCSVM | |
| | | actagatacgcacagaagttccag | | HEALHNHYTQKSLSLSPG | |
| | | ggcagagtcaccatgaccagggac | | | |
| | | acgtctaccagcacagtgtacatg | | | |
| | | gagctgagcagcctgagatctgag | | | |
| | | gacacggccgtgtattactgtgca | | | |
| | | agatcggatggttactacgatgct | | | |
| | | atggactactggggtcaaggaacc | | | |
| | | acagtcaccgtcgagcccaaatct | | | |
| | | tctgacaaaactcacacatgccca | | | |
| | | ccgtgcccagcacctccagccgct | | | |
| | | gcaccgtcagtcttcctcttcccc | | | |
| | | ccaaaacccaaggacaccctcatg | | | |
| | | atctcccggacccctgaggtcaca | | | |
| | | tgcgtggtggtggacgtgagccac | | | |
| | | gaagaccctgaggtcaagttcaac | | | |
| | | tggtacgtggacggcgtggaggtg | | | |
| | | cataatgccaagacaaagccgcgg | | | |
| | | gaggagcagtacaacagcacgtac | | | |
| | | cgtgtggtcagcgtcctcaccgtc | | | |
| | | ctgcaccaggactggctgaatggc | | | |
| | | aaggaatacaagtgcgcggtctcc | | | |
| | | aacaaagccctcccagcccccatc | | | |
| | | gagaaaaccatctccaaagccaaa | | | |
| | | gggcagccccgagaaccacaggtg | | | |
| | | tacaccctgcccccatcccgggat | | | |
| | | gagctgaccaagaaccaggtcagc | | | |
| | | ctgtggtgcctggtcaaaggcttc | | | |
| | | tatccaagcgacatcgccgtggag | | | |
| | | tgggagagcaatgggcagccggag | | | |
| | | aacaactacaagaccacgcctccc | | | |
| | | gtgctggactccgacggctccttc | | | |
| | | ttcctctacagcaagctcaccgtg | | | |
| | | gacaagagcaggtggcagcagggg | | | |
| | | aacgtcttctcatgctccgtgatg | | | |
| | | catgaggctctgcacaaccactac | | | |
| | | acgcagaagagcctctccctgtct | | | |
| | | ccgggt | | | |
| | Anti-CD3 | caagtacaactcgttcaaagtggc | 55 | QVQLVQSGAEVKKPGASVK | 56 |
| | scFv x Fc | gcagaagtaaagaagccaggcgcc | | VSCKASGYTFTRSTMHWVR | |
| | Hole | agtgttaaggtgagctgcaaggca | | QAPGQGLEWIGYINPSSAYT | |
| | (Chain 2) | agcgggtacaccttcacccggtct | | NYAQKFQGRVTLTADKSTST | |
| | | acaatgcactgggtaagacaagca | | AYMELSSLRSEDTAVYYCASP | |
| | | ccagggcaaggactcgaatggatt | | QVHYDYNGFPYWGQGTLVT | |
| | | ggttacatcaacccttcctctgca | | VSSGGGGSGGGGSGGGGSG | |
| | | tacaccaactacgctcaaaagttc | | GGGSDIQMTQSPSSLSASVG | |
| | | cagggccgcgttactttgacagcg | | DRVTITCRASSSVSYMNWYQ | |
| | | gataaatctacatccacggcctat | | QKPGKAPKRWIYDSSKLASG | |
| | | atggaactgtcaagcctcaggagc | | VPSRFSGSGSGTDFTLTISSLQ | |
| | | gaggacacagcggtatattactgt | | PEDFATYYCQQWSRNPPTF | |
| | | gcatctccccaggtccattatgac | | GQGTKVEIKEPKSSDKTHTCP | |
| | | tacaacgggtttccgtactgggga | | PCPAPPAAAPSVFLFPPKPKD | |
| | | caaggaactctggttacagtcagt | | TLMISRTPEVTCVVVDVSHE | |
| | | agcggtggtggaggtagtggtgga | | DPEVKFNWYVDGVEVHNAK | |
| | | ggcggatctggcggcggcggttca | | TKPREEQYNSTYRVVSVLTVL | |
| | | ggaggtggtggatccgatatccag | | HQDWLNGKEYKCAVSNKAL | |
| | | atgacccaaagtccgagctcgttg | | PAPIEKTISKAKGQPREPQVY | |
| | | agtgcaagtgtaggagaccgcgta | | TLPPSRDELTKNQVSLSCAVK | |
| | | acgattacttgcagagcttcaagt | | GFYPSDIAVEWESNGQPEN | |
| | | tccgtatcctacatgaattggtat | | NYKTTPPVLDSDGSFFLVSKL | |
| | | cagcaaaagcctggaaaagcccct | | TVDKSRWQQGNVFSCSVM | |
| | | aagcgctggatatacgattcaagt | | HEALHNRFTQKSLSLSPG | |
| | | aagttggcttctggcgtcccatca | | | |
| | | cggttttctggttcaggttccggt | | | |
| | | acagattttacgctgacaatcagc | | | |
| | | tctctccaaccggaagatttcgca | | | |
| | | acctattactgtcaacaatggtca | | | |
| | | agaaatccgccgacattcgggcag | | | |
| | | ggaacaaaagtcgagataaaagag | | | |
| | | cccaaatcttctgacaaaactcac | | | |
| | | acatgcccaccgtgcccagcacct | | | |
| | | ccagccgctgcaccgtcagtcttc | | | |
| | | ctcttccccccaaaacccaaggac | | | |
| | | accctcatgatctcccggacccct | | | |
| | | gaggtcacatgcgtggtggtggac | | | |
| | | gtgagccacgaagaccctgaggtc | | | |
| | | aagttcaactggtacgtggacggc | | | |
| | | gtggaggtgcataatgccaagaca | | | |
| | | aagccgcgggaggagcagtacaac | | | |
| | | agcacgtaccgtgtggtcagcgtc | | | |
| | | ctcaccgtcctgcaccaggactgg | | | |
| | | ctgaatggcaaggaatacaagtgc | | | |
| | | gcggtctccaacaaagccctccca | | | |
| | | gcccccatcgagaaaaccatctcc | | | |
| | | aaagccaaagggcagccccgagaa | | | |
| | | ccacaggtgtacaccctgccccca | | | |
| | | tcccgggatgagctgaccaagaac | | | |
| | | caggtcagcctgtcttgcgctgtc | | | |
| | | aaaggcttctatccaagcgacatc | | | |
| | | gccgtggagtgggagagcaatggg | | | |
| | | cagccggagaacaactacaagacc | | | |
| | | acgcctcccgtgctggactccgac | | | |
| | | ggctccttcttcctcgttagcaag | | | |
| | | ctcaccgtggacaagagcaggtgg | | | |
| | | cagcaggggaacgtcttctcatgc | | | |
| | | tccgtgatgcatgaggctctgcac | | | |
| | | aaccgtttcacgcagaagagcctc | | | |
| | | tccctgtctccgggt | | | |
| PSMA | Anti PSMA | caggtgcagctggtgcagtctggg | 57 | QVQLVQSGAEVKKPGASVK | 58 |
| 01070 | scFv x Fc | gctgaggtgaagaagcctggggcc | | VSCKASGYTFTDYYMHWVR | |
| Anti | Knob | tcagtgaaggtttcctgcaaggca | | QAPGQGLEWMGYFNPYND | |
| PSMA | (Chain 1) | tctggatacaccttcaccgactac | | YTRYAQKFQGRVTMTRDTS | |
| scFv-Fc; | | tatatgcactgggtgcgacaggcc | | TSTVYMELSSLRSEDTAVYYC | |
| Anti CD3 | | cctggacaagggcttgagtggatg | | ARSDGYYDAMDYWGQGTT | |
| scFv-Fc | | ggatatttcaacccttataatgat | | VTVSSGGGGSGGGGSGGGG | |
| | | tacacacgctacgcacagaagttc | | SGGGGSDIQMTQSPSSLSAS | |
| | | cagggcagagtcaccatgaccagg | | VGDRVTITCRASKSISKYLAW | |
| | | gacacgtccacgagcacagtctac | | YQQKPGKAPKLLIHSGSSLES | |
| | | atggagctgagcagcctgagatct | | GVPSRFSGSGSGTEFTLTISSL | |
| | | gaggacacggccgtgtattactgt | | QPDDFATYYCQQHIEYPWTF | |
| | | gcgagatctgacggctactacgac | | GQGTKVEIKEPKSSDKTHTCP | |
| | | gctatggactactgggggcaaggg | | PCPAPPAAAPSVFLFPPKPKD | |
| | | accacggtcaccgtctcctcgggt | | TLMISRTPEVTCVVVDVSHE | |
| | | ggtggaggtagtggtggaggcgga | | DPEVKFNWYVDGVEVHNAK | |
| | | tctggcggcggcggttcaggaggt | | TKPREEQYNSTYRVVSVLTVL | |
| | | ggtggatccgacatccagatgacc | | HQDWLNGKEYKCAVSNKAL | |
| | | cagaaaccagggaaagcccctaag | | PAPIEKTISKAKGQPREPQVY | |
| | | tctgtaggagacagagtcaccatc | | TLPPSRDELTKNQVSLWCLV | |
| | | acttgccgggccagtaagagtatt | | KGFYPSDIAVEWESNGQPEN | |
| | | agtaagtacttggcctggtatcag | | NYKTTPPVLDSDGSFFLYSKL | |
| | | cagtctccttcctccctgtctgca | | TVDKSRWQQGNVFSCSVM | |
| | | ctcctgatccattctggctccagt | | HEALHNHYTQKSLSLSPG | |
| | | ttggaaagtggggtcccatcaagg | | | |
| | | ttcagcggcagtggatctgggaca | | | |
| | | gaattcactctcaccatcagcagc | | | |
| | | ctgcagcctgatgattttgcaact | | | |
| | | tattactgccaacagcatattgaa | | | |
| | | tatccttggacgttcggccaaggg | | | |
| | | accaaggtggaaatcaaagagccc | | | |
| | | aaatcttctgacaaaactcacaca | | | |
| | | tgcccaccgtgcccagcacctcca | | | |
| | | gccgctgcaccgtcagtcttcctc | | | |
| | | ttccccccaaaacccaaggacacc | | | |
| | | ctcatgatctcccggacccctgag | | | |
| | | gtcacatgcgtggtggtggacgtg | | | |
| | | agccacgaagaccctgaggtcaag | | | |
| | | ttcaactggtacgtggacggcgtg | | | |
| | | gaggtgcataatgccaagacaaag | | | |
| | | ccgcgggaggagcagtacaacagc | | | |
| | | acgtaccgtgtggtcagcgtcctc | | | |
| | | accgtcctgcaccaggactggctg | | | |
| | | aatggcaaggaatacaagtgcgcg | | | |
| | | gtctccaacaaagccctcccagcc | | | |
| | | cccatcgagaaaaccatctccaaa | | | |
| | | gccaaagggcagccccgagaacca | | | |
| | | caggtgtacaccctgcccccatcc | | | |
| | | cgggatgagctgaccaagaaccag | | | |
| | | gtcagcctgtggtgcctggtcaaa | | | |
| | | ggcttctatccaagcgacatcgcc | | | |
| | | gtggagtgggagagcaatgggcag | | | |
| | | ccggagaacaactacaagaccacg | | | |
| | | cctcccgtgctggactccgacggc | | | |
| | | tccttcttcctctacagcaagctc | | | |
| | | accgtggacaagagcaggtggcag | | | |
| | | caggggaacgtcttctcatgctcc | | | |
| | | gtgatgcatgaggctctgcacaac | | | |
| | | cactacacgcagaagagcctctcc | | | |
| | | ctgtctccgggt | | | |
| | Anti CD3 | Same sequernce as | 55 | | |
| | scFv x Fc | PSMA01026 Chain 2 | | | |
| | Hole | | | | |
| | (Chain 2) | | | | |
| PSMA | Anti PSMA | Same sequence as | 57 | | |
| 01071 | scFv x Fc | PSMA01070 Chain 1 | | | |
| Anti | Knob | | | | |
| PSMA | (Chain 1) | | | | |
| scFv x Fc; | Anti PSMA | caggtgcagctggtgcagtctggg | 59 | QVQLVQSGAEVKKPGASVK | 60 |
| Anti PSMA | scFv x Fc | gctgaggtgaagaagcctggggcc | | VSCKASGYTFTDYYMHWVR | |
| scFv x Fc | Hole x | tcagtgaaggtttcctgcaaggca | | QAPGQGLEWMGYFNPYND | |
| x Anti | linker x | tctggatacaccttcaccgactac | | YTRYAQKFQGRVTMTRDTS | |
| CD3 scFv | Anti CD3 | tatatgcactgggtgcgacaggcc | | TSTVYMELSSLRSEDTAVYYC | |
| | scFv | cctggacaagggcttgagtggatg | | ARSDGYYDAMDYWGQGTT | |
| | (Chain 2) | ggatatttcaacccttataatgat | | VTVSSGGGGSGGGGSGGGG | |
| | | tacacacgctacgcacagaagttc | | SGGGGSDIQMTQSPSSLSAS | |
| | | cagggcagagtcaccatgaccagg | | VGDRVTITCRASKSISKYLAW | |
| | | gacacgtccacgagcacagtctac | | YQQKPGKAPKLLIHSGSSLES | |
| | | atggagctgagcagcctgagatct | | GVPSRFSGSGSGTEFTLTISSL | |
| | | gaggacacggccgtgtattactgt | | QPDDFATYYCQQHIEYPWTF | |
| | | gcgagatctgacggctactacgac | | GQGTKVEIKEPKSSDKTHTCP | |
| | | gctatggactactgggggcaaggg | | PCPAPPAAAPSVFLFPPKPKD | |
| | | accacggtcaccgtctcctcgggt | | TLMISRTPEVTCVVVDVSHE | |
| | | ggtggaggtagtggtggaggcgga | | DPEVKFNWYVDGVEVHNAK | |
| | | tctggcggcggcggttcaggaggt | | TKPREEQYNSTYRVVSVLTVL | |
| | | ggtggatccgacatccagatgacc | | HQDWLNGKEYKCAVSNKAL | |
| | | cagtctccttcctccctgtctgca | | PAPIEKTISKAKGQPREPQVY | |
| | | tctgtaggagacagagtcaccatc | | TLPPSRDELTKNQVSLSCAVK | |
| | | acttgccgggccagtaagagtatt | | GFYPSDIAVEWESNGQPEN | |
| | | agtaagtacttggcctggtatcag | | NYKTTPPVLDSDGSFFLVSKL | |
| | | cagaaaccagggaaagcccctaag | | TVDKSRWQQGNVFSCSVM | |
| | | ctcctgatccattctggctccagt | | HEALHNRFTQKSLSLSPGGG | |
| | | ttggaaagtggggtcccatcaagg | | GSPSQVQLVQSGAEVKKPG | |
| | | ttcagcggcagtggatctgggaca | | ASVKVSCKASGYTFTRSTMH | |
| | | gaattcactctcaccatcagcagc | | WVRQAPGQGLEWIGYINPS | |
| | | ctgcagcctgatgattttgcaact | | SAYTNYAQKFQGRVTLTADK | |
| | | tattactgccaacagcatattgaa | | STSTAYMELSSLRSEDTAVYY | |
| | | tatccttggacgttcggccaaggg | | CASPQVHYDYNGFPYWGQ | |
| | | accaaggtggaaatcaaagagccc | | GTLVTVSSGGGGSGGGGSG | |
| | | aaatcttctgacaaaactcacaca | | GGGSGGGGSDIQMTQSPSS | |
| | | tgcccaccgtgcccagcacctcca | | LSASVGDRVTITCRASSSVSY | |
| | | gccgctgcaccgtcagtcttcctc | | MNWYQQKPGKAPKRWIYD | |
| | | ttccccccaaaacccaaggacacc | | SSKLASGVPSRFSGSGSGTDF | |
| | | ctcatgatctcccggacccctgag | | TLTISSLQPEDFATYYCQQWS | |
| | | gtcacatgcgtggtggtggacgtg | | RNPPTFGQGTKVEIKRS | |
| | | agccacgaagaccctgaggtcaag | | | |
| | | ttcaactggtacgtggacggcgtg | | | |
| | | gaggtgcataatgccaagacaaag | | | |
| | | ccgcgggaggagcagtacaacagc | | | |
| | | acgtaccgtgtggtcagcgtcctc | | | |
| | | accgtcctgcaccaggactggctg | | | |
| | | aatggcaaggaatacaagtgcgcg | | | |
| | | gtctccaacaaagccctcccagcc | | | |
| | | cccatcgagaaaaccatctccaaa | | | |
| | | gccaaagggcagccccgagaacca | | | |
| | | caggtgtacaccctgcccccatcc | | | |
| | | cgggatgagctgaccaagaaccag | | | |
| | | gtcagcctgtcttgcgctgtcaaa | | | |
| | | ggcttctatccaagcgacatcgcc | | | |
| | | gtggagtgggagagcaatgggcag | | | |
| | | ccggagaacaactacaagaccacg | | | |
| | | cctcccgtgctggactccgacggc | | | |
| | | tccttcttcctcgttagcaagctc | | | |
| | | accgtggacaagagcaggtggcag | | | |
| | | caggggaacgtcttctcatgctcc | | | |
| | | gtgatgcatgaggctctgcacaac | | | |
| | | cgtttcacgcagaagagcctctcc | | | |
| | | ctgtctccgggcggcgggggatcc | | | |
| | | ccgtcacaagtacaactcgttcaa | | | |
| | | agtggcgcagaagtaaagaagcca | | | |
| | | ggcgccagtgttaaggtgagctgc | | | |
| | | aaggcaagcgggtacaccttcacc | | | |
| | | cggtctacaatgcactgggtaaga | | | |
| | | caagcaccagggcaaggactcgaa | | | |
| | | tggattggttacatcaacccttcc | | | |
| | | tctgcatacaccaactacgctcaa | | | |
| | | aagttccagggccgcgttactttg | | | |
| | | acagcggataaatctacatccacg | | | |
| | | gcctatatggaactgtcaagcctc | | | |
| | | aggagcgaggacacagcggtatat | | | |
| | | tactgtgcatctccccaggtccat | | | |
| | | tatgactacaacgggtttccgtac | | | |
| | | tggggacaaggaactctggttaca | | | |
| | | gtcagtagcggtggtggaggtagt | | | |
| | | ggtggaggcggatctggcggcggc | | | |
| | | ggttcaggaggtggtggatccgat | | | |
| | | atccagatgacccaaagtccgagc | | | |
| | | tcgttgagtgcaagtgtaggagac | | | |
| | | cgcgtaacgattacttgcagagct | | | |
| | | tcaagttccgtatcctacatgaat | | | |
| | | tggtatcagcaaaagcctggaaaa | | | |
| | | gcccctaagcgctggatatacgat | | | |
| | | tcaagtaagttggcttctggcgtc | | | |
| | | ccatcacggttttctggttcaggt | | | |
| | | tccggtacagattttacgctgaca | | | |
| | | atcagctctctccaaccggaagat | | | |
| | | ttcgcaacctattactgtcaacaa | | | |
| | | tggtcaagaaatccgccgacattc | | | |
| | | gggcagggaacaaaagtcgagata | | | |
| | | aaaaggtcacaagtacaactcgtt | | | |
| | | caaagtggcgcagaagtaaagaag | | | |
| | | ccaggcgccagtgttaaggtgagc | | | |
| | | tgcaaggcaagcgggtacaccttc | | | |
| | | acccggtctacaatgcactgggta | | | |
| | | agacaagcaccagggcaaggactc | | | |
| | | gaatggattggttacatcaaccct | | | |
| | | tcctctgcatacaccaactacgct | | | |
| | | caaaagttccagggccgcgttact | | | |
| | | ttgacagcggataaatctacatcc | | | |
| | | acggcctatatggaactgtcaagc | | | |
| | | ctcaggagcgaggacacagcggta | | | |
| | | tattactgtgcatctccccaggtc | | | |
| | | cattatgactacaacgggtttccg | | | |
| | | tactggggacaaggaactctggtt | | | |
| | | acagtcagtagcggtggtggaggt | | | |
| | | agtggtggaggcggatctggcggc | | | |
| | | ggcggttcaggaggtggtggatcc | | | |
| | | gatatccagatgacccaaagtccg | | | |
| | | agctcgttgagtgcaagtgtagga | | | |
| | | gaccgcgtaacgattacttgcaga | | | |
| | | gcttcaagttccgtatcctacatg | | | |
| | | aattggtatcagcaaaagcctgga | | | |
| | | aaagcccctaagcgctggatatac | | | |
| | | gattcaagtaagttggcttctggc | | | |
| | | gtcccatcacggttttctggttca | | | |
| | | ggttccggtacagattttacgctg | | | |
| | | acaatcagctctctccaaccggaa | | | |
| | | gatttcgcaacctattactgtcaa | | | |
| | | caatggtcaagaaatccgccgaca | | | |
| | | ttcgggcagggaacaaaagtcgag | | | |
| | | ataaaagagcccaaatcttctgac | | | |
| | | aaaactcacacatgcccaccgtgc | | | |
| | | ccagcacctccagccgctgcaccg | | | |
| | | tcagtcttcctcttccccccaaaa | | | |
| | | cccaaggacaccctcatgatctcc | | | |
| | | cggacccctgaggtcacatgcgtg | | | |
| | | gtggtggacgtgagccacgaagac | | | |
| | | cctgaggtcaagttcaactggtac | | | |
| | | gtggacggcgtggaggtgcataat | | | |
| | | gccaagacaaagccgcgggaggag | | | |
| | | cagtacaacagcacgtaccgtgtg | | | |
| | | gtcagcgtcctcaccgtcctgcac | | | |
| | | caggactggctgaatggcaaggaa | | | |
| | | tacaagtgcgcggtctccaacaaa | | | |
| | | gccctcccagcccccatcgagaaa | | | |
| | | accatctccaaagccaaagggcag | | | |
| | | ccccgagaaccacaggtgtacacc | | | |
| | | ctgcccccatcccgggatgagctg | | | |
| | | accaagaaccaggtcagcctgtct | | | |
| | | tgcgctgtcaaaggcttctatcca | | | |
| | | agcgacatcgccgtggagtgggag | | | |
| | | agcaatgggcagccggagaacaac | | | |
| | | tacaagaccacgcctcccgtgctg | | | |
| | | gactccgacggctccttcttcctc | | | |
| | | gttagcaagctcaccgtggacaag | | | |
| | | agcaggtggcagcaggggaacgtc | | | |
| | | ttctcatgctccgtgatgcatgag | | | |
| | | gctctgcacaaccgtttcacgcag | | | |
| | | aagagcctctccctgtctccgggt | | | |
| PSMA | Anti PSMA | Same sequence as | 57 | | |
| 01086 | x Fc Knob | PSMA01070 Chain 1 | | | |
| Anti | (Chain 1) | | | | |
| PSMA | Anti PSMA | caggtgcagctggtgcagtctggg | 61 | QVQLVQSGAEVKKPGASVK | |
| scFv x Fc; | x Fc Hole | gctgaggtgaagaagcctggggcc | | VSCKASGYTFTDYYMHWVR | |
| Anti | x Anti | tcagtgaaggtttcctgcaaggca | | QAPGQGLEWMGYFNPYND | |
| PSMA | CD3 | tctggatacaccttcaccgactac | | YTRYAQKFQGRVTMTRDTS | |
| scFv x Fc | (Chain 2) | tatatgcactgggtgcgacaggcc | | TSTVYMELSSLRSEDTAVYYC | |
| x linker | | cctggacaagggcttgagtggatg | | ARSDGYYDAMDYWGQGTT | |
| x Anti | | ggatatttcaacccttataatgat | | VTVSSGGGGGGGGSGGGG | |
| CD3 scFv | | tacacacgctacgcacagaagttc | | SGGGGSDIQMTQSPSSLSAS | |
| | | cagggcagagtcaccatgaccagg | | VGDRVTITCRASKSISKYLAW | |
| | | gacacgtccacgagcacagtctac | | YQQKPGKAPKLLIHSGSSLES | |
| | | atggagctgagcagcctgagatct | | GVPSRFSGSGSGTEFTLTISSL | |
| | | gaggacacggccgtgtattactgt | | QPDDFATYYCQQHIEYPWTF | |
| | | gcgagatctgacggctactacgac | | GQGTKVEIKEPKSSDKTHTCP | |
| | | gctatggactactgggggcaaggg | | PCPAPPAAAPSVFLFPPKPKD | |
| | | accacggtcaccgtctcctcgggt | | TLMISRTPEVTCVVVDVSHE | |
| | | ggtggatccgacatccagatgacc | | DPEVKFNWYVDGVEVHNAK | |
| | | tctggcggcggcggttcaggaggt | | TKPREEQYNSTYRVVSVLTVL | |
| | | ggtggaggtagtggtggaggcgga | | HQDWLNGKEYKCAVSNKAL | |
| | | cagtctccttcctccctgtctgca | | PAPIEKTISKAKGQPREPQVY | |
| | | tctgtaggagacagagtcaccatc | | TLPPSRDELTKNQVSLSCAVK | |
| | | acttgccgggccagtaagagtatt | | GFYPSDIAVEWESNGQPEN | |
| | | agtaagtacttggcctggtatcag | | NYKTTPPVLDSDGSFFLVSKL | |
| | | cagaaaccagggaaagcccctaag | | TVDKSRWQQGNVFSCSVM | |
| | | ctcctgatccattctggctccagt | | HEALHNRFTQKSLSLSPGGG | |
| | | ttggaaagtggggtcccatcaagg | | GSPSDIQMTQSPSSLSASVG | |
| | | ttcagcggcagtggatctgggaca | | DRVTITCRASSSVSYMNWYQ | |
| | | gaattcactctcaccatcagcagc | | QKPGKAPKRWIYDSSKLASG | |
| | | ctgcagcctgatgattttgcaact | | VPSRFSGSGSGTDFTLTISSLQ | |
| | | tattactgccaacagcatattgaa | | PEDFATYYCQQWSRNPPTF | |
| | | tatccttggacgttcggccaaggg | | GQGTKVEIKGGGGSGGGGS | |
| | | accaaggtggaaatcaaagagccc | | GGGGSGGGGSQVQLVQSG | |
| | | aaatcttctgacaaaactcacaca | | AEVKKPGASVKVSCKASGYT | |
| | | tgcccaccgtgcccagcacctcca | | FTRSTMHWVRQAPGQGLE | |
| | | gccgctgcaccgtcagtcttcctc | | WIGYINPSSAYTNYAQKFQG | |
| | | ttccccccaaaacccaaggacacc | | RVTLTADKSTSTAYMELSSLR | |
| | | ctcatgatctcccggacccctgag | | SEDTAVYYCASPQVHYDYNG | |
| | | gtcacatgcgtggtggtggacgtg | | FPYWGQGTLVTVSS | |
| | | agccacgaagaccctgaggtcaag | | | |
| | | ttcaactggtacgtggacggcgtg | | | |
| | | gaggtgcataatgccaagacaaag | | | |
| | | ccgcgggaggagcagtacaacagc | | | |
| | | acgtaccgtgtggtcagcgtcctc | | | |
| | | accgtcctgcaccaggactggctg | | | |
| | | aatggcaaggaatacaagtgcgcg | | | |
| | | gtctccaacaaagccctcccagcc | | | |
| | | cccatcgagaaaaccatctccaaa | | | |
| | | gccaaagggcagccccgagaacca | | | |
| | | caggtgtacaccctgcccccatcc | | | |
| | | cgggatgagctgaccaagaaccag | | | |
| | | gtcagcctgtcttgcgctgtcaaa | | | |
| | | ggcttctatccaagcgacatcgcc | | | |
| | | gtggagtgggagagcaatgggcag | | | |
| | | ccggagaacaactacaagaccacg | | | |
| | | cctcccgtgctggactccgacggc | | | |
| | | tccttcttcctcgttagcaagctc | | | |
| | | accgtggacaagagcaggtggcag | | | |
| | | caggggaacgtcttctcatgctcc | | | |
| | | gtgatgcatgaggctctgcacaac | | | |
| | | cgtttcacgcagaagagcctctcc | | | |
| | | ctgtctccgggcggcgggggatcc | | | |
| | | ccgtcagatatccagatgacccaa | | | |
| | | agtccgagctcgttgagtgcaagt | | | |
| | | gtaggagaccgcgtaacgattact | | | |
| | | tgcagagcttcaagttccgtatcc | | | |
| | | tacatgaattggtatcagcaaaag | | | |
| | | cctggaaaagcccctaagcgctgg | | | |
| | | atatacgattcaagtaagttggct | | | |
| | | tctggcgtcccatcacggttttct | | | |
| | | ggttcaggttccggtacagatttt | | | |
| | | acgctgacaatcagctctctccaa | | | |
| | | ccggaagatttcgcaacctattac | | | |
| | | tgtcaacaatggtcaagaaatccg | | | |
| | | ccgacattcgggcagggaacaaaa | | | |
| | | gtcgagataaaaggcggaggcgga | | | |
| | | agcggaggtgggggctccggaggc | | | |
| | | gggggaagcggcggaggtggctct | | | |
| | | caagtacaactcgttcaaagtggc | | | |
| | | gcagaagtaaagaagccaggcgcc | | | |
| | | agtgttaaggtgagctgcaaggca | | | |
| | | agcgggtacaccttcacccggtct | | | |
| | | acaatgcactgggtaagacaagca | | | |
| | | ccagggcaaggactcgaatggatt | | | |
| | | ggttacatcaacccttcctctgca | | | |
| | | tacaccaactacgctcaaaagttc | | | |
| | | cagggccgcgttactttgacagcg | | | |
| | | gataaatctacatccacggcctat | | | |
| | | atggaactgtcaagcctcaggagc | | | |
| | | gaggacacagcggtatattactgt | | | |
| | | gcatctccccaggtccattatgac | | | |
| | | tacaacgggtttccgtactgggga | | | |
| | | caaggaactctggttacagtcagt | | | |
| | | agc | | | |

| | | DNA | | AA |
|---|---|---|---|---|
| | | SEQ | | SEQ |
| | | ID | | ID |
| Fc | DNA Sequence | NO: | AA Sequence | NO: |
| TSC01007 | tcctcggagcccaaatcttctgacaaaactcac | 63 | SSEPKSSDKTHTCPPCPAPP | 64 |
| PAAdel Fc | acatgcccaccgtgcccagcacctccagccgct | | AAAPSVFLFPPKPKDTLMIS | |
| | gcaccgtcagtcttcctcttccccccaaaaccc | | RTPEVTCVVVDVSHEDPEV | |
| | aaggacaccctcatgatctcccggacccctgag | | KFNWYVDGVEVHNAKTKP | |
| | gtcacatgcgtggtggtggacgtgagccacgaa | | REEQYNSTYRVVSVLTVLH | |
| | gaccctgaggtcaagttcaactggtacgtggac | | QDWLNGKEYKCAVSNKAL | |
| | ggcgtggaggtgcataatgccaagacaaagccg | | PAPIEKTISKAKGQPREPQV | |
| | cgggaggagcagtacaacagcacgtaccgtgtg | | YTLPPSRDELTKNQVSLTCL | |
| | gtcagcgtcctcaccgtcctgcaccaggactgg | | VKGFYPSDIAVEWESNGQ | |
| | ctgaatggcaaggaatacaagtgcgcggtctcc | | PENNYKTTPPVLDSDGSFF | |
| | aacaaagccctcccagcccccatcgagaaaacc | | LYSKLTVDKSRWQQGNVF | |
| | atctccaaagccaaagggcagccccgagaacca | | SCSVMHEALHNHYTQKSL | |
| | caggtgtacaccctgcccccatcccgggatgag | | SLSPG | |
| | ctgaccaagaaccaggtcagcctgacctgcctg | | | |
| | gtcaaaggcttctatccaagcgacatcgccgtg | | | |
| | gagtgggagagcaatgggcagccggagaacaac | | | |
| | tacaagaccacgcctcccgtgctggactccgac | | | |
| | ggctccttcttcctctacagcaagctcaccgtg | | | |
| | gacaagagcaggtggcagcaggggaacgtcttc | | | |
| | tcatgctccgtgatgcatgaggctctgcacaac | | | |
| | cactacacgcagaagagcctctccctgtctccg | | | |
| | ggt | | | |
| PAAdel | GAGCCCAAATCTTCTGACAAAACTCACACATGC | 65 | EPKSSDKTHTCPPCPAPPA | 66 |
| Fc + | CCACCGTGCCCAGCACCTCCAGCCGCTGCACCG | | AAPSVFLFPPKPKDTLMISR | |
| Knob | TCAGTCTTCCTCTTCCCCCCAAAACCCAAGGAC | | TPEVTCVVVDVSHEDPEVK | |
| | ACCCTCATGATCTCCCGGACCCCTGAGGTCACA | | FNWYVDGVEVHNAKTKPR | |
| | TGCGTGGTGGTGGACGTGAGCCACGAAGACCCT | | EEQYNSTYRVVSVLTVLHQ | |
| | GAGGTCAAGTTCAACTGGTACGTGGACGGCGTG | | DWLNGKEYKCAVSNKALP | |
| | GAGGTGCATAATGCCAAGACAAAGCCGCGGGAG | | APIEKTISKAKGQPREPQVY | |
| | GAGCAGTACAACAGCACGTACCGTGTGGTCAGC | | TLPPSRDELTKNQVSLWCL | |
| | GTCCTCACCGTCCTGCACCAGGACTGGCTGAAT | | VKGFYPSDIAVEWESNGQ | |
| | GGCAAGGAATACAAGTGCGCGGTCTCCAACAAA | | PENNYKTTPPVLDSDGSFF | |
| | GCCCTCCCAGCCCCCATCGAGAAAACCATCTCC | | LYSKLTVDKSRWQQGNVF | |
| | AAAGCCAAAGGGCAGCCCCGAGAACCACAGGTG | | SCSVMHEALHNHYTQKSL | |
| | TACACCCTGCCCCCATCCCGGGATGAGCTGACC | | SLSP | |
| | AAGAACCAGGTCAGCCTGTGGTGCCTGGTCAAA | | | |
| | GGCTTCTATCCAAGCGACATCGCCGTGGAGTGG | | | |
| | GAGAGCAATGGGCAGCCGGAGAACAACTACAAG | | | |
| | ACCACGCCTCCCGTGCTGGACTCCGACGGCTCC | | | |
| | TTCTTCCTCTACAGCAAGCTCACCGTGGACAAG | | | |
| | AGCAGGTGGCAGCAGGGGAACGTCTTCTCATGC | | | |
| | TCCGTGATGCATGAGGCTCTGCACAACCACTAC | | | |
| | ACGCAGAAGAGCCTCTCCCTGTCTCCG | | | |
| PAAdel | GAGCCCAAATCTTCTGACAAAACTCACACATGC | 67 | EPKSSDKTHTCPPCPAPPA | 68 |
| Fc + | CCACCGTGCCCAGCACCTCCAGCCGCTGCACCG | | AAPSVFLFPPKPKDTLMISR | |
| Hole | TCAGTCTTCCTCTTCCCCCCAAAACCCAAGGAC | | TPEVTCVVVDVSHEDPEVK | |
| | ACCCTCATGATCTCCCGGACCCCTGAGGTCACA | | FNWYVDGVEVHNAKTKPR | |
| | TGCGTGGTGGTGGACGTGAGCCACGAAGACCCT | | EEQYNSTYRVVSVLTVLHQ | |
| | GAGGTCAAGTTCAACTGGTACGTGGACGGCGTG | | DWLNGKEYKCAVSNKALP | |
| | GAGGTGCATAATGCCAAGACAAAGCCGCGGGAG | | APIEKTISKAKGQPREPQVY | |
| | GAGCAGTACAACAGCACGTACCGTGTGGTCAGC | | TLPPSRDELTKNQVSLSCA | |
| | GTCCTCACCGTCCTGCACCAGGACTGGCTGAAT | | VKGFYPSDIAVEWESNGQ | |
| | GGCAAGGAATACAAGTGCGCGGTCTCCAACAAA | | PENNYKTTPPVLDSDGSFF | |
| | GCCCTCCCAGCCCCCATCGAGAAAACCATCTCC | | LVSKLTVDKSRWQQGNVF | |
| | AAAGCCAAAGGGCAGCCCCGAGAACCACAGGTG | | SCSVMHEALHNHYTQKSL | |
| | TACACCCTGCCCCCATCCCGGGATGAGCTGACC | | SLSPG | |
| | AAGAACCAGGTCAGCCTGTCTTGCGCTGTCAAA | | | |
| | GGCTTCTATCCAAGCGACATCGCCGTGGAGTGG | | | |
| | GAGAGCAATGGGCAGCCGGAGAACAACTACAAG | | | |
| | ACCACGCCTCCCGTGCTGGACTCCGACGGCTCC | | | |
| | TTCTTCCTCGTTAGCAAGCTCACCGTGGACAAG | | | |
| | AGCAGGTGGCAGCAGGGGAACGTCTTCTCATGC | | | |
| | TCCGTGATGCATGAGGCTCTGCACAACCACTAC | | | |
| | ACGCAGAAGAGCCTCTCCCTGTCTCCGGGT | | | |

| | | | | DNA | | AA |
|---|---|---|---|---|---|---|
| | | | | SEQ | | SEQ |
| Construct | | Component | DNA Sequence | ID NO | AA sequence | ID NO |
| PSMA | Anti | HCDR1 | GGATACACCTTCACCGA | 69 | GYTFTDYY | 70 |
| 01107 | PSMA | | CTACTAT | | | |
| Anti-PSMA | scFv | HCDR2 | TTCAACCCTTATAATGA | 71 | FNPYNDYT | 72 |
| scFv x Fc x | | | TTACACA | | | |
| linker x Anti | | HCDR3 | GCGAGATCTGACGGCTA | 73 | ARSDGYYDAMDY | 74 |
| CD3 scFv; | | | CTACGACGCTATGGACT | | | |
| Anti PSMA | | | AC | | | |
| scFv x Fc | | LCDR1 | AAGAGTATTAGTAAGTA | 75 | KSISKY | 76 |
| | | | C | | | |
| | | LCDR2 | TCTGGCTCC | 77 | SGS | 78 |
| | | LCDR3 | CAACAGCATATTGAATA | 79 | QQHIEYPWT | 80 |
| | | | TCCTTGGACG | | | |
| | | V_{H} | CAGGTGCAGCTGGTGC | 81 | QVQLVQSGAEVKKPGAS | 82 |
| | | | AGTCTGGGGCTGAGGT | | VKVSCKASGYTFTDYYM | |
| | | | GAAGAAGCCTGGGGCC | | HWVRQAPGQGLEWM | |
| | | | TCAGTGAAGGTTTCCT | | GYFNPYNDYTRYAQKFQ | |
| | | | GCAAGGCATCTGGATA | | GRVTMTRDTSTSTVYME | |
| | | | CACCTTCACCGACTAC | | LSSLRSEDTAVYYCARSD | |
| | | | TATATGCACTGGGTGC | | GYYDAMDYWGQGTTV | |
| | | | GACAGGCCCCTGGACA | | TVSS | |
| | | | AGGGCTTGAGTGGATG | | | |
| | | | GGATATTTCAACCCTT | | | |
| | | | ATAATGATTACACACG | | | |
| | | | CTACGCACAGAAGTTC | | | |
| | | | CAGGGCAGAGTCACCA | | | |
| | | | TGACCAGGGACACGTC | | | |
| | | | CACGAGCACAGTCTAC | | | |
| | | | ATGGAGCTGAGCAGCC | | | |
| | | | TGAGATCTGAGGACAC | | | |
| | | | GGCCGTGTATTACTGT | | | |
| | | | GCGAGATCTGACGGCT | | | |
| | | | ACTACGACGCTATGGA | | | |
| | | | CTACTGGGGGCAAGGG | | | |
| | | | ACCACGGTCACCGTCT | | | |
| | | | CCTCG | | | |
| | | V_{L} | GACATCCAGATGACCCA | 83 | DIQMTQSPSSLSASVGD | 84 |
| | | | GTCTCCTTCCTCCCTGT | | RVTITCRASKSISKYLAWY | |
| | | | CTGCATCTGTAGGAGAC | | QQKPGKAPKLLIHSGSSL | |
| | | | AGAGTCACCATCACTTG | | ESGVPSRFSGSGSGTEFT | |
| | | | CCGGGCCAGTAAGAGTA | | LTISSLQPDDFATYYCQQ | |
| | | | TTAGTAAGTACTTGGCC | | HIEYPWTFGQGTKVEIK | |
| | | | TGGTATCAGCAGAAACC | | | |
| | | | AGGGAAAGCCCCTAAGC | | | |
| | | | TCCTGATCCATTCTGGC | | | |
| | | | TCCAGTTTGGAAAGTGG | | | |
| | | | GGTCCCATCAAGGTTCA | | | |
| | | | GCGGCAGTGGATCTGGG | | | |
| | | | ACAGAATTCACTCTCAC | | | |
| | | | CATCAGCAGCCTGCAGC | | | |
| | | | CTGATGATTTTGCAACT | | | |
| | | | TATTACTGCCAACAGCA | | | |
| | | | TATTGAATATCCTTGGA | | | |
| | | | CGTTCGGCCAAGGGACC | | | |
| | | | AAGGTGGAAATCAAA | | | |
| | | scFv | CAGGTGCAGCTGGTGCA | 85 | QVQLVQSGAEVKKPGAS | 86 |
| | | | GTCTGGGGCTGAGGTGA | | VKVSCKASGYTFTDYYM | |
| | | | AGAAGCCTGGGGCCTCA | | HWVRQAPGQGLEWM | |
| | | | GTGAAGGTTTCCTGCAA | | GYFNPYNDYTRYAQKFQ | |
| | | | GGCATCTGGATACACCT | | GRVTMTRDTSTSTVYME | |
| | | | TCACCGACTACTATATG | | LSSLRSEDTAVYYCARSD | |
| | | | CACTGGGTGCGACAGGC | | GYYDAMDYWGQGTTV | |
| | | | CCCTGGACAAGGGCTTG | | TVSSGGGGSGGGGSGG | |
| | | | AGTGGATGGGATATTTC | | GGSGGGGSDIQMTQSP | |
| | | | AACCCTTATAATGATTA | | SSLSASVGDRVTITCRAS | |
| | | | CACACGCTACGCACAGA | | KSISKYLAWYQQKPGKA | |
| | | | AGTTCCAGGGCAGAGTC | | PKLLIHSGSSLESGVPSRF | |
| | | | ACCATGACCAGGGACAC | | SGSGSGTEFTLTISSLQPD | |
| | | | GTCCACGAGCACAGTCT | | DFATYYCQQHIEYPWTF | |
| | | | ACATGGAGCTGAGCAGC | | GQGTKVEIK | |
| | | | CTGAGATCTGAGGACAC | | | |
| | | | GGCCGTGTATTACTGTG | | | |
| | | | CGAGATCTGACGGCTAC | | | |
| | | | TACGACGCTATGGACTA | | | |
| | | | CTGGGGGCAAGGGACCA | | | |
| | | | CGGTCACCGTCTCCTCG | | | |
| | | | GGAGGCGGTGGATCAGG | | | |
| | | | CGGTGGAGGCAGCGGAG | | | |
| | | | GAGGTGGCTCCGGTGGC | | | |
| | | | GGAGGGAGCGACATCCA | | | |
| | | | GATGACCCAGTCTCCTT | | | |
| | | | CCTCCCTGTCTGCATCT | | | |
| | | | GTAGGAGACAGAGTCAC | | | |
| | | | CATCACTTGCCGGGCCA | | | |
| | | | GTAAGAGTATTAGTAAG | | | |
| | | | TACTTGGCCTGGTATCA | | | |
| | | | GCAGAAACCAGGGAAAG | | | |
| | | | CCCCTAAGCTCCTGATC | | | |
| | | | CATTCTGGCTCCAGTTT | | | |
| | | | GGAAAGTGGGGTCCCAT | | | |
| | | | CAAGGTTCAGCGGCAGT | | | |
| | | | GGATCTGGGACAGAATT | | | |
| | | | CACTCTCACCATCAGCA | | | |
| | | | GCCTGCAGCCTGATGAT | | | |
| | | | TTTGCAACTTATTACTG | | | |
| | | | CCAACAGCATATTGAAT | | | |
| | | | ATCCTTGGACGTTCGGC | | | |
| | | | CAAGGGACCAAGGTGGA | | | |
| | | | AATCAAA | | | |
| | Anti- | HCDR1 | GGGTACACCTTCACCCG | 87 | GYTFTRST | 88 |
| | CD3 | | GTCTACA | | | |
| | scFv | HCDR2 | ATCAACCCTTCCTCTGC | 89 | INPSSAYT | 90 |
| | | | ATACACC | | | |
| | | HCDR3 | GCATCTCCCCAGGTCCA | 91 | ASPQVHYDYNGFPY | 92 |
| | | | TTATGACTACAACGGGT | | | |
| | | | TTCCGTAC | | | |
| | | LCDR1 | AGTTCCGTATCCTAC | 93 | SSVSY | 94 |
| | | LCDR2 | GATTCAAGT | 95 | DSS | 96 |
| | | LCDR3 | CAACAATGGTCAAGAAA | 97 | QQWSRNPPT | 98 |
| | | | TCCGCCGACA | | | |
| | | V_{H} | CAAGTACAACTCGTTCA | 99 | QVQLVQSGAEVKKPGAS | 100 |
| | | | AAGTGGCGCAGAAGTAA | | VKVSCKASGYTFTRSTM | |
| | | | AGAAGCCAGGCGCCAGT | | HWVRQAPGQGLEWIGY | |
| | | | GTTAAGGTGAGCTGCAA | | INPSSAYTNYAQKFQGR | |
| | | | GGCAAGCGGGTACACCT | | VTLTADKSTSTAYMELSS | |
| | | | TCACCCGGTCTACAATG | | LRSEDTAVYYCASPQVH | |
| | | | CACTGGGTAAGACAAGC | | YDYNGFPYWGQGTLVT | |
| | | | ACCAGGGCAAGGACTCG | | VSS | |
| | | | AATGGATTGGTTACATC | | | |
| | | | AACCCTTCCTCTGCATA | | | |
| | | | CACCAACTACGCTCAAA | | | |
| | | | AGTTCCAGGGCCGCGTT | | | |
| | | | ACTTTGACAGCGGATAA | | | |
| | | | ATCTACATCCACGGCCT | | | |
| | | | ATATGGAACTGTCAAGC | | | |
| | | | CTCAGGAGCGAGGACAC | | | |
| | | | AGCGGTATATTACTGTG | | | |
| | | | CATCTCCCCAGGTCCAT | | | |
| | | | TATGACTACAACGGGTT | | | |
| | | | TCCGTACTGGGGACAAG | | | |
| | | | GAACTCTGGTTACAGTC | | | |
| | | | AGTAGC | | | |
| | | V_{L} | GATATCCAGATGACCCA | 101 | DIQMTQSPSSLSASVGD | 102 |
| | | | AAGTCCGAGCTCGTTGA | | RVT | |
| | | | GTGCAAGTGTAGGAGAC | | ITCRASSSVSYMNWYQQ | |
| | | | CGCGTAACGATTACTTG | | KPGKAPKRWIYDSSKLAS | |
| | | | CAGAGCTTCAAGTTCCG | | GVPSRFSGSGSGTDFTLT | |
| | | | TATCCTACATGAATTGG | | ISSLQPE | |
| | | | TATCAGCAAAAGCCTGG | | DFATYYCQQWSRNPPTF | |
| | | | AAAAGCCCCTAAGCGCT | | GQGTKVEIK | |
| | | | GGATATACGATTCAAGT | | | |
| | | | AAGTTGGCTTCTGGCGT | | | |
| | | | CCCATCACGGTTTTCTG | | | |
| | | | GTTCAGGTTCCGGTACA | | | |
| | | | GATTTTACGCTGACAAT | | | |
| | | | CAGCTCTCTCCAACCGG | | | |
| | | | AAGATTTCGCAACCTAT | | | |
| | | | TACTGTCAACAATGGTC | | | |
| | | | AAGAAATCCGCCGACAT | | | |
| | | | TCGGGCAGGGAACAAAA | | | |
| | | | GTCGAGATAAAA | | | |
| | | scFv | CAAGTACAACTCGTTCA | 103 | QVQLVQSGAEVKKPGAS | 104 |
| | | | AAGTGGCGCAGAAGTAA | | VKVSCKASGYTFTRSTM | |
| | | | AGAAGCCAGGCGCCAGT | | HWVRQAPGQGLEWIGY | |
| | | | GTTAAGGTGAGCTGCAA | | INPSSAYT | |
| | | | GGCAAGCGGGTACACCT | | NYAQKFQGRVTLTADKS | |
| | | | TCACCCGGTCTACAATG | | TST | |
| | | | CACTGGGTAAGACAAGC | | AYMELSSLRSEDTAVYYC | |
| | | | ACCAGGGCAAGGACTCG | | AS | |
| | | | AATGGATTGGTTACATC | | PQVHYDYNGFPYWGQG | |
| | | | AACCCTTCCTCTGCATA | | TLVTVSSGGGGSGGGGS | |
| | | | CACCAACTACGCTCAAA | | GGGGSGGGGSDIQMTQ | |
| | | | AGTTCCAGGGCCGCGTT | | SPSSLSASVGDRVTITCR | |
| | | | ACTTTGACAGCGGATAA | | ASSSVSYMNWYQQKPG | |
| | | | ATCTACATCCACGGCCT | | KAPKRWIYDSSKLASGVP | |
| | | | ATATGGAACTGTCAAGC | | SRFSGSGSGTDFTLTISSL | |
| | | | CTCAGGAGCGAGGACAC | | QPEDFATYYCQQWSRN | |
| | | | AGCGGTATATTACTGTG | | PPTFGQGTKVEIK | |
| | | | CATCTCCCCAGGTCCAT | | | |
| | | | TATGACTACAACGGGTT | | | |
| | | | TCCGTACTGGGGACAAG | | | |
| | | | GAACTCTGGTTACAGTC | | | |
| | | | AGTAGCGGCGGAGGCGG | | | |
| | | | AAGCGGAGGTGGGGGCT | | | |
| | | | CCGGAGGCGGGGGAAGC | | | |
| | | | GGCGGAGGTGGCTCTGA | | | |
| | | | TATCCAGATGACCCAAA | | | |
| | | | GTCCGAGCTCGTTGAGT | | | |
| | | | GCAAGTGTAGGAGACCG | | | |
| | | | CGTAACGATTACTTGCA | | | |
| | | | GAGCTTCAAGTTCCGTA | | | |
| | | | TCCTACATGAATTGGTA | | | |
| | | | TCAGCAAAAGCCTGGAA | | | |
| | | | AAGCCCCTAAGCGCTGG | | | |
| | | | ATATACGATTCAAGTAA | | | |
| | | | GTTGGCTTCTGGCGTCC | | | |
| | | | CATCACGGTTTTCTGGT | | | |
| | | | TCAGGTTCCGGTACAGA | | | |
| | | | TTTTACGCTGACAATCA | | | |
| | | | GCTCTCTCCAACCGGAA | | | |
| | | | GATTTCGCAACCTATTA | | | |
| | | | CTGTCAACAATGGTCAA | | | |
| | | | GAAATCCGCCGACATTC | | | |
| | | | GGGCAGGGAACAAAAGT | | | |
| | | | CGAGATAAAA | | | |
| | Anti PSMA x Fc | | CAGGTGCAGCTGGTGCA | 105 | QVQLVQSGAEVKKPGAS | 106 |
| | Knob x Anti CD3 | | GTCTGGGGCTGAGGTGA | | VKVSCKASGYTFTDYYM | |
| | scFv (Chain 1) | | AGAAGCCTGGGGCCTCA | | HWVRQAPGQGLEWM | |
| | | | GTGAAGGTTTCCTGCAA | | GYFNPYNDYTRYAQKFQ | |
| | | | GGCATCTGGATACACCT | | GRVTMTRDTSTSTVYME | |
| | | | TCACCGACTACTATATG | | LSSLRSEDTAVYYCARSD | |
| | | | CACTGGGTGCGACAGGC | | GYYDAMDYWGQGTTV | |
| | | | CCCTGGACAAGGGCTTG | | TVSSGGGGSGGGGSGG | |
| | | | AGTGGATGGGATATTTC | | GGSGGGGSDIQMTQSP | |
| | | | AACCCTTATAATGATTA | | SSLSASVGDRVTITCRAS | |
| | | | CACACGCTACGCACAGA | | KSISKYLAWYQQKPGKA | |
| | | | AGTTCCAGGGCAGAGTC | | PKLLIHSGSSLESGVPSRF | |
| | | | ACCATGACCAGGGACAC | | SGSGSGTEFTLTISSLQPD | |
| | | | GTCCACGAGCACAGTCT | | DFATYYCQQHIEYPWTF | |
| | | | ACATGGAGCTGAGCAGC | | GQGTKVEIKEPKSSDKTH | |
| | | | CTGAGATCTGAGGACAC | | TCPPCPAPPAAAPSVFLF | |
| | | | GGCCGTGTATTACTGTG | | PPKPKDTLMISRTPEVTC | |
| | | | CGAGATCTGACGGCTAC | | VVVDVSHEDPEVKFNW | |
| | | | TACGACGCTATGGACTA | | YVDGVEVHNAKTKPREE | |
| | | | CTGGGGGCAAGGGACCA | | QYNSTYRVVSVLTVLHQ | |
| | | | CGGTCACCGTCTCCTCG | | DWLNGKEYKCAVSNKAL | |
| | | | GGAGGCGGTGGATCAGG | | PAPIEKTISKAKGQPREP | |
| | | | CGGTGGAGGCAGCGGAG | | QVYTLPPSRDELTKNQVS | |
| | | | GAGGTGGCTCCGGTGGC | | LWCLVKGFYPSDIAVEW | |
| | | | GGAGGGAGCGACATCCA | | ESNGQPENNYKTTPPVL | |
| | | | GATGACCCAGTCTCCTT | | DSDGSFFLYSKLTVDKSR | |
| | | | CCTCCCTGTCTGCATCT | | WQQGNVFSCSVMHEAL | |
| | | | GTAGGAGACAGAGTCAC | | HNHYTQKSLSLSPGGGG | |
| | | | CATCACTTGCCGGGCCA | | SPSQVQLVQSGAEVKKP | |
| | | | GTAAGAGTATTAGTAAG | | GASVKVSCKASGYTFTRS | |
| | | | TACTTGGCCTGGTATCA | | TMHWVRQAPGQGLEW | |
| | | | GCAGAAACCAGGGAAAG | | IGYINPSSAYTNYAQKFQ | |
| | | | CCCCTAAGCTCCTGATC | | GRVTLTADKSTSTAYME | |
| | | | CATTCTGGCTCCAGTTT | | LSSLRSEDTAVYYCASPQ | |
| | | | GGAAAGTGGGGTCCCAT | | VHYDYNGFPYWGQGTL | |
| | | | CAAGGTTCAGCGGCAGT | | VTVSSGGGGSGGGGSG | |
| | | | GGATCTGGGACAGAATT | | GGGSGGGGSDIQMTQS | |
| | | | CACTCTCACCATCAGCA | | PSSLSASVGDRVTITCRA | |
| | | | GCCTGCAGCCTGATGAT | | SSSVSYMNWYQQKPGK | |
| | | | TTTGCAACTTATTACTG | | APKRWIYDSSKLASGVPS | |
| | | | CCAACAGCATATTGAAT | | RFSGSGSGTDFTLTISSLQ | |
| | | | ATCCTTGGACGTTCGGC | | PEDFATYYCQQWSRNPP | |
| | | | CAAGGGACCAAGGTGGA | | TFGQGTKVEIKRS | |
| | | | AATCAAAGAGCCCAAAT | | | |
| | | | CTTCTGACAAAACTCAC | | | |
| | | | ACATGCCCACCGTGCCC | | | |
| | | | AGCACCTCCAGCCGCTG | | | |
| | | | CACCGTCAGTCTTCCTC | | | |
| | | | TTCCCCCCAAAACCCAA | | | |
| | | | GGACACCCTCATGATCT | | | |
| | | | CCCGGACCCCTGAGGTC | | | |
| | | | ACATGCGTGGTGGTGGA | | | |
| | | | CGTGAGCCACGAAGACC | | | |
| | | | CTGAGGTCAAGTTCAAC | | | |
| | | | TGGTACGTGGACGGCGT | | | |
| | | | GGAGGTGCATAATGCCA | | | |
| | | | AGACAAAGCCGCGGGAG | | | |
| | | | GAGCAGTACAACAGCAC | | | |
| | | | GTACCGTGTGGTCAGCG | | | |
| | | | TCCTCACCGTCCTGCAC | | | |
| | | | CAGGACTGGCTGAATGG | | | |
| | | | CAAGGAATACAAGTGCG | | | |
| | | | CGGTCTCCAACAAAGCC | | | |
| | | | CTCCCAGCCCCCATCGA | | | |
| | | | GAAAACCATCTCCAAAG | | | |
| | | | CCAAAGGGCAGCCCCGA | | | |
| | | | GAACCACAGGTGTACAC | | | |
| | | | CCTGCCCCCATCCCGGG | | | |
| | | | ATGAGCTGACCAAGAAC | | | |
| | | | CAGGTCAGCCTGTGGTG | | | |
| | | | CCTGGTCAAAGGCTTCT | | | |
| | | | ATCCAAGCGACATCGCC | | | |
| | | | GTGGAGTGGGAGAGCAA | | | |
| | | | TGGGCAGCCGGAGAACA | | | |
| | | | ACTACAAGACCACGCCT | | | |
| | | | CCCGTGCTGGACTCCGA | | | |
| | | | CGGCTCCTTCTTCCTCT | | | |
| | | | ACAGCAAGCTCACCGTG | | | |
| | | | GACAAGAGCAGGTGGCA | | | |
| | | | GCAGGGGAACGTCTTCT | | | |
| | | | CATGCTCCGTGATGCAT | | | |
| | | | GAGGCTCTGCACAACCA | | | |
| | | | CTACACGCAGAAGAGCC | | | |
| | | | TCTCCCTGTCTCCGGGC | | | |
| | | | GGCGGGGGATCCCCGTC | | | |
| | | | ACAAGTACAACTCGTTC | | | |
| | | | AAAGTGGCGCAGAAGTA | | | |
| | | | AAGAAGCCAGGCGCCAG | | | |
| | | | TGTTAAGGTGAGCTGCA | | | |
| | | | AGGCAAGCGGGTACACC | | | |
| | | | TTCACCCGGTCTACAAT | | | |
| | | | GCACTGGGTAAGACAAG | | | |
| | | | CACCAGGGCAAGGACTC | | | |
| | | | GAATGGATTGGTTACAT | | | |
| | | | CAACCCTTCCTCTGCAT | | | |
| | | | ACACCAACTACGCTCAA | | | |
| | | | AAGTTCCAGGGCCGCGT | | | |
| | | | TACTTTGACAGCGGATA | | | |
| | | | AATCTACATCCACGGCC | | | |
| | | | TATATGGAACTGTCAAG | | | |
| | | | CCTCAGGAGCGAGGACA | | | |
| | | | CAGCGGTATATTACTGT | | | |
| | | | GCATCTCCCCAGGTCCA | | | |
| | | | TTATGACTACAACGGGT | | | |
| | | | TTCCGTACTGGGGACAA | | | |
| | | | GGAACTCTGGTTACAGT | | | |
| | | | CAGTAGCGGCGGAGGCG | | | |
| | | | GAAGCGGAGGTGGGGGC | | | |
| | | | TCCGGAGGCGGGGGAAG | | | |
| | | | CGGCGGAGGTGGCTCTG | | | |
| | | | ATATCCAGATGACCCAA | | | |
| | | | AGTCCGAGCTCGTTGAG | | | |
| | | | TGCAAGTGTAGGAGACC | | | |
| | | | GCGTAACGATTACTTGC | | | |
| | | | AGAGCTTCAAGTTCCGT | | | |
| | | | ATCCTACATGAATTGGT | | | |
| | | | ATCAGCAAAAGCCTGGA | | | |
| | | | AAAGCCCCTAAGCGCTG | | | |
| | | | GATATACGATTCAAGTA | | | |
| | | | AGTTGGCTTCTGGCGTC | | | |
| | | | CCATCACGGTTTTCTGG | | | |
| | | | TTCAGGTTCCGGTACAG | | | |
| | | | ATTTTACGCTGACAATC | | | |
| | | | AGCTCTCTCCAACCGGA | | | |
| | | | AGATTTCGCAACCTATT | | | |
| | | | ACTGTCAACAATGGTCA | | | |
| | | | AGAAATCCGCCGACATT | | | |
| | | | CGGGCAGGGAACAAAAG | | | |
| | | | TCGAGATAAAAAGGTCA | | | |
| | Anti PSMA x Fc | | CAGGTGCAGCTGGTGCA | 107 | QVQLVQSGAEVKKPGAS | 108 |
| | Hole (Chain 2) | | GTCTGGGGCTGAGGTGA | | VKVSCKASGYTFTDYYM | |
| | | | AGAAGCCTGGGGCCTCA | | HWVRQAPGQGLEWM | |
| | | | GTGAAGGTTTCCTGCAA | | GYFNPYNDYTRYAQKFQ | |
| | | | GGCATCTGGATACACCT | | GRVTMTRDTSTSTVYME | |
| | | | TCACCGACTACTATATG | | LSSLRSEDTAVYYCARSD | |
| | | | CACTGGGTGCGACAGGC | | GYYDAMDYWGQGTTV | |
| | | | CCCTGGACAAGGGCTTG | | TVSSGGGGSGGGGSGG | |
| | | | AGTGGATGGGATATTTC | | GGSGGGGSDIQMTQSP | |
| | | | AACCCTTATAATGATTA | | SSLSASVGDRVTITCRAS | |
| | | | CACACGCTACGCACAGA | | KSISKYLAWYQQKPGKA | |
| | | | AGTTCCAGGGCAGAGTC | | PKLLIHSGSSLESGVPSRF | |
| | | | ACCATGACCAGGGACAC | | SGSGSGTEFTLTISSLQPD | |
| | | | GTCCACGAGCACAGTCT | | DFATYYCQQHIEYPWTF | |
| | | | ACATGGAGCTGAGCAGC | | GQGTKVEIKEPKSSDKTH | |
| | | | CTGAGATCTGAGGACAC | | TCPPCPAPPAAAPSVFLF | |
| | | | GGCCGTGTATTACTGTG | | PPKPKDTLMISRTPEVTC | |
| | | | CGAGATCTGACGGCTAC | | VVVDVSHEDPEVKFNW | |
| | | | TACGACGCTATGGACTA | | YVDGVEVHNAKTKPREE | |
| | | | CTGGGGGCAAGGGACCA | | QYNSTYRVVSVLTVLHQ | |
| | | | CGGTCACCGTCTCCTCG | | DWLNGKEYKCAVSNKAL | |
| | | | GGTGGTGGAGGTAGTGG | | PAPIEKTISKAKGQPREP | |
| | | | TGGAGGCGGATCTGGCG | | QVYTLPPSRDELTKNQVS | |
| | | | GCGGCGGTTCAGGAGGT | | LSCAVKGFYPSDIAVEWE | |
| | | | GGTGGATCCGACATCCA | | SNGQPENNYKTTPPVLD | |
| | | | GATGACCCAGTCTCCTT | | SDGSFFLVSKLTVDKSRW | |
| | | | CCTCCCTGTCTGCATCT | | QQGNVFSCSVMHEALH | |
| | | | GTAGGAGACAGAGTCAC | | NHYTQKSLSLSPG | |
| | | | CATCACTTGCCGGGCCA | | | |
| | | | GTAAGAGTATTAGTAAG | | | |
| | | | TACTTGGCCTGGTATCA | | | |
| | | | GCAGAAACCAGGGAAAG | | | |
| | | | CCCCTAAGCTCCTGATC | | | |
| | | | CATTCTGGCTCCAGTTT | | | |
| | | | GGAAAGTGGGGTCCCAT | | | |
| | | | CAAGGTTCAGCGGCAGT | | | |
| | | | GGATCTGGGACAGAATT | | | |
| | | | CACTCTCACCATCAGCA | | | |
| | | | GCCTGCAGCCTGATGAT | | | |
| | | | TTTGCAACTTATTACTG | | | |
| | | | CCAACAGCATATTGAAT | | | |
| | | | ATCCTTGGACGTTCGGC | | | |
| | | | CAAGGGACCAAGGTGGA | | | |
| | | | AATCAAAGAGCCCAAAT | | | |
| | | | CTTCTGACAAAACTCAC | | | |
| | | | ACATGCCCACCGTGCCC | | | |
| | | | AGCACCTCCAGCCGCTG | | | |
| | | | CACCGTCAGTCTTCCTC | | | |
| | | | TTCCCCCCAAAACCCAA | | | |
| | | | GGACACCCTCATGATCT | | | |
| | | | CCCGGACCCCTGAGGTC | | | |
| | | | ACATGCGTGGTGGTGGA | | | |
| | | | CGTGAGCCACGAAGACC | | | |
| | | | CTGAGGTCAAGTTCAAC | | | |
| | | | TGGTACGTGGACGGCGT | | | |
| | | | GGAGGTGCATAATGCCA | | | |
| | | | AGACAAAGCCGCGGGAG | | | |
| | | | GAGCAGTACAACAGCAC | | | |
| | | | GTACCGTGTGGTCAGCG | | | |
| | | | TCCTCACCGTCCTGCAC | | | |
| | | | CAGGACTGGCTGAATGG | | | |
| | | | CAAGGAATACAAGTGCG | | | |
| | | | CGGTCTCCAACAAAGCC | | | |
| | | | CTCCCAGCCCCCATCGA | | | |
| | | | GAAAACCATCTCCAAAG | | | |
| | | | CCAAAGGGCAGCCCCGA | | | |
| | | | GAACCACAGGTGTACAC | | | |
| | | | CCTGCCCCCATCCCGGG | | | |
| | | | ATGAGCTGACCAAGAAC | | | |
| | | | CAGGTCAGCCTGTCTTG | | | |
| | | | CGCTGTCAAAGGCTTCT | | | |
| | | | ATCCAAGCGACATCGCC | | | |
| | | | GTGGAGTGGGAGAGCAA | | | |
| | | | TGGGCAGCCGGAGAACA | | | |
| | | | ACTACAAGACCACGCCT | | | |
| | | | CCCGTGCTGGACTCCGA | | | |
| | | | CGGCTCCTTCTTCCTCG | | | |
| | | | TTAGCAAGCTCACCGTG | | | |
| | | | GACAAGAGCAGGTGGCA | | | |
| | | | GCAGGGGAACGTCTTCT | | | |
| | | | CATGCTCCGTGATGCAT | | | |
| | | | GAGGCTCTGCACAACCA | | | |
| | | | CTACACGCAGAAGAGCC | | | |
| | | | TCTCCCTGTCTCCGGGT | | | |
| PSMA | Anti- | scFv | GATATCCAGATGACCCA | 109 | DIQMTQSPSSLSASVGD | 110 |
| 01108 | CD3 | | AAGTCCGAGCTCGTTGA | | RVTITCRASSSVSYMNW | |
| Anti-PSMA | scFv | | GTGCAAGTGTAGGAGAC | | YQQKPGKAPKRWIYDSS | |
| scFv x Fc x | | | CGCGTAACGATTACTTG | | KLASGVPSRFSGSGSGTD | |
| linker x Anti | | | CAGAGCTTCAAGTTCCG | | FTLTISSLQPEDFATYYCQ | |
| CD3 scFv; | | | TATCCTACATGAATTGG | | QWSRNPPTFGQGTKVEI | |
| Anti PSMA | | | TATCAGCAAAAGCCTGG | | KGGGGSGGGGSGGGGS | |
| scFv x Fc | | | AAAAGCCCCTAAGCGCT | | GGGGSQVQLVQSGAEV | |
| | | | GGATATACGATTCAAGT | | KKPGASVKVSCKASGYTF | |
| | | | AAGTTGGCTTCTGGCGT | | TRSTMHWVRQAPGQG | |
| | | | CCCATCACGGTTTTCTG | | LEWIGYINPSSAYTNYAQ | |
| | | | GTTCAGGTTCCGGTACA | | KFQGRVTLTADKSTSTAY | |
| | | | GATTTTACGCTGACAAT | | MELSSLRSEDTAVYYCAS | |
| | | | CAGCTCTCTCCAACCGG | | PQVHYDYNGFPYWGQG | |
| | | | AAGATTTCGCAACCTAT | | TLVTVSS | |
| | | | TACTGTCAACAATGGTC | | | |
| | | | AAGAAATCCGCCGACAT | | | |
| | | | TCGGGCAGGGAACAAAA | | | |
| | | | GTCGAGATAAAAGGCGG | | | |
| | | | AGGCGGAAGCGGAGGTG | | | |
| | | | GGGGCTCCGGAGGCGGG | | | |
| | | | GGAAGCGGCGGAGGTGG | | | |
| | | | CTCTCAAGTACAACTCG | | | |
| | | | TTCAAAGTGGCGCAGAA | | | |
| | | | GTAAAGAAGCCAGGCGC | | | |
| | | | CAGTGTTAAGGTGAGCT | | | |
| | | | GCAAGGCAAGCGGGTAC | | | |
| | | | ACCTTCACCCGGTCTAC | | | |
| | | | AATGCACTGGGTAAGAC | | | |
| | | | AAGCACCAGGGCAAGGA | | | |
| | | | CTCGAATGGATTGGTTA | | | |
| | | | CATCAACCCTTCCTCTG | | | |
| | | | CATACACCAACTACGCT | | | |
| | | | CAAAAGTTCCAGGGCCG | | | |
| | | | CGTTACTTTGACAGCGG | | | |
| | | | ATAAATCTACATCCACG | | | |
| | | | GCCTATATGGAACTGTC | | | |
| | | | AAGCCTCAGGAGCGAGG | | | |
| | | | ACACAGCGGTATATTAC | | | |
| | | | TGTGCATCTCCCCAGGT | | | |
| | | | CCATTATGACTACAACG | | | |
| | | | GGTTTCCGTACTGGGGA | | | |
| | | | CAAGGAACTCTGGTTAC | | | |
| | | | AGTCAGTAGC | | | |
| | Anti PSMA x Fc | | CAGGTGCAGCTGGTGCA | 111 | QVQLVQSGAEVKKPGAS | 112 |
| | Knob x Anti CD3 | | GTCTGGGGCTGAGGTGA | | VKVSCKASGYTFTDYYM | |
| | scFv (Chain 1) | | AGAAGCCTGGGGCCTCA | | HWVRQAPGQGLEWM | |
| | | | GTGAAGGTTTCCTGCAA | | GYFNPYNDYTRYAQKFQ | |
| | | | GGCATCTGGATACACCT | | GRVTMTRDTSTSTVYME | |
| | | | TCACCGACTACTATATG | | LSSLRSEDTAVYYCARSD | |
| | | | CACTGGGTGCGACAGGC | | GYYDAMDYWGQGTTV | |
| | | | CCCTGGACAAGGGCTTG | | TVSSGGGGSGGGGSGG | |
| | | | AGTGGATGGGATATTTC | | GGSGGGGSDIQMTQSP | |
| | | | AACCCTTATAATGATTA | | SSLSASVGDRVTITCRAS | |
| | | | CACACGCTACGCACAGA | | KSISKYLAWYQQKPGKA | |
| | | | AGTTCCAGGGCAGAGTC | | PKLLIHSGSSLESGVPSRF | |
| | | | ACCATGACCAGGGACAC | | SGSGSGTEFTLTISSLQPD | |
| | | | GTCCACGAGCACAGTCT | | DFATYYCQQHIEYPWTF | |
| | | | ACATGGAGCTGAGCAGC | | GQGTKVEIKEPKSSDKTH | |
| | | | CTGAGATCTGAGGACAC | | TCPPCPAPPAAAPSVFLF | |
| | | | GGCCGTGTATTACTGTG | | PPKPKDTLMISRTPEVTC | |
| | | | CGAGATCTGACGGCTAC | | VVVDVSHEDPEVKFNW | |
| | | | TACGACGCTATGGACTA | | YVDGVEVHNAKTKPREE | |
| | | | CTGGGGGCAAGGGACCA | | QYNSTYRVVSVLTVLHQ | |
| | | | CGGTCACCGTCTCCTCG | | DWLNGKEYKCAVSNKAL | |
| | | | GGAGGCGGTGGATCAGG | | PAPIEKTISKAKGQPREP | |
| | | | CGGTGGAGGCAGCGGAG | | QVYTLPPSRDELTKNQVS | |
| | | | GAGGTGGCTCCGGTGGC | | LWCLVKGFYPSDIAVEW | |
| | | | GGAGGGAGCGACATCCA | | ESNGQPENNYKTTPPVL | |
| | | | GATGACCCAGTCTCCTT | | DSDGSFFLYSKLTVDKSR | |
| | | | CCTCCCTGTCTGCATCT | | WQQGNVFSCSVMHEAL | |
| | | | GTAGGAGACAGAGTCAC | | HNHYTQKSLSLSPGGGG | |
| | | | CATCACTTGCCGGGCCA | | SPSDIQMTQSPSSLSASV | |
| | | | GTAAGAGTATTAGTAAG | | GDRVTITCRASSSVSYM | |
| | | | TACTTGGCCTGGTATCA | | NWYQQKPGKAPKRWIY | |
| | | | GCAGAAACCAGGGAAAG | | DSSKLASGVPSRFSGSGS | |
| | | | CCCCTAAGCTCCTGATC | | GTDFTLTISSLQPEDFATY | |
| | | | CATTCTGGCTCCAGTTT | | YCQQWSRNPPTFGQGT | |
| | | | GGAAAGTGGGGTCCCAT | | KVEIKGGGGSGGGGSG | |
| | | | CAAGGTTCAGCGGCAGT | | GGGSGGGGSQVQLVQS | |
| | | | GGATCTGGGACAGAATT | | GAEVKKPGASVKVSCKA | |
| | | | CACTCTCACCATCAGCA | | SGYTFTRSTMHWVRQA | |
| | | | GCCTGCAGCCTGATGAT | | PGQGLEWIGYINPSSAYT | |
| | | | TTTGCAACTTATTACTG | | NYAQKFQGRVTLTADKS | |
| | | | CCAACAGCATATTGAAT | | TSTAYMELSSLRSEDTAV | |
| | | | ATCCTTGGACGTTCGGC | | YYCASPQVHYDYNGFPY | |
| | | | CAAGGGACCAAGGTGGA | | WGQGTLVTVSS | |
| | | | AATCAAAGAGCCCAAAT | | | |
| | | | CTTCTGACAAAACTCAC | | | |
| | | | ACATGCCCACCGTGCCC | | | |
| | | | AGCACCTCCAGCCGCTG | | | |
| | | | CACCGTCAGTCTTCCTC | | | |
| | | | TTCCCCCCAAAACCCAA | | | |
| | | | GGACACCCTCATGATCT | | | |
| | | | CCCGGACCCCTGAGGTC | | | |
| | | | ACATGCGTGGTGGTGGA | | | |
| | | | CGTGAGCCACGAAGACC | | | |
| | | | CTGAGGTCAAGTTCAAC | | | |
| | | | TGGTACGTGGACGGCGT | | | |
| | | | GGAGGTGCATAATGCCA | | | |
| | | | AGACAAAGCCGCGGGAG | | | |
| | | | GAGCAGTACAACAGCAC | | | |
| | | | GTACCGTGTGGTCAGCG | | | |
| | | | TCCTCACCGTCCTGCAC | | | |
| | | | CAGGACTGGCTGAATGG | | | |
| | | | CAAGGAATACAAGTGCG | | | |
| | | | CGGTCTCCAACAAAGCC | | | |
| | | | CTCCCAGCCCCCATCGA | | | |
| | | | GAAAACCATCTCCAAAG | | | |
| | | | CCAAAGGGCAGCCCCGA | | | |
| | | | GAACCACAGGTGTACAC | | | |
| | | | CCTGCCCCCATCCCGGG | | | |
| | | | ATGAGCTGACCAAGAAC | | | |
| | | | CAGGTCAGCCTGTGGTG | | | |
| | | | CCTGGTCAAAGGCTTCT | | | |
| | | | ATCCAAGCGACATCGCC | | | |
| | | | GTGGAGTGGGAGAGCAA | | | |
| | | | TGGGCAGCCGGAGAACA | | | |
| | | | ACTACAAGACCACGCCT | | | |
| | | | CCCGTGCTGGACTCCGA | | | |
| | | | CGGCTCCTTCTTCCTCT | | | |
| | | | ACAGCAAGCTCACCGTG | | | |
| | | | GACAAGAGCAGGTGGCA | | | |
| | | | GCAGGGGAACGTCTTCT | | | |
| | | | CATGCTCCGTGATGCAT | | | |
| | | | GAGGCTCTGCACAACCA | | | |
| | | | CTACACGCAGAAGAGCC | | | |
| | | | TCTCCCTGTCTCCGGGC | | | |
| | | | GGCGGGGGATCCCCGTC | | | |
| | | | AGATATCCAGATGACCC | | | |
| | | | AAAGTCCGAGCTCGTTG | | | |
| | | | AGTGCAAGTGTAGGAGA | | | |
| | | | CCGCGTAACGATTACTT | | | |
| | | | GCAGAGCTTCAAGTTCC | | | |
| | | | GTATCCTACATGAATTG | | | |
| | | | GTATCAGCAAAAGCCTG | | | |
| | | | GAAAAGCCCCTAAGCGC | | | |
| | | | TGGATATACGATTCAAG | | | |
| | | | TAAGTTGGCTTCTGGCG | | | |
| | | | TCCCATCACGGTTTTCT | | | |
| | | | GGTTCAGGTTCCGGTAC | | | |
| | | | AGATTTTACGCTGACAA | | | |
| | | | TCAGCTCTCTCCAACCG | | | |
| | | | GAAGATTTCGCAACCTA | | | |
| | | | TTACTGTCAACAATGGT | | | |
| | | | CAAGAAATCCGCCGACA | | | |
| | | | TTCGGGCAGGGAACAAA | | | |
| | | | AGTCGAGATAAAAGGCG | | | |
| | | | GAGGCGGAAGCGGAGGT | | | |
| | | | GGGGGCTCCGGAGGCGG | | | |
| | | | GGGAAGCGGCGGAGGTG | | | |
| | | | GCTCTCAAGTACAACTC | | | |
| | | | GTTCAAAGTGGCGCAGA | | | |
| | | | AGTAAAGAAGCCAGGCG | | | |
| | | | CCAGTGTTAAGGTGAGC | | | |
| | | | TGCAAGGCAAGCGGGTA | | | |
| | | | CACCTTCACCCGGTCTA | | | |
| | | | CAATGCACTGGGTAAGA | | | |
| | | | CAAGCACCAGGGCAAGG | | | |
| | | | ACTCGAATGGATTGGTT | | | |
| | | | ACATCAACCCTTCCTCT | | | |
| | | | GCATACACCAACTACGC | | | |
| | | | TCAAAAGTTCCAGGGCC | | | |
| | | | GCGTTACTTTGACAGCG | | | |
| | | | GATAAATCTACATCCAC | | | |
| | | | GGCCTATATGGAACTGT | | | |
| | | | CAAGCCTCAGGAGCGAG | | | |
| | | | GACACAGCGGTATATTA | | | |
| | | | CTGTGCATCTCCCCAGG | | | |
| | | | TCCATTATGACTACAAC | | | |
| | | | GGGTTTCCGTACTGGGG | | | |
| | | | ACAAGGAACTCTGGTTA | | | |
| | | | CAGTCAGTAGC | | | |

| | | |
|---|---|---|
| Anti PSMA x Fc | 107 | 108 |
| Hole (Chain 2) | | |

| | | | | DNA | | AA |
|---|---|---|---|---|---|---|
| | | | | SEQ | | SEQ |
| Construct | | Component | DNA Sequence | ID NO | AA sequence | ID NO |
| PSMA | Anti | HCDR1 | GGATACACCTTCACCG | 69 | GYTFTDYY | 70 |
| 01116 | PSMA | | ACTACTAT | | | |
| Anti-PSMA | scFv | HCDR2 | TTCAACCCTTATAATG | 71 | FNPYNDYT | 72 |
| scFv x Fc x | | | ATTACACA | | | |
| linker x Anti | | HCDR3 | GCGAGATCTGACGGCT | 73 | ARSDGYYDAMDY | 74 |
| CD3 scFv; | | | ACTACGACGCTATGGA | | | |
| Anti PSMA | | | CTAC | | | |
| scFv x Fc | | LCDR1 | AAGAGTATTAGTAAGT | 75 | KSISKY | 76 |
| | | | AC | | | |
| | | LCDR2 | TCTGGCTCC | 77 | SGS | 78 |
| | | LCDR3 | CAACAGCATATTGAAT | 79 | QQHIEYPWT | 80 |
| | | | ATCCTTGGACG | | | |
| | | V_{H} | CAGGTGCAGCTGGTG | 81 | QVQLVQSGAEVKKPGASV | 82 |
| | | | CAGTCTGGGGCTGAG | | KVSCKASGYTFTDYYMHW | |
| | | | GTGAAGAAGCCTGGG | | VRQAPGQGLEWMGYFNP | |
| | | | GCCTCAGTGAAGGTT | | YNDYTRYAQKFQGRVTMT | |
| | | | TCCTGCAAGGCATCT | | RDTSTSTVYMELSSLRSEDT | |
| | | | GGATACACCTTCACC | | AVYYCARSDGYYDAMDY | |
| | | | GACTACTATATGCAC | | WGQGTTVTVSS | |
| | | | TGGGTGCGACAGGCC | | | |
| | | | CCTGGACAAGGGCTT | | | |
| | | | GAGTGGATGGGATAT | | | |
| | | | TTCAACCCTTATAAT | | | |
| | | | GATTACACACGCTAC | | | |
| | | | GCACAGAAGTTCCAG | | | |
| | | | GGCAGAGTCACCATG | | | |
| | | | ACCAGGGACACGTCC | | | |
| | | | ACGAGCACAGTCTAC | | | |
| | | | ATGGAGCTGAGCAGC | | | |
| | | | CTGAGATCTGAGGAC | | | |
| | | | ACGGCCGTGTATTAC | | | |
| | | | TGTGCGAGATCTGAC | | | |
| | | | GGCTACTACGACGCT | | | |
| | | | ATGGACTACTGGGGG | | | |
| | | | CAAGGGACCACGGTC | | | |
| | | | ACCGTCTCCTCG | | | |
| | | V_{L} | GACATCCAGATGACCC | 83 | DIQMTQSPSSLSASVGDRV | 84 |
| | | | AGTCTCCTTCCTCCCT | | TITCRASKSISKYLAWYQQK | |
| | | | GTCTGCATCTGTAGGA | | PGKAPKLLIHSGSSLESGVP | |
| | | | GACAGAGTCACCATCA | | SRFSGSGSGTEFTLTISSLQP | |
| | | | CTTGCCGGGCCAGTAA | | DDFATYYCQQHIEYPWTF | |
| | | | GAGTATTAGTAAGTAC | | GQGTKVEIK | |
| | | | TTGGCCTGGTATCAGC | | | |
| | | | AGAAACCAGGGAAAGC | | | |
| | | | CCCTAAGCTCCTGATC | | | |
| | | | CATTCTGGCTCCAGTT | | | |
| | | | TGGAAAGTGGGGTCCC | | | |
| | | | ATCAAGGTTCAGCGGC | | | |
| | | | AGTGGATCTGGGACAG | | | |
| | | | AATTCACTCTCACCAT | | | |
| | | | CAGCAGCCTGCAGCCT | | | |
| | | | GATGATTTTGCAACTT | | | |
| | | | ATTACTGCCAACAGCA | | | |
| | | | TATTGAATATCCTTGG | | | |
| | | | ACGTTCGGCCAAGGGA | | | |
| | | | CCAAGGTGGAAATCAA | | | |
| | | | A | | | |
| | | scFv | CAGGTGCAGCTGGTGC | 85 | QVQLVQSGAEVKKPGASV | 86 |
| | | | AGTCTGGGGCTGAGGT | | KVSCKASGYTFTDYYMHW | |
| | | | GAAGAAGCCTGGGGCC | | VRQAPGQGLEWMGYFNP | |
| | | | TCAGTGAAGGTTTCCT | | YNDYTRYAQKFQGRVTMT | |
| | | | GCAAGGCATCTGGATA | | RDTSTSTVYMELSSLRSEDT | |
| | | | CACCTTCACCGACTAC | | AVYYCARSDGYYDAMDY | |
| | | | TATATGCACTGGGTGC | | WGQGTTVTVSSGGGGSG | |
| | | | GACAGGCCCCTGGACA | | GGGSGGGGSGGGGSDIQ | |
| | | | AGGGCTTGAGTGGATG | | MTQSPSSLSASVGDRVTIT | |
| | | | GGATATTTCAACCCTT | | CRASKSISKYLAWYQQKPG | |
| | | | ATAATGATTACACACG | | KAPKLLIHSGSSLESGVPSR | |
| | | | CTACGCACAGAAGTTC | | FSGSGSGTEFTLTISSLQPD | |
| | | | CAGGGCAGAGTCACCA | | DFATYYCQQHIEYPWTFG | |
| | | | TGACCAGGGACACGTC | | QGTKVEIK | |
| | | | CACGAGCACAGTCTAC | | | |
| | | | ATGGAGCTGAGCAGCC | | | |
| | | | TGAGATCTGAGGACAC | | | |
| | | | GGCCGTGTATTACTGT | | | |
| | | | GCGAGATCTGACGGCT | | | |
| | | | ACTACGACGCTATGGA | | | |
| | | | CTACTGGGGGCAAGGG | | | |
| | | | ACCACGGTCACCGTCT | | | |
| | | | CCTCGGGAGGCGGTGG | | | |
| | | | ATCAGGCGGTGGAGGC | | | |
| | | | AGCGGAGGAGGTGGCT | | | |
| | | | CCGGTGGCGGAGGGAG | | | |
| | | | CGACATCCAGATGACC | | | |
| | | | CAGTCTCCTTCCTCCC | | | |
| | | | TGTCTGCATCTGTAGG | | | |
| | | | AGACAGAGTCACCATC | | | |
| | | | ACTTGCCGGGCCAGTA | | | |
| | | | AGAGTATTAGTAAGTA | | | |
| | | | CTTGGCCTGGTATCAG | | | |
| | | | CAGAAACCAGGGAAAG | | | |
| | | | CCCCTAAGCTCCTGAT | | | |
| | | | CCATTCTGGCTCCAGT | | | |
| | | | TTGGAAAGTGGGGTCC | | | |
| | | | CATCAAGGTTCAGCGG | | | |
| | | | CAGTGGATCTGGGACA | | | |
| | | | GAATTCACTCTCACCA | | | |
| | | | TCAGCAGCCTGCAGCC | | | |
| | | | TGATGATTTTGCAACT | | | |
| | | | TATTACTGCCAACAGC | | | |
| | | | ATATTGAATATCCTTG | | | |
| | | | GACGTTCGGCCAAGGG | | | |
| | | | ACCAAGGTGGAAATCA | | | |
| | | | AA | | | |
| | Anti- | HCDR1 | GGGTACACCTTCACCC | 87 | GYTFTRST | 88 |
| | CD3 | | GGTCTACA | | | |
| | scFv | HCDR2 | ATCAACCCTTCCTCTG | 89 | INPSSAYT | 90 |
| | | | CATACACC | | | |
| | | HCDR3 | GCATCTCCCCAGGTCC | 91 | ASPQVHYDYNGFPY | 92 |
| | | | ATTATGACTACAACGG | | | |
| | | | GTTTCCGTAC | | | |
| | | LCDR1 | AGTTCCGTATCCTAC | 93 | SSVSY | 94 |
| | | LCDR2 | GATTCAAGT | 95 | DSS | 96 |
| | | LCDR3 | CAACAATGGTCAAGAA | 97 | QQWSRNPPT | 98 |
| | | | ATCCGCCGACA | | | |
| | | V_{H} | CAAGTACAACTCGTTC | 99 | QVQLVQSGAEVKKPGASV | 100 |
| | | | AAAGTGGCGCAGAAGT | | KVSCKASGYTFTRSTMHW | |
| | | | AAAGAAGCCAGGCGCC | | VRQAPGQGLEWIGYINPSS | |
| | | | AGTGTTAAGGTGAGCT | | AYTNYAQKFQGRVTLTAD | |
| | | | GCAAGGCAAGCGGGTA | | KSTSTAYMELSSLRSEDTAV | |
| | | | CACCTTCACCCGGTCT | | YYCASPQVHYDYNGFPYW | |
| | | | ACAATGCACTGGGTAA | | GQGTLVTVSS | |
| | | | GACAAGCACCAGGGCA | | | |
| | | | AGGACTCGAATGGATT | | | |
| | | | GGTTACATCAACCCTT | | | |
| | | | CCTCTGCATACACCAA | | | |
| | | | CTACGCTCAAAAGTTC | | | |
| | | | CAGGGCCGCGTTACTT | | | |
| | | | TGACAGCGGATAAATC | | | |
| | | | TACATCCACGGCCTAT | | | |
| | | | ATGGAACTGTCAAGCC | | | |
| | | | TCAGGAGCGAGGACAC | | | |
| | | | AGCGGTATATTACTGT | | | |
| | | | GCATCTCCCCAGGTCC | | | |
| | | | ATTATGACTACAACGG | | | |
| | | | GTTTCCGTACTGGGGA | | | |
| | | | CAAGGAACTCTGGTTA | | | |
| | | | CAGTCAGTAGC | | | |
| | | V_{L} | GATATCCAGATGACCC | 101 | DIQMTQSPSSLSASVGDRV | 102 |
| | | | AAAGTCCGAGCTCGTT | | T | |
| | | | GAGTGCAAGTGTAGGA | | ITCRASSSVSYMNWYQQK | |
| | | | GACCGCGTAACGATTA | | PGKAPKRWIYDSSKLASGV | |
| | | | CTTGCAGAGCTTCAAG | | PSRFSGSGSGTDFTLTISSL | |
| | | | TTCCGTATCCTACATG | | QPE | |
| | | | AATTGGTATCAGCAAA | | DFATYYCQQWSRNPPTFG | |
| | | | AGCCTGGAAAAGCCCC | | QGTKVEIK | |
| | | | TAAGCGCTGGATATAC | | | |
| | | | GATTCAAGTAAGTTGG | | | |
| | | | CTTCTGGCGTCCCATC | | | |
| | | | ACGGTTTTCTGGTTCA | | | |
| | | | GGTTCCGGTACAGATT | | | |
| | | | TTACGCTGACAATCAG | | | |
| | | | CTCTCTCCAACCGGAA | | | |
| | | | GATTTCGCAACCTATT | | | |
| | | | ACTGTCAACAATGGTC | | | |
| | | | AAGAAATCCGCCGACA | | | |
| | | | TTCGGGCAGGGAACAA | | | |
| | | | AAGTCGAGATAAAA | | | |
| | | scFv | CAAGTACAACTCGTTC | 103 | QVQLVQSGAEVKKPGASV | 104 |
| | | | AAAGTGGCGCAGAAGT | | KVSCKASGYTFTRSTMHW | |
| | | | AAAGAAGCCAGGCGCC | | VRQAPGQGLEWIGYINPSS | |
| | | | AGTGTTAAGGTGAGCT | | AYT | |
| | | | GCAAGGCAAGCGGGTA | | NYAQKFQGRVTLTADKSTS | |
| | | | CACCTTCACCCGGTCT | | T | |
| | | | ACAATGCACTGGGTAA | | AYMELSSLRSEDTAVYYCA | |
| | | | GACAAGCACCAGGGCA | | S | |
| | | | AGGACTCGAATGGATT | | PQVHYDYNGFPYWGQGT | |
| | | | GGTTACATCAACCCTT | | LVTVSSGGGGSGGGGSGG | |
| | | | CCTCTGCATACACCAA | | GGSGGGGSDIQMTQSPSS | |
| | | | CTACGCTCAAAAGTTC | | LSASVGDRVTITCRASSSVS | |
| | | | CAGGGCCGCGTTACTT | | YMNWYQQKPGKAPKRWI | |
| | | | TGACAGCGGATAAATC | | YDSSKLASGVPSRFSGSGS | |
| | | | TACATCCACGGCCTAT | | GTDFTLTISSLQPEDFATYY | |
| | | | ATGGAACTGTCAAGCC | | CQQWSRNPPTFGQGTKV | |
| | | | TCAGGAGCGAGGACAC | | EIK | |
| | | | AGCGGTATATTACTGT | | | |
| | | | GCATCTCCCCAGGTCC | | | |
| | | | ATTATGACTACAACGG | | | |
| | | | GTTTCCGTACTGGGGA | | | |
| | | | CAAGGAACTCTGGTTA | | | |
| | | | CAGTCAGTAGCGGCGG | | | |
| | | | AGGCGGAAGCGGAGGT | | | |
| | | | GGGGGCTCCGGAGGCG | | | |
| | | | GGGGAAGCGGCGGAGG | | | |
| | | | TGGCTCTGATATCCAG | | | |
| | | | ATGACCCAAAGTCCGA | | | |
| | | | GCTCGTTGAGTGCAAG | | | |
| | | | TGTAGGAGACCGCGTA | | | |
| | | | ACGATTACTTGCAGAG | | | |
| | | | CTTCAAGTTCCGTATC | | | |
| | | | CTACATGAATTGGTAT | | | |
| | | | CAGCAAAAGCCTGGAA | | | |
| | | | AAGCCCCTAAGCGCTG | | | |
| | | | GATATACGATTCAAGT | | | |
| | | | AAGTTGGCTTCTGGCG | | | |
| | | | TCCCATCACGGTTTTC | | | |
| | | | TGGTTCAGGTTCCGGT | | | |
| | | | ACAGATTTTACGCTGA | | | |
| | | | CAATCAGCTCTCTCCA | | | |
| | | | ACCGGAAGATTTCGCA | | | |
| | | | ACCTATTACTGTCAAC | | | |
| | | | AATGGTCAAGAAATCC | | | |
| | | | GCCGACATTCGGGCAG | | | |
| | | | GGAACAAAAGTCGAGA | | | |
| | | | TAAAA | | | |
| | Anti PSMA x Fc | | CAGGTGCAGCTGGTGC | 177 | QVQLVQSGAEVKKPGASV | 178 |
| | Knob x | | AGTCTGGGGCTGAGGT | | KVSCKASGYTFTDYYMHW | |
| | Anti CD3 scFv | | GAAGAAGCCTGGGGCC | | VRQAPGQGLEWMGYFNP | |
| | (Chain 1) | | TCAGTGAAGGTTTCCT | | YNDYTRYAQKFQGRVTMT | |
| | | | GCAAGGCATCTGGATA | | RDTSTSTVYMELSSLRSEDT | |
| | | | CACCTTCACCGACTAC | | AVYYCARSDGYYDAMDY | |
| | | | TATATGCACTGGGTGC | | WGQGTTVTVSSGGGGSG | |
| | | | GACAGGCCCCTGGACA | | GGGSGGGGSGGGGSDIQ | |
| | | | AGGGCTTGAGTGGATG | | MTQSPSSLSASVGDRVTIT | |
| | | | GGATATTTCAACCCTT | | CRASKSISKYLAWYQQKPG | |
| | | | ATAATGATTACACACG | | KAPKLLIHSGSSLESGVPSR | |
| | | | CTACGCACAGAAGTTC | | FSGSGSGTEFTLTISSLQPD | |
| | | | CAGGGCAGAGTCACCA | | DFATYYCQQHIEYPWTFG | |
| | | | TGACCAGGGACACGTC | | QGTKVEIKEPKSSDKTHTCP | |
| | | | CACGAGCACAGTCTAC | | PCPAPPAAAPSVFLFPPKPK | |
| | | | ATGGAGCTGAGCAGCC | | DTLMISRTPEVTCVVVDVS | |
| | | | TGAGATCTGAGGACAC | | HEDPEVKFNWYVDGVEVH | |
| | | | GGCCGTGTATTACTGT | | NAKTKPREEQYNSTYRVVS | |
| | | | GCGAGATCTGACGGCT | | VLTVLHQDWLNGKEYKCA | |
| | | | ACTACGACGCTATGGA | | VSNKALPAPIEKTISKAKGQ | |
| | | | CTACTGGGGGCAAGGG | | PREPQVYTLPPSRDELTKN | |
| | | | ACCACGGTCACCGTCT | | QVSLWCLVKGFYPSDIAVE | |
| | | | CCTCGGGAGGCGGTGG | | WESNGQPENNYKTTPPVL | |
| | | | ATCAGGCGGTGGAGGC | | DSDGSFFLYSKLTVDKSRW | |
| | | | AGCGGAGGAGGTGGCT | | QQGNVFSCSVMHEALHN | |
| | | | CCGGTGGCGGAGGGAG | | HYTQKSLSLSPGGGGSPSQ | |
| | | | CGACATCCAGATGACC | | VQLVQSGAEVKKPGASVK | |
| | | | CAGTCTCCTTCCTCCC | | VSCKASGYTFTRSTMHWV | |
| | | | TGTCTGCATCTGTAGG | | RQAPGQGLEWIGYINPSSA | |
| | | | AGACAGAGTCACCATC | | YTNYAQKFQGRVTLTADKS | |
| | | | ACTTGCCGGGCCAGTA | | TSTAYMELSSLRSEDTAVYY | |
| | | | AGAGTATTAGTAAGTA | | CASPQVHYDYNGFPYWG | |
| | | | CTTGGCCTGGTATCAG | | QGTLVTVSSGGGGSGGGG | |
| | | | CAGAAACCAGGGAAAG | | SGGGGSGGGGSDIQMTQ | |
| | | | CCCCTAAGCTCCTGAT | | SPSSLSASVGDRVTITCRAS | |
| | | | CCATTCTGGCTCCAGT | | SSVSYMNWYQQKPGKAP | |
| | | | TTGGAAAGTGGGGTCC | | KRWIYDSSKLASGVPSRFS | |
| | | | CATCAAGGTTCAGCGG | | GSGSGTDFTLTISSLQPEDF | |
| | | | CAGTGGATCTGGGACA | | ATYYCQQWSRNPPTFGQG | |
| | | | GAATTCACTCTCACCA | | TKVEIK | |
| | | | TCAGCAGCCTGCAGCC | | | |
| | | | TGATGATTTTGCAACT | | | |
| | | | TATTACTGCCAACAGC | | | |
| | | | ATATTGAATATCCTTG | | | |
| | | | GACGTTCGGCCAAGGG | | | |
| | | | ACCAAGGTGGAAATCA | | | |
| | | | AAGAGCCCAAATCTTC | | | |
| | | | TGACAAAACTCACACA | | | |
| | | | TGCCCACCGTGCCCAG | | | |
| | | | CACCTCCAGCCGCTGC | | | |
| | | | ACCGTCAGTCTTCCTC | | | |
| | | | TTCCCCCCAAAACCCA | | | |
| | | | AGGACACCCTCATGAT | | | |
| | | | CTCCCGGACCCCTGAG | | | |
| | | | GTCACATGCGTGGTGG | | | |
| | | | TGGACGTGAGCCACGA | | | |
| | | | AGACCCTGAGGTCAAG | | | |
| | | | TTCAACTGGTACGTGG | | | |
| | | | ACGGCGTGGAGGTGCA | | | |
| | | | TAATGCCAAGACAAAG | | | |
| | | | CCGCGGGAGGAGCAGT | | | |
| | | | ACAACAGCACGTACCG | | | |
| | | | TGTGGTCAGCGTCCTC | | | |
| | | | ACCGTCCTGCACCAGG | | | |
| | | | ACTGGCTGAATGGCAA | | | |
| | | | GGAATACAAGTGCGCG | | | |
| | | | GTCTCCAACAAAGCCC | | | |
| | | | TCCCAGCCCCCATCGA | | | |
| | | | GAAAACCATCTCCAAA | | | |
| | | | GCCAAAGGGCAGCCCC | | | |
| | | | GAGAACCACAGGTGTA | | | |
| | | | CACCCTGCCCCCATCC | | | |
| | | | CGGGATGAGCTGACCA | | | |
| | | | AGAACCAGGTCAGCCT | | | |
| | | | GTGGTGCCTGGTCAAA | | | |
| | | | GGCTTCTATCCAAGCG | | | |
| | | | ACATCGCCGTGGAGTG | | | |
| | | | GGAGAGCAATGGGCAG | | | |
| | | | CCGGAGAACAACTACA | | | |
| | | | AGACCACGCCTCCCGT | | | |
| | | | GCTGGACTCCGACGGC | | | |
| | | | TCCTTCTTCCTCTACA | | | |
| | | | GCAAGCTCACCGTGGA | | | |
| | | | CAAGAGCAGGTGGCAG | | | |
| | | | CAGGGGAACGTCTTCT | | | |
| | | | CATGCTCCGTGATGCA | | | |
| | | | TGAGGCTCTGCACAAC | | | |
| | | | CACTACACGCAGAAGA | | | |
| | | | GCCTCTCCCTGTCTCC | | | |
| | | | GGGCGGCGGGGGATCC | | | |
| | | | CCGTCACAAGTACAAC | | | |
| | | | TCGTTCAAAGTGGCGC | | | |
| | | | AGAAGTAAAGAAGCCA | | | |
| | | | GGCGCCAGTGTTAAGG | | | |
| | | | TGAGCTGCAAGGCAAG | | | |
| | | | CGGGTACACCTTCACC | | | |
| | | | CGGTCTACAATGCACT | | | |
| | | | GGGTAAGACAAGCACC | | | |
| | | | AGGGCAAGGACTCGAA | | | |
| | | | TGGATTGGTTACATCA | | | |
| | | | ACCCTTCCTCTGCATA | | | |
| | | | CACCAACTACGCTCAA | | | |
| | | | AAGTTCCAGGGCCGCG | | | |
| | | | TTACTTTGACAGCGGA | | | |
| | | | TAAATCTACATCCACG | | | |
| | | | GCCTATATGGAACTGT | | | |
| | | | CAAGCCTCAGGAGCGA | | | |
| | | | GGACACAGCGGTATAT | | | |
| | | | TACTGTGCATCTCCCC | | | |
| | | | AGGTCCATTATGACTA | | | |
| | | | CAACGGGTTTCCGTAC | | | |
| | | | TGGGGACAAGGAACTC | | | |
| | | | TGGTTACAGTCAGTAG | | | |
| | | | CGGCGGAGGCGGAAGC | | | |
| | | | GGAGGTGGGGGCTCCG | | | |
| | | | GAGGCGGGGGAAGCGG | | | |
| | | | CGGAGGTGGCTCTGAT | | | |
| | | | ATCCAGATGACCCAAA | | | |
| | | | GTCCGAGCTCGTTGAG | | | |
| | | | TGCAAGTGTAGGAGAC | | | |
| | | | CGCGTAACGATTACTT | | | |
| | | | GCAGAGCTTCAAGTTC | | | |
| | | | CGTATCCTACATGAAT | | | |
| | | | TGGTATCAGCAAAAGC | | | |
| | | | CTGGAAAAGCCCCTAA | | | |
| | | | GCGCTGGATATACGAT | | | |
| | | | TCAAGTAAGTTGGCTT | | | |
| | | | CTGGCGTCCCATCACG | | | |
| | | | GTTTTCTGGTTCAGGT | | | |
| | | | TCCGGTACAGATTTTA | | | |
| | | | CGCTGACAATCAGCTC | | | |
| | | | TCTCCAACCGGAAGAT | | | |
| | | | TTCGCAACCTATTACT | | | |
| | | | GTCAACAATGGTCAAG | | | |
| | | | AAATCCGCCGACATTC | | | |
| | | | GGGCAGGGAACAAAAG | | | |
| | | | TCGAGATAAAA | | | |
| | Anti PSMA x Fc Hole | | CAGGTGCAGCTGGTGC | 107 | QVQLVQSGAEVKKPGASV | 108 |
| | (Chain 2) | | AGTCTGGGGCTGAGGT | | KVSCKASGYTFTDYYMHW | |
| | | | GAAGAAGCCTGGGGCC | | VRQAPGQGLEWMGYFNP | |
| | | | TCAGTGAAGGTTTCCT | | YNDYTRYAQKFQGRVTMT | |
| | | | GCAAGGCATCTGGATA | | RDTSTSTVYMELSSLRSEDT | |
| | | | CACCTTCACCGACTAC | | AVYYCARSDGYYDAMDY | |
| | | | TATATGCACTGGGTGC | | WGQGTTVTVSSGGGGSG | |
| | | | GACAGGCCCCTGGACA | | GGGSGGGGSGGGGSDIQ | |
| | | | AGGGCTTGAGTGGATG | | MTQSPSSLSASVGDRVTIT | |
| | | | GGATATTTCAACCCTT | | CRASKSISKYLAWYQQKPG | |
| | | | ATAATGATTACACACG | | KAPKLLIHSGSSLESGVPSR | |
| | | | CTACGCACAGAAGTTC | | FSGSGSGTEFTLTISSLQPD | |
| | | | CAGGGCAGAGTCACCA | | DFATYYCQQHIEYPWTFG | |
| | | | TGACCAGGGACACGTC | | QGTKVEIKEPKSSDKTHTCP | |
| | | | CACGAGCACAGTCTAC | | PCPAPPAAAPSVFLFPPKPK | |
| | | | ATGGAGCTGAGCAGCC | | DTLMISRTPEVTCVVVDVS | |
| | | | TGAGATCTGAGGACAC | | HEDPEVKFNWYVDGVEVH | |
| | | | GGCCGTGTATTACTGT | | NAKTKPREEQYNSTYRVVS | |
| | | | GCGAGATCTGACGGCT | | VLTVLHQDWLNGKEYKCA | |
| | | | ACTACGACGCTATGGA | | VSNKALPAPIEKTISKAKGQ | |
| | | | CTACTGGGGGCAAGGG | | PREPQVYTLPPSRDELTKN | |
| | | | ACCACGGTCACCGTCT | | QVSLSCAVKGFYPSDIAVE | |
| | | | CCTCGGGTGGTGGAGG | | WESNGQPENNYKTTPPVL | |
| | | | TAGTGGTGGAGGCGGA | | DSDGSFFLVSKLTVDKSRW | |
| | | | TCTGGCGGCGGCGGTT | | QQGNVFSCSVMHEALHN | |
| | | | CAGGAGGTGGTGGATC | | HYTQKSLSLSPG | |
| | | | CGACATCCAGATGACC | | | |
| | | | CAGTCTCCTTCCTCCC | | | |
| | | | TGTCTGCATCTGTAGG | | | |
| | | | AGACAGAGTCACCATC | | | |
| | | | ACTTGCCGGGCCAGTA | | | |
| | | | AGAGTATTAGTAAGTA | | | |
| | | | CTTGGCCTGGTATCAG | | | |
| | | | CAGAAACCAGGGAAAG | | | |
| | | | CCCCTAAGCTCCTGAT | | | |
| | | | CCATTCTGGCTCCAGT | | | |
| | | | TTGGAAAGTGGGGTCC | | | |
| | | | CATCAAGGTTCAGCGG | | | |
| | | | CAGTGGATCTGGGACA | | | |
| | | | GAATTCACTCTCACCA | | | |
| | | | TCAGCAGCCTGCAGCC | | | |
| | | | TGATGATTTTGCAACT | | | |
| | | | TATTACTGCCAACAGC | | | |
| | | | ATATTGAATATCCTTG | | | |
| | | | GACGTTCGGCCAAGGG | | | |
| | | | ACCAAGGTGGAAATCA | | | |
| | | | AAGAGCCCAAATCTTC | | | |
| | | | TGACAAAACTCACACA | | | |
| | | | TGCCCACCGTGCCCAG | | | |
| | | | CACCTCCAGCCGCTGC | | | |
| | | | ACCGTCAGTCTTCCTC | | | |
| | | | TTCCCCCCAAAACCCA | | | |
| | | | AGGACACCCTCATGAT | | | |
| | | | CTCCCGGACCCCTGAG | | | |
| | | | GTCACATGCGTGGTGG | | | |
| | | | TGGACGTGAGCCACGA | | | |
| | | | AGACCCTGAGGTCAAG | | | |
| | | | TTCAACTGGTACGTGG | | | |
| | | | ACGGCGTGGAGGTGCA | | | |
| | | | TAATGCCAAGACAAAG | | | |
| | | | CCGCGGGAGGAGCAGT | | | |
| | | | ACAACAGCACGTACCG | | | |
| | | | TGTGGTCAGCGTCCTC | | | |
| | | | ACCGTCCTGCACCAGG | | | |
| | | | ACTGGCTGAATGGCAA | | | |
| | | | GGAATACAAGTGCGCG | | | |
| | | | GTCTCCAACAAAGCCC | | | |
| | | | TCCCAGCCCCCATCGA | | | |
| | | | GAAAACCATCTCCAAA | | | |
| | | | GCCAAAGGGCAGCCCC | | | |
| | | | GAGAACCACAGGTGTA | | | |
| | | | CACCCTGCCCCCATCC | | | |
| | | | CGGGATGAGCTGACCA | | | |
| | | | AGAACCAGGTCAGCCT | | | |
| | | | GTCTTGCGCTGTCAAA | | | |
| | | | GGCTTCTATCCAAGCG | | | |
| | | | ACATCGCCGTGGAGTG | | | |
| | | | GGAGAGCAATGGGCAG | | | |
| | | | CCGGAGAACAACTACA | | | |
| | | | AGACCACGCCTCCCGT | | | |
| | | | GCTGGACTCCGACGGC | | | |
| | | | TCCTTCTTCCTCGTTA | | | |
| | | | GCAAGCTCACCGTGGA | | | |
| | | | CAAGAGCAGGTGGCAG | | | |
| | | | CAGGGGAACGTCTTCT | | | |
| | | | CATGCTCCGTGATGCA | | | |
| | | | TGAGGCTCTGCACAAC | | | |
| | | | CACTACACGCAGAAGA | | | |
| | | | GCCTCTCCCTGTCTCC | | | |
| | | | GGGT | | | |

| | | DNA | | AA |
|---|---|---|---|---|
| | | SEQ | | SEQ |
| Protein | | ID | | ID |
| Name | DNA Sequence | NO: | AA Sequence | NO: |
| mIgG | tcgagtgagcccagagggcccacaatcaagccctg | 179 | SSEPRGPTIKPCPPCKCPAPN | 180 |
| Fc- | tcctccatgcaaatgcccggctccaaatgctgcag | | AAGGPSVFIFPPKIKDVLMISL | |
| human | gtggtccatccgtcttcatcttccctccaaagatc | | SPIVTCVVVDVSEDDPDVQIS | |
| PSMA | aaggatgtactcatgatctccctgagccccatagt | | WFVNNVEVHTAQTQTHRE | |
| ECD | cacatgtgtggtggtggatgtgagcgaggacgacc | | DYNSTLRVVSALPIQHQDW | |
| | cagatgtccagatcagctggtttgtgaacaacgtg | | MSGKEFKCKVNNKDLAAPIE | |
| | gaagtacacacagctcagacacaaacccatagaga | | RTISKPKGSVRAPQVYVLPPP | |
| | ggattacaacagtactctccgggtggtcagtgccc | | EEEMTKKQVTLTCMVTDFM | |
| | tccccatccagcaccaggactggatgagtggcaag | | PEDIYVEWTNNGKTELNYKN | |
| | gagttcaaatgcaaggtcaacaacaaagacctcgc | | TEPVLDSDGSYFMYSKLRVEK | |
| | tgcgcccatcgagagaaccatctcaaaacccaaag | | KNWVERNSYSCSVVHEGLH | |
| | ggtcagtaagagctccacaggtatatgtcttgcct | | NHHTTKSFSRTPGSSNEATNI | |
| | ccaccagaagaagagatgactaagaaacaggtcac | | TPKHNMKAFLDELKAENIKK | |
| | tctgacctgcatggtcacagacttcatgcctgaag | | FLYNFTQIPHLAGTEQNFQL | |
| | acatttacgtggagtggactaacaacgggaaaaca | | AKQIQSQWKEFGLDSVELAH | |
| | gagctaaactacaagaacactgaaccagtcctgga | | YDVLLSYPNKTHPNYISIINED | |
| | ctctgatggttcttacttcatgtacagcaagctga | | GNEIFNTSLFEPPPPGYENVS | |
| | gagtggaaaagaagaactgggtggaaagaaatagc | | DIVPPFSAFSPQGMPEGDLV | |
| | tactcctgttcagtggtccacgagggtctgcacaa | | YVNYARTEDFFKLERDMKIN | |
| | tcaccacacgactaagagcttctcccggactccgg | | CSGKIVIARYGKVFRGNKVK | |
| | gctcctccaatgaagctactaacattactccaaag | | NAQLAGAKGVILYSDPADYF | |
| | cataatatgaaagcatttttggatgaattgaaagc | | APGVKSYPDGWNLPGGGV | |
| | tgagaacatcaagaagttcttatataattttacac | | QRGNILNLNGAGDPLTPGYP | |
| | agataccacatttagcaggaacagaacaaaacttt | | ANEYAYRRGIAEAVGLPSIPV | |
| | cagcttgcaaagcaaattcaatcccagtggaaaga | | HPIGYYDAQKLLEKMGGSAP | |
| | atttggcctggattctgttgagctagcacattatg | | PDSSWRGSLKVPYNVGPGFT | |
| | atgtcctgttgtcctacccaaataagactcatccc | | GNFSTQKVKMHIHSTNEVTR | |
| | aactacatctcaataattaatgaagatggaaatga | | IYNVIGTLRGAVEPDRYVILG | |
| | gattttcaacacatcattatttgaaccacctcctc | | GHRDSWVFGGIDPQSGAAV | |
| | caggatatgaaaatgtttcggatattgtaccacct | | VHEIVRSFGTLKKEGWRPRR | |
| | ttcagtgctttctctcctcaaggaatgccagaggg | | TILFASWDAEEFGLLGSTEW | |
| | cgatctagtgtatgttaactatgcacgaactgaag | | AEENSRLLQERGVAYINADSS | |
| | acttctttaaattggaacgggacatgaaaatcaat | | IEGNYTLRVDCTPLMYSLVH | |
| | tgctctgggaaaattgtaattgccagatatgggaa | | NLTKELKSPDEGFEGKSLYES | |
| | agttttcagaggaaataaggttaaaaatgcccagc | | WTKKSPSPEFSGMPRISKLGS | |
| | tggcaggggccaaaggagtcattctctactccgac | | GNDFEVFFQRLGIASGRARY | |
| | cctgctgactactttgctcctggggtgaagtccta | | TKNWETNKFSGYPLYHSVYE | |
| | tccagatggttggaatcttcctggaggtggtgtcc | | TYELVEKFYDPMFKYHLTVA | |
| | agcgtggaaatatcctaaatctgaatggtgcagga | | QVRGGMVFELANSIVLPFDC | |
| | gaccctctcacaccaggttacccagcaaatgaata | | RDYAVVLRKYADKIYSISMKH | |
| | tgcttataggcgtggaattgcagaggctgttggtc | | PQEMKTYSVSFDSLFSAVKN | |
| | ttccaagtattcctgttcatccaattggatactat | | FTEIASKFSERLQDFDKSNPIV | |
| | gatgcacagaagctcctagaaaaaatgggtggctc | | LRMMNDQLMFLERAFIDPL | |
| | agcaccaccagatagcagctggagaggaagtctca | | GLPDRPFYRHVIYAPSSHNKY | |
| | aagtgccctacaatgttggacctggctttactgga | | AGESFPGIYDALFDIESKVDP | |
| | aacttttctacacaaaaagtcaagatgcacatcca | | SKAWGEVKRQIYVAAFTVQ | |
| | ctctaccaatgaagtgacaagaatttacaatgtga | | AAAETLSEVA | |
| | taggtactctcagaggagcagtggaaccagacaga | | | |
| | tatgtcattctgggaggtcaccgggactcatgggt | | | |
| | gtttggtggtattgaccctcagagtggagcagctg | | | |
| | ttgttcatgaaattgtgaggagctttggaacactg | | | |
| | aaaaaggaagggtggagacctagaagaacaatttt | | | |
| | gtttgcaagctgggatgcagaagaatttggtcttc | | | |
| | ttggttctactgagtgggcagaggagaactcaaga | | | |
| | ctccttcaagagcgtggcgtggcttatattaatgc | | | |
| | tgactcatctatagaaggaaactacactctgagag | | | |
| | ttgattgtacaccgctgatgtacagcttggtacac | | | |
| | aacctaacaaaagagctgaaaagccctgatgaagg | | | |
| | ctttgaaggcaaatctctttatgaaagttggacta | | | |
| | aaaaaagtccttccccagagttcagtggcatgccc | | | |
| | aggataagcaaattgggatctggaaatgattttga | | | |
| | ggtgttcttccaacgacttggaattgcttcaggca | | | |
| | gagcacggtatactaaaaattgggaaacaaacaaa | | | |
| | ttcagcggctatccactgtatcacagtgtctatga | | | |
| | aacatatgagttggtggaaaagttttatgatccaa | | | |
| | tgtttaaatatcacctcactgtggcccaggttcga | | | |
| | ggagggatggtgtttgagctagccaattccatagt | | | |
| | gctcccttttgattgtcgagattatgctgtagttt | | | |
| | taagaaagtatgctgacaaaatctacagtatttct | | | |
| | atgaaacatccacaggaaatgaagacatacagtgt | | | |
| | atcatttgattcacttttttctgcagtaaagaatt | | | |
| | ttacagaaattgcttccaagttcagtgagagactc | | | |
| | caggactttgacaaaagcaacccaatagtattaag | | | |
| | aatgatgaatgatcaactcatgtttctggaaagag | | | |
| | catttattgatccattagggttaccagacaggcct | | | |
| | ttttataggcatgtcatctatgctccaagcagcca | | | |
| | caacaagtatgcaggggagtcattcccaggaattt | | | |
| | atgatgctctgtttgatattgaaagcaaagtggac | | | |
| | ccttccaaggcctggggagaagtgaagagacagat | | | |
| | ttatgttgcagccttcacagtgcaggcagctgcag | | | |
| | agactttgagtgaagtagcc | | | |
| mIgG | tcgagtgagcccagagggcccacaatcaagccctg | 181 | SSEPRGPTIKPCPPCKCPAPN | 182 |
| Fc-cyno | tcctccatgcaaatgcccggctccaaatgctgcag | | AAGGPSVFIFPPKIKDVLMISL | |
| PSMA | gtggtccatccgtcttcatcttccctccaaagatc | | SPIVTCVVVDVSEDDPDVQIS | |
| ECD | aaggatgtactcatgatctccctgagccccatagt | | WFVNNVEVHTAQTQTHRE | |
| | cacatgtgtggtggtggatgtgagcgaggacgacc | | DYNSTLRVVSALPIQHQDW | |
| | cagatgtccagatcagctggtttgtgaacaacgtg | | MSGKEFKCKVNNKDLAAPIE | |
| | gaagtacacacagctcagacacaaacccatagaga | | RTISKPKGSVRAPQVYVLPPP | |
| | ggattacaacagtactctccgggtggtcagtgccc | | EEEMTKKQVTLTCMVTDFM | |
| | tccccatccagcaccaggactggatgagtggcaag | | PEDIYVEWTNNGKTELNYKN | |
| | gagttcaaatgcaaggtcaacaacaaagacctcgc | | TEPVLDSDGSYFMYSKLRVEK | |
| | tgcgcccatcgagagaaccatctcaaaacccaaag | | KNWVERNSYSCSVVHEGLH | |
| | ggtcagtaagagctccacaggtatatgtcttgcct | | NHHTTKSFSRTPGSSSEATNI | |
| | ccaccagaagaagagatgactaagaaacaggtcac | | TPKHNMKAFLDELKAENIKK | |
| | tctgacctgcatggtcacagacttcatgcctgaag | | FLHNFTQIPHLAGTEQNFQL | |
| | acatttacgtggagtggactaacaacgggaaaaca | | AKQIQSQWKEFGLDSVELTH | |
| | gagctaaactacaagaacactgaaccagtcctgga | | YDVLLSYPNKTHPNYISIINED | |
| | ctctgatggttcttacttcatgtacagcaagctga | | GNEIFNTSLFEPPPAGYENVS | |
| | gagtggaaaagaagaactgggtggaaagaaatagc | | DIVPPFSAFSPQGMPEGDLV | |
| | tactcctgttcagtggtccacgagggtctgcacaa | | YVNYARTEDFFKLERDMKIN | |
| | tcaccacacgactaagagcttctcccggactccgg | | CSGKIVIARYGKVFRGNKVK | |
| | gctcctccagtgaagctactaacattactccaaag | | NAQLAGATGVILYSDPADYF | |
| | cataatatgaaagcatttttggatgaactgaaagc | | APGVKSYPDGWNLPGGGV | |
| | tgagaacatcaagaagttcttacataattttacac | | QRGNILNLNGAGDPLTPGYP | |
| | agataccacatttagcaggaacagaacaaaacttt | | ANEYAYRRGMAEAVGLPSIP | |
| | caacttgcaaagcaaattcaatcccagtggaaaga | | VHPIGYYDAQKLLEKMGGSA | |
| | atttggcctggattctgttgagctaactcattatg | | SPDSSWRGSLKVPYNVGPGF | |
| | atgtcctgttgtcctacccaaataagactcatccc | | TGNFSTQKVKMHIHSTSEVT | |
| | aactacatctcaataattaatgaagatggaaatga | | RIYNVIGTLRGAVEPDRYVIL | |
| | gattttcaacacatcattatttgaaccacctcctg | | GGHRDSWVFGGIDPQSGAA | |
| | caggatatgaaaatgtttcggatattgtaccacct | | VVHEIVRSFGTLKKEGWRPR | |
| | ttcagtgctttctctcctcaaggaatgccagaggg | | RTILFASWDAEEFGLLGSTE | |
| | cgatctagtgtatgttaactatgcacgaactgaag | | WAEENSRLLQERGVAYINAD | |
| | acttctttaaattggaacgggacatgaaaatcaat | | SSIEGNYTLRVDCTPLMYSLV | |
| | tgctctgggaaaattgtaattgccagatatgggaa | | YNLTKELESPDEGFEGKSLYE | |
| | agttttcagaggaaataaggttaaaaatgcccagc | | SWTKKSPSPEFSGMPRISKLG | |
| | tggcaggggccacaggagtcattctctactcagac | | SGNDFEVFFQRLGIASGRAR | |
| | cctgctgactactttgctcctggggtaaagtctta | | YTKNWETNKFSSYPLYHSVY | |
| | tccagatggttggaatcttcctggaggtggtgtcc | | ETYELVEKFYDPMFKYHLTVA | |
| | agcgtggaaatatcctaaatctgaatggtgcagga | | QVRGGMVFELANSVVLPFD | |
| | gaccctctcacaccaggttacccagcaaatgaata | | CRDYAVVLRKYADKIYNISMK | |
| | tgcttataggcgtggaatggcagaggctgttggtc | | HPQEMKTYSVSFDSLFSAVK | |
| | ttccaagtattcccgttcatccaattgggtactat | | NFTEIASKFSERLRDFDKSNPI | |
| | gatgcacagaagctcctagaaaaaatgggtggctc | | LLRMMNDQLMFLERAFIDPL | |
| | agcatcaccagatagcagctggagaggaagtctca | | GLPDRPFYRHVIYAPSSHNKY | |
| | aagtgccctacaatgttggacctggctttactgga | | AGESFPGIYDALFDIESKVDP | |
| | aacttttctacacaaaaagtcaagatgcacatcca | | SQAWGEVKRQISIATFTVQA | |
| | ctctaccagtgaagtgacaagaatttacaatgtga | | AAETLSEVA | |
| | taggtactctcagaggagcagtggaaccagacaga | | | |
| | tacgtcattctgggaggtcaccgggactcatgggt | | | |
| | gtttggtggtattgaccctcagagtggagcagctg | | | |
| | ttgttcatgaaattgtgaggagctttggaacgctg | | | |
| | aaaaaggaagggtggagacctagaagaacaatttt | | | |
| | gtttgcaagctgggatgcagaagaatttggtcttc | | | |
| | ttggttctactgaatgggcagaggagaactcaaga | | | |
| | ctccttcaagagcgtggcgtggcttatattaatgc | | | |
| | tgattcgtctatagaaggaaactacactctgagag | | | |
| | ttgattgtacaccactgatgtacagcttggtatac | | | |
| | aacctaacaaaagagctggaaagccctgatgaagg | | | |
| | ctttgaaggcaaatctctttatgaaagttggacta | | | |
| | aaaaaagtccttcccccgagttcagtggcatgccc | | | |
| | aggataagcaaattgggatctggaaatgattttga | | | |
| | ggtgttcttccaacgacttggaattgcctcaggca | | | |
| | gagcacggtatactaaaaattgggaaacaaacaaa | | | |
| | ttcagcagctatccactgtatcacagtgtctatga | | | |
| | gacatatgagttggtggaaaagttttatgatccaa | | | |
| | tgtttaaatatcacctcactgtggcccaggttcga | | | |
| | ggagggatggtgtttgaactagccaattccgtagt | | | |
| | gctcccttttgattgtcgagattatgctgtagttt | | | |
| | taagaaagtatgctgacaaaatctacaatatttct | | | |
| | atgaaacatccacaggaaatgaagacatacagtgt | | | |
| | atcatttgattcacttttttctgcagtaaagaatt | | | |
| | ttacagaaattgcttccaagttcagtgagagactc | | | |
| | cgggactttgacaaaagcaacccaatattattaag | | | |
| | aatgatgaatgatcaactcatgtttctggaaagag | | | |
| | catttattgatccattagggttaccagacagacct | | | |
| | ttttataggcatgtcatctatgctccaagcagcca | | | |
| | caacaagtatgcaggggagtcattcccaggaattt | | | |
| | atgatgctctgtttgatatcgaaagcaaagtggac | | | |
| | ccttcccaggcctggggagaagtgaagagacagat | | | |
| | ttctattgcaaccttcacagtgcaagcagctgcag | | | |
| | agactttgagtgaagtggcc | | | |

1. A bispecific antibody comprising
(a) a first polypeptide from N-terminus to C-terminus comprising (i) a first single chain variable fragment (scFv) that binds to a tumor-associated antigen (TAA), (ii) an immunoglobulin constant region, and (iii) an scFv that binds to CD3; and
(b) a second polypeptide from N-terminus to C-terminus comprising (i) a second scFv that binds to the TAA, and (ii) an immunoglobulin constant region,
wherein the bispecific antibody does not contain a second CD3-binding domain.

2. The bispecific antibody of claim 1, wherein the TAA is PSMA, HER2, or BCMA.

3-26. (canceled)

27. The bispecific antibody of claim 2, wherein the first scFv that binds to PSMA and/or the second scFv that binds to PSMA is capable of binding to cynomolgus PSMA.

28. (canceled)

29. The bispecific antibody of claim 1, wherein the bispecific antibody is capable of binding to the TAA and CD3 simultaneously.

30. (canceled)

31. The bispecific antibody of claim 2, wherein the first scFv that binds to PSMA comprises a variable heavy (VH) complementarity-determining region (CDR)1, VH CDR2, and VH CDR3 comprising the amino acid sequences of SEQ ID NOs: 70, 72, and 74, respectively, and comprises a variable light (VL) CDR1, VL CDR2, and VL CDR3 comprising the amino acid sequences of SEQ ID NOs: 76, 78, and 80, respectively.

32-33. (canceled)

34. The bispecific antibody of claim 1, wherein the scFv that binds to CD3 comprises a VH CDR1, VH CDR2, and VH CDR3 comprising the amino acid sequences of SEQ ID NOs: 88, 90, and 92, respectively, and comprises a VL CDR1, VL CDR2, and VL CDR3 comprising the amino acid sequences of SEQ ID NOs: 94, 96, and 98, respectively.

35-36. (canceled)

37. The bispecific antibody of claim 2, wherein the second scFv that binds to PSMA comprises a VH CDR1, VH CDR2, and VH CDR3 comprising the amino acid sequences of SEQ ID NOs: 70, 72, and 74, respectively, and comprises a VL CDR1, VL CDR2, and VL CDR3 comprising the amino acid sequences of SEQ ID NOs: 76, 78, and 80, respectively.

38-41. (canceled)

42. The bispecific antibody of claim 1, wherein the antibody is capable of promoting expansion of CD8+ T cells and/or CD4+ T cells, activating CD8+ T cells and/or CD4+ T cells, increasing central memory T cells (TCM) and/or effector memory T cells (TEM), decreasing naïve and/or terminally differentiated T cells (Teff), decreasing secretion of IFN-γ, IL-2, IL-6, TNF-α, Granzyme B, IL-10, and/or GM-CSF, and/or increasing signaling of NFκB, NFAT, and/or ERK signaling pathways.

43-47. (canceled)

48. An antibody or antigen-binding fragment thereof comprising a PSMA-binding domain, wherein the PSMA-binding domain comprises a VH and a VL, wherein the VH comprises the amino acid sequence of SEQ ID NO:82 and/or the VL comprises the amino acid sequence of SEQ ID NO:84.

49-50. (canceled)

51. An antibody or antigen-binding fragment thereof comprising a CD3 antigen-binding domain, wherein the CD3 antigen-binding domain comprises a VH and a VL, wherein the VH comprises the amino acid sequence of SEQ ID NO:100 and/or the VL comprises the amino acid sequence of SEQ ID NO:102.

52-72. (canceled)

73. The antibody or antigen-binding fragment thereof of claim 48, wherein the antibody or fragment comprises a polypeptide comprising, in order from amino-terminus to carboxyl-terminus, (i) a first single chain variable fragment (scFv), (ii) a linker, optionally wherein the linker is a hinge region, (iii) an immunoglobulin constant region, and (iv) a second scFv, wherein (a) the first scFv comprises a human CD3 antigen-binding domain, and the second scFv comprises a human PSMA antigen-binding domain or (b) the first scFv comprises a human PSMA antigen-binding domain and the second scFv comprises a human CD3 antigen-binding domain.

74. (canceled)

75. A bispecific antibody comprising:
(a) a first polypeptide from N-terminus to C-terminus comprising (i) a first single chain variable fragment (scFv) that binds to PSMA comprising the amino acid sequence of SEQ ID NO:86, (ii) a linker comprising the amino acid sequence of SEQ ID NO:156, (iii) an immunoglobulin constant region comprising the amino acid sequence of SEQ ID NO:66, and (iv) an scFv that binds to CD3 comprising the amino acid sequence of SEQ ID NO: 104; and
(b) a second polypeptide from N-terminus to C-terminus comprising (i) a second scFv that binds to PSMA comprising the amino acid sequence of SEQ ID NO:86, (ii) a linker comprising the amino acid sequence of SEQ ID NO:156, and (iii) an immunoglobulin constant region comprising the amino acid sequence of SEQ ID NO:68,
wherein the bispecific antibody does not contain a second CD3-binding domain.

76. A bispecific antibody that binds to PSMA and CD3, wherein the bispecific antibody comprises a first polypeptide comprising the amino acid sequence of SEQ ID NO:106, 178, or 112 and a second polypeptide comprising the amino acid sequence of SEQ ID NO:108, and wherein the bispecific antibody only contains one CD3-binding domain.

77. (canceled)

78. A polynucleotide encoding the bispecific antibody of claim 1.

79. A vector comprising the polynucleotide of claim 78, optionally wherein the vector is an expression vector.

80. A host cell comprising the polynucleotide of claim 78.

81. A host cell comprising a combination of polynucleotides that encode the bispecific antibody claim 1.

82-84. (canceled)

85. A method of producing a bispecific antibody that specifically binds to human PSMA and human CD3 comprising culturing the host cell of claim 81 so that the antibody is produced, optionally further comprising recovering the antibody.

86. A method for detecting PSMA and CD3 in a sample, the method comprising contacting said sample with the bispecific antibody of claim 1, optionally wherein the sample comprises cells.

87. A pharmaceutical composition comprising the bispecific antibody of claim 1, and a pharmaceutically acceptable excipient.

88. A method for increasing T cell proliferation comprising contacting a T cell with the bispecific antibody of claim 1.

89-91. (canceled)

92. A method for enhancing an immune response in a subject, the method comprising administering to the subject an effective amount of the bispecific antibody of claim 1.

93. A method for inducing redirected T-cell cytotoxicity (RTCC) against a cell expressing prostate-specific membrane antigen (PSMA), the method comprising
contacting said PSMA-expressing cell with a bispecific antibody of claim 1, and wherein said contacting is under conditions whereby RTCC against the PSMA-expressing cell is induced.

94. A method for treating a disorder in a subject, wherein said disorder is characterized by overexpression of prostate-specific membrane antigen (PSMA), the method comprising administering to the subject a therapeutically effective amount of a bispecific antibody of claim 1.

95-107. (canceled)
